(19) United States
(12) Patent Application Publication
(10) Pub. No.: US 2023/0375530 A1
(43) Pub. Date: Nov. 23, 2023

(54) HUMAN BLOOD-BRAIN BARRIER MODEL FOR IMMUNOLOGICAL STUDIES

(71) Applicants: WISCONSIN ALUMNI RESEARCH FOUNDATION, Madison, WI (US); UNIVERSITY OF BERN, Bern (CH)

(72) Inventors: Eric V. SHUSTA, Madison, WI (US); Hideaki NISHIHARA; Benjamin GASTFRIEND, Madison, WI (US); Sean P. PALECEK, Verona, WI (US); Britta ENGELHARDT, Bern (CH)

(21) Appl. No.: 18/247,218

(22) PCT Filed: Sep. 28, 2021
(86) PCT No.: PCT/US2021/052421

§ 371 Date: Mar. 29, 2023

Related U.S. Application Data
(60) Provisional application No. 63/185,815, filed on May 7, 2021.
(60) Provisional application No. 63/084,980, filed on Sep. 29, 2020.

(51) Int. Cl. G01N 33/50; C12N 5/071

(52) U.S. Cl. CPC G01N 33/5082; C12N 5/0697; C12N 2513/00; C12N 2501/415; C12N 2506/45; C12N 2501/727

(57) ABSTRACT

The present invention provides methods for differentiating brain microvascular endothelial cells having barrier properties and a mature immune phenotype for use in making an in vitro blood-brain barrier (BBB) model. Further, a BBB model having barrier properties and a mature immune phenotype is provided.

### CROSS-REFERENCE TO RELATED APPLICATIONS

This application claims priority to U.S. Provisional Application No. 63/084,980 filed on Sep. 29, 2020 and U.S. Provisional Application No. 63/185,815 filed on May 7, 2021, the contents of which are incorporated by reference in their entireties.

### STATEMENT REGARDING FEDERALLY SPONSORED RESEARCH

This invention was made with government support under NS103844 awarded by the National Institutes of Health. The government has certain rights in the invention.

### BACKGROUND

Under physiological conditions, the blood-brain barrier (BBB) maintains central nervous system (CNS) homeostasis by protecting the CNS from the constantly changing milieu in the bloodstream. The BBB is established by brain microvascular endothelial cells (BMECs), which inhibit free paracellular diffusion of water-soluble molecules by complex tight junctions that connect the endothelial cells (1). Combined with their characteristically low pinocytotic activity and lack of fenestrations, which inhibit transcellular passage of molecules across the BBB, these features establish the physical barrier of the BBB (2). At the same time, the BBB establishes a functional barrier, in which expression of specific transporters and enzymes in BMECs ensures that nutrients pass into the CNS and toxic metabolites are removed from the CNS. In the absence of neuroinflammation, the BBB also limits immune cell trafficking to specific immune cell subsets that ensure CNS immune surveillance (3). Importantly, the segments of the microvasculature mediating immune cell trafficking versus regulating transport of solutes are not identical; while solute transport is predominantly localized to CNS capillaries, immune cell trafficking occurs at the level of CNS post-capillary venules (4, 5).

Current knowledge about the cellular and molecular mechanisms mediating immune cell migration across the BBB during CNS immune surveillance and neuroinflammation have to a large degree been derived from animal models of neuroinflammatory diseases. These studies showed that the migration of immune cells across the BBB follows a multi-step process that is regulated by the sequential interaction of different signaling and adhesion molecules on the BBB endothelium and the immune cells (4). Because of the unique tightness of this vascular bed, immune cell migration across the BBB is characterized by unique adaptations. These range from a predominant role of a4b1-integrin in mediating interaction of T cells with endothelial VCAM-1 (6) to the extended crawling of T cells mediated by endothelial ICAM-1 and ICAM-2 against the direction of the blood flow in search of rare sites permissive for diapedesis across the BBB (7, 8).

There are some limitations when applying knowledge from animal models to humans. It has been observed that levels of some adhesion molecules (e.g., activated leukocyte cell adhesion molecule (ALCAM) or junctional adhesion molecules (JAMs)) are different between rodents and human (9). As a result, there is a significant need for human BBB models that are suitable for the study of immune cell trafficking across the BBB. To this end, human brain endothelial cell lines (e.g., hCMEC/D3 (10)) have been established allowing the study of barrier properties and immune cell trafficking under inflammatory conditions. Unfortunately, these BBB models often fail to establish complex tight junctions and barrier properties characterized by high transendothelial electrical resistance (TEER) and low permeability of soluble tracers (11). Appropriate barrier properties are prerequisite for appropriate modeling of the unique mechanisms involved in T-cell diapedesis across the BBB (12). Primary human brain microvascular endothelial cells (hBMECs) have proven useful to study T-cell/BBB interactions; however, these cells are often not readily available to researchers (12, 13) and they come from non-autologous sources. Recent advances in stem cell technology have allowed derivation of hBMEC-like cells from various stem cell sources including human cord blood-derived endothelial progenitors and human pluripotent stem cells (hPSCs) (14, 15). In particular, patient-sourced human induced pluripotent stem cell (hiPSC)-derived BMEC-like cells uniquely enable the study of BBB dysfunction by providing a scalable and renewable source of BMEC-like cells. For immune cell studies, one could also envision combining hiPSC and autologous immune cells sourced from the same patient cohort. Human iPSC-derived in vitro models of the BBB have been established (14, 16-18) and proven useful for modeling BBB dysfunction in inheritable neurological disorders in vitro (19-21).

Presently available hiPSC-derived in vitro BBB models are well characterized with respect to their barrier properties and expression of BBB specific transporters and efflux pumps (14, 16, 17) and have proven useful for the study of barrier regulation, molecular transport and brain drug delivery. However, much less is known about the immune phenotypes, such as the expression and cytokine-induced upregulation of adhesion molecules, in hiPSC-derived in vitro BBB models. These properties are extremely important when aiming to apply hiPSC-derived in vitro BBB models to study cerebrovascular pathologies involved in multiple sclerosis, stroke or CNS infections, where immune cell trafficking across the BBB critically contributes to disease pathogenesis. Previous reports have suggested that inflammatory stimuli could induce expression of ICAM-1 (17, 22) and VCAM-1 (22) in hiPSC-derived BMEC-like cells, but detailed characterization of the full panel of adhesion molecules shown to mediate the multi-step immune cell migration across the BBB has not been reported. Accordingly, there remains a need in the art for hiPSC-derived in vitro BBB models with an improved immune phenotype.

### SUMMARY

The present invention provides a blood brain barrier model with immune phenotype and methods of producing such a model.

In one aspect, the disclosure method of producing a population of extended endothelial culture method (EECM)-derived brain microvascular endothelial cells (BMECs) (EECM-BMECs) from a population of endothelial progenitor cells, the method comprising: (a) culturing a cell population of CD34+CD31+ endothelial progenitor cells in serum-free endothelial medium on collagen coated surface until confluent; (b) selectively passaging the endothelial cells of (a) in serum-free endothelial medium on collagen surface for at least two passages; (c) culturing the selectively passaged cells of (b) in serum-free endothelial medium until confluent; wherein the confluent monolayer is a population of CD31⁺ EECM-BMECs that express vascular endothelial (VE)-cadherin, ICAM-2, PECAM-1, and three or more adhesion molecules selected from the group consisting of ICAM-1, VCAM-1, E-selectin, P-selectin, and CD99.

In another aspect, the disclosure provides a homogenous population of CD31⁺EECM-BMECs obtained by the method described herein. In some aspects, the CD3⁺EECM-BMECs are characterized by: (a) a lower permeability as compared to naïve endothelial cells; (b) increased expression of claudin-5 as compared to naïve endothelial cells; (c) improved localization of occludin and claudin-5 as compared to naïve endothelial cells; or (d) any combination of (a)-(c).

In a further aspect, the disclosure provides an in vitro blood-brain barrier model for studying immune cell migration and regulation comprising a confluent monolayer of the CD31⁺ EECM-BMECs described herein cultured on surface within a system.

In another aspect, the disclosure provides a method of identifying therapeutic targets for the treatment of neuroinflammatory or neurodegenerative diseases or disorders, the method comprising: (a) contacting the in vitro blood-brain barrier model described herein with a therapeutic target; and (b) determining the disruption and/or restoration of the blood-brain barrier model.

In a further aspect, the disclosure provides a isogenic blood-brain barrier model for a subject having a neuroinflammatory or neurodegenerative disease, the blood-brain barrier model comprising a confluent monolayer of the CD31⁺ EECM-BMECs described herein cultured on a surface, for example, a collagen coated permeable membrane, within a system, wherein the CD31⁺EECM-BMECs are derived from endothelial progenitor cells differentiated from pluripotent stem cells derived from the subject.

In another aspect, the disclosure provides a method for producing a cell population comprising smooth muscle-like cells from a cell population comprising CD34+CD31+ endothelial progenitor cells, the method comprising the steps of: (a) culturing a cell population of CD34+CD31+ endothelial progenitor cells in serum-free endothelial medium on collagen coated surface until confluent; (b) selectively passaging the non-endothelial cells of (a); (c) culturing the non-endothelial cells of (b) for about 6 days to about 10 days to produce PECAM-1⁻/α-smooth muscle actin⁺ smooth muscle-like cells.

In another aspect, the disclosure provides an additional method of producing a population of derived extended endothelial cultured method brain microvascular endothelial cells (EECM-BMECs) from human pluripotent stem cells, the method comprising: (a) contacting cultured human pluripotent stem cells with activator of Wnt/β-catenin signaling for a period of about 2 days; (b) culturing the cells of (a) in the absence of the activator for two to three days; (c) separating the CD34⁺CD31⁺ endothelial progenitor cells from step (b) from the CD34⁻CD31⁻ non-EPCs; (d) culturing the separated CD34+CD31+ endothelial progenitor cells on coated plates in medium comprising an activator of Wnt/β-catenin signaling for about 3 days to about 10 days to produce a confluent monolayer; (e) selectively passaging the cells of step (d) in serum-free endothelial medium on coated plates in medium comprising the activator for at least one additional passage to obtain a confluent population of CD31⁺GLUT1⁺EECM-BMECs having a canonical barrier phenotype. This method produces a population of CD31⁺GLUT1⁺EECM-BMECs of step (e) that express glucose transporter 1 (GLUT1), claudin-5 and have reduced expression of plasmalemma vesicle-associated protein (PLVAP). In some aspects, a homogenous population of CD31⁺BMECs is obtained by this method. The CD31⁺ EECM-BMECs are characterized by: (a) a lower permeability as compared to naïve endothelial cells; (b) increased expression of claudin-5 as compared to naïve endothelial cells; (c) increased expression of GLUT1 as compared to naïve endothelial cells; (d) reduced expression of plasmalemma vesicle-associated protein (PLVAP) as compared to naïve endothelial cells; or (e) any combination of (a)-(d). In some aspects, the activator of Wnt/β-catenin signaling is a Gsk3 inhibitor or one or more Wnt ligands. In some aspects, the Gsk3 inhibitor is selected from the group consisting of CHIR 99021, CHIR 98014, BIO-acetoxime, BIO, LiCl, SB 216763, SB 415286, AR A014418, 1-Azakenpaullone, and Bis-7-indolylmaleimide.

### BRIEF DESCRIPTION OF THE DRAWINGS

FIG. 1 depicts the adhesion molecule phenotype of BMEC-like cells differentiated by the unconditioned medium method (UMM) or the defined method (DMM). Cell surface staining of BMEC-like cells differentiated by UMM (A, C) or DMM (B, D) in the presence or absence of RA for the adhesion molecules ICAM-1, ICAM-2, VCAM-1, P-selectin, E-selectin, CD99, and PECAM-1 was analyzed by flow cytometry. Isotype control, non-stimulated (NS), and 16 h pro-inflammatory cytokine-stimulated condition (10 ng/mL TNF-α+200 IU/mL IFN-γ) are represented in grey, blue, and red, respectively, in a histogram overlay. Representative data from donor 2 are shown. At least 3 independent differentiations were performed in each condition using 2 different hiPSC clones derived from 2 different donors (donor 2, 3) for UMM-differentiated BMEC-like cells and 3 hiPSC clones from 3 donors (donor 1, 2, 3) for DMM-differentiated BMEC-like cells with comparable data observed (e.g., FIG. 7). (E) The Δ geometric mean (MFI staining-MFI isotype) of cell surface adhesion molecules of UMM- or DMM-BMEC-like cells were analyzed by flow cytometry. Displayed are the mean ΔMFI for each donor (donor 1: black, donor 2: red, donor 3: blue). Mean±S.D. from triplicate differentiations were used in a paired students t-test to determine statistically significant changes upon stimulation (P<0.05=*, P<0.01=**).

FIG. 2 depicts the adhesion molecule phenotype and morphology of hiPSC-derived naïve ECs. (A) Cell surface staining of hiPSC-derived naïve ECs for the adhesion molecules ICAM-1, ICAM-2, VCAM-1, P-selectin, E-selectin, CD99, and PECAM-1 was analyzed by flow cytometry. Isotype control, non-stimulated (NS), and 16 h pro-inflammatory cytokine-stimulated condition (1 ng/mL TNF-α+20 IU/mL IFN-γ) are represented in grey, blue, and red, respectively in a histogram overlay. Representative data from donor 2 are shown. Three independent differentiations were performed using three hiPSC clones from three donors (donor 1, 2, 3) showing comparable data (e.g., FIG. 14). (B) The Δ geometric mean (MFI staining-MFI isotype) of cell surface adhesion molecules of naïve ECs were analyzed by flow cytometry. Displayed are the mean ΔMFI for each donor (donor 1: black, donor 2: red, donor 3: blue). Mean±S.D. from triplicate differentiations were used in a paired students t-test to determine statistically significant changes upon stimulation (P<0.05=*). (C) Immunofluorescence staining of hiPSC-derived naïve ECs grown on 0.4 μm pore Transwell filter inserts for ZO-1 (red) or VE-cadherin (red), and nuclei (DAPI, blue) is shown. Arrow depicts VE-cadherin negative cells. Representative data from donor 2 are shown. Each staining is representative of at least three independent differentiations. Three hiPSC clones from three donors (donor 1, 2, 3) were used in this assay and at least three independent differentiations were performed in each donor. Scale bars=100 μm.

FIG. 3 depicts the extended endothelial cell culture method (EECM). (A) Schematic representation of the protocols used for extended endothelial cell culture method (EECM). (B) Phase contrast images of hiPSC-derived naïve ECs before (i) and after (ii) incubation with Accutase, and after detachment of ECs by tapping the plate (iii). Red arrow shows non-ECs remaining attached to the plate. Scale bars=100 μm. (C) Immunofluorescence staining of naïve ECs (passage 1) or EECM-BMEC-like cells (passages 2-4) grown on chamber slides. Junctions were stained for VE-cadherin (red), PECAM-1 (red), ZO-1 (green), claudin-5 (red), or occludin (red), and nuclei were stained with DAPI (blue). Scale bars=50 μm. (D) Immunofluorescence images of EECM-BMEC-like cells (passage 5) grown on 0.4 μm pore Transwell filters. Cell junctions were stained for VE-cadherin (red) or zonula occludens 1 (ZO-1, red), and nuclei were stained with DAPI (blue). Each panel shows a xy en face view of a maximum-intensity projection through the z-axis. The top (xz) and right (yz) side panels indicate the flat morphology of the EECM-BMEC-like cells with staining for cell nuclei and ZO-1 at the same level in the z-axis. Scale bars=20 μm. (C, D) Representative images from donor 2 are shown. Five hiPSCs clones from three donors (donor 1, 2, 3) were used in this assay and at least 3 independent differentiations were performed for each clone producing comparable results. (E) Time-dependent progression of trans-endothelial electrical resistance (TEER) of EECM-BMEC-like cell monolayers derived from donor 2. EECM-BMEC-like cells (passage 2: red, passage 3: green, passage 4: blue, passage 5: orange) were grown to confluency on 0.4 μm pore size Transwell filters for 6 days. Passage 1 naïve ECs show no measurable TEER. Plotted data are mean TEER values±SD. Data are combined from at least three independent differentiations for each passage, each measurement performed in triplicates. (F) Permeability of 0.37 kDa sodium fluorescein (NaFl): naïve ECs (passage 1: black) or EECM-BMEC-like cells (passage 2: red, passage 3: green, passage 4: blue, passage 5: orange) derived from donor 2 were cultured to confluency on 0.4 μm pore size Transwell filters for 6 days and permeability was measured at day 6. Bars show the mean permeability coefficients (Pe)±SD. Data are combined from at least three independent differentiations each performed in triplicate per condition. Statistical analysis: one-way ANOVA followed by Tukey's multiple comparison test. (p<0.0001=****).

FIG. 4 shows the effect of co-culture with astrocytes or pericytes on EECM-BMEC-like cell monolayers. (A) Schematic representation of the protocols for EECM-BMEC-like cells co-cultured with astrocytes or pericytes, or RA treatment is shown. (B) TEER and (C) permeability of sodium fluorescein of EECM-BMEC-like cell monolayers are shown. EECM-BMEC-like cells (from passages 3-5) were seeded onto 0.4 μm pore size Transwell filters and co-cultured for 6 days with bovine pericytes ((bovine PC): light green), a human brain pericyte cell line ((HBPCT): green), a human astrocyte cell line ((hAST): dark red), hiPSC-derived astrocytes ((iPS_AC): pink), or treated with RA for 2 days (purple). Monoculture was used as a control (black). (B) Plotted data are mean TEER values±SD. (C) Bars show the mean permeability coefficients (Pe)±SD. (B, C) Data are from at least three independent differentiations for co-cultures each performed in triplicate using the same hiPSC clone. Representative data from donor 2 are shown. The following clones were tested for each condition: 3 hiPSC clones from 2 donors (donor 1, 2) for bovine PC, 2 hiPSC clones from 2 donors (donor 1, 2) for HBPCT, 2 hiPSC clones from 2 donors (donor 1, 2) for hAST, and 3 hiPSC clones from 2 donors (donor 1, 2) for iPS_AC and yielded comparable results (e.g., FIG. 12). (D) Immunofluorescence staining of EECM-BMEC-like cells cultured on 0.4 μm pore size Transwell filters. EECM-BMEC-like cells were seeded onto Transwell filters and either co-cultured with HBPCT (pericyte) or hiPSC-derived astrocytes over 6 days. Monoculture was used as a control. Junctions were stained for VE-cadherin (red), claudin-5 (red), or occludin (red), and nuclei were stained with DAPI (blue). Representative data from donor 2 are shown. Each staining is representative of at least three independent differentiations performed on three distinct filters. Scale bars=50 μm. (E) The number of nuclei per pre-defined field of view (FOV) of EECM-BMEC-like cells grown on 0.4 μm pore size Transwell filters is shown. ImageJ software was used to automatically count the nuclei. (F) EC shape was analyzed using circularity values (4×π×area/perimeter²) calculated from VE-cadherin immunofluorescence images using ImageJ software. A value of 1.0 indicates a perfect circle. As the value approaches 0, it indicates an increasingly elongated shape of the cell. Each dot represents average circularity values of 10 randomly chosen cells/FOV. Data are shown as mean±SD. Statistical analysis: one-way ANOVA followed by Tukey's multiple comparison test. (p<0.01=**, p<0.0001=****).

FIG. 5 depicts the adhesion molecule phenotype of EECM-BMEC-like cells. (A) Cell surface staining of EECM-BMEC-like cells for the adhesion molecules ICAM-1, ICAM-2, VCAM-1, P-selectin, E-selectin, CD99, and PECAM-1 was analyzed by flow cytometry. Isotype control, non-stimulated (NS), and 16 h pro-inflammatory cytokine-stimulated condition (1 ng/mL TNF-α+20 IU/mL IFN-γ) are represented in grey, blue, and red lines respectively in a histogram overlay. Representative data from donor 3 are shown. Three hiPSC clones from three donors (donor 1, 2, 3) were used in this assay. (B) The Δ geometric mean (MFI staining-MFI isotype) of cell surface adhesion molecules of EECM-BMEC-like cells were analyzed by flow cytometry. Displayed are the mean ΔMFI for each donor (donor 1: black, donor 2: red, donor 3: blue). Mean±S.D. from triplicate differentiations were used in a paired students t-test to determine statistically significant changes upon stimulation (P<0.05=*, P<0.01=**). (C) Immunofluorescence staining of EECM-BMEC-like cell monolayers grown to confluency in chamber slides for ICAM-1 (red), ICAM-2 (red), VCAM-1 (red), P-selectin (red), E-selectin (red), or CD99 (red) are shown. Nuclei were stained with DAPI (blue). Representative data from donor 2 are shown. NS and 0.1 ng/mL TNF-α+2 IU/mL IFN-γ stimulated conditions are shown. Each staining is representative of at least three independent differentiations performed on three distinct chamber slides. Scale bars=50 μm. (D) The number of cell tracker-labeled Th1* cells adherent to non-stimulated (NS), 0.1 ng/mL or 0.1 ng/mL TNF-α+2 IU/mL IFN-γ stimulated EECM-BMEC-like cell monolayers derived from donor 2 were counted after 30 minutes of adhesion under static conditions. Adherent T cells/FOV is the mean number of cells from two fields per well automatically counted using ImageJ. Data are shown as the mean±SD of at least three individual differentiations each performed in triplicate. Statistical analysis: one-way ANOVA followed by Tukey's multiple comparison test. (P<0.0001=****). (E) The number of cell tracker-labeled Th1* cells adherent to 0.1 ng/mL IL-1β stimulated EECM-BMEC-like cell monolayers were measured after 30 minutes of adhesion under static conditions. Adherent T cells/FOV is the mean number of cells from two fields per well automatically counted using ImageJ. EECM-BMEC-like cells derived from donor 2 were preincubated with either anti-human ICAM-1 (10 μg/ml, clone R6.5), anti-human VCAM-1 (10 μg/ml, polyclonal), or isotype controls for 30 minutes at 37° C. Th1* cells were pretreated with either mouse anti-human beta2 integrin (10 μg/ml, clone TS1/18), humanized anti-human α4 integrin IgG4 (10 μg/ml, Natalizumab), or isotype controls for 30 minutes at 37° C. Data are shown as the mean±SD of a representative experiment of three individual differentiations each performed in at least triplicate. Statistical analysis: one-way ANOVA followed by Tukey's multiple comparison test. (P<0.05=*, p<0.0001=****). (F) The numbers of Th1* cells adherent to 0.1 ng/mL IL-10 stimulated EECM-BMEC-like cell monolayers derived from donor 2 under flow conditions were measured. Th1* cells were pretreated with either 10 μg/ml Natalizumab or isotype control for 30 minutes at 37° C. Data are shown as the mean±SD of eight experiments in each condition. Three independently differentiated BMEC-like cell populations derived from donor 2 were used in this study. Statistical analysis: unpaired t-test (P<0.05=*)

FIG. 6 demonstrates that human iPSC-derived smooth muscle-like cells (SMLCs) or conditioned medium enhances VCAM-1 expression on EECM-BMEC-like cells without impairing barrier properties. Cell surface staining of EECM-BMEC-like cells co-cultured with hiPSC-derived SMLCs (A) or conditioned medium from hiPSC-derived SMLC (B) for the adhesion molecules ICAM-1, ICAM-2, VCAM-1, P-selectin, E-selectin, CD99, and PECAM-1 was analyzed by flow cytometry. Isotype control, non-stimulated (NS), and 16 h pro-inflammatory cytokine-stimulated condition (1 ng/mL TNF-α+20 IU/mL IFN-γ) are represented in grey, blue, and red lines respectively in a histogram overlay. Representative data from donor 3 are shown. Three hiPSC clones from three donors (donor 1, 2, 3) were used in this assay (e.g., FIG. 20). (C) The Δ geometric mean (MFI staining-MFI isotype) of cell surface VCAM-1 of EECM-BMEC-like cells were analyzed by flow cytometry. Monocultured EECM-BMEC-like cells, EECM-BMEC-like cells co-cultured with hiPSC-derived SMLCs, or cultivated with conditioned medium from hiPSC-derived SMLC are shown. Each symbol (donor 1: black, donor 2: red, donor 3: blue) shows the mean of at least three independent differentiations (co-culture condition, donor 1: n=3, donor 2: n=4, donor 3: n=7). Statistical analysis: one-way ANOVA followed by Tukey's multiple comparison test. (P<0.05=*). (D) Immunofluorescence staining of EECM-BMEC-like cells derived from donor 3 grown to confluency in chamber slides using conditioned medium from hiPSC-derived SMLCs for ICAM-1 (red), ICAM-2 (red), VCAM-1 (red), P-selectin (red), E-selectin (red), or CD99 (red) are shown. Nuclei were stained with DAPI (blue). NS and 16 h pro-inflammatory cytokine-stimulated condition (0.1 ng/mL TNF-α+2 IU/mL IFN-γ) are shown. Each staining is representative of at least three independent differentiations performed on three distinct chamber slides. Scale bars=50 (E) The number of Th1* cells adherent to 16 h pro-inflammatory cytokine-stimulated (0.1 ng/mL TNF-α+2 IU/mL IFN-γ) EECM-BMEC-like cell monolayers derived from donor 2 cultured in the presence of SMLC-derived conditioned medium, normalized to control condition was measured after 30 minutes of adhesion under static conditions. Adherent T cells/FOV is the mean number of cells from two fields per well automatically counted using ImageJ. EECM-BMEC-like cells were preincubated with either anti-human ICAM-1 (10 μg/ml, clone R6.5), anti-human VCAM-1 (10 μg/ml, polyclonal), or isotype control for 30 minutes at 37° C. Th1* cells were pretreated with either anti-human beta2 integrin (10 μg/ml, clone TS1/18), humanized anti-human α4 integrin IgG4 (10 μg/ml, Natalizumab), or isotype controls for 30 minutes at 37° C. Data are shown as the mean±SD of three individual differentiations each performed in at least triplicates. Statistical analysis: one-way ANOVA followed by Tukey's multiple comparison test (P<0.05=*, p<0.0001=****). (F) Immunofluorescence staining on EECM-BMEC-like cells grown on 0.4 μm pore size Transwell filters co-cultured with SMLCs. Junctions were stained for VE-cadherin (red), PECAM-1 (red), ZO-1 (red), claudin-5 (red), or occludin (red), and nuclei were stained with DAPI (blue). Representative data from donor 2 is shown. Each staining is representative of at least three independent differentiations performed on three distinct filters. Five hiPSCs clones from three donors (donor 1, 2, 3) were used in this assay. Scale bars=50 (G) TEER and (H) permeability of sodium fluorescein of EECM-BMEC-like cell monolayers derived from donor 2 co-cultured with SMLCs or cultured in the presence of SMLC conditioned medium. EECM-BMEC-like cells were grown on 0.4 μm pore size Transwell filters in the presence of either SMLC s (pink) or conditioned medium from SMLCs in the lower chamber (abluminal side) (purple) for 6 days. (G) Plotted data are mean TEER values±SD. (H) Bars show the mean permeability coefficients (Pe)±SD. (G, H) Data are representative of at least three independent differentiations with three filters per conditions. Three hiPSC clones from three donors (donor 1, 2, 3) were used in this assay yielding comparable results. Human CD34⁺ cord-blood stem cell-derived brain like endothelial cells (BLECs, red) were used for a comparison to a previously developed human in vitro BBB model. (I) In vitro live cell imaging of the Th1* cell/EECM-BMEC-like cells interactions under flow. A temporal snapshot illustrating that the different Th1* cell-EECM-BMEC-like cells interactions have been made with several frames taken from a video recorded with a 10× magnification. EECM-BMEC-like cells from donor 2 were cultured in cloning rings placed on collagen IV (10 μg/ml)-coated Ibidi μ-dishes at a density of 75′000/cm². EECM-BMEC-like cells were stimulated with 0.1 ng/ml recombinant human TNF-α+2 IU/mL IFN-γ for 16 h at 37° C. (5% CO₂) diluted in conditioned medium from SMLC. Fluorescencently-labelled Th1* cells were allowed to accumulate on the EECM-BMEC-like cell monolayer at a low flow rate of 0.1 dyne/cm² for 4 min from the first frame after the first Th1* cells appear in the field of view (accumulation phase until 3 min 55 s). After the accumulation phase of precisely 3 min 55 sec, the flow rate was set to 1.5 dyne/cm² for 16 min (shear phase). Each row of images shows a different behavior (diapedesis (blue), crawling (orange), probing (pink)) of the Th1* cells with the EECM-BMEC-like cells. The red arrow shows the direction of flow and the time is displayed on the top left of each image (min:s format). Time-lapse video shows Th1* cell interaction on EECM-BMEC-like cells (data not shown).

FIG. 7 depicts the adhesion molecule phenotype of BMEC-like cells differentiated by unconditioned medium method (UMM) or defined medium method (DMM) from additional hiPSC clone. Cell surface staining of BMEC-like cells differentiated by UMM (A) or DMM (B) in the absence of RA for the adhesion molecules ICAM-1, ICAM-2, VCAM-1, P-selectin, E-selectin, CD99, and PECAM-1 was analyzed by flow cytometry. Isotype control, non-stimulated (NS), and 16 h pro-inflammatory cytokine-stimulated condition (10 ng/mL TNF-α+200 IU/mL IFN-γ) are represented respectively in grey, blue, and red lines in a histogram overlay. Representative data from donor 3 are shown.

FIG. 8 depicts the adhesion molecule phenotype and morphology of naïve ECs from additional hiPSC clone. (A) Cell surface staining of naïve ECs for the adhesion molecules ICAM-1, ICAM-2, VCAM-1, P-selectin, E-selectin, CD99, and PECAM-1 was analyzed by flow cytometry. Isotype control, non-stimulated (NS) and 16 h pro-inflammatory cytokine-stimulated condition (1 ng/mL TNF-α+20 IU/mL IFN-γ) are represented respectively in grey, blue, and red lines in histogram overlays. Representative data from donor 1 is shown. (B) Immunofluorescence staining of naïve ECs grown on 0.4 μm pore Transwell filter for PECAM-1 (red) and smooth muscle actin (green). Nuclei were stained with DAPI (blue). Scale bar=100 μm. (C) Quantification of PECAM-1⁺ and α-smooth muscle actin⁺ cells in immunofluorescence staining images. Bar shows mean±SD of 4 differentiations using IMR90-4 line.

FIG. 9 shows the purity of VE-cadherin⁺ cells at passage 1 and passage 5. Staining of ECs for the adherens junction molecule VE-cadherin was analyzed by flow cytometry. Isotype control, passage 1 and passage 5 ECs are shown. Representative data from donor 1, 2 and 3 are shown. Three different hiPSC clones derived from three individuals (donor 1, 2, 3) were evaluated in this assay.

FIG. 10 shows a western blot analysis of naïve ECs and EECM-BMEC-like cells and barrier characteristics of EECM-BMEC-like cells from an hiPSC line originating from fibroblasts. (A) Western blots of naïve EC (passage 1, p1) and EECM-BMEC-like cells (passage 2 and 3, p2 and p3) probed for β-actin, VE-cadherin, claudin-5, occludin, vWF, ZO-1, and caveolin-1. (B) Quantification of Western blot band intensity. Data are shown as the mean±SD of two wells from one differentiation of the IMR90-4 hiPSC line. Statistical analysis: one-way ANOVA followed by Tukey's multiple comparison test. (C, D) TEER (C) and permeability to sodium fluorescein (D) of EECM-BMEC-like cell monolayers from IMR90-4 hiPSC line is shown. EECM-BMEC-like cells were seeded onto 0.4 μm pore size Transwell filters as monoculture and TEER were measured over 6 days and Pe_{NaFl} was measured at day 6. (C) Plotted data are mean TEER values±SD. (D) Bars show the mean permeability coefficients (Pe)±SD. (E) Immunofluorescence images of EECM-BMEC-like cells from IMR90-4 hiPSC line grown on 0.4 μm pore Transwell filters. Cell junctions were stained for CD31 (green), claudin-5 (green), zonula occludens 1 (ZO-1, green), occludin (green) and nuclei were stained with Hoechst (blue). Scale bars=100 μm.

FIG. 11 depicts the morphology of BMEC-like cells differentiated by the unconditioned medium method (UMM) or the defined medium method (DMM). Immunofluorescence images of UMM- or DMM-differentiated BMEC-like cells grown on 0.4 μm pore Transwell filters. Cell junctions were stained for zonula occludens 1 (ZO-1, green) and nuclei were stained with DAPI (blue). The bottom of each panel shows a maximum-intensity projection through the z-axis. In the top (xz) and right (yz) side images of each panel the apical side of the BMEC-like cells is oriented, respectively, towards the top and the right side. Representative data from donor 2 are shown. Each staining is representative of at least three independent differentiations performed on three distinct filters. Scale bars=20 μm.

FIG. 12 shows the effect of astrocyte or pericyte co-culture and ECM-5 medium on barrier characteristics of EECM-BMEC-like cell monolayers from additional hiPSC clones. TEER (A, C, E) and permeability to sodium fluorescein (B, D, F, G) of EECM-BMEC-like cell monolayers is shown. EECM-BMEC-like cells were seeded onto 0.4 μm pore size Transwell filters and co-cultured for 6 days with bovine pericytes (bovine PC; A and B, light green), a human brain pericyte cell line (HBPCT) (A and B, dark green), a human astrocyte cell line (hAST) (C and D, dark red), hiPSC-derived astrocytes (iPS_AC) (E and F, pink). Monoculture condition was used as a control (black). (A, C, E) Plotted data are mean TEER values±SD. (B, D, F) Bars show the mean permeability coefficients (Pe)±SD. (A-E) Representative data from donor 1 are shown. At least two independent differentiations performed on two distinct filters were used in this study. (G) Permeability of sodium fluorescein of EECM-BMEC-like cell monolayers cultivated in ECM-5 medium in the presence and absence of bovine pericytes. EECM-BMEC-like cells from donor 2 (passage 3-5) were seeded onto 0.4 μm pore size Transwell filters and co-cultured for 6 days with bovine pericytes. Monoculture condition using ECM-5 medium was used as a control (black). Bars show the mean permeability coefficients (Pe)±SD. Data are overlaid from at least three independent differentiations each performed in triplicate per conditions. Statistical analysis: unpaired t-test (P<0.01=**)

FIG. 13 shows the effect of RA pretreatment or pericyte conditioned medium on EPCs. (A) TEER and (B) permeability of sodium fluorescein of EECM-BMEC-like cells from donor 2 at passage 2 are shown. EPCs (after EPC purification by MACS) were cultured in the presence or absence of RA for 3 days (brown) or bovine pericyte conditioned medium (PCM) for 6 days (green) and then EECM-BMEC-like cells at passage 2 were cultured on 0.4 μm pore size Transwell filters over 6 days. Data are from three independent differentiations for RA treatment each performed in at least triplicate, and from two independent differentiations for PCM condition each performed in duplicate. Statistical analysis: one-way ANOVA followed by Tukey's multiple comparison test. (p<0.0001=****).

FIG. 14 shows immunofluorescence staining of stimulated and control EECM-BMEC-like cells from additional hiPSC clones. (A, B) Immunofluorescence staining of EECM-BMEC-like cells from donor 3 (A and B) seeded in a chamber slide for ICAM-1 (red), ICAM-2 (red), VCAM-1 (red), P-selectin (red), E-selectin (red), or CD99 (red). Nuclei were stained with DAPI (blue). NS and 0.1 ng/mL TNF-α+2 IU/mL IFN-γ-(A) or 0.1 ng/mL IL-1β-(B) stimulated conditions are shown. Scale bars=50 μm. Each staining is representative of at least three independent differentiations performed on three distinct chamber slides.

FIG. 15 shows cell surface expression of adhesion molecules on different human in vitro BBB models. The Δ geometric mean (MFI staining-MFI isotype) of cell surface staining for adhesion molecules on different human in vitro BBB models were analyzed by flow cytometry. Each symbol shows results from different donor (donor 1: black, donor 2: red, donor 3: blue). Human CD34⁺ cord-blood stem cell-derived brain like endothelial cells (BLECs, orange) and primary human brain microvascular endothelial cells (pHBMECs, magenta) were used for a comparison to DMM-BMECs, UMM-BMECs and EECM-BMECs. Data for naïve ECs are also shown.

FIG. 16 depicts the extended adhesion molecule phenotype of EECM-BMEC-like cells. Immunofluorescence staining of EECM-BMEC-like cells from donor 3 seeded in a chamber slide for ALCAM (red) and MCAM (red) is shown. Nuclei were stained with DAPI (blue). NS and 0.1 ng/mL TNF-α+2 IU/mL IFN-γ-stimulated conditions are shown. Scale bars=50 μm. Each staining is representative of at least three independent differentiations performed on three distinct chamber slides.

FIG. 17 shows the similar effects of TNF-α/IFN-γ or IL-1β for cell surface ICAM-1 and VCAM-1 staining of EECM-BMEC-like cells. Cell surface staining of EECM-BMEC-like cell monoculture for the adhesion molecules ICAM-1, VCAM-1, was analyzed by flow cytometry. Isotype control and 16 h pro-inflammatory cytokine-stimulated condition (1 ng/mL TNF-α+20 IU/mL IFN-γ or 1 ng/mL IL-1β) are represented respectively in grey, red, and green lines in a histogram overlay. Representative data from donor 2 are shown.

FIG. 18 depicts the concentration dependent induction of ICAM-1 and VCAM-1 on EECM-BMEC-like cells by pro-inflammatory cytokines. Immunofluorescence staining of EECM-BMEC-like cells from donor 2 seeded in a chamber slide for phalloidin (green), ICAM-1 (red), and VCAM-1 (red) is shown. Nuclei were stained with DAPI (blue). NS and different concentrations of TNF-α+IFN-γ-stimulated conditions are shown. Scale bars=50 μm. Each staining is representative of at least three independent differentiations performed on three distinct chamber slides.

FIG. 19 shows immunofluorescence staining of hiPSCs-derived smooth muscle-like cells (SMLCs). Immunofluorescence staining of hiPSC-derived smooth muscle-like cells (SMLCs) from donor 2 seeded in a chamber slide for α-smooth muscle actin (SMA), N-cadherin, NG-2, ZO-1, or Vimentin, or from the IMR90-4 line stained for calponin (green) and SM22α (red). Nuclei were stained with DAPI (blue). Scale bars=50 μm. Each staining is representative of at least three independent differentiations performed on three distinct chamber slides.

FIG. 20 depicts the adhesion molecule phenotype of EECM-BMEC-like cells from additional hiPSC clones. (A-C) Cell surface staining of EECM-differentiated BMEC monoculture (A), SMLC co-culture (B), or SMLC conditioned medium (C) for the adhesion molecules ICAM-1, ICAM-2, VCAM-1, P-selectin, E-selectin, CD99, and PECAM-1 was analyzed by flow cytometry. Isotype control, non-stimulated (NS), and 16 h pro-inflammatory cytokine-stimulated condition (1 ng/mL TNF-α+20 IU/mL IFN-γ) are represented respectively in grey, blue, and red lines in a histogram overlay. Representative data from donor 1 (left) and donor 2 (right) are shown.

FIG. 21 shows the similar effects of TNF-α/IFN-γ or IL-1β for cell surface ICAM-1 and VCAM-1 of EECM-BMEC-like cells with SMLC co-culture. Cell surface staining of EECM-BMEC-like cells with SMLC co-culture for the adhesion molecules ICAM-1, VCAM-1, was analyzed by flow cytometry. Isotype control and 16 h pro-inflammatory cytokine-stimulated condition (1 ng/mL TNF-α+20 IU/mL IFN-γ or 1 ng/mL IL-1β) are represented respectively in grey, red, and green lines in a histogram overlay. Representative data from donor 3 are shown.

FIG. 22 shows immunofluorescence staining of stimulated and control EECM-BMEC-like cells from additional hiPSC clones. Immunofluorescence staining of EECM-BMEC-like cells grown on 0.4 μm pore size Transwell filters and co-cultured with SMLCs for VCAM-1 (red) are shown. Nuclei were stained with DAPI (blue). NS and 16 h pro-inflammatory cytokine-stimulated condition (1 ng/mL TNF-α+20 IU/mL IFN-γ) are shown. Representative image from each donor (donor 1, 2, 3) are shown. Each staining is representative of at least three independent differentiations performed on three distinct chamber slides. Scale bars=50 μm.

FIG. 23. hPSC-derived endothelial progenitors as a model for studying Wnt-mediated barriergenesis. (A) Overview of the endothelial differentiation and Wnt treatment protocol. (B) Immunocytochemistry analysis of CD34 and CD31 expression in D5 EPCs prior to MACS. Hoechst nuclear counterstain is overlaid in the merged image. Scale bars: 200 μm. (C) Flow cytometry analysis of CD34 and CD31 expression in D5 EPCs prior to MACS. (D) Immunocytochemistry analysis of β-catenin and GLUT-1 expression in Passage 1 ECs treated with Wnt3a or control. Hoechst nuclear counterstain is overlaid. Arrowheads indicate smooth muscle-like cells (SMLCs). Scale bars: 200 (E) Quantification of the percentage of GLUT-1⁺ ECs in control- and Wnt3a-treated conditions. Points represent replicate wells from 2 independent differentiations of the IMR90-4 line, each differentiation indicated with a different color. Bars indicate mean values. P-value: Two-way ANOVA. (F) Smooth-muscle like cells (SMLCs). Immunocytochemistry analysis of calponin and smooth muscle protein 22-α (SM22α) in Passage 1 cultures containing ECs and SMLCs. Hoechst nuclear counterstain is overlaid in the merged image. Dashed area in (F) indicates an EC colony. Scale bars: 200 μm.

FIG. 24. Effect of Wnt ligands and pathway modulators on endothelial properties. (A) Immunocytochemistry analysis of claudin-5, caveolin-1, and GLUT-1 expression in Passage 1 ECs treated with Wnt3a, Wnt3a+R-spondin 1 (Rspo1), Wnt7a, Wnt7b, Wnt7a+Wnt7b, Wnt7a+Wnt7b+Rspo1, CHIR, or control. Hoechst nuclear counterstain is overlaid in the merged images. Dashed boxes indicate fields displayed in (B). Scale bars: 200 μm. (B) Immunocytochemistry analysis of GLUT-1 expression in the fields indicated with dashed boxes in (A) from the control and Wnt7a+Wnt7b conditions. To visualize weak GLUT-1 immunoreactivity in Wnt7a+Wnt7b-treated ECs, a linear brightness/contrast adjustment was applied identically to the three fields but differs from that of the images shown in (A). Arrowheads indicate GLUT-1⁺ ECs. (C) Quantification of images from the conditions described in (A) for percentage of ECs (claudin-5⁺ cells relative to total nuclei), GLUT-1⁺ ECs (relative to total claudin-5⁺ ECs), and mean fluorescence intensity of claudin-5, caveolin-1, and GLUT-1 normalized to Hoechst mean fluorescence intensity within the area of claudin-5⁺ ECs only. Points represent replicate wells from one differentiation of the IMR90-4 line and bars indicate mean values. For the fluorescence intensity plots, values were normalized such that the mean of the control condition equals 1. P-values: ANOVA followed by Dunnett's test versus control.

FIG. 25. Dose-dependent effects of CHIR on endothelial properties. (A) Immunocytochemistry analysis of claudin-5, caveolin-1, and GLUT-1 expression in Passage 1 ECs treated with 2 μM, 4 μM, or 6 μM CHIR, or DMSO vehicle control. Hoechst nuclear counterstain is overlaid in the merged images. Scale bars: 200 μm. (B) Quantification of images from the conditions described in (A) for number of ECs per 20× field and percentage of ECs (claudin-5⁺ cells relative to total nuclei). Points represent replicate wells from one differentiation of the IMR90-4 line and bars indicate mean values. P-values: ANOVA followed by Tukey's HSD test. (C) Quantification of claudin-5, caveolin-1, and GLUT-1 mean fluorescence intensity normalized to Hoechst mean fluorescence intensity within the area of claudin-5⁺ ECs only. Points represent replicate wells from one differentiation of the IMR90-4 line. Bars indicate mean values, with values normalized such that the mean of the DMSO condition equals 1. P-values: ANOVA followed by Tukey's HSD test.

FIG. 26. CHIR-mediated effects in an additional hPSC line. (A) Immunocytochemistry analysis of claudin-5, caveolin-1, and GLUT-1 expression in Passage 1 ECs differentiated from the WTC11 iPSC line treated with 2 μM, 4 μM, or 6 μM CHIR, or DMSO vehicle control. Hoechst nuclear counterstain is overlaid in the merged images. Scale bars: 200 μm. (B) Quantification of images from the conditions described in (A) for number of ECs per 20× field and percentage of ECs (claudin-5⁺ cells relative to total nuclei). Points represent replicate wells from 1-2 differentiations of the WTC11 line and bars indicate mean values, each differentiation indicated with a different color. P-values: Two-way ANOVA followed by Tukey's HSD test. (C) Quantification of claudin-5, caveolin-1, and GLUT-1 mean fluorescence intensity normalized to Hoechst mean fluorescence intensity within the area of claudin-5⁺ ECs only. Points represent replicate wells from 1-2 differentiations of the WTC11 line, each differentiation indicated with a different color. Bars indicate mean values, with values normalized within each differentiation such that the mean of the DMSO condition equals 1. P-values: Two-way ANOVA followed by Tukey's HSD test on unnormalized data.

FIG. 27. β-catenin-dependence of CHIR-mediated GLUT-1 induction. (A) Immunocytochemistry analysis of GLUT-1 expression in Passage 1 ECs treated with DMSO, CHIR, or CHIR+doxycycline (Dox) at 1, 2, or 4 μg/mL. Images from the H9-7TGP-ishcat2, 19-9-11-7TGP-ishcat3, and IMR90-4 lines are shown. Hoechst nuclear counterstain is overlaid. Dashed lines indicate borders between EC colonies and SMLCs in the DMSO condition. Scale bars: 200 (B) Quantification of images from the conditions described in (A) for GLUT-1 mean fluorescence intensity normalized to Hoechst mean fluorescence intensity within the area of ECs only. At left, points represent replicate wells from one differentiation of the H9-7TGP-ishcat line (green) and one differentiation of the 19-9-11-7TGP-ishcat3 line (orange). Bars indicate mean values, with values normalized within each differentiation such that the mean of the DMSO condition equals 1. P-values: Two-way ANOVA followed by Tukey's HSD test on unnormalized data. At right, points represent replicate wells from one differentiation of the IMR90-4 line. Bars indicate mean values, with values normalized such that the mean of the DMSO condition equals 1. P-values: ANOVA followed by Tukey's HSD test.

FIG. 28. Effect of CHIR on endothelial cell proliferation. (A) Immunocytochemistry analysis of CD31 and Ki67 expression in Passage 1 ECs treated with DMSO or CHIR. Hoechst nuclear counterstain is overlaid. Dashed lines indicate borders between the EC colony and SMLCs in the DMSO condition. Arrowheads indicate examples of Ki67⁺ ECs. Scale bars: 200 (B) Quantification of the percentage of Ki67⁺ ECs in DMSO- and CHIR-treated conditions. Points represent replicate wells from one differentiation of the WTC11 line, and bars indicate mean values. P-value: Student's t test. (C) Flow cytometry-based cell cycle analysis. Representative plots of Vybrant DyeCycle Green Stain abundance in CD31⁺ cells from Passage 1 cultures treated with DMSO or CHIR. (D) Quantification of the percentage of S/G2/M phase ECs. Points represent replicate wells from one differentiation of the WTC11 line, and bars indicate mean values. P-value: Student's t test.

FIG. 29. Effect of neural rosette- and astrocyte-conditioned media on endothelial properties. (A) Immunocytochemistry analysis of claudin-5, caveolin-1, and GLUT-1 expression in Passage 1 ECs treated with DMSO, CHIR, neural rosette-conditioned medium (NR-CM), or astrocyte-conditioned medium (Astro-CM). Hoechst nuclear counterstain is overlaid in the merged images. Dashed boxes indicate fields displayed in (B). Scale bars: 200 μm. (B) Immunocytochemistry analysis of GLUT-1 expression in the fields indicated with dashed boxes in (A). A linear brightness/contrast adjustment was applied identically to the four fields but differs from that of the images shown in (A). (C) Quantification of images from the conditions described in (A) for number of ECs per 20× field and percentage of ECs (claudin-5⁺ cells relative to total nuclei). Points represent replicate wells from two independent differentiations of the IMR90-4 line, each differentiation indicated with a different color. Bars indicate mean values. P-values: Two-way ANOVA followed by Tukey's HSD test. (D) Quantification of claudin-5, caveolin-1, and GLUT-1 mean fluorescence intensity normalized to Hoechst mean fluorescence intensity within the area of claudin-5⁺ ECs only. Points represent replicate wells from two independent differentiations of the IMR90-4 line, each differentiation indicated with a different color. Bars indicate mean values, with values normalized within each differentiation such that the mean of the DMSO condition equals 1. P-values: Two-way ANOVA followed by Tukey's HSD test on unnormalized data.

FIG. 30. Effect of CHIR on endothelial PLVAP expression. (A) Confocal immunocytochemistry analysis of VE-cadherin and PLVAP expression in Passage 1 ECs treated with DMSO or CHIR. Hoechst nuclear counterstain is overlaid. Eight serial confocal Z-slices with 1 μm spacing are shown. Scale bars: 50 μm. (B) Quantification of PLVAP and VE-cadherin area under the curve (AUC) of mean fluorescence intensity versus Z-position normalized to Hoechst AUC. Points represent replicate wells from 3 independent differentiations of the IMR90-4 line, each differentiation indicated with a different color. Bars indicate mean values, with values normalized within each differentiation such that the mean of the DMSO condition equals 1. P-values: Two-way ANOVA on unnormalized data.

FIG. 31. Effect of CHIR on protein expression in Passage 1 and Passage 3 ECs. (A) Western blots of Passage 1 ECs treated with DMSO or CHIR probed for GLUT-1, claudin-5, PLVAP, VE-cadherin, LSR, and β-actin. (B) Quantification of Western blots of Passage 1 ECs. GLUT-1, claudin-5, PLVAP, VE-cadherin, and LSR band intensities were normalized to β-actin band intensity. Points represent replicate wells from 2-3 independent differentiations of the IMR90-4 line, each differentiation indicated with a different color. Bars indicate mean values, with values normalized within each differentiation such that the mean of the DMSO condition equals 1. P-values: Two-way ANOVA on unnormalized data. (C) Western blots of Passage 3 ECs treated with DMSO or CHIR probed for GLUT-1, claudin-5, PLVAP, VE-cadherin, LSR, and β-actin. (D) Quantification of Western blots of Passage 3 ECs. GLUT-1, claudin-5, PLVAP, VE-cadherin, and LSR band intensities were normalized to β-actin band intensity. Points represent replicate wells from 2 independent differentiations of the IMR90-4 line, each differentiation indicated with a different color. Bars indicate mean values, with values normalized within each differentiation such that the mean of the DMSO condition equals 1. P-values: Two-way ANOVA on unnormalized data.

FIG. 32. Functional properties of CHIR- and DMSO-treated ECs. (A) Flow cytometry analysis of CD31 expression in Passage 1 ECs following the dextran internalization assay. CD31⁺ cells were gated for further analysis. (B) Flow cytometry analysis of 10 kDa dextran-Alexa Fluor 488 (AF488) abundance in CD31⁺ cells. Cells were treated with DMSO or CHIR for 6 d prior to the assay. Representative plots from cells incubated with dextran for 2 h at 4° C. (left) and 37° C. (right) are shown. (C) Quantification of 10 kDa dextran-AF488 geometric mean fluorescence intensity in CD31⁺ cells. Treatment and assay conditions were as described in (B). Points represent replicate wells from 3 independent differentiations of the IMR90-4 line, each differentiation indicated with a different color. Bars indicate mean values. P-values: Two-way ANOVA followed by Tukey's HSD test. (D) Quantification of the coefficient of variation (CV) of 10 kDa dextran-AF488 fluorescence intensity in CD31⁺ cells. Points represent replicate wells from 3 independent differentiations of the IMR90-4 line, each differentiation indicated with a different color. Bars indicate mean values. P-value: Two-way ANOVA. (E) Transendothelial electrical resistance (TEER) of Passage 3 ECs. The x-axis indicates the number of days after seeding cells on Transwell inserts. Points represent replicate wells from three independent differentiations of the IMR90-4 line, each differentiation indicated with a different shape. P-value: Two-way ANOVA. (F) Permeability of Passage 3 ECs to sodium fluorescein. Points represent replicate wells from two independent differentiations of the IMR90-4 line, each differentiation indicated with a different color. Bars indicate mean values. P-value: Two-way ANOVA.

FIG. 33. Effect of CHIR treatment in matured endothelium. (A) Overview of the endothelial differentiation, extended culture, and CHIR treatment protocol. (B) Immunocytochemistry analysis of claudin-5 and GLUT-1 expression in ECs treated with DMSO or CHIR as outlined in (A). Images from the IMR90-4 and H9-CDH5-eGFP lines are shown. Hoechst nuclear counterstain is overlaid. Scale bars: 200 μm. (C) Quantification of images from the conditions described in (B) for GLUT-1 and claudin-5 mean fluorescence intensity normalized to Hoechst mean fluorescence intensity within the area of claudin-5⁺ ECs only. Points represent replicate wells from one differentiation of the IMR90-4 line (orange) and one differentiation of the H9-CDH5-eGFP line (blue). Bars indicate mean values, with values normalized within each differentiation such that the mean of the DMSO condition equals 1. P-values: Two-way ANOVA on unnormalized data. (D) Quantification of images from the conditions described in (B) for number of ECs per 20× field. Points represent replicate wells from one differentiation of the IMR90-4 line (orange) and one differentiation of the H9-CDH5-eGFP line (blue). Bars indicate mean values. P-value: Two-way ANOVA.

FIG. 34. RNA-seq of DMSO-, CHIR-, or Wnt7a/b-treated ECs. (A) Principal component analysis of EC and SMLC whole-transcriptome data subject to variance stabilizing transformation by DESeq2. Points from Passage 1 ECs represent cells from 4 independent differentiations of the IMR90-4 line, points from Passage 3 ECs represent cells from 3 independent differentiations of the IMR90-4 line, and points from SMLCs represent 2 independent differentiations of the IMR90-4 line. Points are colored based on treatment: DMSO (blue), CHIR (orange), or Wnt7a/b (red). Data are plotted in the space of the first two principal components, with the percentage of variance explained by principal component 1 (PC1) and principal component 2 (PC2) shown in axis labels. (B) Heat map of transcript abundance [log₂(TPM+1)] for endothelial, mesenchymal, and epithelial genes across all samples. (C) Differential expression analysis of Passage 1 CHIR-treated ECs compared to Passage 1 DMSO-treated ECs. Differentially expressed genes (adjusted P-values <0.05, DESeq2 Wald test with Benjamini-Hochberg correction) are highlighted in green (upregulated) and red (downregulated). The number of upregulated, downregulated, and non-significant (ns) genes are shown in the legend. (D) Transcript abundance (TPM) of Wnt-regulated, barrier-related genes in Passage 1 DMSO- and CHIR-treated ECs. Points represent cells from 4 independent differentiations of the IMR90-4 line and lines connect points from matched differentiations. All genes shown were differentially expressed (adjusted P-values <0.05, DESeq2 Wald test with Benjamini-Hochberg correction). (E) Venn diagrams illustrating the number of genes identified as upregulated or downregulated (adjusted P-values <0.05, DESeq2 Wald test with Benjamini-Hochberg correction) in Passage 1 ECs treated with CHIR versus DMSO compared to Wnt7a/b versus DMSO (left), or ECs treated with CHIR versus DMSO at Passage 1 compared to Passage 3 (right).

FIG. 35. RNA-seq differential expression analyses. (A-C) Differential expression analysis of Passage 3 CHIR-treated ECs compared to Passage 3 DMSO-treated ECs (A), Passage 1 Wnt7a/b-treated ECs compared to Passage 1 DMSO-treated ECs (B), and Passage 3 DMSO-treated ECs compared to Passage 1 DMSO-treated ECs (C). Differentially expressed genes (adjusted P-values <0.05, DESeq2 Wald test with Benjamini-Hochberg correction) are highlighted in green (upregulated) and red (downregulated). The number of upregulated, downregulated, and non-significant (ns) genes are shown in the legends. (D) Heat map of transcript abundance [log₂(TPM+1)] for BBB genes encompassing tight junctions, vesicle trafficking components, and solute carriers, and efflux transporters. Solute carrier and efflux transporter genes that were expressed in human brain ECs at an average of >100 TPM in a meta-analysis of scRNA-seq datasets (Gastfriend et al., 2021. Integrative analysis of the human brain mural cell transcriptome. J Cereb Blood Flow Metab, In press) are included. At right, arrows indicate directionality of change for differentially expressed genes (adjusted P-values <0.05, DESeq2 Wald test with Benjamini-Hochberg correction) for the four comparisons shown above. Changes with expected directionality for gain of CNS EC character have arrows highlighted in green.

FIG. 36. Expression of Wnt pathway components in naïve ECs. Abundance of transcripts (in transcripts per million, TPM) encoding Wnt receptors, co-receptors, and other pathway components in Passage 1 DMSO-treated ECs. Points represent cells from 4 independent differentiations of the IMR90-4 line. Bars indicate mean values. ADGRA2 is also known as GPR124.

FIG. 37. Identification of concordantly Wnt-regulated CNS EC-associated genes in RNA-seq data. (A) Differential expression analysis of P7 murine brain ECs compared to liver, lung, or kidney ECs (Sabbagh et al., 2018. Transcriptional and epigenomic landscapes of CNS and non-CNS vascular endothelial cells. Elife 7:1-44). Differentially expressed genes (adjusted P-values <0.05, DESeq2 Wald test with Benjamini-Hochberg correction) are highlighted in green (up, brain-enriched) and red (down, brain-depleted). The number of up, down, and non-significant (ns) genes are shown in the legends. (B) Venn diagrams illustrating the number of genes identified as brain EC-enriched (left) or brain EC-depleted (right) versus liver, lung, or kidney ECs (adjusted P-values <0.05, DESeq2 Wald test with Benjamini-Hochberg correction). The 1094 genes enriched in brain ECs compared to each other organ, and the 506 genes depleted in brain ECs compared to each other organ, were used for subsequent analysis of the effects of Wnt activation in the various experimental contexts. (C-F) In each plot, the x-axis indicates average log 2(fold change) of gene expression in brain ECs compared to liver, lung, and kidney ECs for the 1094 brain EC-enriched genes and 506 brain EC-depleted genes described in (B) with known mouse-human homology. Homologous human gene names are shown. The y-axes indicate differential expression [log 2(fold change)] in Passage 1 CHIR-treated ECs compared to Passage 1 DMSO-treated ECs (C), in adult mouse pituitary ECs with stabilized β-catenin (gain-of-function, GOF) compared to controls (D) (Wang et al., 2019. Beta-catenin signaling regulates barrier-specific gene expression in circumventricular organ and ocular vasculatures. Elife 8:1-36), in adult mouse liver ECs with stabilized β-catenin compared to controls (E) (Munji et al., 2019), or in cultured adult mouse brain ECs with stabilized β-catenin compared to controls (F) (Sabbagh et al., 2020. A genome-wide view of the de-differentiation of central nervous system endothelial cells in culture. Elife 9:1-19). Points are highlighted in blue if concordantly-regulated (upregulated in both comparisons or downregulated in both comparisons). Genes were identified as upregulated or downregulated based on adjusted P-values <0.05, DESeq2 Wald test with Benjamini-Hochberg correction. (G) Venn diagrams illustrating the number of brain EC-enriched genes concordantly upregulated with β-catenin GOF (top) and the number of brain EC-depleted genes concordantly downregulated with β-catenin GOF (bottom) for the four comparisons shown in (C-F).

### DETAILED DESCRIPTION

In the present application, the inventors provide a new in vitro blood-brain barrier model that not only has blood-brain barrier properties, but also has a mature immune phenotype that allows the model to be used for studying immune regulation and function at the blood-brain barrier.

The human induced pluripotent stem cell (hiPSC)-derived blood-brain barrier (BBB) models that have been established to date lack expression of key adhesion molecules involved in immune cell migration across the BBB in vivo. In the Examples, the inventors demonstrate that two different, previously established methods of for differentiating hiPSCs to BMEC-like cell for blood-brain barrier models (i.e., co-differentiation unconditioned medium method (UMM) (14, 16, 23) and chemically-defined medium method (DMM) (17)) yield BMEC-like cells lacking expression of several key adhesion molecules known to be involved in immune cell migration across the BBB in vivo.

To address the lack of these adhesion molecules in currently available hiPSC-derived BBB models, the inventors developed a new protocol to differentiate hiPSC-derived endothelial progenitor cells (EPCs) to BMEC-like cells that display an improved, mature immune phenotype, while also displaying barrier properties similar to those observed for primary human brain endothelial cells (24-28). Specifically, the inventors developed the extended endothelial cell culture method (EECM), which differentiates hiPSC-derived endothelial progenitor cells to brain microvascular endothelial cell (BMEC)-like cells with good barrier properties and mature tight junctions. Importantly, EECM-BMEC-like cells exhibited constitutive cell surface expression of ICAM-1, ICAM-2 and E-selectin, along with additional adhesion molecules important for immune phenotype as described further herein. Pro-inflammatory cytokine stimulation increased cell surface expression of ICAM-1 and induced cell surface expression of P-selectin and VCAM-1. Coculture of EECM-BMEC-like cells with hiPSC-derived smooth muscle-like cells or their conditioned medium further increased induction of VCAM-1. Functional expression of endothelial ICAM-1 and VCAM-1 was confirmed by T-cell interaction with EECM-BMEC-like cells. Thus, the present invention provides the first hiPSC-derived BBB model that faithfully reproduces the required molecular repertoire needed for the study of immune cell trafficking across the BBB.

### EECM-Derived BMEC Populations

The present invention provides a method of producing a population of extended endothelial culture method (EECM)-derived brain microvascular endothelial cells (BMECs) (EECM-BMECs) from a population of endothelial progenitor cells. These cells can be used to form in vitro blood-brain barrier (BBB) models that have both a mature immune phenotype and BBB properties. These BBB models can be used for studying immune modulation at the BBB and for studying and discovering therapeutics that may be useful for treatment of neuroinflammatory diseases or neurological diseases that have an inflammatory or immune component, as described further herein. The cells used with the present invention may be isogenic, allowing for isogenic disease modeling. The methods are described in more detail below. In brief, the methods involves first culturing endothelial progenitor cells in endothelial medium on a collagen coated surface (e.g., plates, membrane, insert, etc.), and then serial passaging (e.g., at least two passages) the resulting cells in serum-free endothelial medium on a collagen coated surface until a homogeneous confluent monolayer of CD31⁺ EECM-BMECs is produced.

The cells produced by the methods of the present invention are EECM-derived CD31⁺ BMECs. The terms “EECM-derived CD31⁺ MECs,” “EECM-derived CD31⁺MEC-like cells,” or “EECM-BMECs” or “EECM-BMEC-like cells” are used interchangeably herein to refer to the cells derived by the disclosed methods. The EECM-BMECs, when grown into a confluent monolayer, exhibit both a mature immune phenotype and blood-brain barrier properties, which are each discussed in turn below. The monolayers are homogenous, comprising, for example, more than 90% EECM-derived CD31⁺ BMECs, preferably more than 95% CD31⁺ BMECs, or alternatively at least 98% CD31⁺ BMECs.

First, the EECM-BMECs made by the methods described herein are useful in that they have a mature immune phenotype not found in the BMECs used in previously disclosed in vitro BBB models. Importantly, after extended culture, the resulting EECM-BMEC-like cells retained an immune cell adhesion molecule profile similar to primary BMECs (26, 27, 59), which are known to regulate immune cell adhesion and migration across the BBB. These EECM-BMECs expressed ICAM-1, ICAM-2, E-selectin, P-selectin, CD99, and PECAM-1. Further, pro-inflammatory cytokine stimulation of these EECM-BMECs led to upregulation of ICAM-1 and P-selectin and induction of VCAM-1 that can be further enhanced by co-culture with smooth muscle-like cells (SMLC) or by treating the EECM-BMECs with SMLC-derived conditioned medium. The functionality of both ICAM-1 and VCAM-1 in the BBB model were tested, which confirmed that blockade of these molecules result in reduced CD4+ helper T cell adhesion. EECM-BMEC-like cells demonstrated pro-inflammatory cytokine-inducible expression of VCAM-1, a prerequisite for studying immune cell interactions with the BBB. Further, the EECM-BMECs further express blood brain barrier markers as described more below.

As used herein, the term “mature immune phenotype” refers to the expression in the BMECs of three or more adhesion molecules that play a role in immune cell adhesion and transport across the BBB. The three or more adhesion molecules are also referred to as immune cell adhesion molecules in the art.

In some embodiments, the EECM-derived CD31⁺ BMECs in addition to the expressing the vascular endothelial (VE)-cadherin and ICAM-2 and PECAM-1, the EECM-BMECs further express three or more adhesion molecules selected from the group consisting of ICAM-1, VCAM-1, E-selectin, P-selectin, and CD99. The adhesion molecules are also referred to in the art as “endothelial cell adhesion molecules”. Not to be bound by any theory, but these molecules are thought to be involved in adhesion and migration of peripheral blood T lymphocyte across blood-brain barrier during physiological and inflammatory conditions such as immune-mediated CNS diseases. As such, expression of one or more, preferably three or more of these adhesion molecules on the EECM-derived BMECs provides a BBB model that may be used to study multi-step immune cell trafficking across the BBB. In some embodiments, the cells express four or more adhesion molecules selected from the group consisting of ICAM-1, VCAM-1, E-selectin, P-selectin, and CD99, or alternatively express all five of the adhesion molecules ICAM-1, VCAM-1, E-selectin, P-selectin, and CD99.

In a preferred embodiment, the EECM-derived CD31⁺ BMECs have a mature adhesion molecule phenotype that is characterized by the combined expression of at least one IgCAM family member (i.e., ICAM-1 and/or VCAM-1), at least one selectin (i.e., E-selectin and/or P-selectin), and CD99. In another embodiment, the EECM-BMECs have two or more IgCAM members (i.e., ICAM-1 and VCAM-1), and E-selectin and P-selectin, and CD99.

In some embodiments, the EECM-BMECs generated by the methods described herein further express activated leukocyte cell adhesion molecule (ALCAM), and melanoma cell adhesion molecule (MCAM), both cell adhesion molecules found on blood-brain barrier endothelial cells and involved in leukocyte transmigration across this endothelium.

Second, the EECM-BMECs made by the methods described herein are useful in that they have blood-brain barrier (BBB) properties, including physical barrier properties, well-developed tight junctions, selective permeability, moderate TEER, a flat cellular morphology, and junctional architecture characteristic of primary BMECs. Importantly, these BBB properties enable the study of BBB characteristics in vitro. The terms “tight junction,” “occluding junction,” or “zonulae occludentes” describe multi-protein junctional complexes between the membranes of two neighboring cells that prevent leakage of solutes and water across the cellular monolayer. In the Examples, the inventors perform an immunocytochemical analysis of claudin-5 and occludin to demonstrate that the monolayers described herein have continuous tight junctions, providing improved barrier properties as opposed to naïve endothelial cells.

As described, the EECM-BMECs described herein have both a mature immune phenotype and BBB properties, including the expression of BBB marker proteins. Specifically, the population of EECM-derived CD31⁺ BMECs described herein express one or more blood-brain barrier (BBB) markers selected from the group consisting of occludin, claudin-5, zonula occludens-1 (ZO-1), Von Willebrand factor (vWF), and caveolin-1. As used herein, the term “blood-brain barrier markers,” refers to markers that are associated with brain microvascular endothelial cells, e.g. tight junction markers (e.g., occludin, claudin-5, zonula occludens-1), and additional markers that have been associated with brain microvasculature that play a role in the blood-brain barrier, (e.g., Von Willebrand factor and caveolin-1). In some embodiments, the EECM-BMECs express at least two markers selected from occludin, claudin-5, ZO-1, vWF, and caveolin-1. In some embodiments, the EECM-BMECs express at least three markers selected from occludin, claudin-5, ZO-1, vWF, and caveolin-1. Alternatively, the EECM-BMECs express at least four markers selected occludin, claudin-5, ZO-1, vWF, and caveolin-1. Still alternatively, the EECM-BMECs express all five of the markers selected from occludin, claudin-5, ZO-1, vWF, and caveolin-1.

Suitably, in the population of EECM-derived CD31⁺ BMECs, at least 90% of the cells express one or more of the BBB markers and three or more of the adhesion molecule, preferably at least 95% of the cells in the population express at least one BBB marker and at least three adhesion molecules. Alternatively, at least 98% of the cells express at least one BBB marker and at least three adhesion molecules.

One skilled in the art would understand that the homogenous population of EECM-derived CD31⁺ BMECs may express one or more BBB marker and the three or more adhesion markers at slightly different levels and still be considered a homogenous population if the population has the characteristics as described herein. Thus, in some embodiments, a homogenous population comprises at least 90% EECM-BMECs, at least 95% EECM-BMECs, at least 98% EECM-BMECs, alternatively at least 99% EECM-BMECs.

The EECM-derived CD31⁺ BMECs disclosed herein may have BBB properties that include one or more of the following characteristics: (a) a lower permeability as compared to naïve endothelial cells; (b) increased expression of one or more tight junction proteins (e.g., claudin-5, occudin, or ZO-1) as compared to naïve endothelial cells; (c) improved localization of occludin and claudin-5 to tight junctions between cells as compared to naïve endothelial cells; (d) a TEER that is considered to provide barrier properties (e.g., a TEER above 60 Ω×cm²). As used herein, the term “naïve endothelial cells” refers to endothelial cells that have not been subjected to the methods of the present invention and do not express the desired mature immune phenotype, and include the progenitor endothelial cells described below.

The endothelial cells lining brain microvessels that separate the blood from the brain parenchyma serve as an interface for the exchange of nutrients, gasses, and metabolites between the blood and the brain, and also serve as a barrier that prevents neurotoxic components of plasma and xenobiotics (including therapeutics) from entering the brain. Thus, one important BBB property of the EECM-BMECs of the present invention is the physical permeability of a confluent monolayer of these cells. The permeability of this monolayer refers to the ability of the cells to allow selective transport of molecules across the cell monolayer, which is highly regulated as compared to endothelial cells in other parts of the body. Permeability of this monolayer can be characterized using a number of known methods that can be readily carried out by one skilled in the art. For example, permeability can be characterized in vitro by the ability of the monolayer of cells to allow transport of a labeled-compound across the monolayer. One suitable method of measuring permeability involves measuring the amount of sodium fluorescein that traverses across the cell monolayer. Suitably, in some embodiments, the cell monolayer of the present invention has a permeability to sodium fluorescein below about 1×10⁻³ cm/min. In another suitable embodiment, the monolayer has a permeability to Lucifer yellow below about 1×10⁻³ cm/min. Monolayers having higher permeabilities fail to establish good barrier properties. Other molecules for determining permeability are known in the art, and the threshold for such molecules will depend on the molecule's size and molecular weight. Thus, the threshold used with other molecules may differ from that established for sodium fluorescein. Permeability of the monolayer may also be measured in terms of transendothelial electrical resistance (TEER) values. TEER values of the present EECM-derived CD31+ BMECs are about 60 Ω×cm², for example, and may be in the range of about 50-180 Ω×cm² or greater or 60-80 Ω×cm².

### Methods of Making EECM-Derived BMECS Populations

The present invention provides methods of producing a population of extended endothelial culture method (EECM) derived CD31+ brain microvascular endothelial cells (BMECs) from a population of endothelial progenitor cells. The methods involve first culturing the endothelial progenitor cells in serum-free endothelial medium, and then serial passaging (e.g., at least two passages) the resulting cells in serum-free endothelial medium on collagen coated plates until a homogeneous confluent monolayer of CD31⁺ EECM-BMECs is produced.

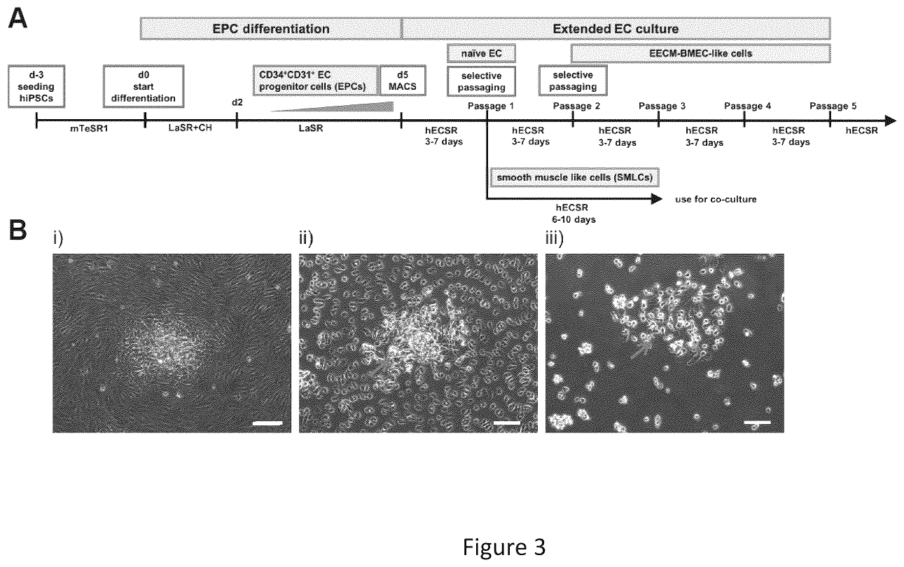

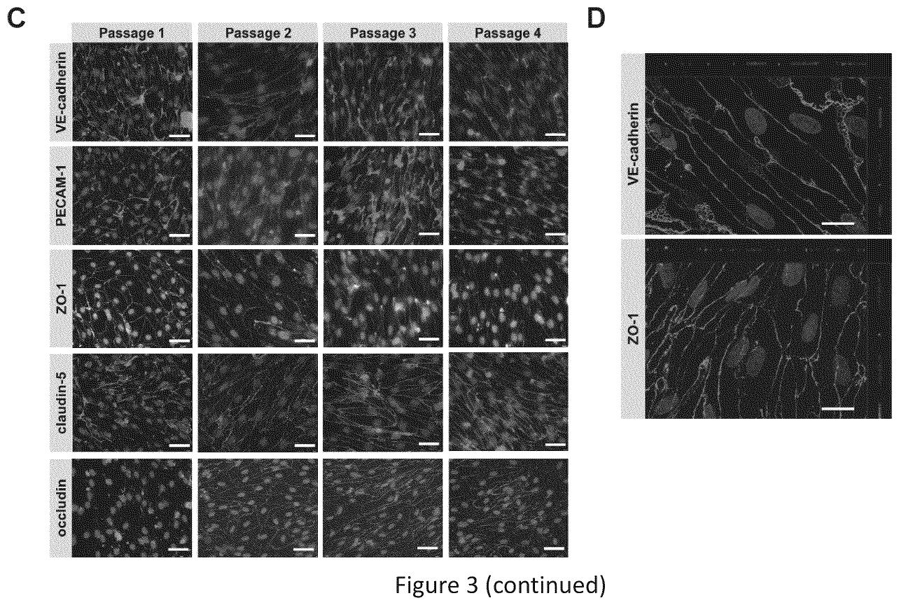

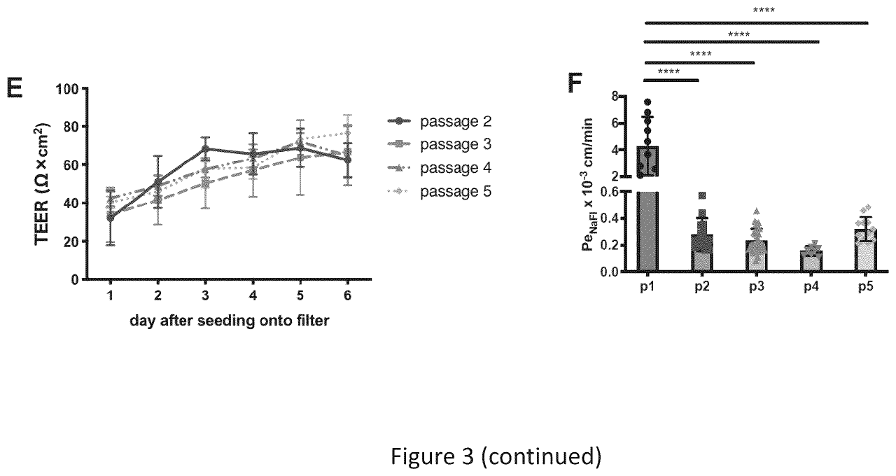

In one embodiment, the method comprises: (a) culturing a cell population of CD34+CD31+ endothelial progenitor cells in serum-free endothelial medium on collagen coated surface until confluent; (b) selectively passaging the endothelial cells of (a) in serum-free endothelial medium on collagen surface for at least two passages; (c) culturing the selectively passaged cells of (b) in serum-free endothelial medium until confluent. This method produces a confluent monolayer comprising a population of a population of CD31⁺ EECM-BMECs that express vascular endothelial (VE)-cadherin, ICAM-2, PECAM-1, and three or more adhesion molecules selected from the group consisting of ICAM-1, VCAM-1, E-selectin, P-selectin, and CD99. For example, FIG. 3A depicts a schematic representation of the protocols used for extended endothelial cell culture method (EECM).

The term “selectively”, “selective”, “selection”, and “selecting” are used herein interchangeably as understood in the art to describe the process of choosing one subset based on defined characteristics from a heterogeneous population. The selective passaging of endothelial cells in step (b) may be performed using any suitable method known to select endothelial-like cells from the non-endothelial like cells. In one suitable method (described in the Examples below) cells are treated with a detachment solution (e.g., proteases and/or collagenases) to loosen the cells from the cell culture surface, and the cells that are first to be released from the surface are separated from the more adherent non-endothelial cells. Suitable detachment solutions are known in the art and include, for example, one or more proteolytic or collagenolytic enzymes. One exemplary detachment solution is Accutase™ (Innovative Cell Technologies, Inc., San Diego, CA). Thus, in some embodiments, selectively passaging comprises: detaching and collecting the endothelial cells from the culture plate, wherein the non-endothelial cells are not-detached from the tissue culture plate.

However, other methods of selectively passaging endothelial cells are also contemplated, for example, fluorescence-activated cell sorting (FACS), magnetic bead sorting, etc. Thus, in some embodiments, selectively passaging comprises enriching for PECAM-1 positive (PECAM-1⁺) endothelial cells as opposed to PECAM-1 negative (PECAM-1⁻) smooth muscle-like cells, e.g., using a cell sorting method. Other markers of endothelial cells can also be used for this selection step including, for example, VE-cadherin.

The methods described herein include serial passaging the endothelial-like cells for at least two passages. The term “passaging” is intended to have the standard meaning used in the art of tissue culture, i.e., the harvesting a confluent cell population from a surface and reseeding at a lower concentration into a daughter cell culture (i.e. subculturing). Each subsequent round of harvesting and reseeding is considered a “passage.” The EECM methods described herein provide at least two passages of the EC-like cells in serum free endothelial medium, and may comprise at least three passages, at least four passages, at least five passages, at least six passages, etc. In some embodiments, the method comprises repeating step (b) for three or more passages to produce a confluent monolayer. Suitably, the cells are passaged, in total, from about two (2) passages to about six (6) passages. The total number of passages required to reach a homogenous monolayer may depend on the initial population of progenitor endothelial cells and can be readily determined by one skilled in the art. The number of passages required may also depend on the subject from which the cells have been obtained. In one embodiment, a suitable number of passages includes 2-6 passages. Advantageously, the chosen number of passages will produce a homogenous monolayer of EECM-derived BMECs as described herein, wherein at least 90% of the cells are BMECs, preferably wherein at least 95% of the cells are BMECs, alternatively wherein at least 98% of the cells are BMECs. The term “confluent monolayer” is a term known in the art to refer to cells in tissue culture that form a cohesive sheet comprising of a single cell layer filing the entire surface area of the bottom of the culture area (e.g., dish).

Suitable serum-free endothelial medium are known in the art and include, but are not limited to, human endothelial serum free medium (hESFM, Thermo Fisher Scientific, Waltham, MA, USA). Specifically, in the Examples, the inventors utilized hESFM supplemented with B-27 (1×) and FGF2 (10 ng/ml) and referred to as “hECSR.” The inventors have determined that the medium selection is crucial, as they have found that other commercially available media (i.e., EGM-2, ECM-5) do not work in the disclosed methods.

In some embodiments of the methods, the culturing in step (a) comprises: (i) culturing the cell population in serum-free endothelial medium on collagen coated surface in the presence of a ROCK inhibitor, optionally for 24 hours; and (ii) removing the ROCK inhibitor and culturing the cells of (i) in serum-free endothelial medium on collagen coated surface until confluent.

Suitable ROCK inhibitors are known in the art and include, but are not limited to, for example, Y-27632, thiazovivin, fasudil, GSK429286A, RKI-1447, Azaindole 1, Hydroxyfasudil, and Y-39983. In preferred embodiments, the ROCK inhibitor is Y-27632 (Selleckchem, Houston, TX, USA). Suitably, Y-27632 is used at a concentration of about 5-10 μM. The endothelial progenitor cells may be cultured for about 24 hours in the presence of the ROCK inhibitor, after which time the ROCK inhibitor is removed and the medium is replaced with serum-free endothelial medium.

In the methods described herein, the endothelial progenitor cells are cultured on surfaces that are coated with collagen. Suitable forms of collagen for use in coating the plates are known in the art and include collagen I-IV. Preferably, the collagen is collagen IV (i.e., the form of collagen that localizes to endothelial basement membranes). The inventors surprisingly discovered that collagen preferentially differentiates the cells into EECM-BMECs. In contrast, when fibronectin was used as a coating, it induced an increase in non-endothelial cell differentiation (e.g., smooth muscle-like cells) as opposed to BMECs. Plates, membranes, or filter inserts (e.g., Corning Transwells®) can be coated with collagen using standard methods known in the art. Alternatively, plates or filter inserts that are pre-coated with collagen can be commercially obtained. In some embodiments, the plates, membranes or filter inserts are coated with an alternative substrate coating, such as vitronectin, Synthemax™ (Corning), or Matrigel® (Corning).

For development and use as a blood-brain barrier model, the method preferably comprises culturing the cells on a surface within a system, preferably a permeable surface. Suitable surfaces are known in the art and include, but are not limited to, for example, permeable support, collagen coated permeable membrane, filter, polymer, mesh, matrices, membranes, hydrogel based substrates, among others. The surface may be within a tissue culture system or microfluidic device or other configurations in which a blood-brain barrier may be observed. Suitable permeable supports are known in the art and include, tissue culture plate inserts, porous and permeable membranes, transwell systems (e.g., Corning Transwells®) and the like. The permeable support allows for the study of translocation of factors across the EECM-derived BMEC monolayer, which forms the BBB model with the mature immune phenotype. Suitable tissue culture plates, systems and inserts are readily commercially available and known in the art. The permeable support may be coated with collagen prior to seeding of the cells of the present invention. The permeable support also allows for the co-culturing of cells without physical contacting of the cells, e.g., so conditioned media from the co-cultured cells can be shared between the two cell types without the two cell types being in physical contact.

In some embodiments, steps (b)-(c) of the methods described herein include culturing the cells in serum-free endothelial medium that comprises conditioned medium from smooth muscle-like cells (SMLCs). As demonstrated in the Examples, the addition of conditioned medium from SMLCs results in the EECM-BMECs expressing increased levels of VCAM-1 on their surface. “Conditioned medium” refers to medium harvested from cells after they have been cultured in it for at least 1 day. Conditioned medium contains metabolites, growth factors and extracellular matrix proteins secreted into the medium by the cultured cells. In this embodiment, the conditioned medium is harvested from a culture of SMLCs.

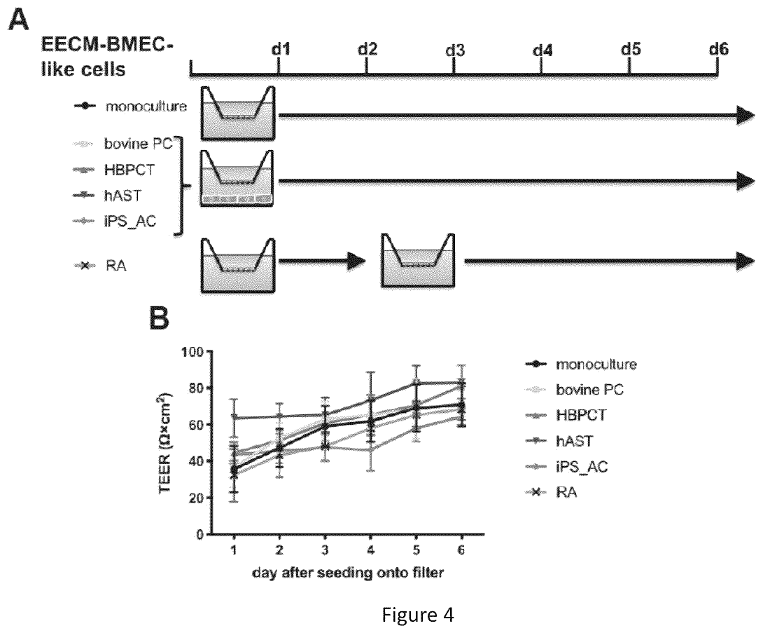

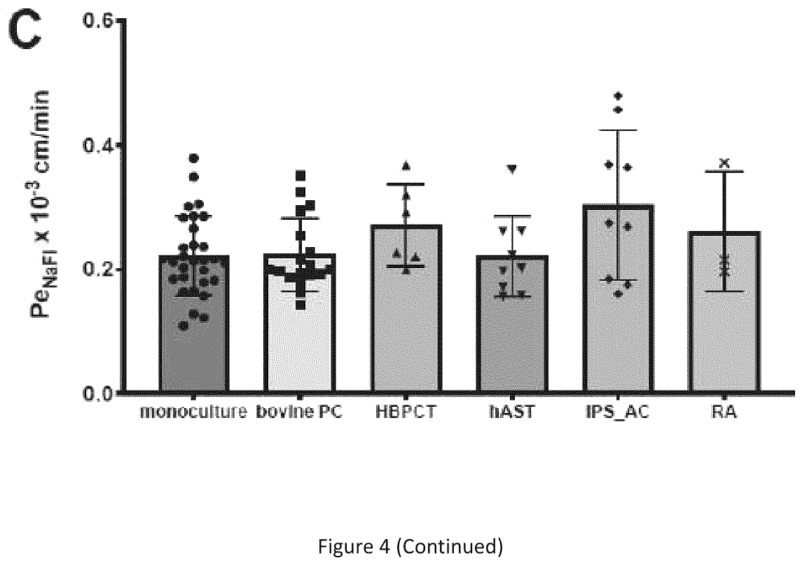

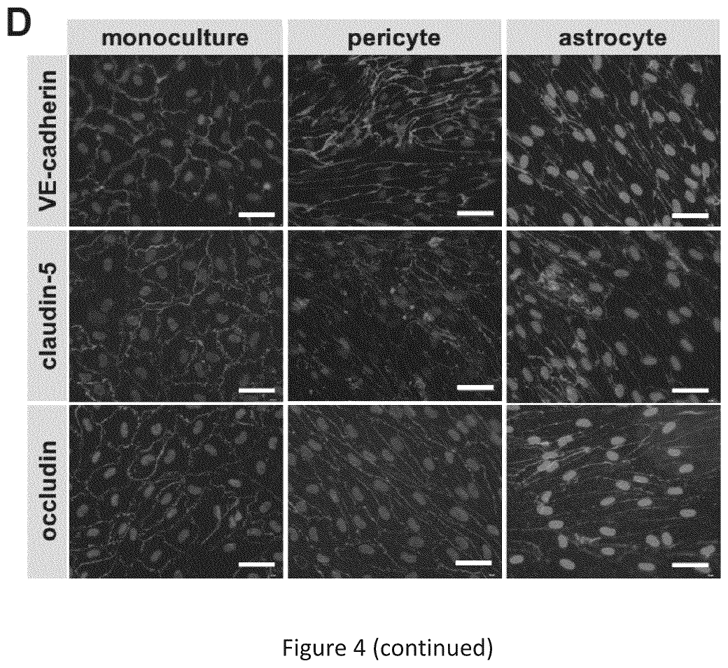

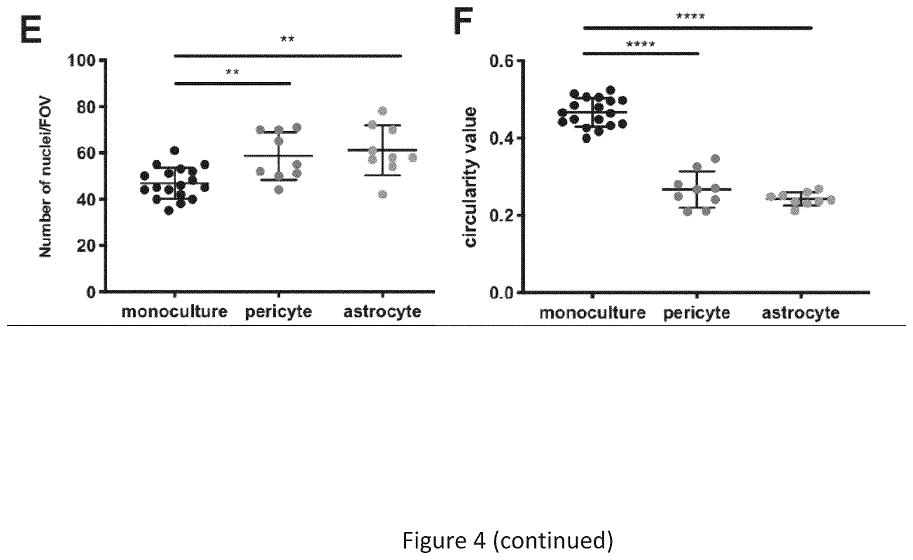

In another suitable embodiment, the conditioned medium from SMLCs is provided by co-culturing the EECM-BMECs with SMLCs. Suitably, a transwell plate, permeable membrane, or other tissue culture insert is used to separate the layer of EECM-BMECs from the SMLCs. The SMLCs may be cultured on the bottom of the tissue culture surface which is separated from the EECM-BMECs by a permeable membrane/filter (i.e. transwell system, e.g., the EECM-BMECs is cultured on the permeable membrane/filter insert and the SMLCs on the bottom of the well) (see, e.g., FIG. 4A) or may be cultured on the other side of the basolateral side of permeable membrane/filter insert, such that the BMECs and SMLCs maintain direct contact through the membrane/filter pores. Suitable size pores for the permeable membrane are known in the art, for example, include 0.4 micro-meter pores. Thus, in some embodiments, steps (b)-(e) are performed in co-culture with smooth muscle-like cells (SMLCs).

The methods described herein produce a confluent monolayer comprising a population of CD31⁺ EECM-BMECs that express three or more, four or more, or all five of the adhesion molecules selected from the group consisting of ICAM-1, VCAM-1, E-selectin, P-selectin, and CD99; and one or more, two or more, three or more, four or more, or all five or the blood-brain barrier markers selected from the group consisting of occludin, claudin-5, zonula occludens-1 (ZO-1), Von Willebrand factor (vWF), and caveolin-1. In one embodiment, the CD31⁺ EECM-BMECs express the adhesion molecules ICAM-1, VCAM-1, E-selectin, P-selectin, and CD99 and the blood-brain barrier markers occludin, claudin-5, ZO-1, vWF, and caveolin-1.

In some embodiments, the EECM-BMECs derived by the methods described herein constitutively express ICAM-1, ICAM-2, and E-selectin.

In some embodiments, the methods further comprise contacting the CD31⁺ EECM-BMECs with at least one pro-inflammatory cytokine during culturing, wherein the contacting with the at least one pro-inflammatory cytokine increases the expression of ICAM-1, P-selectin, VCAM-1 or a combination thereof on the surface of the CD31⁺ EECM-BMECs. Suitable pro-inflammatory cytokines are known in the art and include, but are not limited to, for example, tumor necrosis factor α (TNF-α), interferon γ (IFN-γ), among others. The CD31⁺ EECM-BMECs may be contacted with the pro-inflammatory cytokine at any step of the methods described herein.

Example 2 describes a second embodiment of the present invention in methods of deriving GLUT1+ EECM-BMECs. An additional method of producing BMEC-like cells for use in a blood brain barrier model that have a CNS EC phenotype having barrier properties is provided. The inventors found that Wnt activation in hPSC-derived, naïve endothelial progenitors drives development of a CNS EC-like phenotype, including the canonical GLUT-1, claudin-5, and PLVAP expression effects. The method comprises culturing the cells in the presence of CHIR 99021, a small molecule agonist of Wnt/θ-catenin signaling. The produced BMECs displayed a CHIR-upregulated expression of known CNS EC transcripts, including LEF1, APCDD1, AXIN2, SLC2A1, CLDN5, LSR, ABCG2, SOX7, and ZIC3 and downregulated PLVAP, FABP4, and SMAD6.

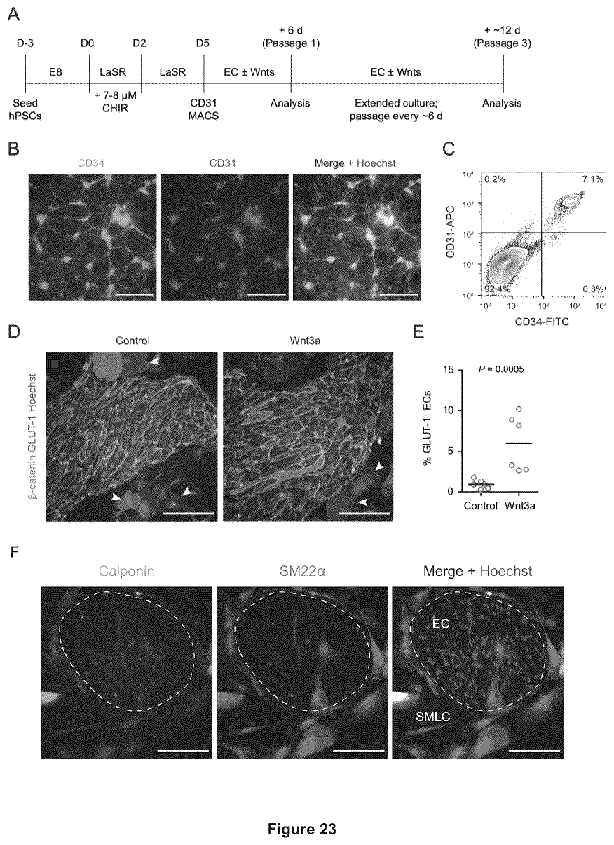

The protocol to produce BMECs by this second method is briefly depicted in Example 2, FIG. 23A. In one embodiment, the method comprises: (a) contacting cultured human pluripotent stem cells with activator of Wnt/β-catenin signaling for a period of about 2 days; (b) culturing the cells of (a) in the absence of the activator for two to three days; (c) separating the CD34+CD31+ endothelial progenitor cells from step (b) from the CD34−CD31− non-EPCs; (d) culturing the separated CD34+CD31+ endothelial progenitor cells on coated plates in medium comprising an activator of Wnt/β-catenin signaling for about 3 days to about 10 days to produce a confluent monolayer; and (e) selectively passaging the cells of step (d) in serum-free endothelial medium on coated plates in medium comprising the activator on coated plates for at least one additional passage to obtain a confluent population of CD31⁺GLUT1⁺EECM-BMECs having a canonical barrier phenotype. Specifically, this population of CD31⁺GLUT1⁺EECM-BMECs expresses glucose transporter 1 (GLUT1), claudin-5 and have reduced expression of plasmalemma vesicle-associated protein (PLVAP). Preferably the cells in step (e) are selectively passaged for at least two passages, alternatively at least three passages.

Activators of Wnt/β-catenin signaling are known and understood in the art. In one embodiment, the activator of Wnt/β-catenin signaling is a Gsk3 inhibitor. In some embodiments, activation of Wnt/β-catenin signaling is achieved by inhibition of Gsk3β (“Gsk3”) phosphotransferase activity or Gsk3 binding interactions. Gsk3 inhibition can be achieved in a variety of ways including, but not limited to, providing small molecule Gsk inhibitors that inhibit Gsk3 phosphotransferase activity, RNA interference knockdown of Gsk3, and overexpression of dominant negative form of Gsk3. Dominant negative forms of Gsk3 are known in the art as described, e.g., in Hagen et at (2002), J Biol Chem, 277(26):23330-23335, which describes a Gsk3 comprising a R96A mutation.

Suitable small molecule Gsk3 inhibitors include, but are not limited to, CHIR 99021 (CAS No. 252917-06-9), CHIR 98014 (CAS No. 556813-39-9), BIO-acetoxime (CAS No. 667463-85-6), BIO (CAS No. 667463-62-9), LiCl, SB 216763 (CAS No. 280744-09-4), SB 415286 (CAS No. 264218-23-7), AR A014418 (CAS No. 487021-52-3), 1-Azakenpaullone (CAS No. 676596-65-9), Bis-7-indolylmaleimide (CAS No. 133052-90-1), and any combinations thereof. In some embodiments, any of CHIR 99021, CHIR 98014, and BIO-acetoxime are used to inhibit Gsk3 in the differentiation methods described herein. In another example, the Gsk3 inhibitor is selected from the group consisting of CHIR 99021, CHIR 98014, BIO-acetoxime, BIO, LiCl, SB 216763, SB 415286, AR A014418, 1-Azakenpaullone, and Bis-7-indolylmaleimide. In a preferred embodiment, the Gsk3 inhibitor is CHIR 99021.

In one embodiment, the small molecule Gsk3 inhibitor to be used is CHIR99021. Suitable concentrations of the inhibitor can be determined by one skilled in the art. For example, in one embodiment, the CHIR99021 can be used at a concentration ranging from about 4 μM to about 15 μM, e.g., about 4 μM, 5 μM, 6 μM, 7 μM, 8 μM, 9 μM, 10 μM, 12 μM, or another concentration of CHIR99021 from about 4 μM to about 15 μM, preferably from about 6 μM to about 8 μM. In another embodiment, the small molecule Gsk3 inhibitor to be used at a concentration ranging from about 4.0 μM to about 8.0 μM, e.g., about 4.5 μM, 5.0 μM, 5.5 μM, 6.0 μM, 6.7 μM, 7.0 μM, or another concentration of from about 4.0 μM to about 8.0 μM.

In other embodiments, Gsk3 activity is inhibited by RNA interference knockdown of Gsk3. For example, Gsk3 expression levels can be knocked-down using commercially available siRNAs against Gsk3, e.g., SignalSilence® GSK3α/β siRNA (catalog #6301 from Cell Signaling Technology®, Danvers, Mass.), or a retroviral vector with an inducible expression cassette for Gsk3, e.g., a commercially available Tet-inducible retroviral RNAi system from Clontech (Mountain View, Calif.) Catalog No. 630926, or a cumate-inducible system from Systems Biosciences, Inc. (Mountain View, Calif), e.g., the SparQ® system, catalog no. QM200PA-2.

In other embodiments, activation of Wnt/β-catenin signaling is achieved by treatment with one or more Wnt ligand(s). For example, the ligands Wnt3a, Wnt7a, or Wnt7b, or combinations thereof, can be used at concentrations ranging from about 10 ng/mL to about 200 ng/mL.

This method described herein produces a homogenous population of CD31⁺BMECs which can be used for a blood brain barrier system described herein. The CD31⁺ EECM-BMECs are characterized by (a) a lower permeability as compared to naïve endothelial cells; (b) increased expression of claudin-5 as compared to naïve endothelial cells; (c) increased expression of GLUT1 as compared to naïve endothelial cells; (d) reduced expression of plasmalemma vesicle-associated protein (PLVAP) as compared to naïve endothelial cells; or (e) any combination of (a)-(d). These cells came be used alone or in combination with the cells derived by the other methods of the invention to provide an in vitro blood-brain barrier model for studying immune cell migration and regulation. The BBB model comprises a confluent monolayer of the population of CD31+GLUT+ BMECs cultured on surface, preferably a permeable surface within a system. A suitable surface includes, for example, a collagen coated permeable membrane within a tissue culture system or device.

The term “endothelial progenitor cells,” as used herein, refers to CD34+CD31+ progenitors made by the method described in the Examples and shown in the first part of FIG. 3A. Specifically, in some embodiments, the endothelial progenitor cells of step (a) are differentiated from pluripotent stem cells. The method of producing endothelial progenitor cells from pluripotent stem cells, including induced pluripotent stem cells, is described in U.S. Pat. No. 9,290,741, the contents of which are incorporated by reference in its entirety. Briefly, the method for generating endothelial progenitor cells from human pluripotent stem cells, comprises: (i) contacting cultured human pluripotent stem cells with an activator of Wnt/β-catenin signaling for a period of about two days in a cell culture medium suitable for maintenance of human endothelial cells, but substantially free of exogenous growth factors; and (ii) obtaining a cell population comprising endothelial cells by culturing the contacted cells in the absence of the activator, for at least about three days to about ten days, in a cell culture medium suitable for maintenance of human endothelial cells but substantially free of exogenous growth factors. Suitable cell culture medium are known in the art, and include, for example, Advanced™ DMEM, VcG-Advanced™ DMEM, LaSR medium, Advanced™ DMEM-F12; VcG-Advanced™ DMEM-F12, StemPro® 34 medium, Advanced™ RPMI, and VcG-Advanced™ RPMI. The endothelial progenitor cells are CD34+CD31+. The cells may also be characterized as CD34⁺CD31⁺vWF⁺CD144⁺ endothelial progenitor cells. Other suitable methods of deriving the CD34+CD31+ endothelial progenitor cells from pluripotent stem cells may be used and are contemplated in the methods described herein.

As used herein, the term “human pluripotent stem cell” (hPSC) means a cell capable of differentiating into cells of all three germ layers. Examples of hPSCs suitable for the methods described herein include human embryonic stem cells (hESCs) and human induced pluripotent stem cells (hiPSCs). As used herein, “iPS cells” refer to cells that are substantially genetically identical to their respective differentiated somatic cell of origin and display characteristics similar to higher potency cells, such as ES cells, as described herein. The cells can be obtained by reprogramming non-pluripotent (e.g., multipotent or somatic) cells. As used herein, “iPS cell derivation” means reprogramming a somatic cell to become pluripotent.

The methods of the present disclosure also produce the unique byproduct of the non-endothelial cells that are not selected in the methods described herein. These non-endothelial cells can be differentiated into smooth muscle-like cells (SMLCs), which can be used in turn to augment the expression of adhesion molecules on the EECM-derived BMECs by either co-culture or use of their conditioned medium.

Thus, in one embodiment, the present disclosure provides methods for producing a cell population comprising smooth muscle-like cells from a cell population comprising CD34+CD31+ endothelial progenitor cells. The methods comprise the steps of: (a) culturing a cell population of CD34+CD31+ endothelial progenitor cells in serum-free endothelial medium on collagen coated surface until confluent; (b) selectively passaging the non-endothelial cells of (a); and (c) culturing the non-endothelial cells of (b) for about 6 days to about 10 days to produce PECAM-1-/α-smooth muscle actin+ smooth muscle-like cells (SMLCs). In some embodiments, the non-endothelial cells are cultured on a surface coated with fibronectin.

In some embodiments of these methods for producing smooth muscle-like cells, the culturing in step (a) comprises: (i) culturing the cell population in serum-free endothelial medium on collagen coated surface in the presence of a ROCK inhibitor, optionally for 24 hours; and (ii) removing the ROCK inhibitor and culturing the cells of (i) in serum-free endothelial medium on collagen coated surface until confluent.

The present disclosure further provides (1) homogenous populations of CD31⁺ EECM-BMECs obtained by the methods disclosed herein, and (2) cell populations comprising PECAM-1⁻/α-smooth muscle actin⁺ smooth muscle-like cells produced by the methods disclosed herein.

### In Vitro Blood-Brain Barrier Model and Methods of Use

The present disclosure provides an improved in vitro blood-brain barrier (BBB) model that has both BBB properties and a mature immune phenotype that is lacking in previously disclosed in vitro BBB models. In the Examples, the inventors demonstrate that two well-established protocols (co-differentiation unconditioned medium method (UMM) (14, 16, 23) and chemically-defined medium method (DMM) (17)) for differentiating hiPSCs to BMEC-like cell models both yield BMEC-like cells lacking expression of several key adhesion molecules known to be involved in immune cell migration across the BBB in vivo. The present BBB model addresses this specific shortcoming by differentiating hiPSC-derived endothelial progenitor cells (EPCs) to BMEC-like cells that display an improved immune phenotype, while still also displaying barrier properties similar to those observed for primary human brain endothelial cells (24-28). Importantly, these EECM-BMECs faithfully reproduce the required molecular repertoire needed for the study of immune cell trafficking across the BBB.

Because of the unique tightness of the BBB vascular bed, immune cell migration across the BBB requires unique adaptations. These adaptations range from the predominant role of α4 β1-integrin in mediating interaction of T cells with endothelial VCAM-1 (6) to the extended crawling of T cells mediated by endothelial ICAM-1 and ICAM-2 against the direction of blood flow in search of rare sites permissive for diapedesis across the BBB (7, 8). The present disclosure provides an in vitro BBB model that can be used to study this multi-step process of immune cell migration across the BBB, especially in inflammatory neurological diseases.

Thus, in one embodiment, the disclosure provides an in vitro blood-brain barrier model for studying immune cell migration and regulation. The model comprises a confluent, homogeneous monolayer of the CD31⁺ EECM-BMECs described herein cultured on surface, preferably a permeable surface, for example, a collagen coated permeable membrane within a tissue culture system. In some embodiments, the EECM-BMECs are produced by the methods described herein. Suitably, the BBB model has barrier properties considered appropriate for an in vitro BBB model, for example, having a permeability for sodium fluorescein of less than about 1×10⁻³ cm/min.

The central nervous system (CNS) is an immune privileged site where the brain barriers form compartments with different accessibility to immune cells. The CNS is virtually resistant to generating immune reactions to antigens locally deposited in the CNS parenchyma. Thus, in the absence of neuroinflammation, the BBB normally limits immune cell trafficking to specific immune cell subsets to ensure CNS immune surveillance. However, in the presence of neuroinflammation, there is a breakdown of this barrier to immune cells which can result in localized inflammation and cellular degradation, and/or dysregulation of the CNS immune surveillance mechanism. BBB model of the present invention may be used to study the effect of neuroinflammation on immune cell trafficking, and to help identify therapeutics that may restore the barrier properties of the BBB to immune cells.

Thus, the in vitro blood-brain barrier models described herein may also be used to identify therapeutics and their immunomodulatory effects on the BBB, especially in neuroinflammatory diseases. For example, in one embodiment, the BBB model may be used for screening and identifying compounds that may alter the immunological properties or immune phenotype of the BBB. Alternatively, the BBB model may be used for identifying therapeutic targets for the treatment of neuroinflammatory diseases, neurodegenerative diseases, or disorders involving immune components. Thus, methods of identifying therapeutic targets for the treatment of such diseases and disorders are also provided. These methods comprise: (a) contacting the in vitro blood-brain barrier model of any one of claims 19-21 with a therapeutic target; and (b) determining the disruption and/or restoration of the blood-brain barrier model.

In some embodiments, the blood-brain barrier models of the present invention are isogenic models comprising isogenic cells. As used herein, the terms “isogenic model” or “autologous model” are used to refer to a model made from cells that are selected and engineered to accurately model the genetics of a specific patient or healthy individual in vitro. An isogenic EECM-BMEC cell population or BBB model may be made by using patient-specific iPSCs. Patient-specific iPSCs can be made by isolating cells (e.g., somatic cells) from a patient and reprograming the cells to iPSCs. The isogenic iPSCs can then be differentiated into progenitor ECs to be used in the methods provided herein. The resulting isogenic model can be used to study immune cell migration across a particular patient's blood-brain barrier.

In one embodiment, the isogenic blood-brain barrier model is specific to a subject having a neuroinflammatory or neurodegenerative disease. The blood-brain barrier model comprises a confluent monolayer of the CD31+EECM-BMECs described herein cultured on a surface, preferably a permeable surface (e.g., collagen coated permeable membrane). In this model, the CD31+ EECM-BMECs are derived from endothelial progenitor cells differentiated from pluripotent stem cells derived from the subject. These isogenic BBB models could be used to screen for therapeutics that may improve the BBB properties and result in reduced symptoms of the neuroinflammatory or neurodegenerative disease.

Exemplary neuroinflammatory or neurodegenerative diseases that may be studied using the disclosed in vitro BBB model include, but are not limited to, for example, multiple sclerosis, stroke, meningitis, and sepsis, among others. The expression and regulation of adhesion molecules are extremely important when aiming to apply hiPSC-derived in vitro BBB models to study cerebrovascular pathologies involved in multiple sclerosis, stroke or CNS infections, where immune cell trafficking across the BBB critically contributes to disease pathogenesis. Previous reports have suggested that inflammatory stimuli could induce expression of ICAM-1 (17, 22) and VCAM-1 (22) in hiPSC-derived BMEC-like cells, but detailed characterization of the full panel of adhesion molecules shown to mediate the multi-step immune cell migration across the BBB has not been reported. Thus, the BBB model of the present invention may provide key insights that contribute to the understanding of the regulation and dysregulation of adhesion molecules in neuroinflammation and disease progression.

Many of these neuroinflammatory or neurodegenerative diseases involve immune cell extravasation at the blood-brain barrier. Extravasation is the process in which cells, i.e., immune cells, flowing in a vascular vessel interact with the endothelial luminal side, adhere to it in a multi-step process which ultimately leads to their crossing the endothelial barrier to reach a target site, guided by various types of stimulation. This process represents a key step in several pathologic conditions. For example, leukocytes typically extravasate in inflammatory conditions and although the inflammatory response is fundamental to fight infection and in wound healing, the persistency of an active immune response is involved in several pathologies and chronic inflammatory disorders. Thus, in some embodiments, the in vitro blood-brain barrier model described herein may be used to study multi-step immune cell extravasation across the blood-brain barrier by using the cells described herein on a permeable membrane under flow conditions (e.g., conditions that mimic the blood flow). The blood brain barrier model can be used to study the interaction of immune cells with the molecules expressed by the blood brain barrier, and allow for a better understanding of the mechanism by which immune cells are able to migrate across the blood brain barrier.

### Kits

The present invention further provides kits for carrying out the methods as described herein. In one embodiment, the disclosure provides a kit for differentiating endothelial progenitor cells (EPCs) to EECM-BMECs. The kit comprises serum-free endothelial culture medium, a collagen coated surface, and instructions describing the method of differentiating the EPCs. The kit may further comprise endothelial progenitor cells (EPCs). In another embodiment, the kit further comprises iPSCs and instructions on differentiating the iPSCs into EPCs. In another embodiment, a kit for making an isogenic BBB model is provided. The kit comprises the components described herein for differentiating iPSCs derived from a subject into EECM-BMECs.

In a further embodiment, the kit provides an allogenic in vitro BBB model as described herein. In yet another embodiment, the kit provides EECM-derived BMECs and a permeable support that can be used to prepare a BBB model. In some embodiments, the EECM-derived BMECs are cryopreserved, e.g., stored at −80° C. or less, prior to use in the kit. The kit further provides instructions for use in forming a BBB model.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

All publications, patent applications, patents, and other references mentioned herein are incorporated by reference in their entirety. In the case of conflict, the present specification, including definitions, will control.

Other features and advantages of the invention will be apparent from the description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

### EXAMPLES

### Example 1: Derivation of EECM-BMEC and a Blood Brain Barrier Model System

Human induced pluripotent stem cell (hiPSC)-derived blood-brain barrier (BBB) models established to date lack expression of key adhesion molecules involved in immune cell migration across the BBB in vivo. In the following Example, the inventors introduce the extended endothelial cell culture method (EECM), which differentiates hiPSC-derived endothelial progenitor cells to brain microvascular endothelial cell (BMEC)-like cells with good barrier properties and mature tight junctions. Importantly, EECM-BMEC-like cells exhibited constitutive cell surface expression of ICAM-1, ICAM-2 and E-selectin. Pro-inflammatory cytokine stimulation increased cell surface expression of ICAM-1 and induced cell surface expression of P-selectin and VCAM-1. Coculture of EECM-BMEC-like cells with hiPSC-derived smooth muscle-like cells or their conditioned medium further increased induction of VCAM-1. Functional expression of endothelial ICAM-1 and VCAM-1 was confirmed by T-cell interaction with EECM-BMEC-like cells. Taken together, we introduce the first hiPSC-derived BBB model that displays an adhesion molecule phenotype that is suitable for the study of immune cell interactions.

### Materials and Methods:

### Human Induced Pluripotent Stem Cells

Human induced pluripotent stem cells (hiPSCs) were reprogrammed, expanded and characterized for pluripotency and differentiation capacity as described before (29). In brief, erythroblasts from three donors (age/sex: donor 1: 27/F, donor 2: 50/M, donor 3: 49/F) were reprogrammed by nucleofection of plasmids encoding for OCT4, shRNA-p53, SOX2, KLF4, L-Myc and Lin28. Human iPSCs were cultured using ReproTeSR medium (STEMCELL Technologies, Grenoble, France) and expanded using StemMACS iPSC-Brew XF medium (Miltenyi Biotec, Bergisch Gladbach, Germany). Five hiPSC clones from three donors (donor 1: 2 clones, donor 2: 2 clones, donor 3: 1 clone) were used in this study. For some experiments, we also used the IMR90-4 iPSC line (30). Human iPSCs were maintained on Matrigel (Corning)-coated plates in mTeSR1 medium (STEMCELL Technologies, Grenoble, France).

### Brain-Like Endothelial Cells (BLECs) and Primary BMECs

Brain-like endothelial cells (BLECs) were obtained exactly as described before (15, 31). In brief, CD34⁺ cells were isolated from human umbilical cord blood and differentiated to endothelial cells in ECM basal medium (ScienCell, Carlsbad, CA 92008, USA) supplemented with 20% (v/v) fetal bovine serum (FBS; Life Technologies, Carlsbad, CA, USA) and 50 ng/mL of VEGF165 (PeproTech Inc., Rocky Hill, NJ, USA). To induce a BBB phenotype, CD34⁺ cell-derived endothelial cells were cultured on Transwell filters (0.4 μm pore size, polycarbonate membrane, Corning 3401; New York, NY, USA) and start co-culture with bovine pericytes at the same day for 6 days using ECM-5 medium (ECM basal medium (ScienCell) supplemented with 5% FBS, 1% endothelial cell growth supplement (ECGS; ScienCell), and 50 mg/mL gentamycin (Biochrom AG, Berlin, Germany)). Primary human microvascular endothelial cells (pHBMECs) derived from autopsy tissue were purchased from PELOBiotech (Planegg/Martinsried, Germany). HBMECs were grown in endothelial cell medium supplemented with fetal bovine serum, endothelial cell growth supplement and penicillin/streptomycin solution (ScienCell Research Laboratories).

Differentiation of hiPSCs into Brain Microvascular Endothelial-Like Cells (BMEC-Like Cells)

Unconditioned medium method (UMM) (14, 16) and defined medium method (DMM) (17) were slightly modified and used to differentiate brain microvascular endothelial-like cells (BMEC-like cells). In brief, hiPSCs were seeded onto a Matrigel-coated plate in mTeSR1 supplemented with 10 μM ROCK inhibitor Y-27632 (Selleckchem, Houston, TX, USA) for 24 hours (day −3 or day −4). Seeding densities at day −3 or day −4 were optimized at 8,000/cm² to 21,000/cm² for UMM, and between 35,000/cm² to 150,000/cm² for DMM depending on donor and passage number (Table 1), to obtain transendothelial electrical resistance (TEER) of more than 2,000 Ω×cm² and permeability to sodium fluorescein (NaFl, 376.3 Da) less than 0.6×10⁻⁴ cm/min. hiPSCs were then expanded in mTeSR1 medium for 2-3 days depending on hiPSC clone. At day 0, cells were induced to differentiate using specific medium for each method: For UMM, medium was switched to unconditioned medium (DMEM/F12 with 20% Knockout serum replacement, 1× non-essential amino acids, 0.5× Glutamax, and 0.1 mM β-mercaptoethanol) and changed every day for 6 days. For DMM, medium was switched to DeSR1 (DMEM/F12 with 1× non-essential amino acids, 0.5× Glutamax, and 0.1 mM β-mercaptoethanol) supplemented with 6 μM CHIR99021 (Selleckchem, Houston, TX, USA) and cultured for 24 hours. Then the medium was switched to DeSR2 (DeSR1 plus 1×B27 supplement) every day for another 5 days. At day 6, for both UMM and DMM, medium was switched to hECSR (human endothelial serum free medium (hESFM, Thermo Fisher Scientific, Waltham, MA, USA) supplemented with 1×B27 and 20 ng/mL bFGF) and cultured for 2 days. For retinoic acid (RA) treatment, 10 μM RA was added to hECSR medium. At day 8, cells were replated onto collagen IV (400 μg/ml)/fibronectin (100 μg/ml)- or Matrigel-coated, respectively for UMM and DMM, Transwell filters (0.4 μm pore size, polycarbonate membrane, Corning 3401) or culture plates at 1,000,000/cm² in hECSR medium. At day 9, the medium was switched to hECSR2 (hECSR lacking bFGF or RA) and cells were used for assays at day 10.

TABLE 1
Seeding densities of hiPSCs clones time until confluency employing
the extended endothelial cell culture method (EECM)

| | | | | Average range of time |
|---|---|---|---|---|
| | UMM | DMM | EECM | for become confluent |
| Clone name | (/cm²) | (/cm²) | (/cm²) | in EECM (day) |
| Donor 1 | 21,000 | 150,000 | 100,000 | 5-7 |
| Donor 2 | 10,000 | 36,000 | 26,000 | 3-5 |
| Donor 3 | 10,000 | 36,000 | 21,000 | 3-5 |
| IMR90-4 | 10,000 | 36,000 | 20,000 | 4-8 |

Differentiation of hiPSCs into Endothelial Progenitor Cells (EPCs).

CD34⁺ CD31⁺ endothelial progenitor cells (EPCs) were differentiated from hiPSCs as described (32, 33). In brief, hiPSCs were seeded onto a Matrigel-coated plate in mTeSR1 supplemented with 5 μM ROCK inhibitor Y-27632 for 24 hours (day −3). Seeding densities at day −3 were optimized between 16′000/cm² to 132′000/cm² depending on donor and passage in order to obtain high number of CD34⁺ CD31⁺ EPCs. At day 0 and day 1, medium was changed to LaSR basal medium (Advanced DMEM/F12, 2.5 mM GlutaMAX, and 60 μg/ml ascorbic acid) supplemented with 8 μM CHIR99021. At day 2, medium was switched to LaSR basal medium and changed every day for another 3 days. At day 5, CD31⁺ EPCs were purified using FITC-conjugated human CD31 antibody (Miltenyi Biotec, clone AC128) and EasySep Human FITC Positive Selection Kit II (STEMCELL Technologies) with an Easy Sep Magnet kit (STEMCELL Technologies). Purified EPCs were seeded onto collagen IV/fibronectin-coated Transwell filters at a density of 100′000/cm² or collagen-IV (10 μg/ml)-coated culture plates at a density of 10′000-20′000/cm² in hECSR medium and used for assay or further extended EC culture.

### Extended Endothelial Cell Culture Method (EECM) and Culture of Smooth Muscle-Like Cells (SMLC)

EPCs purified as described above were plated on collagen IV (10 μg/ml)-coated plates in hECSR medium in the presence of 5 μM ROCK inhibitor Y-27632 of 10′000-20′000/cm² as described before (33). Twenty-four hours later, medium was changed to hECSR without ROCK inhibitor and then medium was changed every 2 days. Once the cells reached 100% confluency (FIG. 3B (i)), the cells were passaged using Accutase (Innovative Cell Technologies, San Diego, CA, USA). In detail, medium was removed and 1 ml of Accutase was added to the 6-well plate and cells were carefully observed under an inverted microscope (Zeiss Axiovert 25). Once endothelial cells started to become round (FIG. 3B (ii)), the endothelial cells were physically detached by tapping the 6-well plate (FIG. 3B (iii)). Since endothelial cells detached earlier than non-endothelial cells, preferentially detached endothelial cells were collected in a 15 ml conical tube containing 4 ml of hECSR and centrifuged, resuspended and seeded onto collagen IV (400 μg/ml)/fibronectin (100 μg/ml)-coated Transwell filters at a density of 100′000/cm² or collagen IV (10 μg/ml)-coated culture plates at a density of 10′000-20′000/cm² in hECSR medium. These cell densities were found optimal for achieving closed monolayers. Coating with collagen IV/fibronectin for Transwell filters was chosen as it was previously shown to be optimal for growing primary BMECs and for UMM-BMEC-like cells. Here we found that combination of collagen IV and fibronectin also enhanced adhesion of both EECM-BMEC-like cells and SMLCs. Therefore, we used only collagen IV (10 μg/ml)-coated culture plates during the selective passaging process to aid in SMLC depletion (FIG. 3A). For clarity, we refer to D5 CD34⁺CD31⁺ cells as EPCs, and refer to cells that transitioned from EPCs to ECs after 6 days of culture in the EC culture medium as naïve ECs, and refer to “EECM-BMEC-like cells” starting at passage 2 (FIG. 3A). In some experiments, EECM-BMEC-like cells were cultured using ECM-5 medium. Human iPSC-derived smooth muscle-like cells (SMLCs) were obtained after harvesting preferentially detached endothelial cells (FIG. 3B (c)) by adding 2 ml of hECSR medium to 6-well plate and culturing approximately 6-10 days until confluence. Culture medium was collected every 2 days for SMLC-derived conditioned medium (CM). List of materials used in the EECM-BMEC-like cell differentiation protocol is shown in Table 2.

TABLE 2
Material list for EECM-BMEC-like cell protocol

| Material | Company | Catalog No. |
|---|---|---|
| ROCK inhibitor Y-27632 | Tocris | 1254 |
| Human fibroblast growth factor 2 | Tocris | 233-FB-500 |
| Accutase | Innovative Cell | AT104-500 |
| | Technologies | |
| CHIR99021 | Selleckchem | S1263 |
| 40 μmFalcon cell strainer | Falcon | 352340 |
| CD31-FITC antibody (clone | Miltenyi Biotec | 130-110-668 |
| AC390) | | |
| L-ascorbic acid | Sigma | A4403-5G |
| EasySepFITC Positive Selection | Stemcell Technologies | #18558 |
| Kit | | |
| Easy SepMagnet | Stemcell Technologies | #18000 |
| advanced DMEM/F-12 | Life Technologies | 12634 |
| Human endothelial serum-free | Thermo Fisher | 11111-044 |
| medium (hESFM) | | |
| B27, 50× | Thermo Fisher | 17504044 |
| Water, sterile, cell culture | Sigma | W3500 |
| Collagen IV from human placenta | Sigma | C5533 |
| Fibronectin from bovine plasma | Sigma | F1141 |

Co-Culture of BMEC-Like Cells with Pericytes, Astrocytes, or Smooth Muscle-Like Cells (SMLC)

Bovine pericytes (15), the human brain pericyte cell line (HBPCT) (34), the human astrocyte cell line (hAST) (35), and hiPSC-derived astrocytes (iPS_AC) (29) were obtained and maintained as previously described. Since HBPCT and hAST cell lines were immortalized with a temperature dependent SV40-antigen, these cells were expanded at 33° C. and switched to 37° C. when seeded for co-culture with EECM-BEMC-like cells. HBPCT and hAST cells were shown before to stop proliferating two days after the temperature switch (36). Generally, pericytes and astrocytes were seeded at a density of 26′000/cm² for 2 days before starting the co-culture with EECM-BMEC-like cells using hECSR and incubated at 37° C. (5% CO₂) to become confluent. SMLCs were seeded at a density of 13′000/cm² one day before starting the co-culture using hECSR. EECM-BMEC-like cells were seeded on collagen IV (400 μg/ml)/fibronectin (100 μg/ml)-coated Transwell inserts at a density of 100′000/cm² and the co-culture was initiated on the same day. Co-cultures were maintained for 6 days using hECSR for both the apical and basolateral compartment, with medium changes every other day. Transendothelial electrical resistance (TEER) was measured using a Volt-Ohm-Meter (Millicell ERS-2, MERSSTX01-electrode). To calculate the net resistance in Ω×cm² of the cell monolayers, TEER value of an empty filter was subtracted from each measurement and TEER values in Ω were multiplied by the surface area of the filters (1.12 cm²) as follows: TEER (Ω×cm²)=(cell monolayer resistance (Ω)−empty Transwell filter resistance (Ω))×surface area (cm²).

Treatment of EPCs with Retinoic Acid (RA) or Pericyte Conditioned Medium (PCM)

EPCs purified as described above were plated on collagen-IV (10 μg/ml)-coated plates in respective medium. For RA treatment, 10 μM RA was added to hECSR for 3 days and then switched to hECSR medium without RA. For pericyte conditioned medium (PCM) treatment, bovine pericytes were cultured in hECSR medium at a density of 26′000/cm² and culture medium was collected 24 hours later and used as PCM. EPCs were treated with PCM for 6 days. After 6 days of culture, EPCs were detached with Accutase and seeded onto collagen IV/fibronectin-coated Transwell filters at a density of 100′000/cm² in hECSR medium. TEER values were measured over 6 days and permeability assay was done at day 6 after seeding onto filters.

### Investigation of Cell Surface Expression of Adhesion Molecules by Flow Cytometry

BMEC-like cells differentiated by UMM, DMM, or EECM and naïve ECs were cultured on collagen IV (10 μg/ml) coated plates at a density of 10′000/cm² or collagen IV (400 μg/ml)/fibronectin (100 μg/ml)-coated Transwell inserts at a density of 100′000/cm² in respective media. Some wells were stimulated with 10 ng/mL of recombinant human TNF-α (R&D systems, 210TA, Minneapolis, MN, USA) and 200 IU/mL recombinant human IFN-γ (R&D systems, 285IF) for UMM- or DMM-differentiated BMEC-like cells, and 1 ng/mL of recombinant human TNF-α and 20 IU/mL recombinant human IFN-γ, or 1 ng/ml of recombinant human IL-1β (R&D systems, 201-LB/CF) for EECM-BMEC-like cells or naïve ECs for 16 h at 37° C. (5% CO₂). Stimulated and nonstimulated control cells were gently detached with Accutase, washed, and resuspended in FACS-buffer (DPBS (1×), 2.5% FBS, 1% NaN₃). Then, 1×10⁵ cells per well were transferred to a 96-well microtiter plate and incubated 20 min on ice with the fluorochrome-conjugated antibodies or respective isotype controls (Table 3). After staining, cells were washed twice with DPBS and measured with an Attune NxT Flow Cytometer (Thermo Fisher Scientific). Data were analyzed using FlowJo 10 software (Tree Star, Ashland, OR, USA).

TABLE 3
Fluorophore labelled antibodies and isotype controls for flow cytometry analysis

| Antibodies | Fluorophore | Clone | Source | Cat. N. | Isotype |
|---|---|---|---|---|---|
| Anti-human ICAM-1 | BV421 | HA58 | BD Biosciences | 564077 | m-IgG1, κ |
| Anti-human ICAM-2 | PE | CBR-IC2/2 | BD Biosciences | 558080 | m-IgG2a, κ |
| Anti-human VCAM-1 | FITC | 51-10C9 | BD Biosciences | 551146 | m-IgG1, κ |
| Anti-human P-selectin | BV510 | AC1.2 | BD Biosciences | 743756 | m-IgG1, κ |
| Anti-human E-selectin | APC | 68-5H11 | BD Biosciences | 551144 | m-IgG1, κ |
| Anti-human CD99 | PE-Cy7 | 3B2/TA8 | Biolegend | 371314 | m-IgG2a, κ |
| Anti-human PECAM-1 | APC-Cy7 | WM59 | BD Biosciences | 563653 | m-IgG1, κ |
| Anti-human VE-cadherin | PerCP/Cy5.5 | 55-7H1 | BD Biosciences | 561566 | m-IgG1, κ |

| Isotype controls | Fluorophore | Clone | Source | Cat. N. |
|---|---|---|---|---|
| m-IgG1, κ | APC | MOPC-21 | BD Biosciences | 555751 |
| m-IgG1, κ | BV421 | X40 | BD Biosciences | 562438 |
| m-IgG2a, κ | PE | eBM2a | Invitrogen | 12-4724-42 |
| m-IgG1, κ | FITC | MOPC-21 | Biolegend | 400108 |
| m-IgG1, κ | BV510 | X40 | BD Biosciences | 562946 |
| m-IgG2a, κ | PE-Cy7 | MOPC-173 | Biolegend | 400232 |
| m-IgG1, κ | PerCP/Cy5.5 | MOPC-21 | Biolegend | 400150 |
| m-IgG1, κ | APC-Cy7 | MOPC-21 | BD Biosciences | 557873 |

### Immunofluorescence Staining

BMEC-like cells differentiated by UMM, DMM, or EECM, naïve ECs, and SMLCs were cultured on Transwell inserts as described above or collagen IV (10 μg/ml) coated chamber slides (Thermo Fisher Scientific). Same amounts of proinflammatory cytokine (1 ng/mL TNF-α+20 IU/mL IFN-γ or 1 ng/mL IL-1β) for Transwell inserts disrupted the EECM-BMEC-like cell monolayers grown on chamber slides; therefore we titrated and used 0.1 ng/mL TNF-α+2 IU/mL IFN-γ or 0.1 ng/ml IL-1β) for stimulation of EECM-BMEC-like cells cultured on chamber slides. To stain for claudin-5, occludin, VE-cadherin, PECAM-1, ZO-1, and N-cadherin cells were fixed with −20° C. precooled methanol for 20 seconds and rehydrated during subsequent washing steps in PBS. Cells were blocked and permeabilized with 5% skimmed milk containing 0.1% Triton X-100 for 10 minutes at RT, and then stained with primary antibodies diluted in 5% skimmed milk in PBS for 1 hour at RT as described (16, 17, 37). For staining of the adhesion molecules ICAM-1, ICAM-2, VCAM-1, ALCAM, MCAM, primary antibodies were diluted in hECSR medium, added to live cells, and incubated at 37° C. (5% CO₂) for 15 minutes. This allowed for selective detection of cell surface-expressed adhesion molecules, which are functionally available for immune cell interaction. After washing with PBS, cells were fixed with 1% (w/v) formaldehyde in PBS for 10 minutes at RT. Cells were washed with PBS and blocked with 5% skimmed milk in PBS for 10 minutes at RT and then incubated with secondary antibodies for 1 h at RT as described (37). For the staining of P-selectin, E-selectin, CD99, α-smooth muscle actin, NG2, and vimentin, cells were first fixed with 1% (w/v) formaldehyde in PBS for 10 minutes at RT and blocked and permeabilized with 5% skimmed milk in PBS containing 0.1% Triton X-100 for 10 minutes at RT. Cells were then incubated with primary antibodies for 1 hour at RT diluted in 5% skimmed milk in PBS. To stain for calponin and SM22α, cells were fixed with 4% (w/v) formaldehyde in PBS, blocked and permeabilized with 3% BSA in PBS containing 0.1% Triton X-100 for 1 h at RT, and then stained with primary antibodies at 4° C. overnight as described (38). After three washes, cells were incubated with respective secondary antibodies for 1 hour at RT. Nuclei were stained with DAPI at 1 μg/mL or Hoechst 33342 at 4 μM. After washing with DPBS, cell monolayers on filters or chamber slides were mounted with Mowiol (Sigma-Aldrich, St. Louis, MO, USA). Images were acquired using a Nikon Eclipse E600 microscope or Nikon Eclipse Ti2-E microscope using the Nikon NIS-Elements BR3.10 software (Nikon, Tokyo, Japan) or inverted research microscope Axio observer Z1 (63× immersion oil, Apotome mode, Zeiss) equipped with a camera (Zeiss Axiocam MRm) and the ZEN-blue software. List of antibodies used in this study is shown in Table 4. Quantification of EC shape was performed using VE-cadherin immunofluorescence images by an observer blind to the experimental groups. For each image, FIJI (ImageJ) software was used to trace ten cell outlines, and the Shape Descriptors tool under the Set Measurements function of FIJI was used to calculate circularity values (4×π×area/perimeter²); average cell circularity for each image is reported.

TABLE 4
Antibody list for immunofluorescence staining

| | | | | Cat. | |
|---|---|---|---|---|---|
| Antibodies | Fixative | Clone | Source | No. | Secondary Antibody |
| VE-cadherin | MeOH | F-8 | Santa Cruz | sc-9989 | Cy3 AffiniPure F(ab′)₂ |
| | | | | | Fragment Goat Anti- |
| | | | | | Mouse IgG |
| PECAM-1 | MeOH | MEM-05 | Invitrogen | 37-0700 | Cy3 AffiniPure F(ab′)₂ |
| | | | | | Fragment Goat Anti- |
| | | | | | Mouse IgG |
| ZO-1 | MeOH | polyclonal | Invitrogen | 40-2200 | Cy3 AffiniPure Donkey |
| | | | | | Anti-Rabbit IgG (H + L) or |
| | | | | | Alexa Fluor ™ 488 |
| | | | | | donkey anti-rabbit IgG |
| | | | | | (H + L) |
| Claudin-5 | MeOH | 4C3C2 | Invitrogen | 35-2500 | Cy3 AffiniPure F(ab′)₂ |
| | | | | | Fragment Goat Anti- |
| | | | | | Mouse IgG |
| Occludin | MeOH | OC-3F10 | Invitrogen | 33-1500 | Cy3 AffiniPure F(ab′)₂ |
| | | | | | Fragment Goat Anti- |
| | | | | | Mouse IgG |
| ICAM-1 | live | HA58 | Biolegend | 353102 | Cy3 AffiniPure F(ab′)₂ |
| | | | | | Fragment Goat Anti- |
| | | | | | Mouse IgG |
| ICAM-2 | live | CBR-IC2/2 | FITZGERALD | 10R-7606 | Cy3 AffiniPure F(ab′)₂ |
| | | | | | Fragment Goat Anti- |
| | | | | | Mouse IgG |
| VCAM-1 | live | 51-10C9 | BD | 555645 | Cy3 AffiniPure F(ab′)₂ |
| | | | Biosciences | | Fragment Goat Anti- |
| | | | | | Mouse IgG |
| CD99 | 1% PFA | Hec-2 | provided from | | Cy3 AffiniPure F(ab′)₂ |
| | | | William A Muller lab | | Fragment Goat Anti- |
| | | | | | Mouse IgG |
| E-selectin | 1% PFA | HAE-1f | Biolegend | 336002 | Cy3 AffiniPure F(ab′)₂ |
| | | | | | Fragment Goat Anti- |
| | | | | | Mouse IgG |
| P-selectin | 1% PFA | AK4 | Santa Cruz | sc-19996 | Cy AffiniPure F(ab′)₂ |
| | | | | | Fragment Goat Anti- |
| | | | | | Mouse IgG |
| Smooth | 1% PFA | 1A4 | Thermo | MS-113-P | Alexa Fluor 488 Goat |
| muscle actin | | | Scientific | | Anti-Mouse IgG |
| NG-2 | 1% PFA | polyclonal | Millipore | AB5320 | Cy3 AffiniPure Donkey |
| | | | | | Anti-Rabbit IgG (H + L) |
| N-Cadherin | MeOH | 32 | BD | 610920 | Cy ™ AffiniPure F(ab′)₂ |
| | | | Transduction | | Fragment Goat Anti- |
| | | | Laboratories | | Mouse IgG |
| Vimentin | 1% PFA | polyclonal | Millipore | AB1620 | Cy3 AffiniPure Donkey |
| | | | | | Anti-Goat IgG (H + L) |
| Calponin | 4% PFA | hCP | Sigma | C2687 | Alexa Fluor 488 Goat |
| | | | | | Anti-Mouse IgG |
| SM22a | 4% PFA | polyclonal | Abcam | ab14106 | Alexa Fluor 647 Goat |
| | | | | | Anti-Rabbit IgG |
| MCAM | live | 2107 | Prothena | NB-0268 | Cy3 AffiniPure Goat |
| | | | | | Anti-Rat IgG (H + L) |
| ALCAM | live | polyclonal | R&D system | AF1172 | Cy3 AffiniPure Donkey |
| | | | | | Anti-Goat IgG (H + L) |
| Phalloidin | 1% PFA | | Sigma P5282 | | |

### Western Blotting

Naïve ECs (passage 1) and EECM-BMEC-like cells (passages 2 and 3) were lysed with RIPA buffer supplemented with Halt protease inhibitor cocktail (Thermo Scientific, 78430). The BCA assay (Pierce BCA Protein Assay Kit: Thermo Scientific, 23227) was used to determine protein concentration and 23 μg of protein resolved on 4-12% tris-glycine gels. Proteins were transferred to nitrocellulose membranes and blocked for 1 hour at room temperature in tris-buffered saline supplemented with 0.1% Tween 20 (TBST) and 5% nonfat dry milk. Membranes were incubated with primary antibodies (Table 5) diluted in TBST+5% nonfat dry milk overnight at 4° C. Membranes were washed 5× with TBST and incubated with infrared-labelled secondary antibodies (Table 5) diluted in TBST+5% nonfat dry milk for 1 hour at room temperature. Membranes were washed 5× with TBST and imaged using a LI-COR Odyssey Classic (9120). Quantification of band intensity was performed using LI-COR Image Studio software.

TABLE 5
Antibody list for Western blotting

| Antibodies | Clone | Source | Cat. No. | Secondary Antibody |
|---|---|---|---|---|
| VE-cadherin | BV9 | Santa Cruz | sc-52751 | LI-COR IRDye 800CW Goat |
| | | | | anti-Mouse IgG: 926-32210 |
| β-actin | 13E5 | Cell Signaling | 4970S | LI-COR IRDye 680RD Goat |
| | | Technology | | anti-Rabbit IgG: 926-68071 |
| ZO-1 | polyclonal | Invitrogen | 40-2200 | LI-COR IRDye 680RD Goat |
| | | | | anti-Rabbit IgG: 926-68071 |
| Claudin-5 | 4C3C2 | Invitrogen | 35-2500 | LI-COR IRDye 800CW Goat |
| | | | | anti-Mouse IgG: 926-32210 |
| Occludin | OC-3F10 | Invitrogen | 33-1500 | LI-COR IRDye 800CW Goat |
| | | | | anti-Mouse IgG: 926-32210 |
| vWF | polyclonal | Dako | A0082 | LI-COR IRDye 680RD Goat |
| | | | | anti-Rabbit IgG: 926-68071 |
| Caveolin-1 | polyclonal | Cell Signaling | 3238S | LI-COR IRDye 680RD Goat |
| | | Technology | | anti-Rabbit IgG: 926-68071 |

### Permeability (Pe) Assay

Permeability of EC monolayers was assessed by measuring the clearance of sodium fluorescein (NaFl, 376.3 Da, Sigma-Aldrich) as previously described (16). Briefly, NaFl was added to the upper compartment of the Transwell inserts at a concentration of 10 μM. Medium samples containing fluorescent tracer that had diffused across the monolayers were collected from the bottom well every 15 min for a total of 60 min, and fluorescence intensity was measured in a Tecan Infinite M1000 multi-well reader (Tecan Trading AG, Männedorf, Switzerland). The clearance principle was used to calculate the permeability coefficient (Pe) and to obtain a concentration-independent transport parameter as previously described in detail (16). This method includes blank filters without cells as a control to measure clearance across the filter membrane for appropriate calculation of the permeability coefficient across the endothelium (Pe). The experiments were done in triplicates for each condition.

### Human Th1* Cells

Human CD4⁺ T cells were isolated and sorted as previously described (39, 40). In brief, human Th1* cells were isolated by fluorescence activated cell sorting according to their specific expression pattern of chemokine receptors (CXCR3⁺CCR4⁻CCR6⁺) from the peripheral blood of healthy donors. T cells were expanded for 20 days with periodic re-stimulation with 1 μg/mL phytohaemagglutinin, irradiated allogeneic peripheral blood mononuclear cells, and human interleukin 2 (IL-2, 500 IU/mL) as previously described (39, 40). After 20 days of expansion, T cells were frozen and stored in liquid nitrogen until employed in the experiments. T cells were thawed one day prior to the respective experiment and labeled with 1 μM CellTracker Green (CMFDA Dye, Life technologies) at 37° C. (5% CO₂) for 30 min on the day of the experiment. After labelling, T cells were washed and dead cells were removed by Ficoll-Hypaque gradient (780 g, 20 min, 20° C.). T cells were washed twice and resuspended in migration assay medium (DMEM supplemented with 5% FBS, 4 mM L-Glutamine, 25 mM HEPES) at concentrations as described below.

### Adhesion Assay Under Static Condition

EECM-BMEC-like cells were cultured on collagen-IV (10 μg/ml)-coated 16-well chamber slides (Thermo Fisher Scientific) at a density of 100′000/cm² in hECSR medium or conditioned medium (CM) derived from SMLCs. EECM-BMEC-like cells were stimulated with 0.1 ng/ml recombinant human IL-1β or 0.1 ng/mL TNF-α+2 IU/mL IFN-γ for 16 h at 37° C. (5% CO₂), or maintained as nonstimulated controls. EECM-BMEC-like cell monolayers were washed twice with migration assay medium and 20′000 Th1* cells were added on top of EECM-BMEC-like cell monolayers and incubated at RT for 30 minutes using a rocking platform. Chamber slides were gently washed twice and fixed with 2.5% glutaraldehyde for 2 hours on ice. Chamber slides were then washed with DPBS and adherent fluorescencently-labelled Th1* cells per pre-defined field of view (FOV) were analyzed by fluorescence microscopy (Nikon Eclipse E600) and FIJI software (Version 2.0.0, Image J, USA). The average number of adherent cells/FOV was determined counting two fields per well. Assays were performed in at least triplicates for each condition.

### T Cell Arrest to EECM-BMEC-Like Cells Under Flow

EECM-BMEC-like cells were seeded in cloning rings placed on collagen-IV (10 μg/ml) coated Ibidi μ-dishes (Ibidi) at a density of 100′000/cm². EECM-BMEC-like cells were stimulated with 0.1 ng/ml recombinant human IL-1β for 16 h at 37° C. (5% CO₂). Live cell imaging was done as described (41). In brief, Th1* cells were perfused over EECM-BMEC-like cell monolayers and allowed to accumulate for 4 min using low shear stress (0.1 dyn/cm²). Thereafter, flow was increased to physiological shear stress (1.5 dyn/cm²). Thirty seconds after increasing shear stress, the number of arrested Th1* cells was counted.

### In Vitro Live Cell Imaging

EECM-BMEC-like cells were cultured in cloning rings placed on collagen IV (10 μg/ml)-coated Ibidi μ-dishes (Ibidi) at a density of 75′000/cm². EECM-BMEC-like cells were stimulated with 0.1 ng/ml recombinant human TNF-α+2 IU/mL IFN-γ for 16 h at 37° C. (5% CO₂) diluted in conditioned medium from SMLC. Fluorescencently-labelled Th1* cells were allowed to accumulate on the EECM-BMEC-like cell monolayer at a low flow rate of 0.1 dyne/cm² for 4 min from the first frame after the first Th1* cells appeared in the field of view (accumulation phase until 3 min55 s). After the accumulation phase of precisely 3 min 55 sec, the flow rate was set to the physiological level of 1.5 dyne/cm² for 16 min (shear phase). The dynamic T-cell interactions with the EECM-BMEC-like cell monolayers under the physiological flow were recorded at a 10× magnification with a Zeiss Axiocam MRm camera. During the recording, 1 image was acquired every 5 s, then the video was exported with a frame rate of 30 images/s. T-cell behavior on the EECM-BMEC-like cell monolayer was categorized as described previously (37). In brief, T cells found to polarize upon arrest and to migrate across the EECM-BMEC-like cells monolayer with or without prior crawling or probing on the EECM-BMEC-like cells were categorized as “diapedesis”. T cells that crawled on the surface of the EECM-BMEC-like cells for the entire observation time were categorized as “crawling”. T cells that remained stationary without displacing beyond a distance exceeding their own diameter and presenting dynamic cellular protrusions were categorized as “probing”.

### Statistical Analysis

Statistical analyses comprising calculation of degrees of freedom were done using GraphPad Prism 7 software (Graphpad software, La Jolla, CA, USA). Data are shown as the mean±SD. To compare two groups, statistical significance was assessed by paired or unpaired t-test, while more groups were analyzed by one-way ANOVA followed by Tukey's multiple comparison test (p<0.05=*, p<0.01=**, p<0.001=***, p<0.0001=****). The respective statistical methodology used for each assay is specified in corresponding Brief Description of the Drawings.

### Results:

### BMEC-Like Cells Differentiated by Unconditioned Medium Method (UMM) or Defined Medium Method (DMM) are not Suitable for Studying Immune Cell Interactions

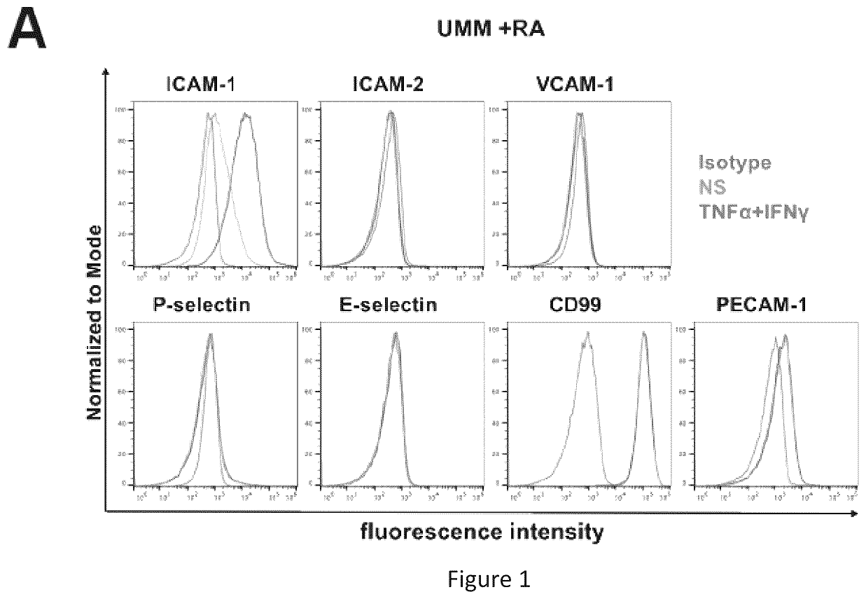

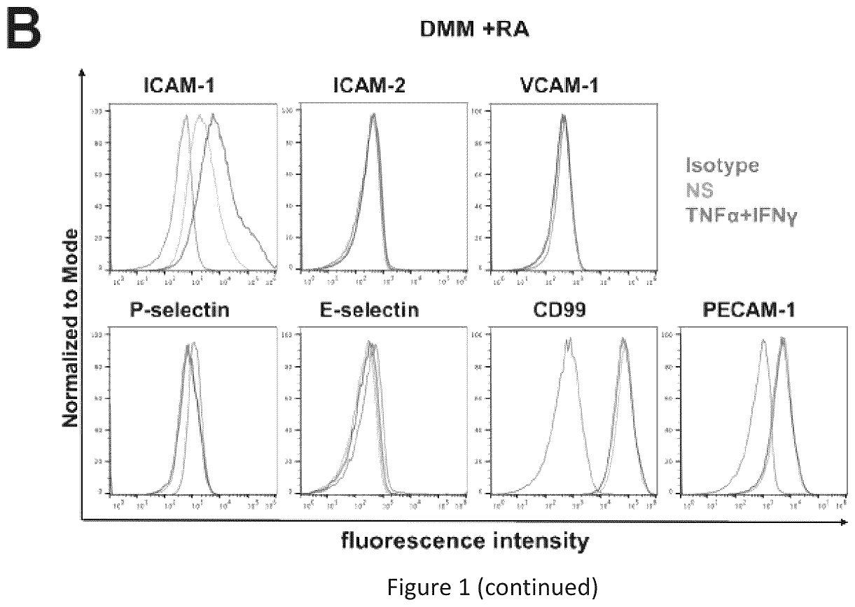

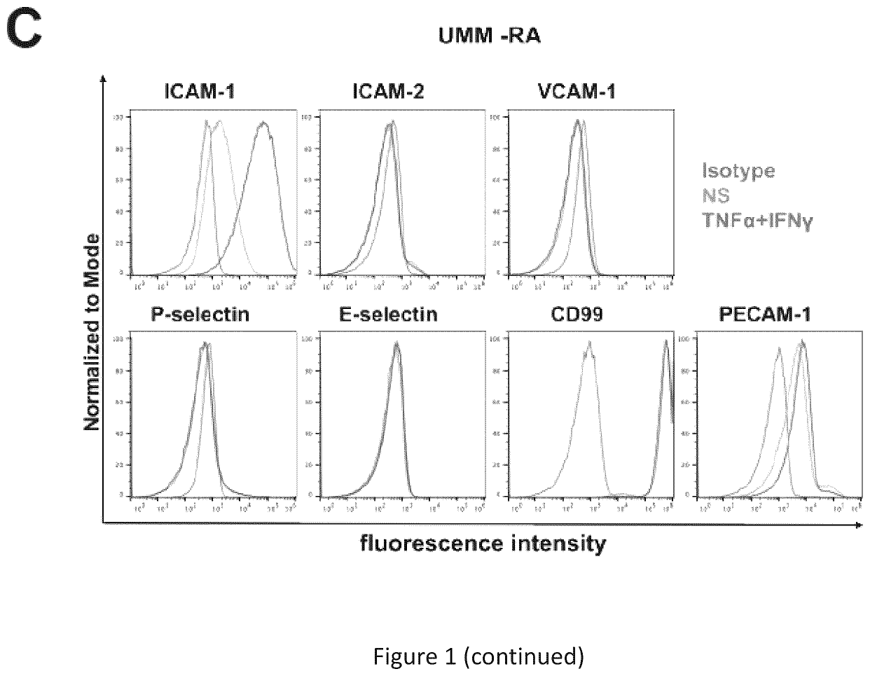

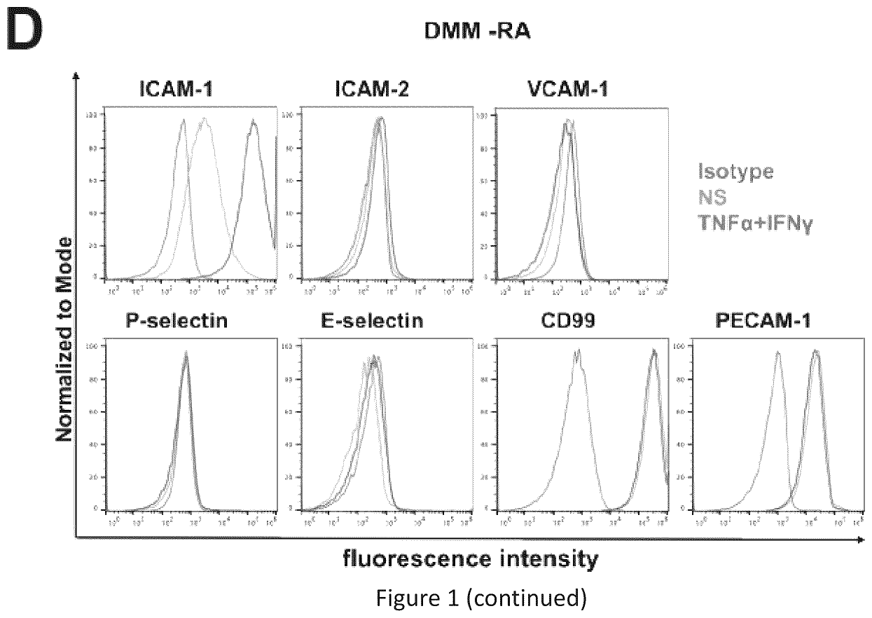

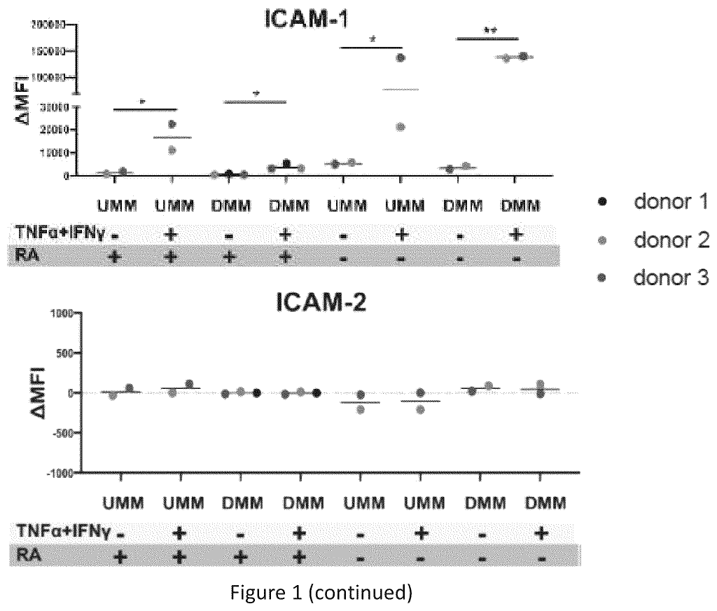

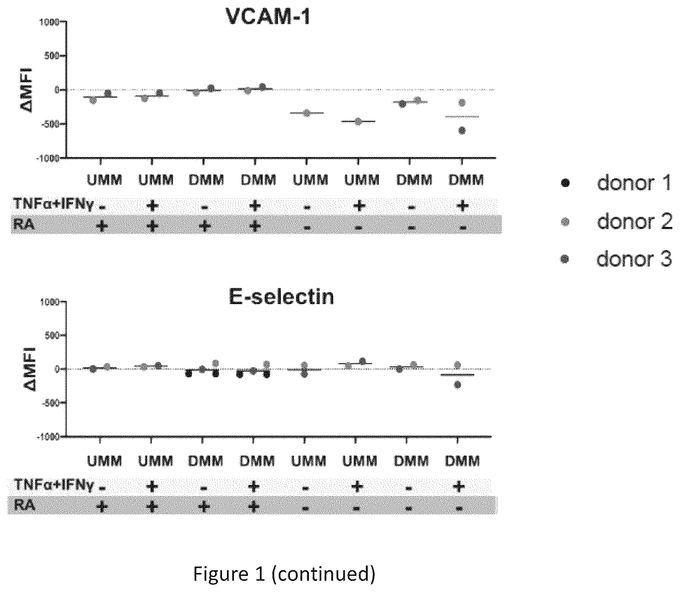

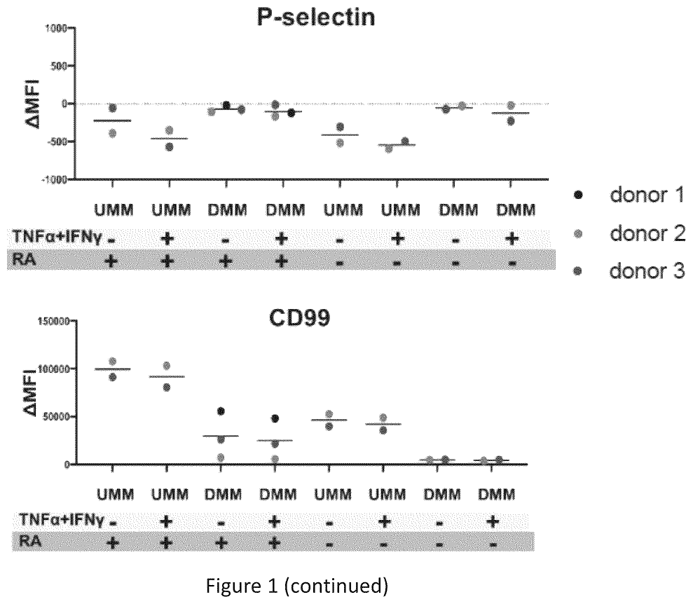

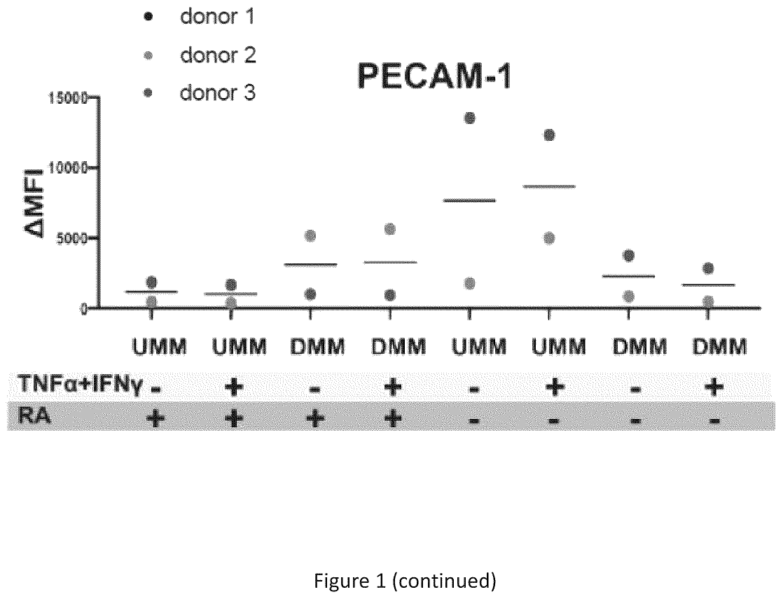

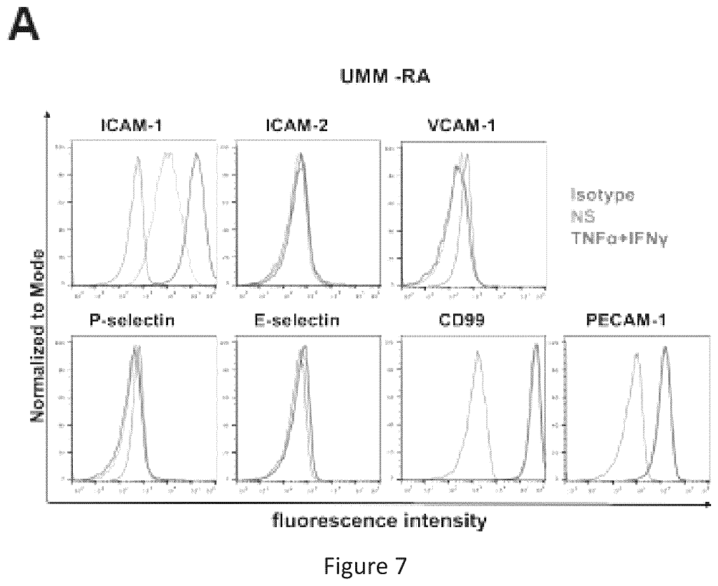

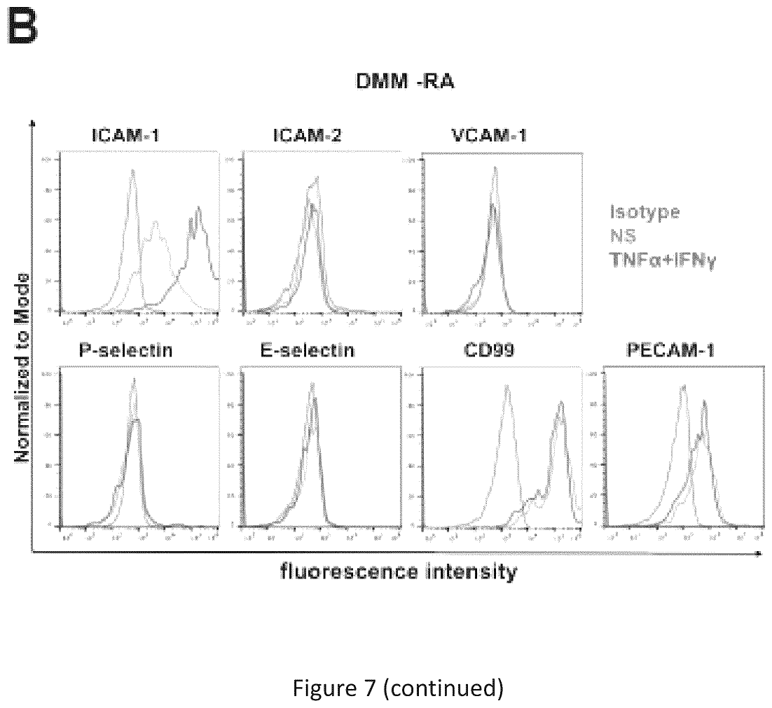

We first explored if BMEC-like cells differentiated by the UMM or DMM express and display cytokine-induced upregulation of adhesion molecules described for the BBB in vivo (4). To this end, we performed flow cytometry analysis of non-stimulated (NS) or proinflammatory cytokine-stimulated (10 ng/mL TNF-α+200 IU/mL IFN-γ) BMEC-like cells differentiated by UMM or DMM for the BBB adhesion molecules ICAM-1, ICAM-2, VCAM-1, P-selectin, E-selectin, and the junctional molecules CD99 and PECAM-1. UMM- and DMM-differentiated BMEC-like cells expressed CD99, PECAM-1 and ICAM-1, the latter only being further increased after stimulation with pro-inflammatory cytokines (FIG. 1A, B, E). At the same time, UMM- and DMM-differentiated BMEC-like cells did not stain positive for ICAM-2, VCAM-1, E-selectin, or P-selectin. Since RA has been reported to inhibit upregulation of endothelial VCAM-1 in vitro (42), we asked if omitting RA treatment influences the adhesion molecule profile of UMM- and DMM-differentiated BMEC-like cells. Completely omitting RA treatment during the whole differentiation process allowed for enhanced cytokine-induced upregulation of ICAM-1 on UMM- or DMM-differentiated BMEC-like cells, but did not affect lack of detection of P-selectin, E-selectin, ICAM-2, and VCAM-1 (FIG. 1C, D, E; FIG. 7). Thus, the well-established methods for differentiating hiPSC-derived BMEC-like cells are not well suited for modeling immune cell interactions at the BBB.

hiPSC-Derived ECs Display a BBB Immune Phenotype but Lack Barrier Properties

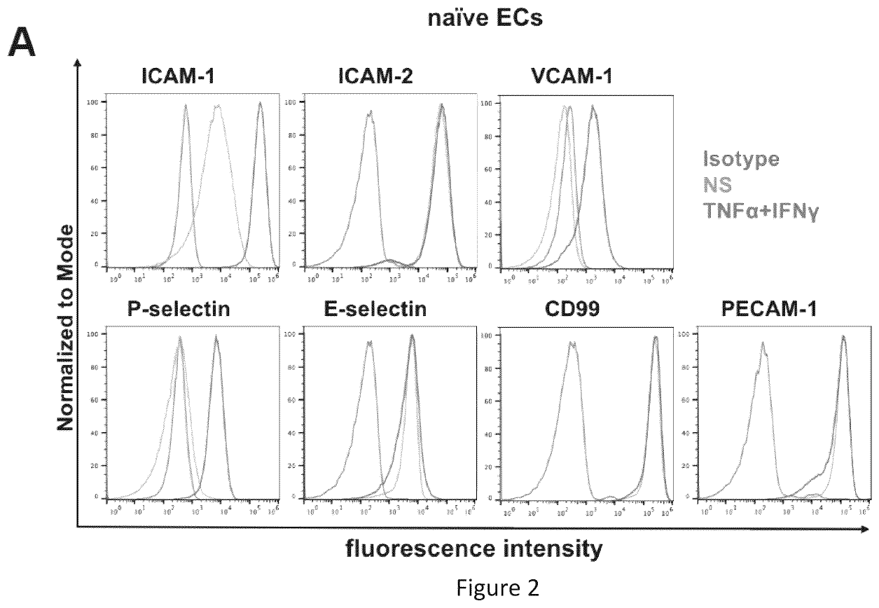

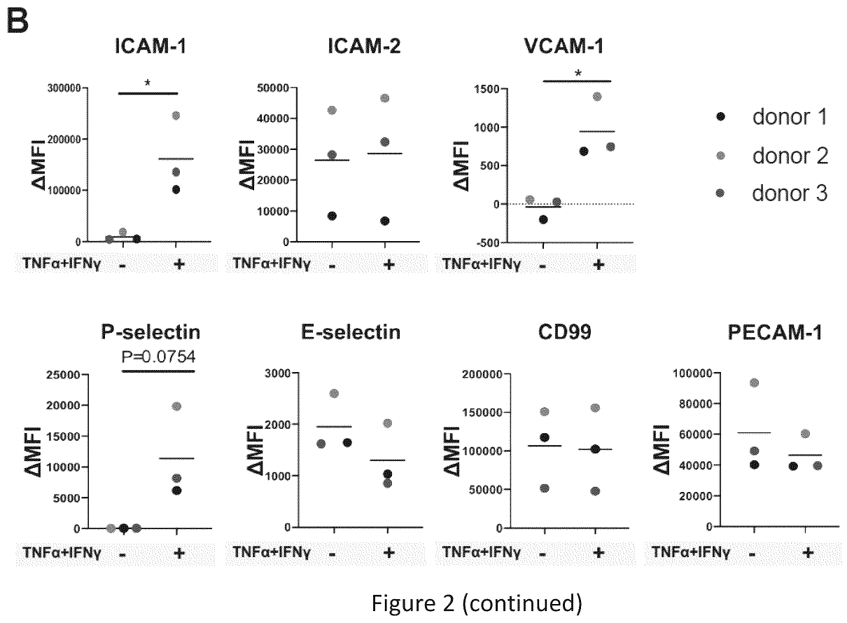

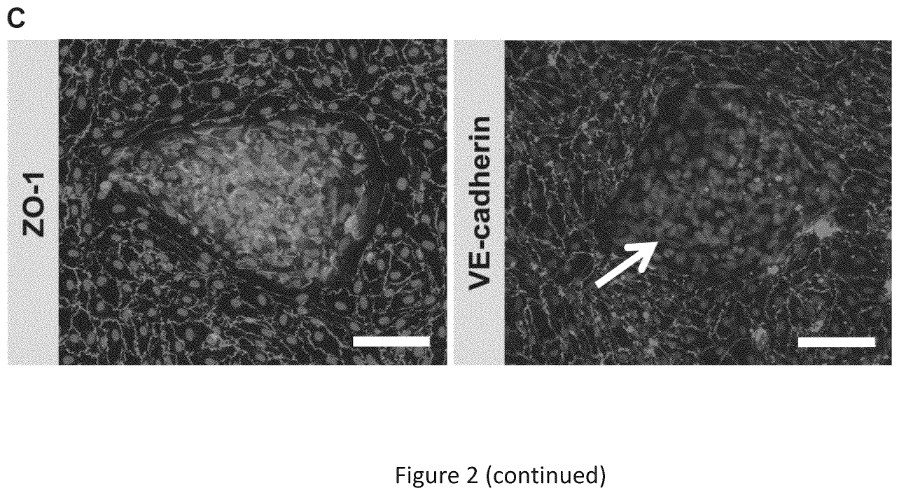

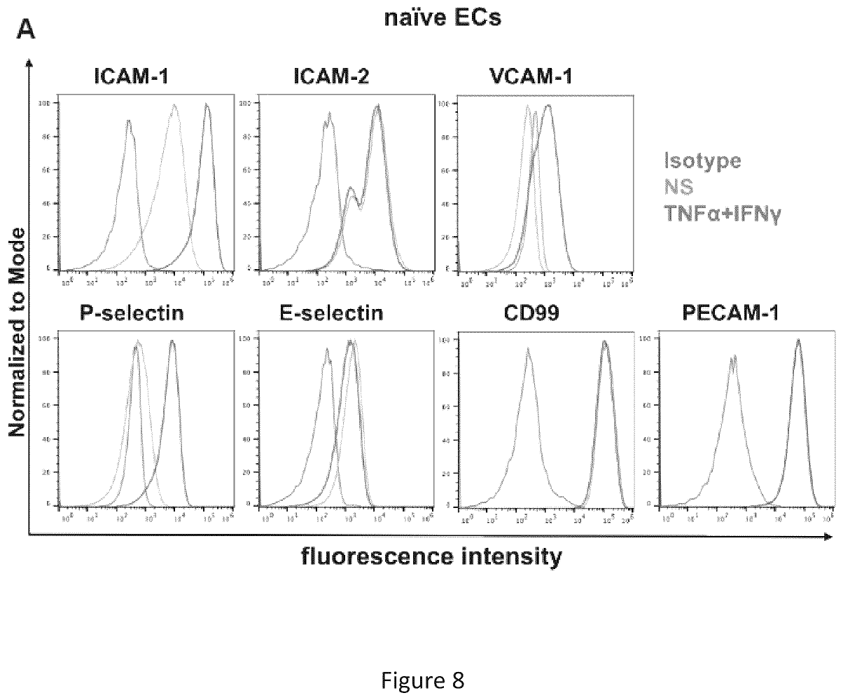

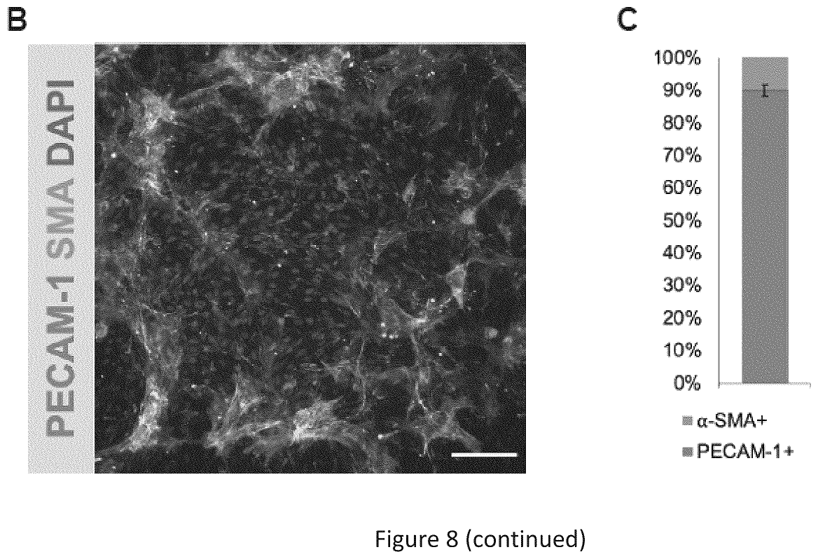

We have previously shown that human CD34⁺ cord-blood stem cells can be differentiated into CD34⁺ endothelial progenitor cells (EPCs) and finally by co-culture with pericytes into brain-like endothelial cells (BLECs) that display endothelial adhesion molecule expression similar to that observed in vivo (15, 31). Therefore, we first asked if hiPSC-derived naïve ECs could exhibit a mature immune phenotype with respect to expression of endothelial adhesion molecules. To this end, we generated CD34⁺CD31⁺ EPCs from hiPSCs using an established protocol (FIG. 3A) (32, 33) and transitioned them to naïve ECs by 6 days of culture in the EC culture medium (hECSR medium: human endothelial serum-free medium+1×B-27 supplement+20 ng/ml bFGF) and investigated cell surface expression of adhesion molecules. Under NS conditions, hiPSC-derived ECs expressed ICAM-1, ICAM-2, E-selectin, CD99, and PECAM-1, while under pro-inflammatory cytokine (1 ng/mL TNF-α+20 IU/mL IFN-γ)-stimulated conditions, ICAM-1 was upregulated, as expected (FIG. 2A, B, FIG. 8A). In addition, we found that hiPSC-derived ECs expressed VCAM-1 and P-selectin upon pro-inflammatory cytokine stimulation (FIG. 2A, B, FIG. 8A). Thus, these naïve ECs may be suitable for modeling aspects of the BBB immune phenotype. However, since these ECs were not specified to a BMEC-like fate, barrier characteristics of hiPSC-derived naïve ECs as measured by transendothelial electrical resistance (TEER) and permeability to the small molecule tracer sodium fluorescein with an average molecular weight of 0.37 kDa (Pe_{NaFl}), were minimal. We failed to detect a measurable TEER across the naïve EC monolayer and detected a high permeability to sodium fluorescein (FIG. 3F, P1; Pe_{NaFl}: 2-10×10⁻³ cm/min). Thus, while hiPSC-derived ECs exhibit improved adhesion molecule expression compared to UMM- and DMM-differentiated BMEC-like cells, they fail to establish barrier properties characteristic of the BBB.

Extended Passaging Drives Naïve hiPSC-Derived ECs to BMEC-Like Cells

Since studying immune cell interactions at the BBB requires both adhesion molecule phenotypes and tight junction phenotypes critical to the process of T-cell diapedesis, we next aimed to identify strategies to drive the development of barrier properties in hiP SC-derived EC monolayers. When EPCs were transitioned to ECs, a mixed population of ZO-1⁺/VE-cadherin⁺ ECs and VE-cadherin^{neg} cells resulted (FIG. 2C). Thus, despite the presence of junction-associated ZO-1− and adherens junction-associated VE-cadherin-expressing cells, even a minimal barrier could not be detected as a complete EC monolayer could not form. Since EPCs have been reported to have the capacity to differentiate to both ECs and smooth muscle cells, depending on the medium used for differentiation (32), PECAM-1^{neg} cells were tested to determine if they expressed α-smooth muscle actin (α-SMA). Indeed, the majority of PECAM-1^{neg} cells stained positive for α-SMA (FIG. 8B, C), underscoring that a fraction of the EPCs differentiated to smooth muscle-like cells (SMLCs) despite being cultured in EC-inductive medium.

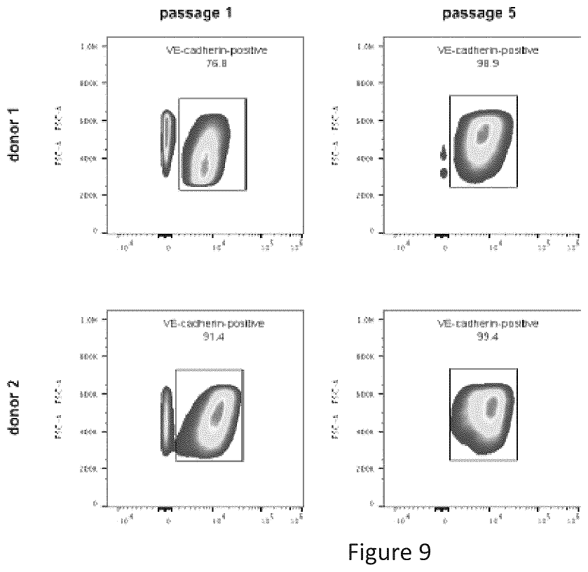

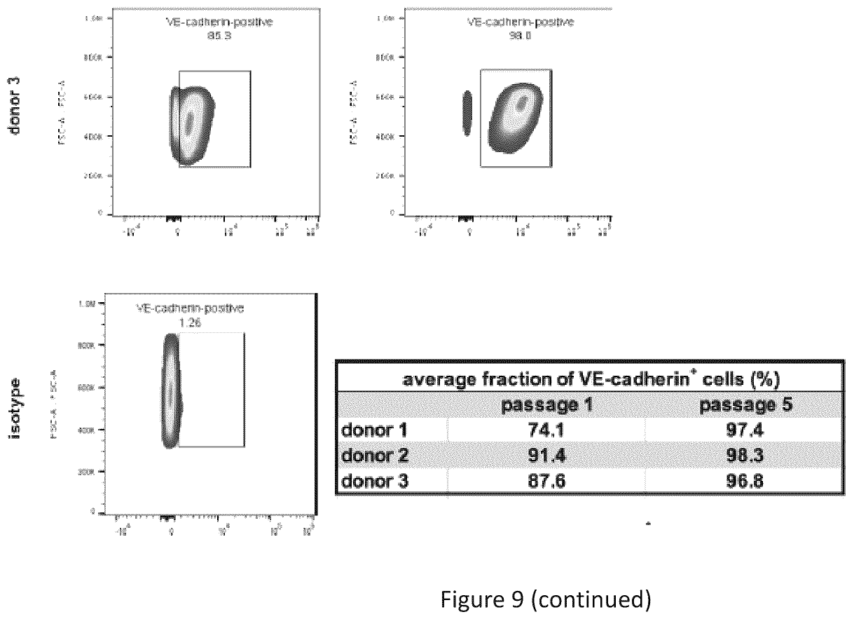

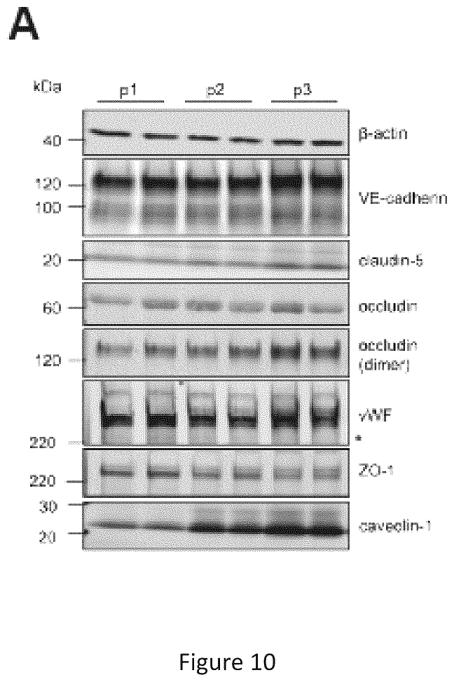

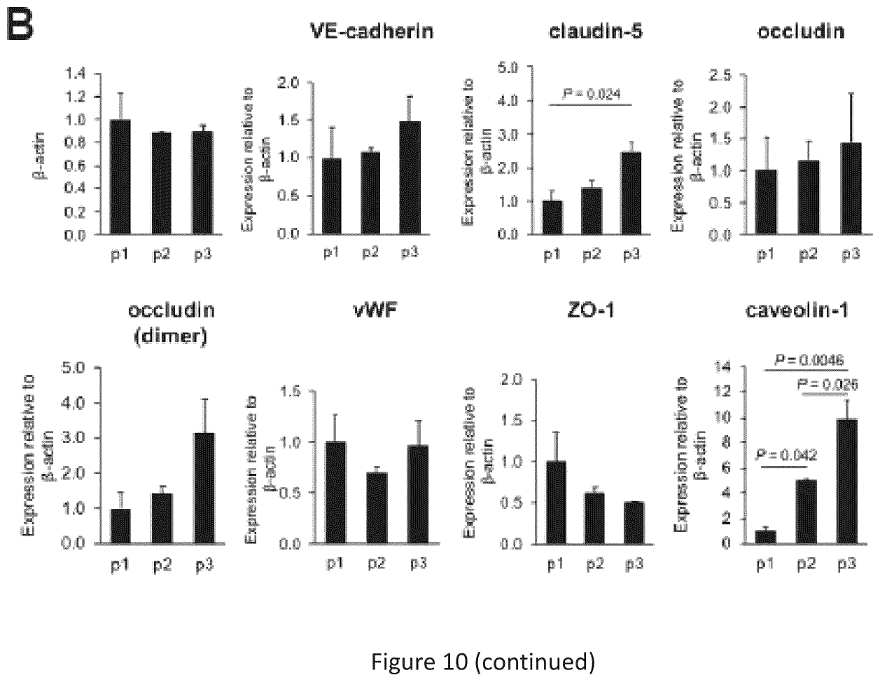

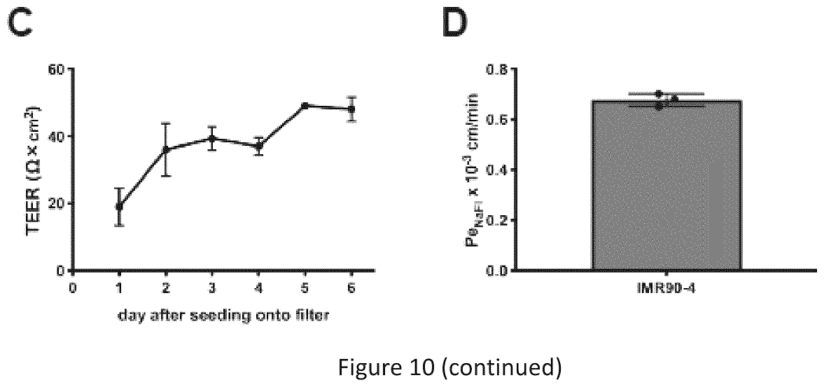

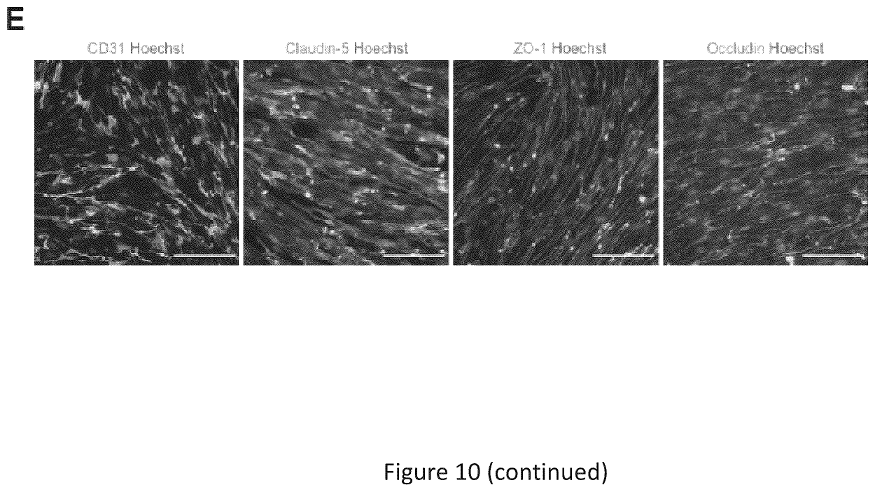

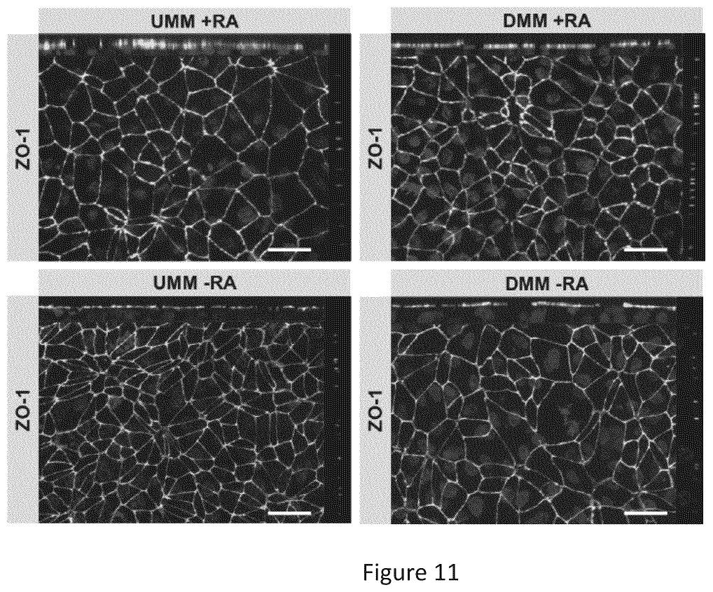

We therefore aimed to generate pure naïve EC monolayers to test the hypothesis that reducing SMLC contamination in the EC monolayers would improve their basal barrier properties. Careful microscopic observation of the EC cultures during the detachment process when performing standard culture passaging showed that ECs detached earlier compared to non-ECs (FIG. 3B). Selective passaging of the preferentially-detached ECs yielded EC monolayers with enriched purity that by passage 2, developed elevated TEER (at day 6 after passaging: 62.4±9.0 Ω×cm²) and ˜10-fold lower permeability (Pe_{NaFl}=(0.279±0.124)×10⁻³ cm/min) when compared to naïve ECs (FIG. 3E, F). These ECs formed by the extended endothelial cell culture method (EECM), also maintain barrier characteristics over several passages. EECM-differentiated ECs showed increased purity of VE-cadherin⁺ cells (FIG. 9) and stable barrier characteristics with TEER above 60 Ω×cm² and low permeability to sodium fluorescein (Pe_{NaFl}<(0.32±0.1)×10⁻³ cm/min) until at least passage 5 (FIG. 3E, F). The barrier properties of EECM-differentiated ECs are comparable to or even tighter than those measured in primary human brain microvascular endothelial cells (13, 28), and similar to those observed in BLECs differentiated from primary CD34⁺ progenitors (15). Correlating with the enhanced barrier properties upon passage, the passage 2-4 EECM-differentiated ECs exhibited increased expression of claudin-5 and improved junctional localization of both occludin and claudin-5 when compared to passage 1 naïve ECs (FIG. 3C). Western blotting confirmed expression of adherens and tight junction-associated proteins (VE-cadherin, occludin, claudin-5, ZO-1), Von Willebrand factor (vWF), and caveolin-1, with claudin-5 abundance increasing after extended passage culture (FIG. 10A, B). Thus, improved barrier properties as measured by TEER and Pe_{NaFl} are likely a result of improved EC purity and improved expression and localization of important BBB tight junction proteins occludin and claudin-5. We further confirmed that the EECM differentiation protocol works independently of hiPSC origin using the fibroblast-reprogrammed IMR90-4 line (FIG. 10C-E). In addition, immunofluorescence staining of passage 5 ECs showed that these ECs develop a flat morphology with flat nuclei which overall resembles the typical morphology of brain endothelial cells (FIG. 3C, D, FIG. 11). Since EECM-differentiated ECs formed a diffusion barrier as marked by a low Pe_{NaFl} value, and showed cellular and junctional morphology reminiscent of that observed for BBB endothelium in vivo, we term the cells generated by this new protocol EECM-BMEC-like cells.

### Astrocytes and Pericytes Affect Morphology but not Diffusion Barrier Characteristics of EECM-BMEC-Like Cells

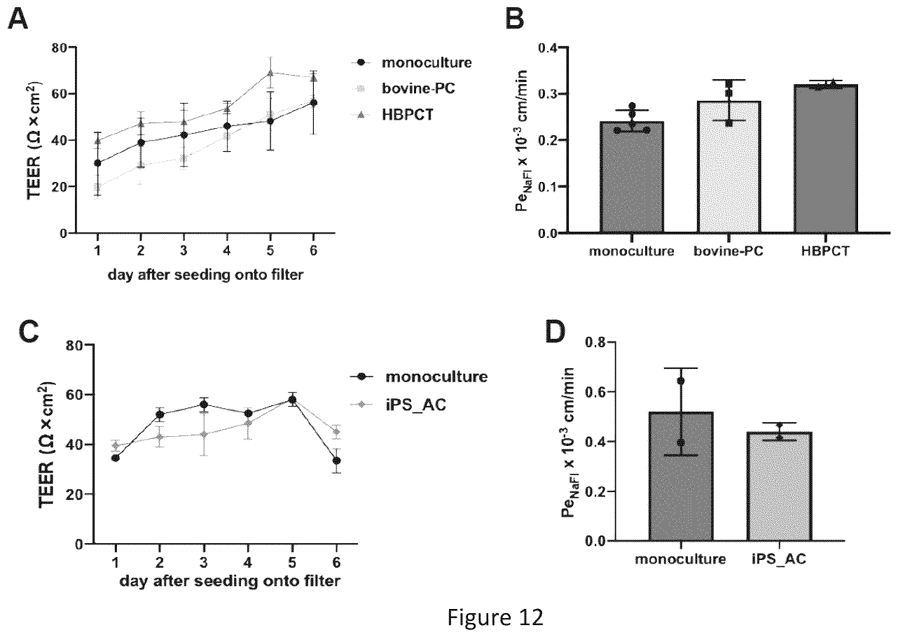

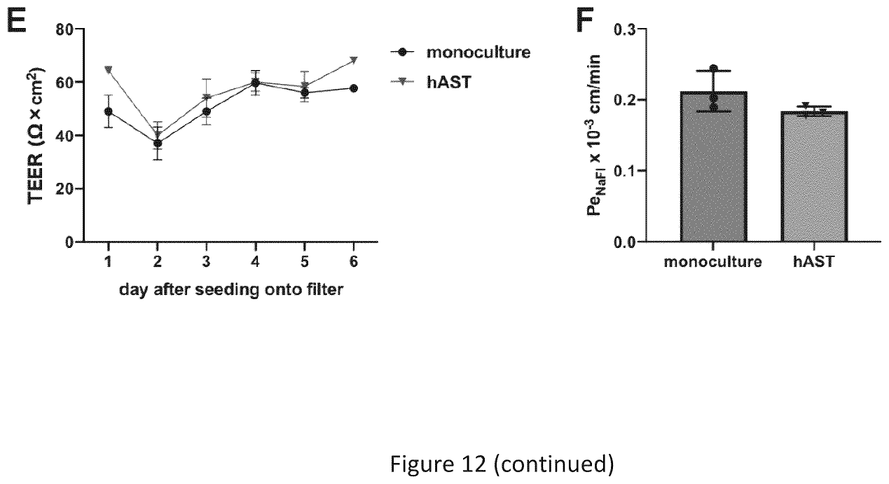

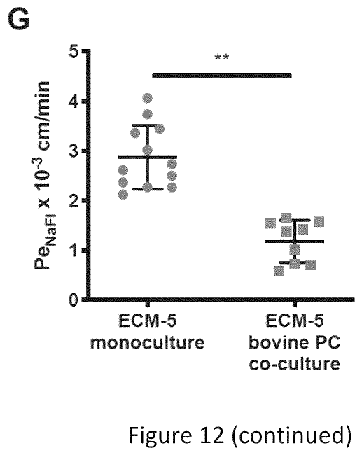

Barrier properties of brain endothelial cells are not intrinsic and rather rely on developmental cues and continuous cross-talk with cells from the neurovascular unit such as pericytes and astrocytes. Indeed, previous observations from us and others have shown that astrocyte or pericyte co-culture can induce barrier characteristics in BLECs and hiPSC-derived BMEC-like cells (15, 38, 43). To determine if barrier properties of EECM-BMEC-like cells (passages 3-5) could be improved by co-culture with astrocytes or pericytes, we grew EECM-BMEC-like cells (passages 3-5) on Transwell filters and co-cultured them for 6 days with bovine pericytes (bovine-PC, (15)), a human brain pericyte cell line (HBPCT, (34)), a human astrocyte cell line (hAST, (35)), or hiPSC-derived astrocytes (iPSC_AC, (29)) in the lower chamber (FIG. 4A). We also tested RA treatment for the first 2 days of passage 3 since RA has been reported to improve barrier properties of hiPSC-derived BMEC-like cells (23). Barrier characteristics of monocultured EECM-BMEC-like cells were not enhanced by co-culture or RA treatment as indicated by indistinguishable TEER and NaFl permeability measurements (FIG. 4B, C; FIG. 12). Immunofluorescent detection of VE-cadherin, claudin-5, and occludin did not reveal any obvious differences in staining levels or junctional localization upon co-culture with hiPSC-derived astrocytes or the human brain pericyte cell line. However, EECM-BMEC-like cells exhibited a higher density cell monolayer and a more elongated morphology compared to the monoculture condition (FIG. 4D-F). Thus, EECM-BMEC-like cells respond to factors secreted by astrocytes and pericytes, but these factors did not enhance diffusion barrier properties.

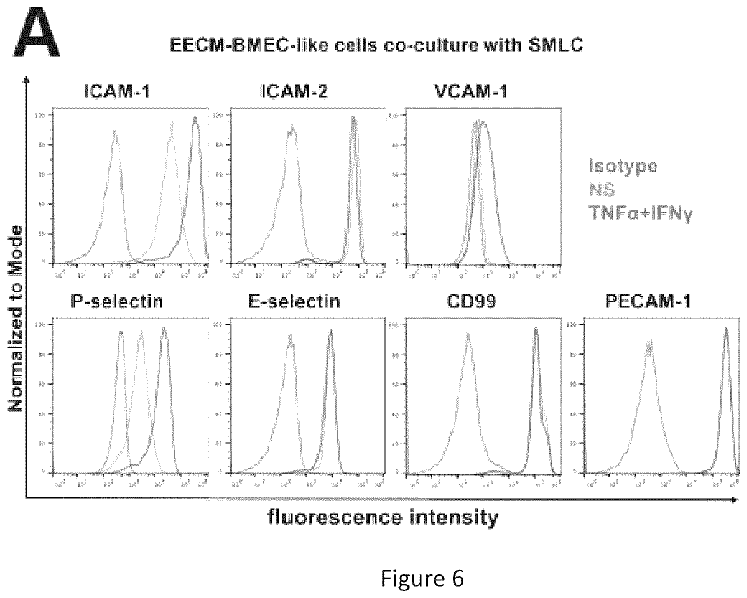

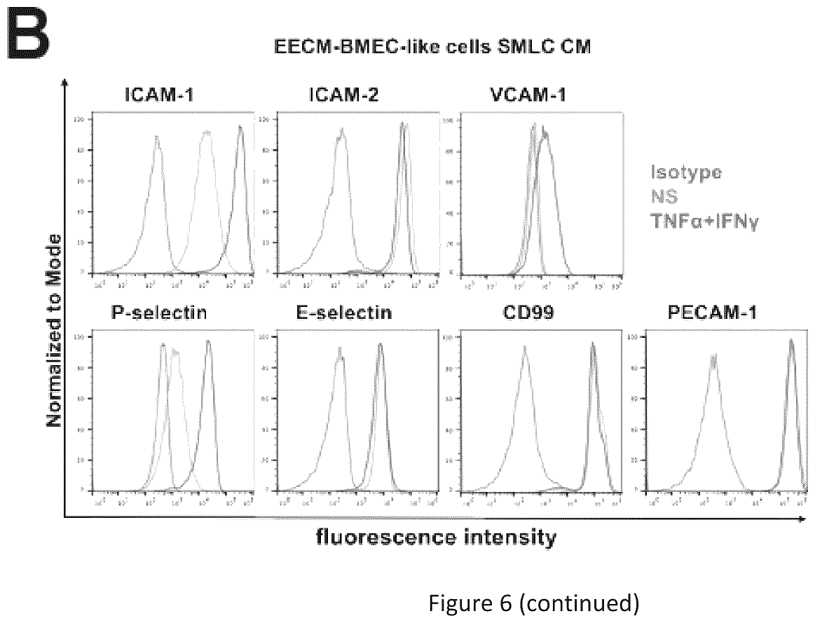

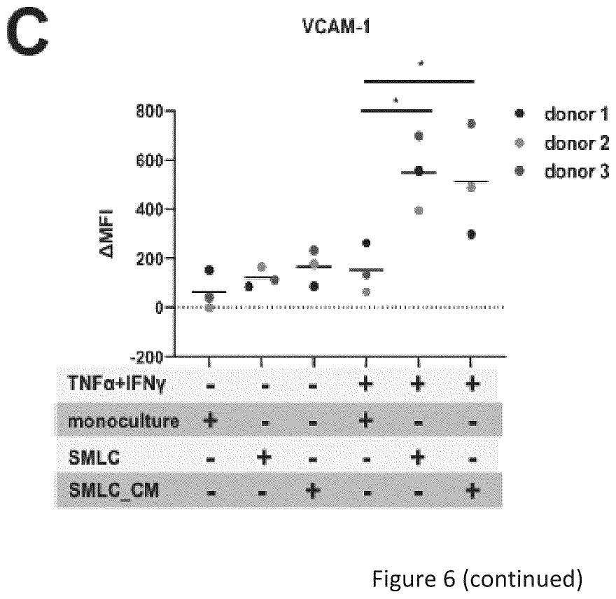

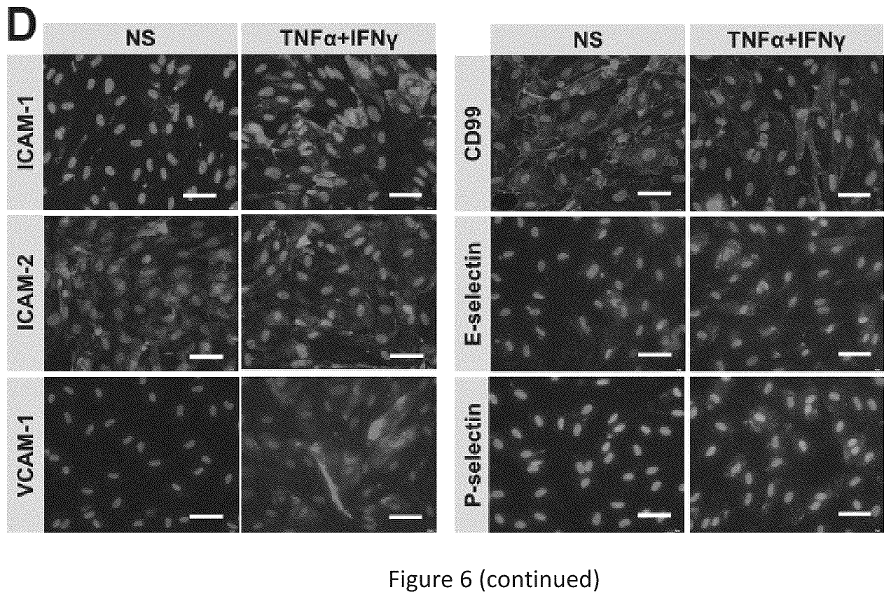

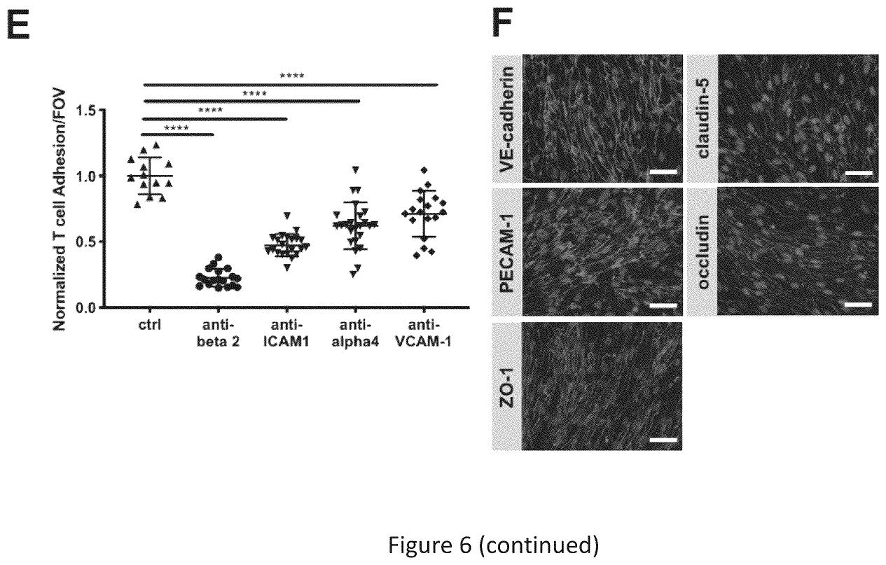

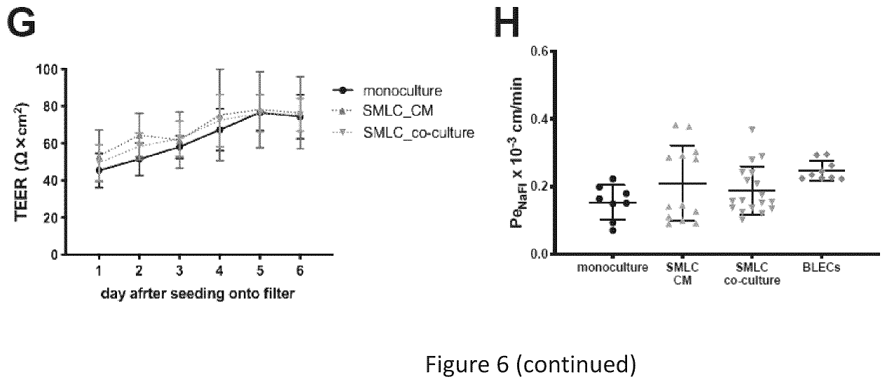

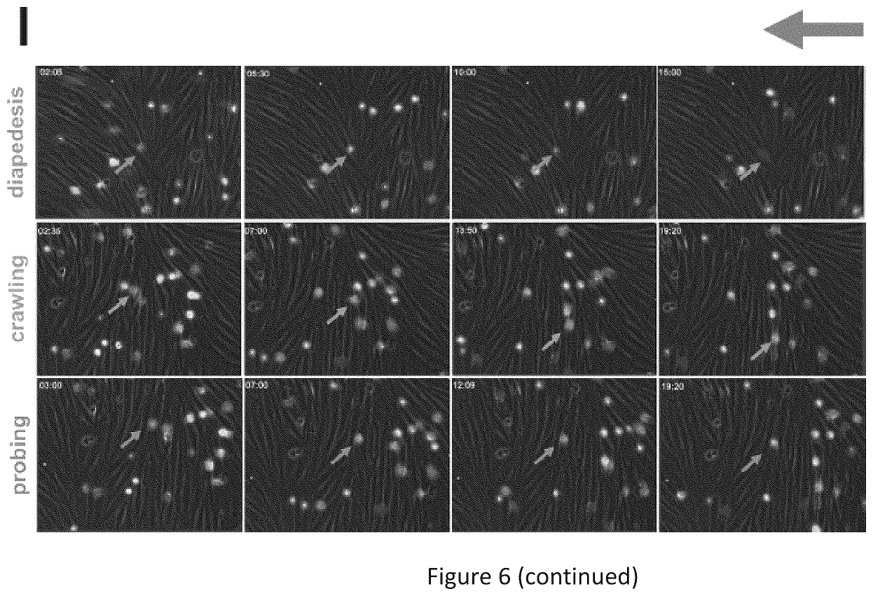

To understand the relevance of culture medium composition on barrier properties of EECM-BMEC-like cells, we also employed ECM-5 medium, which we have previously used in the human CD34⁺ cord-blood stem cells-derived BLECs model (15). Culturing EECM-BMEC-like cells (passage 3-5) in the presence of bovine pericytes in ECM-5 medium improved barrier properties as shown by the significant reduction of their permeability to NaFl when compared to monoculture conditions (FIG. 6G). However, irrespective of monoculture or co-culture, Pe_{NaFl} was extremely high when compared to EECM-BMEC-like cells cultured in hECSR medium (FIG. 12G vs FIG. 4C; ECM-5 monoculture 2.875±0.641×10⁻³ cm/min, ECM-5 bovine pericyte co-culture 1.180±0.424×10⁻³ cm/min, hECSR monoculture 0.221±0.064×10⁻³ cm/min, hECSR bovine pericyte co-culture 0.223±0.058×10⁻³ cm/min). Thus, hESCR medium plays an important role in driving barrier formation in EECM-BMEC-like cells.

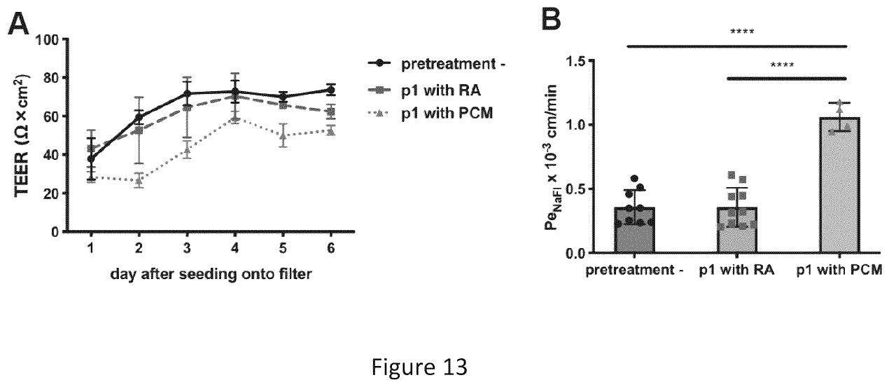

We next tested if differentiating cells in the potentially more plastic, early stages of EPC-to-EC transition (i.e., first several days of culture after EPC purification by MACS, FIG. 3A) were more responsive to RA treatment or secreted factors present in pericyte-conditioned medium (PCM). After EPC purification, EPCs were cultured with either RA for 3 days or PCM for 6 days and then upon reaching passage 2, the resulting ECs were seeded onto Transwell filters. Comparing the TEER and permeability for NaFl, we found that neither RA nor PCM pre-treatment improved barrier properties. Instead, PCM pre-treatment increased the Pe_{NaFl} of the EECM-BMEC-like cell monolayer (FIG. 13). Given the minimal impact of co-cultured astrocytes, pericytes and RA treatment on the barrier properties in EECM-BMEC-like cells, these conditions were not further investigated.

### Adhesion Molecule Phenotype of EECM-BMEC-Like Cells

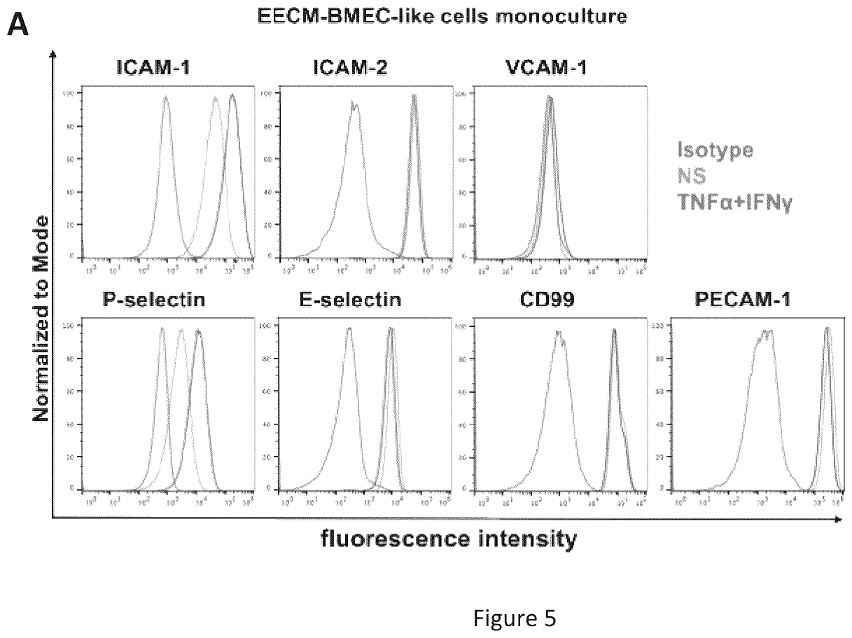

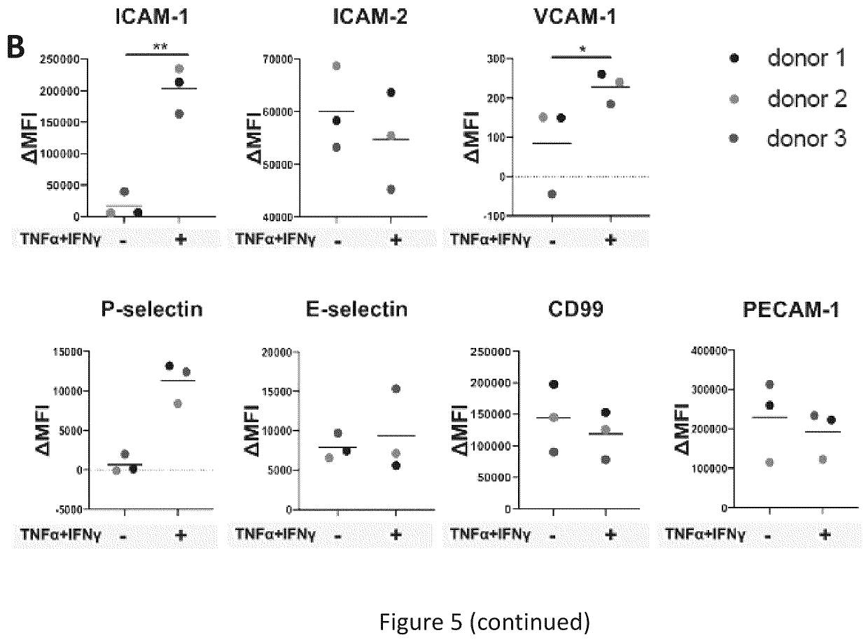

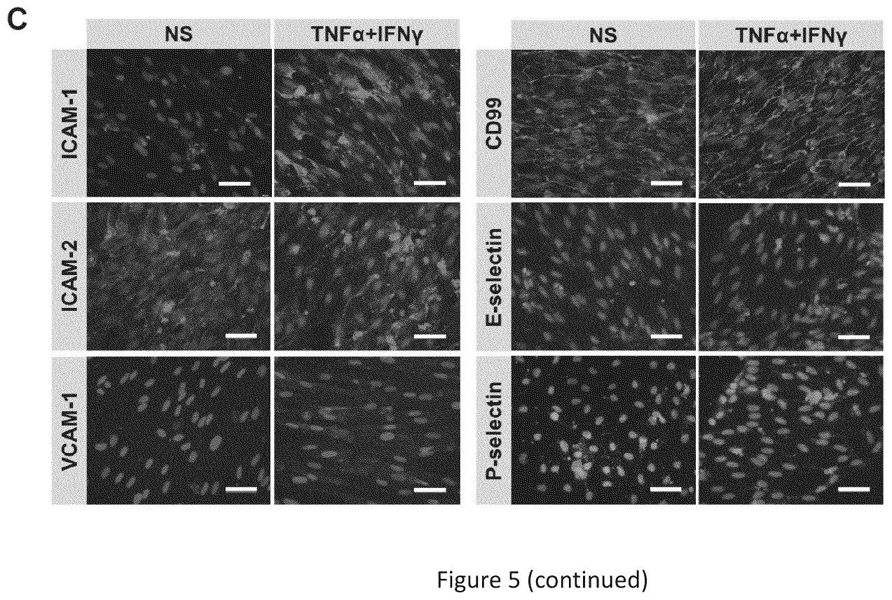

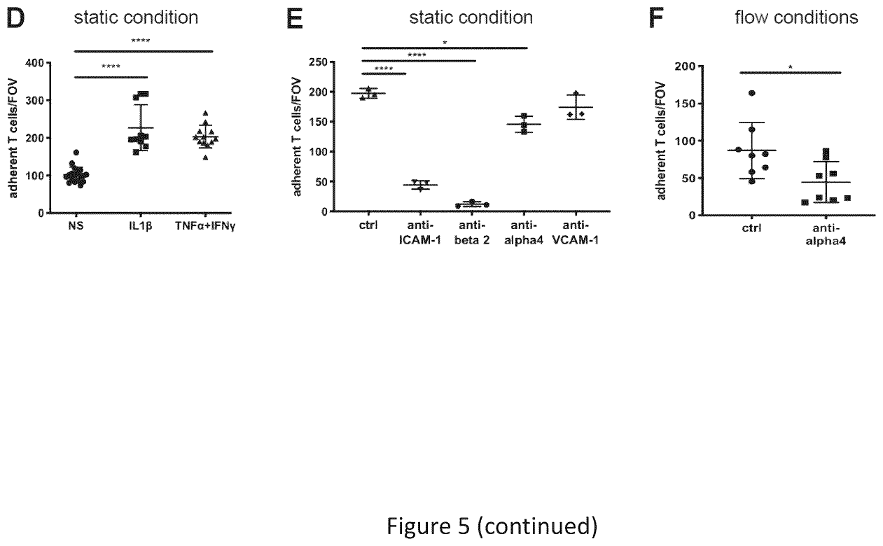

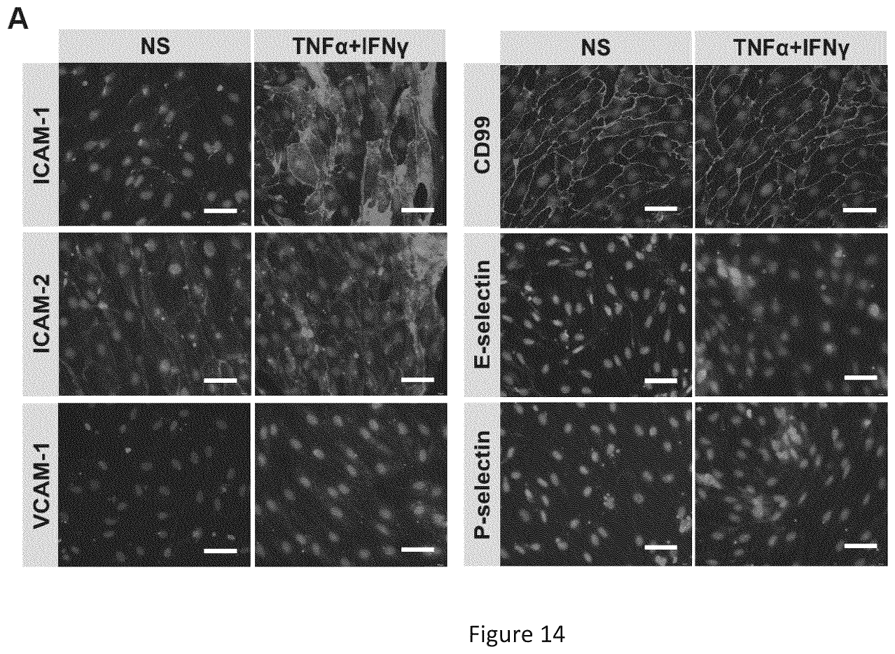

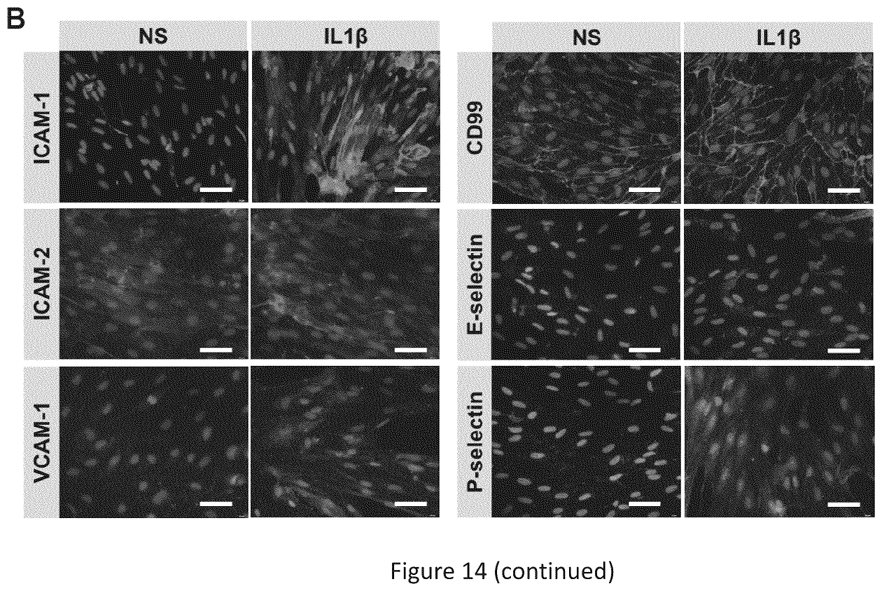

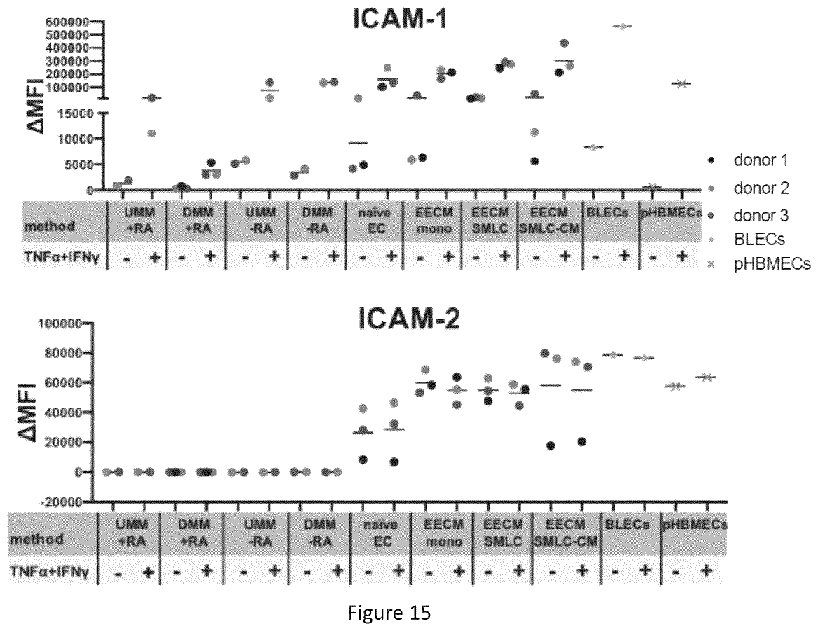

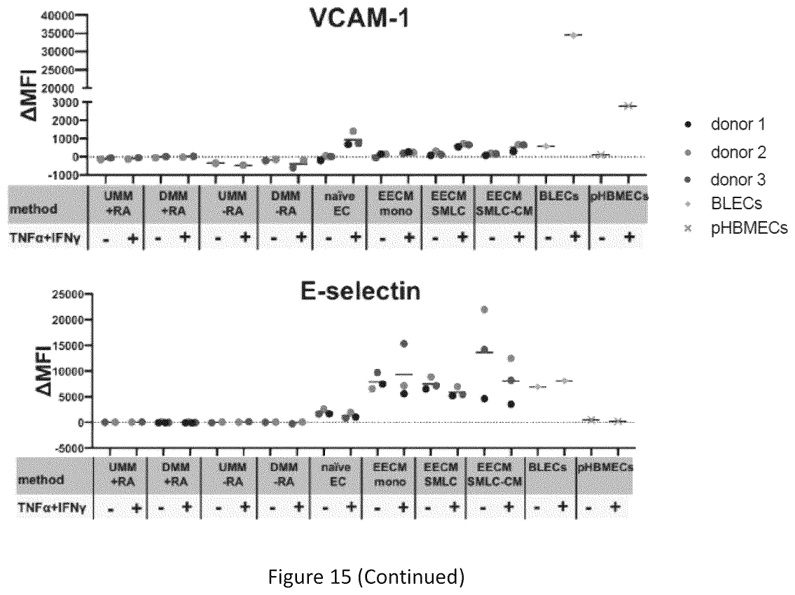

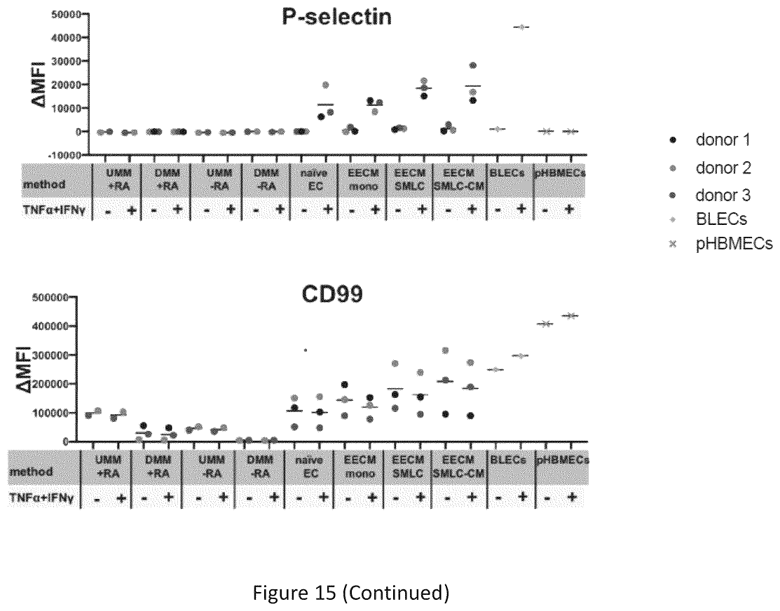

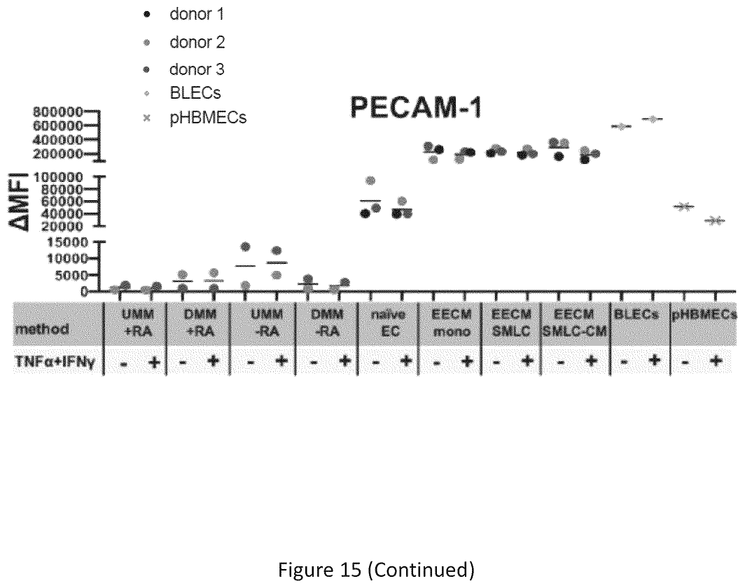

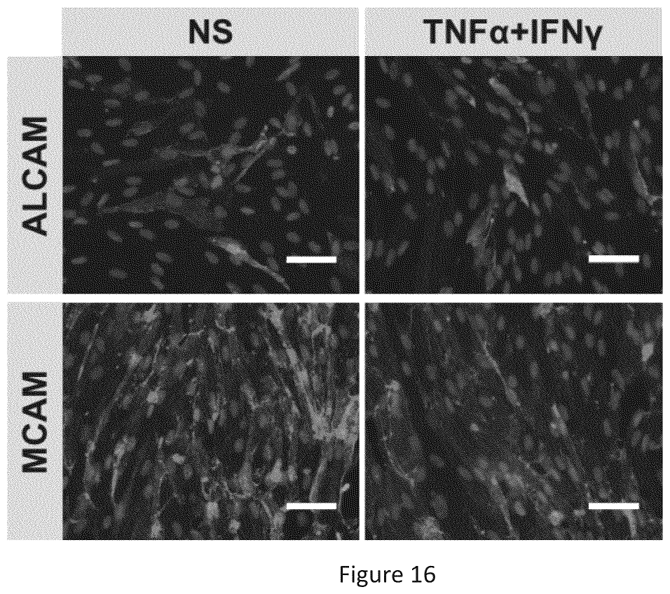

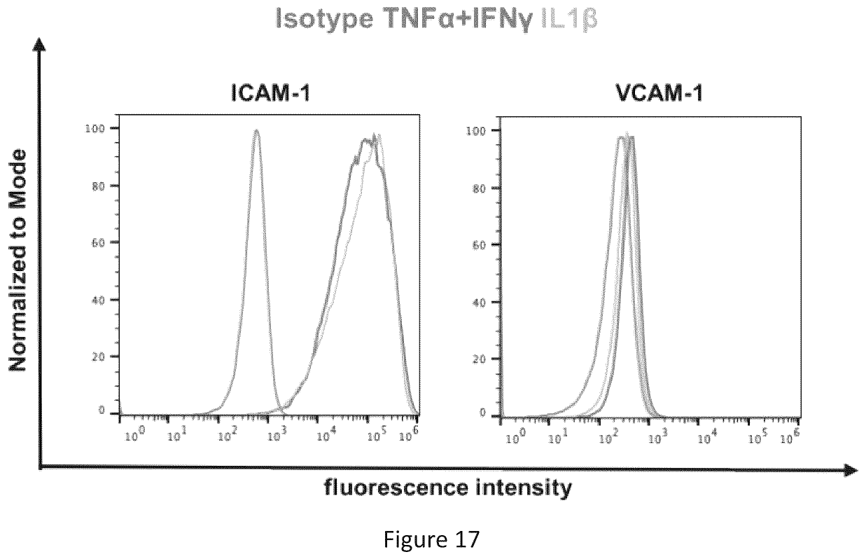

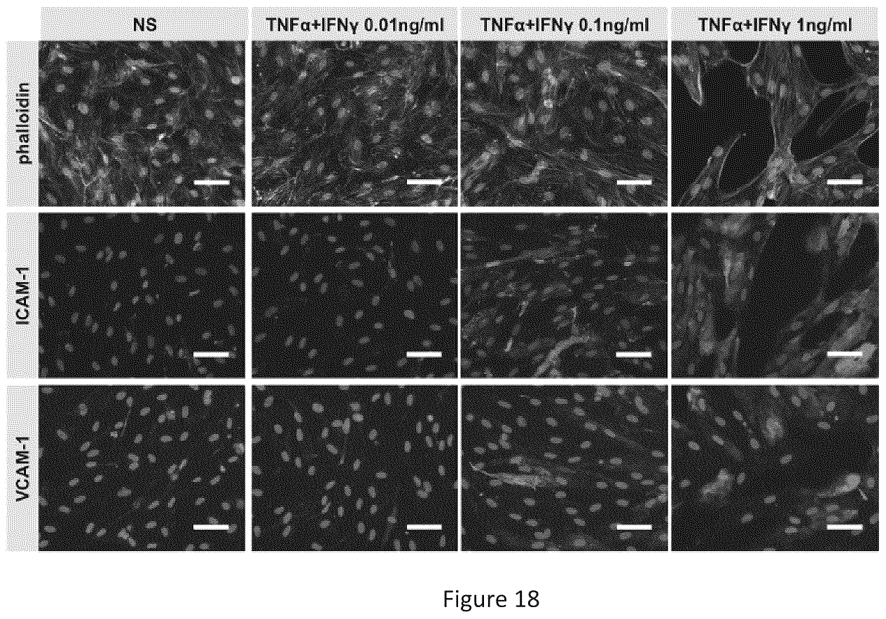

We next asked if the extended passaging of naïve ECs to EECM-BMEC-like cells affected the adhesion molecule repertoire observed for naïve ECs in FIG. 2. Much like naïve ECs, EECM-BMEC-like cells expressed cell surface ICAM-1, ICAM-2, CD99, PECAM-1, E-selectin, and P-selectin under NS conditions (FIG. 5A, B, C; FIG. 14). Proinflammatory cytokine (1 ng/mL TNF-α+20 IU/mL IFN-γ) stimulation induced upregulated expression of ICAM-1 and P-selectin, but did not change the levels of ICAM-2 and CD99 (FIGS. 5A and 5B). With this adhesion molecule phenotype, EECM-BMEC-like cells differ substantially from UMM- or DMM-differentiated BMEC-like cells and resemble brain like endothelial cells (BLECs, (15)) and primary human brain endothelial cells previously shown to express BBB adhesion molecules (44) (FIG. 15). We also confirmed ALCAM and MCAM expression by EECM-BMEC-like cells using immunostaining (FIG. 16). In contrast to the findings with naïve ECs, we found only subtle upregulation of VCAM-1 on TNF-α/IFN-γ-stimulated EECM-BMEC-like cells (FIG. 5A, B, C and FIG. 14A) or 1 ng/mL IL-1β-stimulated EECM-BMEC-like cells (FIG. 14B). TNF-α/IFN-γ-or IL-1β-stimulated EECM-BMEC-like cells showed similar levels of cell surface ICAM-1 and VCAM-1 (FIG. 17). Moreover, increasing the concentration of proinflammatory cytokines or using different combinations thereof resulted in disruption of EECM-BMEC-like cell monolayers (FIG. 18) rather than increasing the endothelial surface staining for ICAM-1 and VCAM-1.

### EECM-BMEC-Like Cells Express VCAM-1 and Functional ICAM-1

Since EECM-BMEC-like cells possess both barrier and immune phenotypes that would potentially be valuable for investigating immune cell interactions with the BBB, we wished to determine the functional relevance of endothelial ICAM-1 and VCAM-1 on EECM-BMEC-like cells. To this end, we investigated the adhesion of Th1* cells on NS and TNF-α/IFN-γ-stimulated and IL-1β-stimulated EECM-BMEC-like cells (passages 3-5) under static conditions. Cytokine stimulation significantly increased the numbers of Th1* cells adhering to EECM-BMEC-like cell monolayers (FIG. 5D). Antibody-mediated blocking of endothelial ICAM-1 and its respective β2-integrin ligand on Th1* cells significantly reduced adhesion of Th1* cells to cytokine stimulated EECM-BMEC-like cells (FIG. 5E). Antibody-mediated blocking of a4b1-integrins on Th1* cells also reduced adhesion of Th1* cells to cytokine stimulated EECM-BMEC-like cells, but blocking of the endothelial a4b1-integrin ligand VCAM-1 did not significantly reduce Th1* adhesion (FIG. 5E). a4b1-integrin-mediated arrest of Th1* cells was also confirmed on cytokine-stimulated EECM-BMEC-like cells under physiological flow (FIG. 5F). Taken together, these data demonstrate that EECM-BMEC-like cells express functional ICAM-1 supporting β2-integrin-dependent adhesion of Th1* cells. Further, EECM-BMEC-like cells also support a4b1-integrin-mediated arrest of Th1* cells, where VCAM-1 does not visibly contribute to this process.

### Smooth Muscle-Like Cells (SMLCs) Induce Robust Functional VCAM-1 Expression on EECM-BMEC-Like Cells

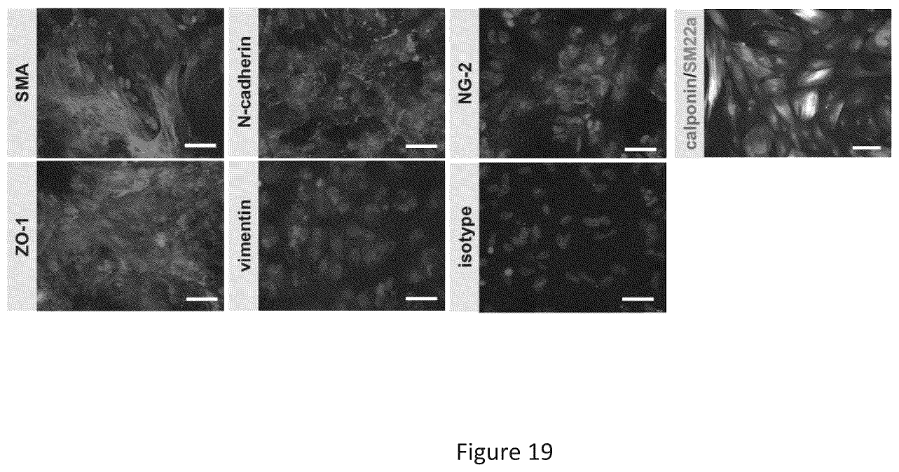

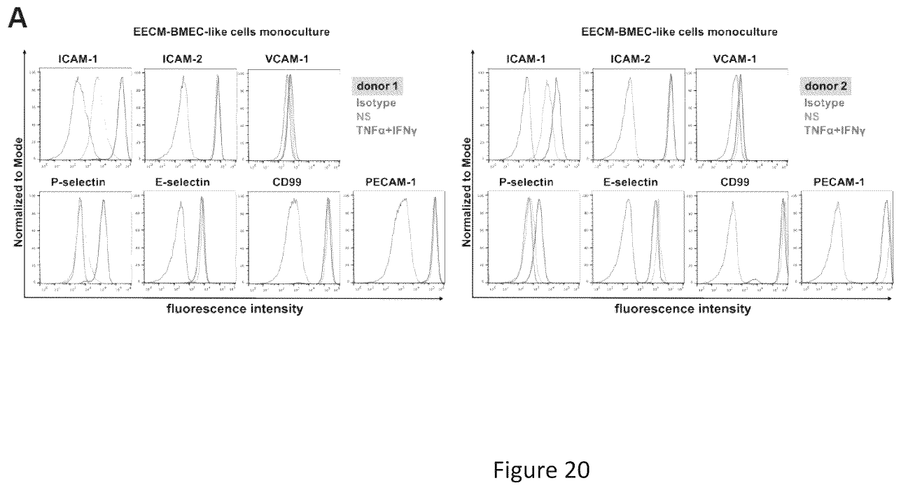

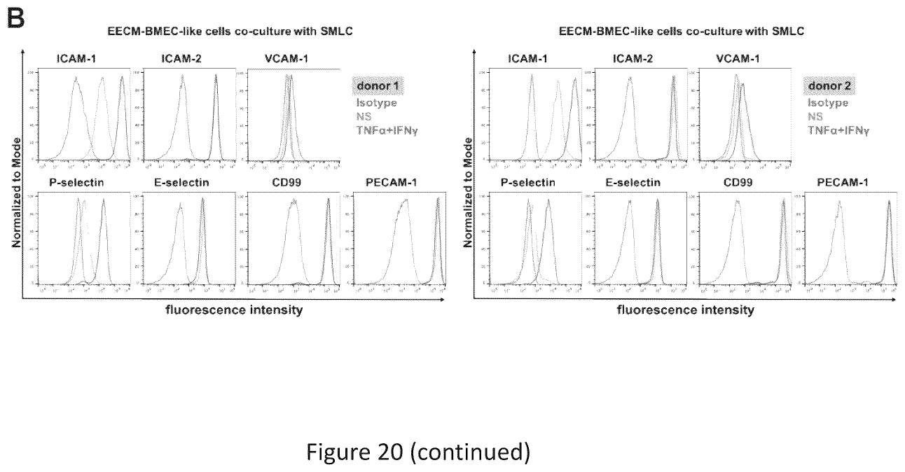

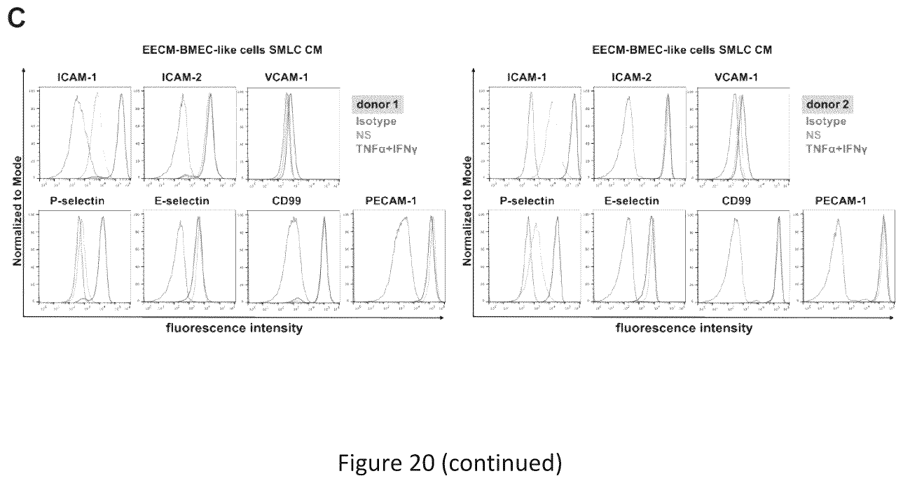

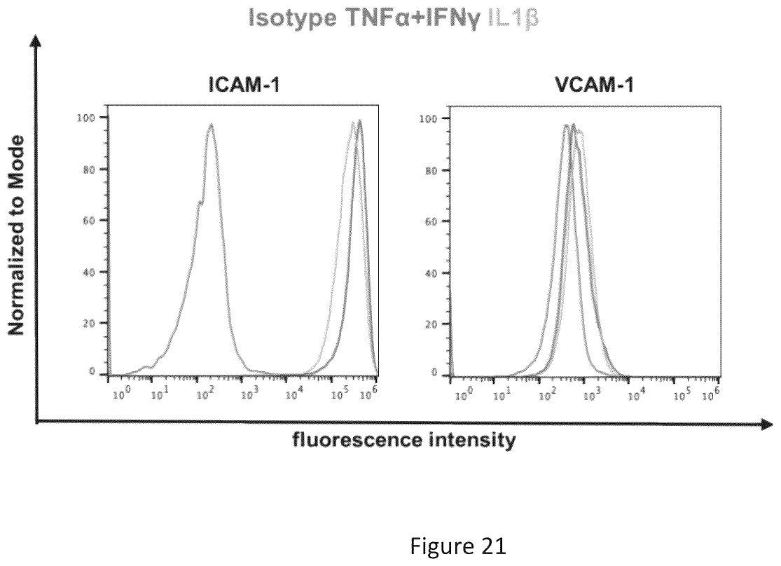

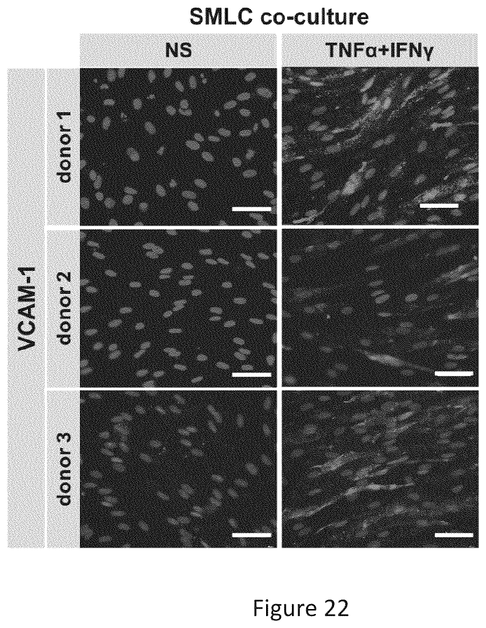

As the a4-integrin/VCAM-1 interaction, in addition to LFA-1/ICAM-1, has been reported to mediate CNS entry of T and B cells in CNS autoimmunity (6, 45), we explored further strategies to enhance expression of VCAM-1 on EECM-BMEC-like cells. In peripheral endothelial cell cultures, co-culture with smooth muscle cells was shown to enhance endothelial VCAM-1 expression (46). Therefore, we asked if hiPSC-derived SMLCs could increase the expression of VCAM-1 in EECM-BMEC-like cells. Human iPSC-derived smooth muscle-like cells (SMLCs) were obtained by continued culture of adherent cells after selective detachment of endothelial cells for derivation of EECM-BMEC-like cells (FIG. 3A, B). These SMLCs expressed proteins characteristic of smooth muscle cells, including α-SMA, calponin, and smooth muscle protein-22a (FIG. 19). Next, the SMLCs were co-cultured with EECM-BMEC-like cells (passage 3-5) on Transwell filters for 6 days and VCAM-1 staining on EECM-BMEC-like cells was compared before and after cytokine stimulation. We found that co-culture of EECM-BMEC-like cells with SMLCs significantly increased cell surface expression of VCAM-1 after TNF-α/IFN-γ-stimulation, while there was no overt change to cell surface levels of ICAM-1, ICAM-2, PECAM-1, CD99, E-selectin or P-selectin under either NS or stimulated conditions compared with monocultured EECM-BMEC-like cells (FIG. 6A vs. FIG. 5A and FIG. 6C; FIG. 20). IL-1β-stimulation also showed similar effects on cell surface ICAM-1 and VCAM-1 staining of SMLC co-cultured EECM-BMEC-like cells (FIG. 21). Interestingly, the SMLC co-culture could be replaced by providing conditioned medium (CM) from SMLCs to the abluminal side of EECM-BMEC-like cells monolayers (FIG. 6B; FIG. 20). Immunofluorescence staining of EECM-BMEC-like cells cultured in the presence of SMLC co-culture or SMLC-derived CM confirmed increased induction of VCAM-1 after cytokine stimulation (FIG. 6D; FIG. 22).

Considering the SMLC-enhanced cell surface expression of VCAM-1 on EECM-BMEC-like cells, we next asked if VCAM-1 would now play a role in Th1* cell adhesion to EECM-BMEC-like cells. As observed in the adhesion assays with monocultured EECM-BMEC-like cells, antibody-mediated blocking of endothelial ICAM-1 or its Th1* ligand, β2-integrin, significantly reduced adhesion to SMLC-derived CM enhanced EECM-BMEC-like cells (FIG. 6E). However, in key contrast to monocultured EECM-BMEC-like cells, antibody-mediated blocking of endothelial VCAM-1 in addition to its ligand, a4-integrin, on Th1* cells significantly decreased T cell adhesion to EECM-BMEC-like cells indicating that SMLC-derived CM enhanced VCAM-1 expression on EECM-BMEC-like cells to levels of functional relevance.

Since smooth muscle cells can increase endothelial permeability in the periphery (46), we also examined if SMLC derived factors would impair barrier properties of the EECM-BMEC-like cells. Although both co-culture of EECM-BMEC-like cells with SMLC or SMLC-derived CM enhanced variability of permeability measures, there was no significant effect on barrier characteristics of EECM-BMEC-like cells as determined by TEER values or permeability to NaFl (FIG. 6G, H). Furthermore, co-culture of EECM-BMEC-like cells with SMLC did not change the junctional localization of VE-cadherin, PECAM-1, ZO-1, claudin-5, or occludin (FIG. 6F). Taken together, SMLC-enhanced EECM-BMEC-like cells retain the barrier properties while displaying an optimized cell adhesion molecule phenotype, thus making them a highly suitable model to study immune cell interactions with the human BBB in vitro. To verify their capability to fully support immune cell migration, we investigated the interaction of human Th1* cells superfused over TNF-α/IFN-γ stimulated EECM-BMEC-like cell monolayers under physiological flow. In vitro live cell imaging demonstrated that Th1* cells arrest and polarize on EECM-BMEC-like cell monolayers. This was followed by Th1* crawling and probing prior to crossing the EECM-BMEC-like cell monolayers (FIG. 61), as observed by us previously on CD34⁺ derived BLECs (37). The EECM-BMEC-like cells thus allow for the observation of the individual steps of the multi-step T-cell extravasation cascade across the BBB.

### Discussion

We set out to identify a human in vitro BBB model that is suitable for the study of BBB-immune cell interactions, which are important in CNS immune surveillance and neuroinflammatory diseases (3, 4, 47, 48). Human BBB models have been generated from diverse sources, including primary or immortalized brain ECs (10, 49, 50) and cord blood-derived endothelial progenitors (15, 51). We focused, however, on models generated from hiPSCs, as they offer benefits in scalability and the potential for studying immune cell interactions in an autologous fashion. We first evaluated well-established protocols for generating hiPSC-derived BMEC-like cells via the UMM and DMM (17, 23). These, and similar methods generate cells that achieve very high TEER, express known BBB solute and efflux transporters, and yield molecular permeabilities that correlate well with in vivo observations and these BMEC-like cells are therefore well suited to study small and large molecule permeability (14, 17, 21, 23, 52-55). However, we found that BMEC-like cells differentiated by the UMM and DMM do not express all adhesion molecules shown to mediate the interaction of immune cells with the BBB (4, 8, 48, 56-58), and thus an alternative approach is required to examine such interactions. We therefore evaluated the immune cell adhesion molecule profile of naïve ECs differentiated from hiPSC-derived EPCs (32), and observed basal or pro-inflammatory cytokine-inducible expression of ICAM-1, ICAM-2, VCAM-1, E-selectin, P-selectin, CD99 and PECAM-1. However, these cells did not develop a passive barrier characteristic of the BBB. By contrast, after extended culture, the resulting cells displayed well-developed tight junctions, moderate TEER, and a flat cellular morphology and junctional architecture characteristic of primary BMECs. Importantly, after extended culture, the resulting cells retained an immune cell adhesion molecule profile similar to primary BMECs (26, 27, 59), and expressed ICAM-1, ICAM-2, E-selectin, P-selectin, CD99, and PECAM-1. Pro-inflammatory cytokine stimulation led to upregulation of ICAM-1 and P-selectin and induction of VCAM-1 that could be further enhanced by SMLC co-culture or SMLC-derived conditioned medium treatment. Non-inflammatory expression of E-selectin on EECM-BMEC-like cells may be due to mechanisms involved in growth of EECM-BMEC-like cells as previously reported (60) and must be considered when using EECM-BMEC-like cells to study immune cell trafficking across the non-inflamed BBB, which does not show constitutive E-selectin expression in vivo.

We confirmed the functionality of ICAM-1 and VCAM-1 in our model by employing blocking antibodies and observed reduced Th1* cell adhesion. While EECM-BMEC-like cells have weaker barrier properties than UMM- or DMM-differentiated BMEC-like cells, they are similar to other human stem cell-derived models (15, 18, 51) and primary mouse BMECs (61, 62), and have stronger barrier properties than immortalized human or rodent cell lines (10, 50, 63). EECM-BMEC-like cells are similar to recently reported hiPSC-derived brain capillary-like endothelial cells (BCLECs) (18) as both proceed through an EPC intermediate and develop a moderate TEER of approximately 60-80 Ω×cm². However, in contrast to the work of Praça et al. (18) we found that EECM-BMEC-like cells demonstrated pro-inflammatory cytokine-inducible expression of VCAM-1, a prerequisite for studying immune cell interactions with the BBB (4, 6). Taken together, while other hiPSC-derived BBB models are likely better suited to evaluate drug permeability, drug efflux, and nutrient transport, EECM-BMEC-like cells have a robust adhesion molecule profile, functionally interact with Th1* cells, and have good paracellular barrier properties, making this model well-suited to study immune cell-BBB interactions.

The EECM-BMEC-like cell differentiation protocol was adapted from an existing method to generate CD34⁺CD31⁺ EPCs from hiPSCs via small molecule activation of canonical Wnt signaling (32). Prior work demonstrated that culturing these endothelial progenitors for 10 days in EGM-2, a widely-used endothelial cell medium that contains serum and several growth factors (EGF, IGF, bFGF, and VEGF), yielded ECs with TNFα-inducible expression of ICAM-1 and TEER of approximately 40 Ω×cm² (32). We therefore asked whether hECSR, a serum-free medium containing B-27 supplement and bFGF that has been previously used to culture BMEC-like cells (17), would enhance the barrier phenotype of the resulting ECs without compromising the robust immune cell adhesion molecule profile we observed. We found that after extended culture, the resulting ECs developed increased TEER of 60-80 Ω×cm² and reduced NaFl permeability, phenotypes attributable both to improved EC monolayer purity and improvements to claudin-5 and occludin expression and junctional localization. Importantly, the presence of continuous localization of occludin to tight junctions, a hallmark of BBB endothelium (64), suggests specification of these naïve ECs to a barrier forming, BMEC-like phenotype. RA treatment or co-culture of the resulting EECM-BMEC-like cells with pericytes or astrocytes did not lead to further improvements in barrier properties, and conversely, culturing these cells in serum- and mitogen-rich ECM-5 medium yielded markedly higher sodium fluorescein permeability. In contrast, Praca et al. (18) generated brain capillary-like endothelial cells (BCLECs) from similar hiPSC-derived mesodermal endothelial progenitors using EGM-2 medium, and found that VEGF, Wnt3a, and RA supplementation increased TEER from approximately 20 to 60 Ω×cm², decreased Lucifer yellow permeability, increased claudin-5 abundance, and increased efflux transporter and solute carrier transcript expression (18). Together, these observations suggest a strong dependence of endothelial barrier properties on basal cell culture medium, and that extended culture in minimal hECSR medium is sufficient to generate cells with a robust paracellular barrier in the absence of co-culture or additional exogenous factors. We note, however, that co-culture or supplementation of Wnts, RA, or other factors with known roles in BBB development (65, 66) could potentially improve aspects of BBB phenotype in EECM-BMEC-like cells, but the barrier properties, junctional architecture, and immune cell adhesion molecule profile of the present model is well suited for the intended application. In fact, a major advantage of the EECM-BMEC-like model is the presence of functional VCAM-1, which plays a major role in the capture of T cells on endothelium via α4β1 integrin binding (4, 6). While recent work demonstrated TNFα-inducible expression of VCAM-1 in BMEC-like cells differentiated via the UMM, maintained under flow in collagen channels, and assayed after fixation and permeabilization (22), we did not observe cell surface VCAM-1 expression in UMM or DMM-differentiated BMEC-like cells. Further, cytokine-inducible expression of VCAM-1 in ECs differentiated from hiPSC-derived endothelial progenitors has been variable, with some reports of positive (67) and negative (18, 68) results and our monocultured EECM-BMEC-like cells only exhibited a modest response to inflammatory stimulation. Given the importance of mural cells in endothelial function and previous observations that smooth muscle cell-EC interactions induce VCAM-1 in peripheral ECs (46), we asked whether hiPSC-derived SMLCs could modulate VCAM-1 expression. Indeed, we found that co-culturing EECM-BMEC-like cells with hiPSC-derived SMLCs or SMLC-derived conditioned medium improved VCAM-1 inducibility on EECM-BMEC-like cells without significantly influencing barrier properties. Furthermore, VCAM-1 functionally contributed to Th1* cell adhesion to SMLC-enhanced EECM-BMEC-like cells as assessed using a VCAM-1 blocking antibody. Importantly, the SMLCs are a byproduct generated during the EECM-BMEC-like cell differentiation and as such, do not require independent differentiation of multiple cell types in parallel and are derived from the same hiPSC line as the EECM-BMEC-like cells, which should make this system readily applied to isogenic disease modeling. Taken together, EECM-BMEC-like cells represent a novel hiPSC-derived in vitro model of the BBB possessing key molecular and functional attributes required to evaluate interactions of immune cells with the BBB.

Cellular and molecular mechanisms mediating immune cell trafficking across the BBB have largely been studied in mouse models (4). Human in vitro BBB models have however been essential to confirm the translational importance of these mechanisms and verify novel therapeutic targets for inhibiting immune cell trafficking into the CNS in neuroinflammatory diseases in humans (12, 31, 62). Primary human brain microvascular endothelial cells (hBMECs) have proven especially useful to study immune cell/BBB interactions and have allowed the identification of additional molecules expressed on the BBB like ALCAM (12, 26), MCAM (69), and ninjurin-1 (70, 71), which seem to contribute to the recruitment of pathogenic T cells and myeloid cells into the CNS in the context of neuroinflammation. However, primary human brain microvascular endothelial cells mainly originate from surgical specimens, limiting the ability to establish patient-derived in vitro BBB model from diseases where biopsy or surgery is not common. The contribution of BBB alterations mediating immune cell trafficking has been largely studied in autopsy brain samples. Indeed adhesion molecules like ICAM-1, VCAM-1, ALCAM, MCAM, and ninjurin-1 were reported to be upregulated on brain endothelial cells in neuroinflammatory diseases such as multiple sclerosis (12, 26, 59, 69-72). However, direct contribution of these endothelial adhesion molecules to disease pathogenesis remains unknown since autopsy brain samples mainly reflect advanced stages of the disease and robust and reproducible functional assays using autopsy samples is quite difficult. Our novel EECM-BMEC-like cells, which possess proper adhesion molecules, open the field to study functional contributions of patient-derived brain endothelium in mediating altered immune cell migration into the CNS. Furthermore, isolating immune cells from the very same donors will facilitate the study immune cell migration across the BBB in an entirely autologous fashion. This will eventually also allow for novel observations on how antigen-specific processes may contribute to immune cell/BBB interactions including BBB disruption or antigen presentation by brain endothelium in human neurological disorders (73).

In conclusion, EECM-BMEC-like cells establish an in vitro BBB model that resembles primary brain microvascular endothelial cells in morphology, molecular junctional architecture, diffusion barrier characteristics, and adhesion molecule profile. This is thus the first model specifically adapted to study the role of the BBB in CNS immunity including immune cell migration across the BBB during CNS immune surveillance or inflammation. Employing patient-derived or genetically modified hiPSCs in concert with the EECM-BMEC-like cell differentiation protocol will facilitate study of the contribution of BBB impairment to CNS disorders ranging from multiple sclerosis to Alzheimer's disease. Employing immune cells from the very same donors will enable the unique opportunity to study immune cell interactions with the BBB in an autologous fashion.

### EXAMPLE 1 REFERENCES

1. Tietz, S., and Engelhardt, B. (2015) Brain barriers: Crosstalk between complex tight junctions and adherens junctions. The Journal of cell biology 209, 493-506
2. Engelhardt, B., and Sorokin, L. (2009) The blood-brain and the blood-cerebrospinal fluid barriers: function and dysfunction. Seminars in immunopathology 31, 497-511
3. Engelhardt, B., Vajkoczy, P., and Weller, R. O. (2017) The movers and shapers in immune privilege of the CNS. Nature immunology 18, 123-131
4. Engelhardt, B., and Ransohoff, R. M. (2012) Capture, crawl, cross: the T cell code to breach the blood-brain barriers. Trends in immunology 33, 579-589
5. Sweeney, M. D., Zhao, Z., Montagne, A., Nelson, A. R., and Zlokovic, B. V. (2019) Blood-Brain Barrier: From Physiology to Disease and Back. Physiological reviews 99, 21-78
6. Vajkoczy, P., Laschinger, M., and Engelhardt, B. (2001) Alpha4-integrin-VCAM-1 binding mediates G protein-independent capture of encephalitogenic T cell blasts to CNS white matter microvessels. The Journal of clinical investigation 108, 557-565
7. Abadier, M., Haghayegh Jahromi, N., Cardoso Alves, L., Boscacci, R., Vestweber, D., Barnum, S., Deutsch, U., Engelhardt, B., and Lyck, R. (2015) Cell surface levels of endothelial ICAM-1 influence the transcellular or paracellular T-cell diapedesis across the blood-brain barrier. European journal of immunology 45, 1043-1058
8. Bartholomaus, I., Kawakami, N., Odoardi, F., Schlager, C., Miljkovic, D., Ellwart, J. W., Klinkert, W. E., Flugel-Koch, C., Issekutz, T. B., Wekerle, H., and Flugel, A. (2009) Effector T cell interactions with meningeal vascular structures in nascent autoimmune CNS lesions. Nature 462, 94-98
9. Lecuyer, M. A., Saint-Laurent, O., Bourbonniere, L., Larouche, S., Larochelle, C., Michel, L., Charabati, M., Abadier, M., Zandee, S., Haghayegh Jahromi, N., Gowing, E., Pittet, C., Lyck, R.,
Engelhardt, B., and Prat, A. (2017) Dual role of ALCAM in neuroinflammation and blood-brain barrier homeostasis. Proceedings of the National Academy of Sciences of the United States of America
10. Weksler, B. B., Subileau, E. A., Perriere, N., Charneau, P., Holloway, K., Leveque, M., Tricoire-Leignel, H., Nicotra, A., Bourdoulous, S., Turowski, P., Male, D. K., Roux, F., Greenwood, J., Romero, I. A., and Couraud, P. O. (2005) Blood-brain barrier-specific properties of a human adult brain endothelial cell line. FASEB journal: official publication of the Federation of American Societies for Experimental Biology 19, 1872-1874
11. Helms, H. C., Abbott, N. J., Burek, M., Cecchelli, R., Couraud, P. O., Deli, M. A., Forster, C., Galla, H. J., Romero, I. A., Shusta, E. V., Stebbins, M. J., Vandenhaute, E., Weksler, B., and Brodin, B. (2016) In vitro models of the blood-brain barrier: An overview of commonly used brain endothelial cell culture models and guidelines for their use. Journal of cerebral blood flow and metabolism: official journal of the International Society of Cerebral Blood Flow and Metabolism 36, 862-890
12. Lyck, R., Lecuyer, M. A., Abadier, M., Wyss, C. B., Matti, C., Rosito, M., Enzmann, G., Zeis, T., Michel, L., Garcia Martin, A. B., Sallusto, F., Gosselet, F., Deutsch, U., Weiner, J. A., Schaeren-Wiemers, N., Prat, A., and Engelhardt, B. (2016) ALCAM (CD166) is involved in extravasation of monocytes rather than T cells across the blood-brain barrier. Journal of cerebral blood flow and metabolism: official journal of the International Society of Cerebral Blood Flow and Metabolism, 271678x16678639
13. Alvarez, J. I., Dodelet-Devillers, A., Kebir, H., Ifergan, I., Fabre, P. J., Terouz, S., Sabbagh, M., Wosik, K., Bourbonniere, L., Bernard, M., van Horssen, J., de Vries, H. E., Charron, F., and Prat, A. (2011) The Hedgehog pathway promotes blood-brain barrier integrity and CNS immune quiescence. Science (New York, N.Y.) 334, 1727-1731
14. Lippmann, E. S., Azarin, S. M., Kay, J. E., Nessler, R. A., Wilson, H. K., Al-Ahmad, A., Palecek, S. P., and Shusta, E. V. (2012) Derivation of blood-brain barrier endothelial cells from human pluripotent stem cells. Nature biotechnology 30, 783-791
15. Cecchelli, R., Aday, S., Sevin, E., Almeida, C., Culot, M., Dehouck, L., Coisne, C., Engelhardt, B., Dehouck, M. P., and Ferreira, L. (2014) A stable and reproducible human blood-brain barrier model derived from hematopoietic stem cells. PloS one 9, e99733
16. Stebbins, M. J., Wilson, H. K., Canfield, S. G., Qian, T., Palecek, S. P., and Shusta, E. V. (2016) Differentiation and characterization of human pluripotent stem cell-derived brain microvascular endothelial cells. Methods (San Diego, Calif.) 101, 93-102
17. Qian, T., Maguire, S. E., Canfield, S. G., Bao, X., Olson, W. R., Shusta, E. V., and Palecek, S. P. (2017) Directed differentiation of human pluripotent stem cells to blood-brain barrier endothelial cells. Science advances 3, e1701679
18. Praca, C., Rosa, S. C., Sevin, E., Cecchelli, R., Dehouck, M. P., and Ferreira, L. S. (2019) Derivation of Brain Capillary-like Endothelial Cells from Human Pluripotent Stem Cell-Derived Endothelial Progenitor Cells. Stem cell reports 13, 599-611
19. Lim, R. G., Quan, C., Reyes-Ortiz, A. M., Lutz, S. E., Kedaigle, A. J., Gipson, T. A., Wu, J., Vatine, G. D., Stocksdale, J., Casale, M. S., Svendsen, C. N., Fraenkel, E., Housman, D. E., Agalliu, D., and Thompson, L. M. (2017) Huntington's Disease iPSC-Derived Brain Microvascular Endothelial Cells Reveal WNT-Mediated Angiogenic and Blood-Brain Barrier Deficits. Cell reports 19, 1365-1377
20. Vatine, G. D., Al-Ahmad, A., Barriga, B. K., Svendsen, S., Salim, A., Garcia, L., Garcia, V. J., Ho, R., Yucer, N., Qian, T., Lim, R. G., Wu, J., Thompson, L. M., Spivia, W. R., Chen, Z., Van Eyk, J., Palecek, S. P., Refetoff, S., Shusta, E. V., and Svendsen, C. N. (2017) Modeling
Psychomotor Retardation using iPSCs from MCT8-Deficient Patients Indicates a Prominent Role for the Blood-Brain Barrier. Cell stem cell 20, 831-843.e835
21. Vatine, G. D., Barrile, R., Workman, M. J., Sances, S., Barriga, B. K., Rahnama, M., Barthakur, S., Kasendra, M., Lucchesi, C., Kerns, J., Wen, N., Spivia, W. R., Chen, Z., Van Eyk, J., and Svendsen, C. N. (2019) Human iPSC-Derived Blood-Brain Barrier Chips Enable Disease Modeling and Personalized Medicine Applications. Cell stem cell 24, 995-1005.e1006
22. Linville, R. M., DeStefano, J. G., Sklar, M. B., Xu, Z., Farrell, A. M., Bogorad, M. I., Chu, C., Walczak, P., Cheng, L., Mahairaki, V., Whartenby, K. A., Calabresi, P. A., and Searson, P. C. (2019) Human iPSC-derived blood-brain barrier microvessels: validation of barrier function and endothelial cell behavior. Biomaterials 190-191, 24-37
23. Lippmann, E. S., Al-Ahmad, A., Azarin, S. M., Palecek, S. P., and Shusta, E. V. (2014) A retinoic acid-enhanced, multicellular human blood-brain barrier model derived from stem cell sources. Scientific reports 4, 4160
24. Urich, E., Lazic, S. E., Molnos, J., Wells, I., and Freskgård, P. O. (2012) Transcriptional profiling of human brain endothelial cells reveals key properties crucial for predictive in vitro blood-brain barrier models. PloS one 7, e38149
25. Malin, D., Strekalova, E., Petrovic, V., Deal, A. M., Al Ahmad, A., Adamo, B., Miller, C. R., Ugolkov, A., Livasy, C., Fritchie, K., Hamilton, E., Blackwell, K., Geradts, J., Ewend, M., Carey, L., Shusta, E. V., Anders, C. K., and Cryns, V. L. (2014) aB-crystallin: a novel regulator of breast cancer metastasis to the brain. Clinical cancer research: an official journal of the American Association for Cancer Research 20, 56-67
26. Cayrol, R., Wosik, K., Berard, J. L., Dodelet-Devillers, A., Ifergan, I., Kebir, H., Haqqani, A. S., Kreymborg, K., Krug, S., Moumdjian, R., Bouthillier, A., Becher, B., Arbour, N., David, S., Stanimirovic, D., and Prat, A. (2008) Activated leukocyte cell adhesion molecule promotes leukocyte trafficking into the central nervous system. Nature immunology 9, 137-145
27. Cayrol, R., Haqqani, A. S., Ifergan, I., Dodelet-Devillers, A., and Prat, A. (2011) Isolation of human brain endothelial cells and characterization of lipid raft-associated proteins by mass spectroscopy. Methods Mol Biol 686, 275-295
28. Podjaski, C., Alvarez, J. I., Bourbonniere, L., Larouche, S., Terouz, S., Bin, J. M., Lecuyer, M. A., Saint-Laurent, O., Larochelle, C., Darlington, P. J., Arbour, N., Antel, J. P., Kennedy, T. E., and Prat, A. (2015) Netrin 1 regulates blood-brain barrier function and neuroinflammation. Brain: a journal of neurology 138, 1598-1612
29. Perriot, S., Mathias, A., Perriard, G., Canales, M., Jonkmans, N., Merienne, N., Meunier, C., El Kassar, L., Perrier, A. L., Laplaud, D. A., Schluep, M., Deglon, N., and Du Pasquier, R. (2018) Human Induced Pluripotent Stem Cell-Derived Astrocytes Are Differentially Activated by Multiple Sclerosis-Associated Cytokines. Stem cell reports 11, 1199-1210
30. Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., Slukvin, II, and Thomson, J. A. (2007) Induced pluripotent stem cell lines derived from human somatic cells. Science (New York, N.Y.) 318, 1917-1920
31. Nishihara, H., Soldati, S., Mossu, A., Rosito, M., Rudolph, H., Muller, W. A., Latorre, D., Sallusto, F., Sospedra, M., Martin, R., Ishikawa, H., Tenenbaum, T., Schroten, H., Gosselet, F., and Engelhardt, B. (2020) Human CD4(+) T cell subsets differ in their abilities to cross endothelial and epithelial brain barriers in vitro. Fluids and barriers of the CNS 17, 3
32. Lian, X., Bao, X., Al-Ahmad, A., Liu, J., Wu, Y., Dong, W., Dunn, K. K., Shusta, E. V., and Palecek, S. P. (2014) Efficient differentiation of human pluripotent stem cells to endothelial progenitors via small-molecule activation of WNT signaling. Stem cell reports 3, 804-816
33. Bao, X., Lian, X., and Palecek, S. P. (2016) Directed Endothelial Progenitor Differentiation from Human Pluripotent Stem Cells Via Wnt Activation Under Defined Conditions. Methods Mol Biol 1481, 183-196
34. Shimizu, F., Sano, Y., Abe, M. A., Maeda, T., Ohtsuki, S., Terasaki, T., and Kanda, T. (2011) Peripheral nerve pericytes modify the blood-nerve barrier function and tight junctional molecules through the secretion of various soluble factors. Journal of cellular physiology 226, 255-266
35. Haruki, H., Sano, Y., Shimizu, F., Omoto, M., Tasaki, A., Oishi, M., Koga, M., Saito, K., Takahashi, T., Nakada, T., and Kanda, T. (2013) NMO sera down-regulate AQP4 in human astrocyte and induce cytotoxicity independent of complement. Journal of the neurological sciences 331, 136-144
36. Sano, Y., Shimizu, F., Abe, M., Maeda, T., Kashiwamura, Y., Ohtsuki, S., Terasaki, T., Obinata, M., Kajiwara, K., Fujii, M., Suzuki, M., and Kanda, T. (2010) Establishment of a new conditionally immortalized human brain microvascular endothelial cell line retaining an in vivo blood-brain barrier function. Journal of cellular physiology 225, 519-528
37. Mossu, A., Rosito, M., Khire, T., Li Chung, H., Nishihara, H., Gruber, I., Luke, E., Dehouck, L., Sallusto, F., Gosselet, F., McGrath, J. L., and Engelhardt, B. (2018) A silicon nanomembrane platform for the visualization of immune cell trafficking across the human blood-brain barrier under flow. Journal of cerebral blood flow and metabolism: official journal of the International Society of Cerebral Blood Flow and Metabolism, 271678x18820584
38. Stebbins, M. J., Gastfriend, B. D., Canfield, S. G., Lee, M. S., Richards, D., Faubion, M. G., Li, W. J., Daneman, R., Palecek, S. P., and Shusta, E. V. (2019) Human pluripotent stem cell-derived brain pericyte-like cells induce blood-brain barrier properties. Science advances 5, eaau7375
39. Engen, S. A., Valen Rukke, H., Becattini, S., Jarrossay, D., Blix, I. J., Petersen, F. C., Sallusto, F., and Schenck, K. (2014) The oral commensal Streptococcus mitis shows a mixed memory Th cell signature that is similar to and cross-reactive with Streptococcus pneumoniae. PloS one 9, e104306
40. Sallusto, F., Schaerli, P., Loetscher, P., Schaniel, C., Lenig, D., Mackay, C. R., Qin, S., and Lanzavecchia, A. (1998) Rapid and coordinated switch in chemokine receptor expression during dendritic cell maturation. European journal of immunology 28, 2760-2769
41. Coisne, C., Lyck, R., and Engelhardt, B. (2013) Live cell imaging techniques to study T cell trafficking across the blood-brain barrier in vitro and in vivo. Fluids and barriers of the CNS 10, 7
42. Mizee, M. R., Nijland, P. G., van der Pol, S. M., Drexhage, J. A., van Het Hof, B., Mebius, R., van der Valk, P., van Horssen, J., Reijerkerk, A., and de Vries, H. E. (2014) Astrocyte-derived retinoic acid: a novel regulator of blood-brain barrier function in multiple sclerosis. Acta neuropathologica 128, 691-703
43. Canfield, S. G., Stebbins, M. J., Morales, B. S., Asai, S. W., Vatine, G. D., Svendsen, C. N., Palecek, S. P., and Shusta, E. V. (2017) An isogenic blood-brain barrier model comprising brain endothelial cells, astrocytes, and neurons derived from human induced pluripotent stem cells. J Neurochem 140, 874-888
44. Herich, S., Schneider-Hohendorf, T., Rohlmann, A., Khaleghi Ghadiri, M., Schulte-Mecklenbeck, A., Zondler, L., Janoschka, C., Ostkamp, P., Richter, J., Breuer, J., Dimitrov, S., Rammensee, H. G., Grauer, O. M., Klotz, L., Gross, C. C., Stummer, W., Missler, M., Zarbock, A., Vestweber, D., Wiendl, H., and Schwab, N. (2019) Human CCR5high effector memory cells perform CNS parenchymal immune surveillance via GZMK-mediated transendothelial diapedesis. Brain: a journal of neurology
45. Alter, A., Duddy, M., Hebert, S., Biernacki, K., Prat, A., Antel, J. P., Yong, V. W., Nuttall, R. K., Pennington, C. J., Edwards, D. R., and Bar-Or, A. (2003) Determinants of human B cell migration across brain endothelial cells. Journal of immunology 170, 4497-4505
46. Chang, S. F., Chen, L. J., Lee, P. L., Lee, D. Y., Chien, S., and Chiu, J. J. (2014) Different modes of endothelial-smooth muscle cell interaction elicit differential beta-catenin phosphorylations and endothelial functions. Proceedings of the National Academy of Sciences of the United States of America 111, 1855-1860
47. Lopes Pinheiro, M. A., Kooij, G., Mizee, M. R., Kamermans, A., Enzmann, G., Lyck, R., Schwaninger, M., Engelhardt, B., and de Vries, H. E. (2016) Immune cell trafficking across the barriers of the central nervous system in multiple sclerosis and stroke. Biochimica et biophysica acta 1862, 461-471
48. Engelhardt, B., Carare, R. O., Bechmann, I., Flugel, A., Laman, J. D., and Weller, R. O. (2016) Vascular, glial, and lymphatic immune gateways of the central nervous system. Acta neuropathologica 132, 317-338
49. Bernas, M. J., Cardoso, F. L., Daley, S. K., Weinand, M. E., Campos, A. R., Ferreira, A. J., Hoying, J. B., Witte, M. H., Brites, D., Persidsky, Y., Ramirez, S. H., and Brito, M. A. (2010) Establishment of primary cultures of human brain microvascular endothelial cells to provide an in vitro cellular model of the blood-brain barrier. Nature protocols 5, 1265-1272
50. Eigenmann, D. E., Xue, G., Kim, K. S., Moses, A. V., Hamburger, M., and Oufir, M. (2013) Comparative study of four immortalized human brain capillary endothelial cell lines, hCMEC/D3, hBMEC, TY10, and BB19, and optimization of culture conditions, for an in vitro blood-brain barrier model for drug permeability studies. Fluids and barriers of the CNS 10, 33
51. Boyer-Di Ponio, J., El-Ayoubi, F., Glacial, F., Ganeshamoorthy, K., Driancourt, C., Godet, M., Perriere, N., Guillevic, O., Couraud, P. O., and Uzan, G. (2014) Instruction of circulating endothelial progenitors in vitro towards specialized blood-brain barrier and arterial phenotypes. PloS one 9, e84179
52. Clark, P. A., Al-Ahmad, A. J., Qian, T., Zhang, R. R., Wilson, H. K., Weichert, J. P., Palecek, S. P., Kuo, J. S., and Shusta, E. V. (2016) Analysis of Cancer-Targeting Alkylphosphocholine Analogue Permeability Characteristics Using a Human Induced Pluripotent Stem Cell Blood-Brain Barrier Model. Molecular pharmaceutics 13, 3341-3349
53. Hollmann, E. K., Bailey, A. K., Potharazu, A. V., Neely, M. D., Bowman, A. B., and Lippmann, E. S. (2017) Accelerated differentiation of human induced pluripotent stem cells to blood-brain barrier endothelial cells. Fluids and barriers of the CNS 14, 9
54. Ribecco-Lutkiewicz, M., Sodja, C., Haukenfrers, J., Haqqani, A. S., Ly, D., Zachar, P., Baumann, E., Ball, M., Huang, J., Rukhlova, M., Martina, M., Liu, Q., Stanimirovic, D., Jezierski, A., and Bani-Yaghoub, M. (2018) A novel human induced pluripotent stem cell blood-brain barrier model: Applicability to study antibody-triggered receptor-mediated transcytosis. Scientific reports 8, 1873
55. Neal, E. H., Marinelli, N. A., Shi, Y., McClatchey, P. M., Balotin, K. M., Gullett, D. R., Hagerla, K. A., Bowman, A. B., Ess, K. C., Wikswo, J. P., and Lippmann, E. S. (2019) A Simplified, Fully Defined Differentiation Scheme for Producing Blood-Brain Barrier Endothelial Cells from Human iPSCs. Stem cell reports 12, 1380-1388
56. Kerfoot, S. M., Norman, M. U., Lapointe, B. M., Bonder, C. S., Zbytnuik, L., and Kubes, P. (2006) Reevaluation of P-selectin and alpha 4 integrin as targets for the treatment of experimental autoimmune encephalomyelitis. Journal of immunology 176, 6225-6234
57. Schlager, C., Korner, H., Krueger, M., Vidoli, S., Haberl, M., Mielke, D., Brylla, E., Issekutz, T., Cabanas, C., Nelson, P. J., Ziemssen, T., Rohde, V., Bechmann, I., Lodygin, D., Odoardi, F., and Flugel, A. (2016) Effector T-cell trafficking between the leptomeninges and the cerebrospinal fluid. Nature 530, 349-353
58. Steiner, O., Coisne, C., Cecchelli, R., Boscacci, R., Deutsch, U., Engelhardt, B., and Lyck, R. (2010) Differential roles for endothelial ICAM-1, ICAM-2, and VCAM-1 in shear-resistant T cell arrest, polarization, and directed crawling on blood-brain barrier endothelium. Journal of immunology 185, 4846-4855
59. Alvarez, J. I., Cayrol, R., and Prat, A. (2011) Disruption of central nervous system barriers in multiple sclerosis. Biochimica et biophysica acta 1812, 252-264
60. Luo, J., Paranya, G., and Bischoff, J. (1999) Noninflammatory expression of E-selectin is regulated by cell growth. Blood 93, 3785-3791
61. Castro Dias, M., Coisne, C., Lazarevic, I., Baden, P., Hata, M., Iwamoto, N., Francisco, D. M. F., Vanlandewijck, M., He, L., Baier, F. A., Stroka, D., Bruggmann, R., Lyck, R., Enzmann, G., Deutsch, U., Betsholtz, C., Furuse, M., Tsukita, S., and Engelhardt, B. (2019) Claudin-3-deficient C57BL/6J mice display intact brain barriers. Scientific reports 9, 203
62. Wimmer, I., Tietz, S., Nishihara, H., Deutsch, U., Sallusto, F., Gosselet, F., Lyck, R., Muller, W. A., Lassmann, H., and Engelhardt, B. (2019) PECAM-1 Stabilizes Blood-Brain Barrier Integrity and Favors Paracellular T-Cell Diapedesis Across the Blood-Brain Barrier During Neuroinflammation. Frontiers in immunology 10
63. Steiner, O., Coisne, C., Engelhardt, B., and Lyck, R. (2011) Comparison of immortalized bEndS and primary mouse brain microvascular endothelial cells as in vitro blood-brain barrier models for the study of T cell extravasation. Journal of cerebral blood flow and metabolism: official journal of the International Society of Cerebral Blood Flow and Metabolism 31, 315-327
64. Hirase, T., Staddon, J. M., Saitou, M., Ando-Akatsuka, Y., Itoh, M., Furuse, M., Fujimoto, K., Tsukita, S., and Rubin, L. L. (1997) Occludin as a possible determinant of tight junction permeability in endothelial cells. Journal of cell science 110 (Pt 14), 1603-1613
65. Liebner, S., Corada, M., Bangsow, T., Babbage, J., Taddei, A., Czupalla, C. J., Reis, M., Felici, A., Wolburg, H., Fruttiger, M., Taketo, M. M., von Melchner, H., Plate, K. H., Gerhardt, H., and Dejana, E. (2008) Wnt/beta-catenin signaling controls development of the blood-brain barrier. The Journal of cell biology 183, 409-417
66. Bonney, S., Harrison-Uy, S., Mishra, S., MacPherson, A. M., Choe, Y., Li, D., Jaminet, S. C., Fruttiger, M., Pleasure, S. J., and Siegenthaler, J. A. (2016) Diverse Functions of Retinoic Acid in Brain Vascular Development. The Journal of neuroscience: the official journal of the Society for Neuroscience 36, 7786-7801
67. Vazao, H., Rosa, S., Barata, T., Costa, R., Pitrez, P. R., Honorio, I., de Vries, M. R., Papatsenko, D., Benedito, R., Saris, D., Khademhosseini, A., Quax, P. H., Pereira, C. F., Mercader, N., Fernandes, H., and Ferreira, L. (2017) High-throughput identification of small molecules that affect human embryonic vascular development. Proceedings of the National Academy of Sciences of the United States of America 114, E3022-e3031
68. Halaidych, O. V., Freund, C., van den Hil, F., Salvatori, D. C. F., Riminucci, M., Mummery, C. L., and Orlova, V. V. (2018) Inflammatory Responses and Barrier Function of Endothelial Cells Derived from Human Induced Pluripotent Stem Cells. Stem cell reports 10, 1642-1656
69. Larochelle, C., Cayrol, R., Kebir, H., Alvarez, J. I., Lecuyer, M. A., Ifergan, I., Viel, E., Bourbonniere, L., Beauseigle, D., Terouz, S., Hachehouche, L., Gendron, S., Poirier, J., Jobin, C., Duquette, P., Flanagan, K., Yednock, T., Arbour, N., and Prat, A. (2012) Melanoma cell adhesion molecule identifies encephalitogenic T lymphocytes and promotes their recruitment to the central nervous system. Brain: a journal of neurology 135, 2906-2924
70. Ifergan, I., Kebir, H., Terouz, S., Alvarez, J. I., Lecuyer, M. A., Gendron, S., Bourbonniere, L., Dunay, I. R., Bouthillier, A., Moumdjian, R., Fontana, A., Haqqani, A., Klopstein, A., Prinz, M., Lopez-Vales, R., Birchler, T., and Prat, A. (2011) Role of Ninjurin-1 in the migration of myeloid cells to central nervous system inflammatory lesions. Annals of neurology 70, 751-763
71. Odoardi, F., Sie, C., Streyl, K., Ulaganathan, V. K., Schlager, C., Lodygin, D., Heckelsmiller, K., Nietfeld, W., Ellwart, J., Klinkert, W. E., Lottaz, C., Nosov, M., Brinkmann, V., Spang, R., Lehrach, H., Vingron, M., Wekerle, H., Flugel-Koch, C., and Flugel, A. (2012) T cells become licensed in the lung to enter the central nervous system. Nature 488, 675-679
72. Alvarez, J. I., Saint-Laurent, O., Godschalk, A., Terouz, S., Briels, C., Larouche, S., Bourbonniere, L., Larochelle, C., and Prat, A. (2015) Focal disturbances in the blood-brain barrier are associated with formation of neuroinflammatory lesions. Neurobiology of disease 74, 14-24
73. Lopes Pinheiro, M. A., Kamermans, A., Garcia-Vallejo, J. J., van Het Hof, B., Wierts, L., O'Toole, T., Boeve, D., Verstege, M., van der Pol, S. M., van Kooyk, Y., de Vries, H. E., and Unger, W. W. (2016) Internalization and presentation of myelin antigens by the brain endothelium guides antigen-specific T cell migration. eLife 5

### Example 2: Wnt Signaling Mediates Acquisition of Blood-Brain Barrier Properties in Naïve Endothelium Derived from Human Pluripotent Stem Cells

Example 2 demonstrates and additional embodiment of the present invention. In this example, the inventors provide an additional method of producing BMEC-like cells for use in a blood brain barrier model that have a CNS EC phenotype having barrier properties. The inventors found that Wnt activation in hPSC-derived, naïve endothelial progenitors drives development of a CNS EC-like phenotype. The inventors found that many aspects of the CNS EC phenotype, including the canonical GLUT-1, claudin-5, and PLVAP expression effects, were regulated by CHIR 99021, a small molecule agonist of Wnt/β-catenin signaling. Wnt ligands and conditioned media from neural cells produced a more limited response, as did CHIR treatment in matured ECs. Whole-transcriptome analysis revealed definitive endothelial identity of the resulting cells and CHIR-upregulated expression of known CNS EC transcripts, including LEF1, APCDD1, AXIN2, SLC2A1, CLDN5, LSR, ABCG2, SOX7, and ZIC3. The inventors also observed an unexpected CHIR-mediated upregulation of caveolin-1, which did not, however, correlate with increased uptake of a dextran tracer. The inventors found cell-autonomous Wnt activation in hPSC-derived naïve endothelial progenitors is sufficient to establish many aspects of the CNS barrier EC phenotype, and established a model system for further systematic investigation of putative barriergenic cues.

Briefly, the protocol to produce endothelial progenitor cells (EPCs) from hPSCs is depicted in FIG. 23A. To achieve mesoderm specification, this method employs an initial activation of Wnt/β-catenin signaling with CHIR 99021 (CHIR), a small molecule inhibitor of glycogen synthase kinase-3 (GSK-3), which results in inhibition of GSK-3β-mediated β-catenin degradation. After 5 days of expansion, the resulting cultures contained a mixed population of CD34⁺CD31⁺ EPCs and CD34⁻CD31⁻ non-EPCs (FIG. 23B-C). Magnetic-activated cell sorting (MACS) was used to isolate CD31⁺ cells from this mixed culture and these cells were plated on collagen IV-coated plates in hESCR medium. The inventors demonstrated this method could induce GLUT-1 expression in the resulting ECs. After 6 days of treatment, a significant increase in the fraction of GLUT-1⁺ ECs in Wnt3a-treated cultures compared to controls was observed (FIG. 23D-E). Consistent with previous observations (Example 1), the inventors also detected a population of calponin⁺ smooth muscle protein 22-α⁺ putative smooth muscle-like cells (SMLCs) outside the endothelial colonies (FIG. 23F) and these SMLCs expressed GLUT-1 in both control and Wnt3a-treated conditions (FIG. 23D). Molecular hallmarks of CNS EC barriergenesis are (i) acquisition of expression of the glucose transporter GLUT-1, (ii) loss of plasmalemma vesicle-associated protein (PLVAP), and (iii) upregulation of claudin-5 (Cho et al., 2017; Daneman et al., 2009; Kuhnert et al., 2010; Umans et al., 2017; Wang et al., 2019), which was demonstrated in the EC derived cells of this method.

CHIR treatment robustly induced several canonical CNS EC molecular phenotypes, including a marked induction of GLUT-1, upregulation of claudin-5, and downregulation of PLVAP, which correlated with differential gene expression in RNA-seq data. Further, using RNA-seq and Western blotting, the inventors also identified LSR (angulin-1) as CHIR-induced in this system, supporting the notion that this highly CNS EC-enriched tricellular tight junction protein (Daneman et al., 2010a; Sohet et al., 2015) is Wnt-regulated. In RNA-seq data, the inventors observed differential expression of known CNS EC-enriched/depleted and Wnt-regulated genes including upregulated LEF1, AXIN2, APCDDJ, ABCG2, SOX7, and ZIC3 and downregulated PLVAP, FABP4, and SMAD6.

Endothelial cells (ECs) in the central nervous system (CNS) acquire their specialized blood-brain barrier (BBB) properties in response to extrinsic signals, with Wnt/β-catenin signaling coordinating multiple aspects of this process. Our knowledge of CNS EC development has been advanced largely by animal models, and human pluripotent stem cells (hPSCs) offer the opportunity to examine BBB development in an in vitro human system. Here we show that activation of Wnt signaling in hPSC-derived naïve endothelial progenitors, but not in matured ECs, leads to robust acquisition of canonical BBB phenotypes including expression of GLUT-1, increased claudin-5, decreased PLVAP and decreased permeability. RNA-seq revealed a transcriptome profile resembling ECs with CNS-like characteristics, including Wnt-upregulated expression of LEF1, APCDD1, and ZIC3. Together, our work defines effects of Wnt activation in naïve ECs and establishes an improved hPSC-based model for interrogation of CNS barriergenesis.

In the central nervous system (CNS), vascular endothelial cells (ECs) are highly specialized, with complex tight junctions, expression of a spectrum of nutrient and efflux transporters, low rates of vesicle trafficking, no fenestrae, and low expression of immune cell adhesion molecules (Reese and Karnovsky, 1967; Obermeier et al., 2013). ECs bearing these attributes, often referred to as the blood-brain barrier (BBB), work in concert with the other brain barriers to facilitate the tight regulation of the CNS microenvironment required for proper neuronal function (Daneman and Engelhardt, 2017; Profaci et al., 2020). During development, the Wnt/β-catenin signaling pathway drives both CNS angiogenesis, during which vascular sprouts originating from the perineural vascular plexus invade the developing neural tube, and the coupled process of barriergenesis by which resulting ECs begin to acquire BBB properties (Liebner et al., 2008; Stenman et al., 2008; Daneman et al., 2009; Engelhardt and Liebner, 2014; Umans et al., 2017). Specifically, neural progenitor-derived Wnt7a and Wnt7b ligands signal through Frizzled receptors and the obligate co-receptors RECK and GPR124 (ADGRA2) on endothelial cells (Kuhnert et al., 2010; Cullen et al., 2011; Vanhollebeke et al., 2015; Cho et al., 2017; Eubelen et al., 2018; Vallon et al., 2018). Other ligands function analogously in the retina (Norrin) (Ye et al., 2009; Wang et al., 2012) and potentially in the dorsal neural tube (Daneman et al., 2009). Furthermore, Wnt/β-catenin signaling is required for maintenance of CNS EC barrier properties in adulthood (Tran et al., 2016), with astrocytes as a major source of Wnt7 ligands (He et al., 2018; Vanlandewijck et al., 2018; Guérit et al., 2021).

Molecular hallmarks of Wnt-mediated CNS EC barriergenesis are (i) acquisition of glucose transporter GLUT-1 expression, (ii) loss of plasmalemma vesicle-associated protein (PLVAP), and (iii) upregulation of claudin-5 (Daneman et al., 2009; Kuhnert et al., 2010; Cho et al., 2017; Umans et al., 2017; Wang et al., 2019). Notably, the Wnt-mediated switch between the “leaky” EC phenotype (GLUT-1⁻ PLVAP⁺ claudin-5^{low}) and the barrier EC phenotype (GLUT-1⁺ PLVAP⁻ claudin-5^{high}) correlates with reduced permeability to molecular tracers (Wang et al., 2012; Cho et al., 2017) and is conserved in multiple contexts. For instance, medulloblastomas that produce Wnt-inhibitory factors have leaky vessels (Phoenix et al., 2016). Moreover, vasculature perfusing circumventricular organs is leaky due to low levels of Wnt signaling (Benz et al., 2019; Wang et al., 2019). Notably, ectopic activation of Wnt in ECs of circumventricular organs induces GLUT-1 and suppresses PLVAP (Benz et al., 2019; Wang et al., 2019). However, similar ectopic activation of Wnt in liver and lung ECs produces only very minor barriergenic effects (Munji et al., 2019), and Wnt activation in cultured primary mouse brain ECs does not prevent culture-induced loss of barrier-associated gene expression (Sabbagh and Nathans, 2020). The reasons for the apparent context-dependent impacts of Wnt activation in ECs remain unclear and motivate systematic examination of this process in a simplified model system. Further, given species differences in brain EC transporter expression (Uchida et al., 2011), drug permeability (Syvänen et al., 2009), and gene expression (Song et al., 2020), this process warrants investigation in human cells to complement mouse in vivo studies.

Prior studies have evaluated the impact of Wnt activation in immortalized human brain ECs and observed only modest effects on barrier phenotype (Paolinelli et al., 2013; Laksitorini et al., 2019). Combined with the aforementioned deficits observed in primary adult mouse brain endothelial cells that are not rescued by ectopic Wnt activation (Sabbagh and Nathans, 2020), one possibility is that mature, adult endothelium is largely refractory to Wnt activation, and that Wnt responsiveness is a property of immature endothelial cells analogous to those in the perineural vascular plexus. Human pluripotent stem cells (hPSCs) offer a potential human model system for investigation of molecular mechanisms of BBB phenotype acquisition. However, currently available hPSC-based models of CNS endothelial-like cells are not well suited for modeling the BBB developmental progression as they do not follow a developmentally-relevant differentiation trajectory, lack definitive endothelial identity, or have been incompletely characterized with respect to the role of developmental signaling pathways (Lippmann et al., 2020; Workman and Svendsen, 2020). As a potential alternative, hPSCs can also be used to generate immature, naïve endothelial progenitors (Lian et al., 2014) that could be used to better explore the induction of BBB phenotypes. For example, we recently reported that extended culture of such hPSC-derived endothelial progenitors in a minimal medium yielded ECs with improved BBB tight junction protein expression and localization which led to improved paracellular barrier properties (Nishihara et al., 2020). However, as shown below, these cells exhibit high expression of PLVAP and little expression of GLUT-1, indicating the need for additional cues to drive CNS EC specification.

In this work, we tested the hypothesis that activation of Wnt/β-catenin signaling in hPSC-derived, naïve endothelial progenitors would drive development of a CNS EC-like phenotype. We found that many aspects of the CNS EC phenotype, including the canonical GLUT-1, claudin-5, and PLVAP expression effects, were regulated by CHIR 99021, a small molecule agonist of Wnt/β-catenin signaling. Wnt ligands and conditioned media from neural progenitors produced a more limited response, as did CHIR treatment in matured ECs. Whole-transcriptome analysis revealed definitive endothelial identity of the resulting cells and CHIR-upregulated expression of known CNS EC transcripts, including LEF1, APCDD1, AXIN2, SLC2A1, CLDN5, LSR, ABCG2, SOX7, and ZIC3. We also observed an unexpected CHIR-mediated upregulation of caveolin-1, which did not, however, correlate with increased uptake of a dextran tracer. Thus, we provide evidence that Wnt activation in hPSC-derived naïve endothelial progenitors is sufficient to induce many aspects of the CNS barrier EC phenotype, and we establish a model system for further systematic investigation of putative barriergenic cues.

### Materials and Methods:

TABLE 6
Resources

| Reagent | | Source or | | Additional |
|---|---|---|---|---|
| type | Designation | reference | Identifier | information |
| Cell line | iPSC: IMR90-4 | Available from | RRID: | |
| | | WiCell; (Jaffe | CVCL_C437 | |
| | | et al., 2008) | | |
| Cell line | iPSC: WTC11 | Available from | RRID: | |
| | | Gladstone | CVCL_Y803 | |
| | | Institutes; | | |
| | | (Kreitzer et al., | | |
| | | 2013) | | |
| Cell line | iPSC: CS03iCTRn2 | Available from | | |
| | | Cedars Sinai | | |
| | | iPSC Core | | |
| Cell line | iPSC: 19-9-11-7TGP- | Laboratory | | |
| | ishcat3 | stock | | |
| Cell line | hESC: H9-7TGP-ishcat2 | Laboratory | | |
| | | stock | | |
| | | (Lian et al., | | |
| | | 2013) | | |
| Cell line | hESC: H9-CDH5-eGFP | Laboratory | | |
| | | stock | | |
| | | (Bao et al., | | |
| | | 2017) | | |
| Antibody | Anti-CD31-FITC | Miltenyi Biotec | Cat# 130-117- | |
| | (mouse IgG1, clone | | 390 | |
| | AC128) | | | |
| Antibody | Anti-CD31-APC (mouse | Miltenyi Biotec | Cat# 130-119- | |
| | IgG1, clone AC128) | | 891 | |
| Antibody | Anti-CD34-FITC | Miltenyi Biotec | Cat# 130-113- | |
| | (mouse IgG2a, clone | | 178 | |
| | AC136) | | | |
| Antibody | Anti-β-catenin-Alexa | BD Biosciences | Cat# 562505 | 1:100 (ICC) |
| | Fluor 488 (mouse IgG1, | | | |
| | clone 14) | | | |
| Antibody | Anti-GLUT-1 (mouse | Invitrogen | Cat# MA5- | 1:100 (ICC) |
| | IgG2a, clone SPM498) | | 11315 | 1:500 (WB) |
| Antibody | Anti-calponin (mouse | Sigma-Aldrich | Cat# C2687 | 1:15000 |
| | IgG1, clone hCP) | | | (ICC) |
| Antibody | Anti-SM22α (rabbit | Abcam | Cat# ab10146 | 1:1000 (ICC) |
| | polyclonal) | | | |
| Antibody | Anti-claudin-5 (mouse | Invitrogen | Cat# 35-2500 | 1:100 (ICC) |
| | IgG1, clone 4C3C2) | | | 1:500 (WB) |
| Antibody | Anti-caveolin-1 (rabbit | Cell Signaling | Cat# 3238 | 1:500 (ICC) |
| | polyclonal) | Technology | | |
| Antibody | Anti-CD31 (rabbit | Lab Vision | Cat# RB- | 1:100 (ICC) |
| | polyclonal) | | 10333-P | |
| Antibody | Anti-Ki67 (mouse IgG1, | BD Biosciences | Cat# 550609 | 1:100 (ICC) |
| | clone B56) | | | |
| Antibody | Anti-VE-cadherin | Santa Cruz | Cat# sc-52751 | 1:100 (ICC) |
| | (mouse IgG2a, clone | Biotechnology | | 1:250 (WB) |
| | BV9) | | | |
| Antibody | Anti-β-actin (rabbit IgG, | Cell Signaling | Cat# 4970 | 1:1000 (WB) |
| | clone 13E5) | Technology | | |
| Antibody | Anti-PLVAP (rabbit | Prestige | Cat# | 1:200 (ICC) |
| | polyclonal) | Antibodies | HPA002279 | 1:250 (WB) |
| Antibody | Anti-LSR (rabbit | Prestige | Cat# | 1:250 (WB) |
| | polyclonal) | Antibodies | HPA007270 | |
| Antibody | Alexa Fluor 488 goat | Invitrogen | Cat# A-11001 | 1:200 (ICC) |
| | anti-mouse IgG | | | |
| Antibody | Alexa Fluor 647 goat | Invitrogen | Cat# A-21245 | 1:200 (ICC) |
| | anti-rabbit IgG | | | |
| Antibody | Alexa Fluor 488 goat | Invitrogen | Cat# A-21121 | 1:200 (ICC) |
| | anti-mouse IgG1 | | | |
| Antibody | Alexa Fluor 647 goat | Invitrogen | Cat# A-21241 | 1:200 (ICC) |
| | anti-mouse IgG2a | | | |
| Antibody | Alexa Fluor 555 goat | Invitrogen | Cat# A-21428 | 1:200 (ICC) |
| | anti-rabbit IgG | | | |
| Antibody | IRDye 800CW goat | LI-COR | Cat# 926-32210 | 1:5000 (WB) |
| | anti-mouse IgG | Biosciences | | |
| Antibody | IRDye 800CW goat | LI-COR | Cat# 926-32211 | 1:5000 (WB) |
| | anti-rabbit IgG | Biosciences | | |
| Antibody | IRDye 680RD goat anti- | LI-COR | Cat# 926-68071 | 1:5000 (WB) |
| | rabbit IgG | Biosciences | | |
| Commercial | RNeasy Plus Micro Kit | Qiagen | Cat# 74034 | |
| assay or kit | | | | |
| Chemical | CHIR 99021 | Tocris | Cat# 4423 | |
| compound | | | | |
| or drug | | | | |
| Chemical | Vybrant DyeCycle | Invitrogen | Cat# V35004 | |
| compound | Green Stain | | | |
| or drug | | | | |
| Chemical | Dextran, Alexa Fluor | Invitrogen | Cat# D22910 | |
| compound | 488; 10,000 MW, | | | |
| or drug | Anionic, Fixable | | | |
| Software or | RSEM | (Li and Dewey, | | v1.3.3 |
| algorithm | | 2011) | | |
| Software or | Bowtie2 | (Langmead and | | v2.4.2 |
| algorithm | | Salzberg, 2012) | | |
| Software or | R | R Foundation | | v3.6.3 |
| algorithm | | | | |
| Software or | DESeq2 | (Love et al., | | v1.26.0 |
| algorithm | | 2014) | | |
| Software or | biomaRt | (Durinck et al., | | v2.42.1 |
| algorithm | | 2009) | | |
| Software or | FIJI/ImageJ | (Schindelin et | | v2.0.0-rc-68 |
| algorithm | | al., 2019) | | |
| Software or | Image Studio | LI-COR | | v5.2 |
| algorithm | | Biosciences | | |
| Software or | FlowJo | BD Biosciences | | v10.7.1 |
| algorithm | | | | |
| Software or | JMP Pro | SAS Institute | | v14.0.0 |
| algorithm | | | | |
| Software or | Prism | GraphPad | | v5.0.1 |
| algorithm | | Software | | |

hPSC Maintenance

Tissue culture plates were coated with Matrigel, Growth Factor Reduced (Corning, Glendale, AZ). A 2.5 mg aliquot of Matrigel was thawed and resuspended in 30 mL DMEM/F-12 (Life Technologies, Carlsbad, CA), and the resulting solution used to coat plates at 8.7 μg/cm² (1 mL per well for 6-well plates; 0.5 mL per well for 12-well plates). Plates were incubated at 37° C. for at least 1 h prior to use. hPSCs were maintained on Matrigel-coated plates in E8 medium (STEMCELL Technologies, Vancouver, Canada) at 37° C., 5% CO₂. hPSC lines used were: IMR90-4 iPSC, WTC11 iPSC, CS03iCTRn2 iPSC, H9-CDH5-eGFP hESC, H9-7TGP-ishcat2 hESC, and 19-9-11-7TGP-ishcat3 iPSC. Medium was changed daily. When hPSC colonies began to touch, typically at approximately 70-80% confluence, cells were passaged using Versene (Life Technologies). Briefly, cells were washed once with Versene, then incubated with Versene for 7 min at 37° C. Versene was removed and cells were dissociated into colonies by gentle spraying with E8 medium. Cells were transferred at a split ratio of 1:12 to a new Matrigel-coated plate containing E8 medium.

### Endothelial Progenitor Cell Differentiation

EPCs were differentiated according previously published protocols (Lian et al., 2014; Bao et al., 2016; Nishihara et al., 2020) with slight modifications. On day −3 (D−3), hPSCs were treated with Accutase (Innovative Cell Technologies, San Diego, CA) for 7 min at 37° C. The resulting single cell suspension was transferred to 4× volume of DMEM/F-12 (Life Technologies) and centrifuged for 5 min, 200×g. Cell number was quantified using a hemocytometer. Cells were resuspended in E8 medium supplemented with 10 μM ROCK inhibitor Y-27632 dihydrochloride (Tocris, Bristol, United Kingdom) and seeded on Matrigel-coated 12-well plates at a density of (1.5-2.5)×10⁴ cells/cm², 1 mL per well. Cells were maintained at 37° C., 5% CO₂. On the following two days (D−2 and D−1), the medium was replaced with E8 medium. The following day (DO), differentiation was initiated by changing the medium to LaSR medium (Advanced DMEM/F-12 [Life Technologies], 2.5 mM GlutaMAX [Life Technologies], and 60 μg/mL 1-ascorbic acid 2-phosphate magnesium [Sigma-Aldrich, St. Louis, MO]) supplemented with 7-8 μM CHIR 99021 (Tocris), 2 mL per well. The following day (D1), medium was replaced with LaSR medium supplemented with 7-8 μM CHIR 99021, 2 mL per well. On the following three days (D2, D3, and D4), the medium was replaced with pre-warmed LaSR medium (without CHIR), 2 mL per well.

On D5, EPCs were isolated using CD31 magnetic activated cell sorting (MACS). Cells were treated with Accutase for 15-20 min at 37° C. The resulting cell suspension was passed through a 40 μm cell strainer into an equal volume of DMEM (Life Technologies) supplemented with 10% FBS (Peak Serum, Wellington, CO) and centrifuged for 5 min, 200×g. Cell number was quantified using a hemocytometer. Cells were resuspended in MACS buffer (Dulbecco's phosphate buffered saline without Ca and Mg [DPBS; Life Technologies] supplemented with 0.5% bovine serum albumin [Sigma-Aldrich] and 2 mM EDTA [Sigma-Aldrich]) at a concentration of 10⁷ cells per 100 μL. The CD31-FITC antibody (Miltenyi Biotec, Auburn, CA) was added to the cell suspension at a dilution of 1:50. The cell suspension was incubated for 30 min at room temperature, protected from light. The cell suspension was brought to a volume of 15 mL with MACS buffer and centrifuged for 5 min, 200×g. The supernatant was aspirated and the pellet resuspended in MACS buffer at a concentration of 10⁷ cells per 100 μL. The FITC Selection Cocktail from the EasySep Human FITC Positive Selection Kit (STEMCELL Technologies) was added at a dilution of 1:10 and the cell suspension was incubated for 20 min at room temperature, protected from light. The Dextran RapidSpheres (magnetic particles) solution from the Selection Kit was added at a dilution of 1:20 and the cell suspension was incubated for an additional 15 min at room temperature.

The cell suspension was brought to a total volume of 2.5 mL with MACS buffer (for total cell number less than 2×10⁸, the approximate maximum yield from two 12-well plates; for a larger number of plates/cells, a total volume of 5 mL was used). 2.5 mL of cell suspension was transferred to a sterile 5 mL round-bottom flow cytometry tube and placed in the EasySep magnet (STEMCELL Technologies) for 5 min. The magnet was inverted to pour off the supernatant, the flow tube removed, the retained cells resuspended in 2.5 mL of MACS buffer, and the flow tube placed back in the magnet for 5 min. This step was repeated 3 times, and the resulting cell suspension transferred to a centrifuge tube, and centrifuged for 5 min, 200×g. Cell number was quantified using a hemocytometer. Resulting EPCs were used directly for experiments as described below or cryopreserved in hECSR medium supplemented with 30% FBS and 10% DMSO for later use. hESCR medium is Human endothelial serum-free medium (Life Technologies) supplemented with 1×B-27 supplement (Life Technologies) and 20 ng/ml FGF2 (Waisman Biomanufacturing, Madison, WI).

### Neural Rosette Differentiation

Neural rosettes were differentiated according to a previously published protocol (Lippmann et al., 2014) with slight modifications. On D-1, IMR90-4 hPSCs were dissociated with Accutase and seeded on Matrigel-coated plates in E8 medium supplemented with ROCK inhibitor as described above, except the cell seeding density was 5×10⁵ cells/cm². The following day (DO), medium was replaced with E6 medium (DMEM/F-12 supplemented with 64 mg/L 1-ascorbic acid 2-phosphate magnesium, 14 μg/L sodium selenium, 543 mg/L sodium bicarbonate, 19.4 mg/L insulin [Roche, Penzberg, Germany], and 10.7 mg/L holo-transferrin [Sigma-Aldrich]) prepared according to (Chen et al., 2011). Medium was replaced daily with E6 medium on D1 through D5. On D6, medium was replaced with hECSR medium lacking FGF2. The following day (D7), the resulting neural rosette-conditioned medium (NR-CM) was harvested and stored at 4° C., and fresh hECSR medium lacking FGF2 was replaced. NR-CM was likewise harvested on D8, D9, and D10. The resulting NR-CM aliquots were pooled, passed through a 0.2 μm filter, supplemented with 20 ng/mL FGF2, and used for experiments as described below.

### Astrocyte Differentiation

Astrocytes were differentiated via an hPSC-derived EZ sphere intermediate according to previously published protocols (Ebert et al., 2013; Sareen et al., 2014; Canfield et al., 2017). Briefly, CS03iCTRn2 hPSCs were dissociated with Versene and colonies were transferred to an ultra-low attachment T-25 flask containing EZ sphere culture medium (a mixture of DMEM and F-12 medium in a 7:3 ratio supplemented with 1×B-27 supplement minus vitamin A [Life Technologies], 2 μg/mL heparin [Sigma-Aldrich], 100 ng/mL EGF [Peprotech], 100 ng/mL FGF2, and 1× antibiotic-antimycotic [Life Technologies]). Half of the volume of EZ sphere culture medium was replaced on Mondays, Wednesdays, and Fridays. EZ spheres were passaged every Friday by mechanical dissociation with 2-4 passes on a McIlwain Tissue Chopper (Campden Instruments, Loughborough, United Kingdom), with half of the resulting aggregates returned to the flask and half discarded. To convert EZ spheres into astrospheres, which are neural stem cell aggregates with enhanced astrocyte differentiation potential, medium was changed to DMEM/F-12 supplemented with 1× N-2 supplement (Life Technologies), 2 μg/mL heparin, 1× MEM-non-essential amino acids solution (Life Technologies), and 0.5 μM all-trans retinoic acid (Sigma-Aldrich) and replaced daily for 11 days. The resulting spheres were passaged as described above and returned to EZ sphere culture medium, which was replaced on Mondays, Wednesdays, and Fridays. Astrospheres were passaged on Fridays as described above and cultured for at least 30 passages prior to initiating astrocyte differentiation. To differentiate astrocytes, astrospheres were treated with Accutase for 10-15 min at 37° C., followed by gentle pipetting to dissociate and singularize the cells. The resulting single cell suspension was transferred to 4× volume of DMEM/F-12 and centrifuged for 5 min, 200×g. Cell number was quantified using a hemocytometer. Cells were resuspended in EZ sphere culture medium and seeded on Matrigel-coated plates at approximately 2.5×10⁴ cells/cm². The following day, medium was changed to astrocyte differentiation medium (DMEM/F-12 supplemented with 1× N-2 supplement, 2 μg/mL heparin, and 1× MEM-non-essential amino acids solution). This medium was replaced every other day for 2 weeks. Medium was then replaced with hECSR medium lacking FGF2. The following day, the resulting astrocyte-conditioned medium (Astro-CM) was harvested and stored at 4° C., and fresh hECSR medium lacking FGF2 was replaced. Astro-CM was likewise harvested on the following 3 days. The resulting Astro-CM aliquots were pooled, passed through a 0.2 μm filter, supplemented with 20 ng/mL FGF2, and used for experiments as described below.

### Endothelial Cell Culture and Treatment

Collagen IV (Sigma-Aldrich) was dissolved in 0.5 mg/mL acetic acid to a final concentration of 1 mg/mL. Collagen IV-coated plates were prepared by diluting a volume of this stock solution 1:100 in water, adding the resulting solution to tissue culture plates, or #1.5 glass bottom plates (Cellvis, Sunnyvale, CA) for cells intended for confocal imaging (1 mL per well for 6-well plates, 0.5 mL per well for 12-well plates, 0.25 mL per well for 24-well plates), and incubating the plates for 1 h at RT. Collagen IV coating solution was removed and EPCs obtained as described above were suspended in hECSR medium and plated at approximately 3×10⁴ cells/cm². In some experiments, cells were suspended in NR-CM or Astro-CM. In some experiments, ligands and small molecules were added to hECSR medium: CHIR 99021 (Tocris) was used at 4 μM except where indicated; DMSO (Sigma-Aldrich) was used as a vehicle control for CHIR; Wnt3a (R&D Systems) was used at 20 ng/mL; Wnt7a (Peprotech, Rocky Hill, NJ) was used at 50 ng/mL; Wnt7b (Abnova, Tapei, Taiwan) was used at 50 ng/mL; R-spondin 1 (Rspo1; Peprotech) was used at 50 ng/mL; doxycycline was used at 1, 2, or 4 μg/mL. The hECSR medium or CM, including any ligands or small molecules, was replaced every other day until confluent (typically 6 days). We denote this time point “Passage 1.”

For extended culture, ECs were selectively dissociated and replated as previously described (Nishihara et al., 2020). Cells were incubated with Accutase until endothelial cells appeared round, typically 2-3 min at 37° C. The plate was tapped to release the ECs while SMLCs remained attached, and the EC-enriched cell suspension transferred to 4× volume of DMEM/F-12 and centrifuged for 5 min, 200×g. Cells were resuspended in hECSR medium and seeded on a new collagen IV-coated plate at approximately 3×10⁴ cells/cm². hECSR medium was replaced every other day until confluent (typically 6 days). The selective dissociation and seeding described above was repeated, and hECSR medium was again replaced every other day until confluent (typically 6 days). We denote this time point “Passage 3.” In one experiment, these steps were repeated for another two passages. Except where indicated, CHIR 99021 or vehicle (DMSO) was included in the hECSR medium for the entire duration of culture.

### RNA-seq

RNA-seq was performed on ECs and SMLCs from the IMR90-4 hPSC line. Four independent differentiations were performed, with DMSO-, CHIR-, and Wnt7a/b-treated ECs at Passage 1 analyzed from all four differentiations. DMSO- and CHIR-treated ECs at Passage 3 were analyzed from three of the four differentiations. DMSO-treated SMLCs at Passage 1 were analyzed from two of the four differentiations. Fluorescence-activated cell sorting (FACS) was used to isolate CD31⁺ ECs and CD31⁻ SMLCs from mixed Passage 1 cultures. Cells were incubated with Accutase for 10 min at 37° C., passed through 40 μm cell strainers into 4× volume of DMEM/F-12, and centrifuged for 5 min, 200×g. Cells were resuspended in MACS buffer and incubated with CD31-APC antibody (Miltenyi Biotec) for 30 min at 4° C., protected from light. The cell suspension was brought to a volume of 15 mL with MACS buffer and centrifuged at 4° C. for 5 min, 200×g. Cells were resuspended in MACS buffer containing 2 μg/mL 4′,6-diamidino-2-phenylindole (DAPI; Life Technologies). A BD FACSAria III Cell Sorter (BD Biosciences, San Jose, CA) was used to isolate DAPI⁻CD31⁺ cells (live ECs) and DAPI⁻CD31⁻ cells (live SMLCs). The resulting cell suspensions were centrifuged at 4° C. for 5 min, 200×g, and cell pellets immediately processed for RNA extraction as described below.

RNA was isolated using the RNeasy Plus Micro Kit (Qiagen, Germantown, MD). Buffer RLT Plus supplemented with 1% β-mercaptoethanol was used to lyse cells (pellets from FACS of Passage 1 cells, or directly on plates for Passage 3 ECs). Lysates were passed through gDNA Eliminator spin columns, loaded onto RNeasy MinElute spin columns, washed with provided buffers according to manufacturer instructions, and eluted with RNase-free water. Sample concentrations were determined using a NanoDrop spectrophotometer (Thermo Scientific, Waltham, MA) and RNA quality assayed using an Agilent 2100 Bioanalyzer with Agilent RNA 6000 Pico Kit (Agilent, Santa Clara, CA). First-strand cDNA synthesis was performed using the SMART-Seq v4 Ultra Low Input RNA kit (Takara Bio, Mountain View, CA) with 5 ng input RNA followed by 9 cycles of PCR amplification and library preparation using the Nextera XT DNA Library Prep Kit (Illumina, San Diego, CA). Sequencing was performed on a NovaSeq 6000 (Illumina), with approximately 40-60 million 150 bp paired-end reads obtained for each sample.

FASTQ files were aligned to the human genome (hg38) and transcript abundances quantified using RSEM (v1.3.3) (Li and Dewey, 2011) calling bowtie2 (v2.4.2) (Langmead and Salzberg, 2012). Estimated counts from RSEM were input to DESeq2 (v1.26.0) (Love et al., 2014) implemented in R (v3.6.3) for differential expression analysis. Elsewhere, transcript abundances are presented as transcripts per million (TPM). Differentiation pairing as described above was included in the DESeq2 designs. The Wald test with Benjamini-Hochberg correction was used to generate adjusted P-values. Principal component analysis was performed on counts after the DESeq2 variance stabilizing transformation. Bulk RNA-seq data from the literature (FASTQ files; see Table 7) were obtained from the Gene Expression Omnibus (GEO). These FASTQ files were aligned to the mouse genome (mm10) and transcript abundances quantified as described above. DESeq2 was used for differential expression analysis as described above. For direct comparison of human and mouse data, the biomaRt package (v2.42.1) (Durinck et al., 2009) and Ensembl database (Yates et al., 2019) was used to map human gene names to mouse homologs. Venn diagrams were generated using the tool available at bioinformatice.psb.ugent.be/webtools/Venn/. To identify solute carrier and efflux transporter genes highly expressed at the human BBB in vivo, we used five human brain scRNA-seq datasets (see Table 7) integrated in a previous meta-analysis (Gastfriend et al., 2021). SLC and ABC genes with average expression greater than 100 TPM in endothelial cells across the five independent datasets were selected.

RNA-seq data have been deposited in GEO under accession number GSE173206 (National Center for Biotechnology Information (NCBI), U.S.A.).

### Immunocytochemistry

Immunocytochemistry was performed in 24-well plates. Cells were washed once with 500 μL DPBS and fixed with 500 μL cold (−20° C.) methanol for 5 min, except cells intended for calponin/SM22a and CD31/Ki67 detection, which were fixed with 500 μL of 4% paraformaldehyde for 15 min. Cells were washed three times with 500 μL DPBS and blocked in 150 μL DPBS supplemented with 10% goat serum (Life Technologies) for 1 h at room temperature, except cells intended for calponin/SM22α detection, which were blocked and permeabilized in DPBS supplemented with 3% BSA and 0.1% Triton X-100, or cells intended for CD31/Ki67 detection, which were blocked and permeabilized in DPBS supplemented with 5% non-fat dry milk and 0.4% Triton X-100. Primary antibodies diluted in 150 μL of the above blocking solutions (see Table 6 for antibody information) were added to cells and incubated overnight at 4° C. on a rocking platform. Cells were washed three times with 500 μL DPBS. Secondary antibodies diluted in 150 μL of the above blocking solutions (see Table 6 for antibody information) were added to cells and incubated for 1 h at room temperature on a rocking platform, protected from light. Cells were washed three times with 500 μL DPBS, followed by 5 min incubation with 500 μL DPBS plus 4 μM Hoechst 33342 (Life Technologies). Images were acquired using an Eclipse Ti2-E epifluorescence microscope (Nikon, Tokyo, Japan) with a 20× objective or an A1R-Si+ confocal microscope (Nikon) with a 100× oil objective. Confocal images were acquired with 1 μm slice spacing.

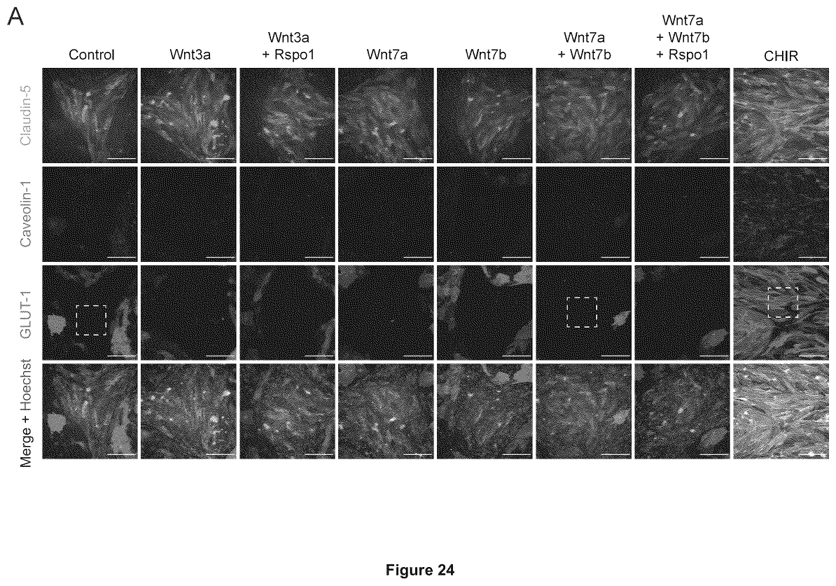

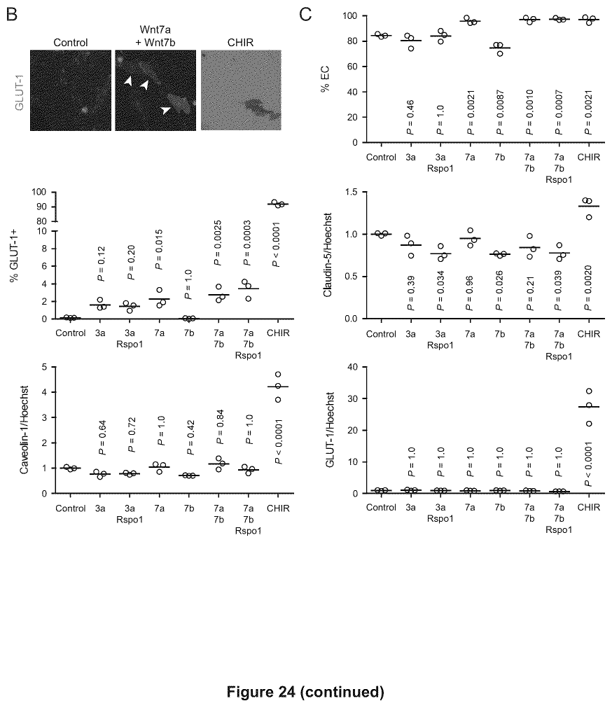

Images were analyzed using FIJI (ImageJ) software. For epifluorescence images, 5 fields (20×) were analyzed per well, with 3-4 wells per treatment condition. For quantification of cell number, EC colonies were manually outlined, and the Analyze Particles function was used to estimate the number of nuclei within the EC colonies. Nuclei outside the EC colonies were manually counted. EC purity (% EC) was calculated as the number of nuclei within EC colonies relative to total nuclei. To estimate % GLUT-1⁺ ECs, cells within the EC colonies with membrane-localized GLUT-1 immunoreactivity (e.g., arrowheads in FIG. 24B) were manually counted. To estimate % Ki67⁺ ECs, cells within the EC colonies with at least one nuclear-localized Ki67 punctum were manually counted. For quantification of fluorescence intensity in epifluorescence images, EC colonies were manually outlined, and the Measure function was used to obtain the mean fluorescence intensity for each image channel (fluorophore). A cell-free area of the plate was similarly quantified for background subtraction. Following background subtraction, the mean fluorescence intensity of each protein of interest was normalized to the mean fluorescence intensity of Hoechst to correct for effects of cell density. For confocal images, 3-4 fields (100×) containing only VE-cadherin⁺ ECs were analyzed per well, with 4 wells per treatment condition. The first slice with visible nuclei (closest to glass) was defined as Z=0, and the Measure function was used to obtain the mean fluorescence intensity for each image channel (fluorophore) in each slice from Z=0 to Z=7 μm. A cell-free area of the plate was similarly quantified for background subtraction. After background subtraction, to approximate total abundance (area under the fluorescence versus Z curve, AUC) for each channel, mean fluorescence intensities were summed across all slices. AUC for the proteins of interest were normalized to Hoechst AUC.

### Cell Cycle Analysis

Passage 1 cultures were dissociated by treatment with Accutase for 10 min at 37° C. Cell suspensions were passed through 40 μm cell strainers into 4× volume of DMEM/F-12 and centrifuged for 5 min, 200×g. Approximately 5×10⁵ cells per replicate were resuspended in MACS buffer and incubated with the CD31-APC antibody (Miltenyi Biotec) for 30 min at 4° C., protected from light. Cell suspensions were brought to a volume of 5 mL with MACS buffer and centrifuged at 4° C. for 5 min, 200×g. Cells were resuspended in 500 μL MACS buffer containing 2 μg/mL DAPI and 0.5 μL Vybrant DyeCycle Green Stain (Invitrogen) and incubated at room temperature for 1 h, protected from light. Cells were analyzed on an Attune NxT flow cytometer (Invitrogen). FlowJo software (BD Biosciences) was used to gate CD31⁺ cells and quantify the percentage of S/G2/M phase cells.

### Western Blotting

To enrich samples from Passage 1 cultures for ECs, the Accutase-based selective dissociation method described above was employed. Dissociated cells were centrifuged for 5 min, 200×g, and resulting cell pellets were lysed in RIPA buffer (Rockland Immunochemicals, Pottstown, PA) supplemented with 1× Halt Protease Inhibitor Cocktail (Thermo Scientific). Passage 3 cells were lysed with the above buffer directly on plates. Lysates were centrifuged at 4° C. for 5 min, 14,000×g, and protein concentration in supernatants quantified using the Pierce BCA Protein Assay Kit (Thermo Scientific). Equal amounts of protein were diluted to equal volume with water, mixed with sample buffer, and heated at 95° C. for 5 min, except lysates intended for GLUT-1 Western blotting, which were not heated. Samples were resolved on 4-12% Tris-Glycine gels and transferred to nitrocellulose membranes. Membranes were blocked for 1 h in tris-buffered saline plus 0.1% Tween-20 (TBST) supplemented with 5% non-fat dry milk. Primary antibodies (see Table 6 for antibody information) diluted in TBST plus 5% non-fat dry milk were added to membranes and incubated overnight at 4° C. on a rocking platform. Membranes were washed five times with TBST. Secondary antibodies (see Table 6 for antibody information) diluted in TBST were added to membranes and incubated for 1 h at room temperature on a rocking platform, protected from light. Membranes were washed five times with TBST and imaged using an Odyssey 9120 (LI-COR, Lincoln, NE). Band intensities were quantified using Image Studio software (LI-COR).

### Dextran Accumulation Assay

A fixable, Alexa Fluor 488-conjugated dextran with an average molecular weight of kDa (Invitrogen) was used as a tracer to estimate total fluid-phase endocytosis. Dextran was added at 10 μM to the medium of Passage 1 cultures. Plates were incubated on rotating platforms at 37° C. or 4° C. for 2 h. Medium was removed and cells were washed once with DPBS, and then incubated with Accutase for 10 min at 37° C. Cell suspensions were passed through 40 μm cell strainers into 4× volume of DMEM/F-12 and centrifuged for 5 min, 200×g. Cells were resuspended in MACS buffer and incubated with the CD31-APC antibody (Miltenyi Biotec) for 30 min at 4° C., protected from light. Cell suspensions were brought to a volume of 5 mL with MACS buffer and centrifuged at 4° C. for 5 min, 200×g. Pellets were resuspended in DPBS supplemented with 4% paraformaldehyde and incubated for 15 min at room temperature, protected from light. Cells were centrifuged for 5 min, 200×g. Pellets were resuspended in MACS buffer and analyzed on a BD FACSCalibur flow cytometer (BD Biosciences). FlowJo software was used to gate CD31⁺ cells and quantify geometric mean fluorescence intensity and coefficient of variation (CV) of dextran.

### Transendothelial Electrical Resistance and Sodium Fluorescein Permeability

Transwell inserts (6.5 mm diameter with 0.4 μm pore polyester filters) (Corning) were coated with 50 μl of a solution of collagen IV (400 μg/mL) and fibronectin (100 μg/mL) in water for 4 h at 37° C. Passage 3 DMSO- and CHIR-treated ECs were seeded on Transwell inserts at 10⁵ cells/cm² in hECSR medium supplemented with DMSO or CHIR. Medium volumes were 200 μL for the apical chamber and 800 μL for the basolateral chamber. Beginning the day after seeding, TEER was measured daily for 6 days using an EVOM2 epithelial voltohmmeter with STX2 chopstick electrodes (World Precision Instruments, Sarasota, FL). Medium was replaced every other day. TEER values were corrected by subtracting the resistance of a collagen IV/fibronectin-coated Transwell insert without cells and multiplying by the filter surface area of 0.33 cm². Permeability of endothelial monolayers to sodium fluorescein was assessed 6 days after seeding cells on Transwell inserts. Medium in both apical and basolateral chambers was replaced and cells returned to the incubator for 1 h. Medium in apical chambers, including the apical chamber of a collagen IV/fibronectin-coated Transwell insert without cells, was then replaced with medium supplemented with 10 μM sodium fluorescein (Sigma Aldrich), and plates placed on an orbital platform in an incubator. At 15, 30, 45, and 60 min, an 80 μL sample of the basolateral chamber medium was withdrawn from each Transwell, transferred to a 96-well plate, and 80 μL fresh medium replaced in the basolateral chamber of each Transwell. At 60 min, an 80 μL sample of apical chamber medium was also withdrawn from each Transwell and transferred to the 96-well plate. 80 μL of medium lacking sodium fluorescein was also transferred to the 96-well plate for background subtraction. Fluorescence intensity of all samples was measured using an Infinite M1000 PRO plate reader (Tecan, Mannedorf, Switzerland) with 485 nm excitation and 530 nm emission wavelengths. Background-subtracted fluorescence intensity values at the 30, 45, and 60 min timepoints were corrected for sampling-induced dilution as previously described (Stebbins et al., 2016). The endothelial permeability coefficient (Pₑ), which is a concentration-independent parameter corrected for the permeability of a cell-free Transwell insert, was calculated as previously described (Stebbins et al., 2016).

### Statistics

Individual wells of cultured cells that underwent identical experimental treatments are defined as replicates, and all key experiments were repeated using multiple independent hPSC differentiations. Detailed information about replication strategy is provided in figure legends. Student's t test was used for comparison of means from two experimental groups. One-way analysis of variance (ANOVA) was used for comparison of means from three or more experimental groups, followed by Dunnett's post-hoc test for comparison of multiple treatments to a single control, or Tukey's honest significant difference (HSD) post-hoc test for multiple pairwise comparisons. When data from multiple differentiations were combined, two-way ANOVA (one factor being the experimental treatment and one factor being the differentiation) was used for comparison of means to achieve blocking of differentiation-based variability, followed by post-hoc tests as described above if more than two experimental treatments were compared. For fluorescence intensities (a.u.), two-way ANOVA was performed prior to normalization of these values to the control group within each differentiation (for visualization in plots). Statistical tests were performed in JMP Pro (v15.0.0). For RNA-seq differential expression analysis, the DESeq2 Wald test with Benjamini-Hochberg correction was used to calculate P-values. Descriptions of the statistical tests used are provided in figure legends.

TABLE 7
Published Datasets

| Citation | Description | Identifiers | Source |
|---|---|---|---|
| (Sabbagh et al., | Adult mouse | GSM3040844 | www.ncbi.nlm.nih.gov/ |
| 2018) | postnatal day 7 | GSM3040845 | geo/query/acc.cgi?acc=GSE111839 |
| | brain, liver, lung, | GSM3040852 | |
| | and kidney ECs | GSM3040853 | |
| | | GSM3040858 | |
| | | GSM3040859 | |
| | | GSM3040864 | |
| | | GSM3040865 | |
| (Munji et al., | Adult mouse | GSM2498580 | www.ncbi.nlm.nih.gov/ |
| 2019) | liver ECs | GSM2498581 | geo/query/acc.cgi?acc=GSE95201 |
| | (controls and β- | GSM2498582 | |
| | catenin gain-of- | GSM2498583 | |
| | function) | GSM2498584 | |
| | | GSM2498585 | |
| | | GSM2498586 | |
| | | GSM2498587 | |
| (Wang et al., | Adult mouse | GSM3455653 | www.ncbi.nlm.nih.gov/ |
| 2019) | anterior and | GSM3455654 | geo/query/acc.cgi?acc=GSE122117 |
| | posterior | GSM3455657 | |
| | pituitary ECs | GSM3455658 | |
| | (controls and β- | GSM3455661 | |
| | catenin gain-of- | GSM3455662 | |
| | function) | GSM3455665 | |
| | | GSM3455666 | |
| (Sabbagh and | Adult mouse | GSM4160534 | www.ncbi.nlm.nih.gov/ |
| Nathans, 2020) | brain ECs | GSM4160535 | geo/query/acc.cgi?acc=GSE118731 |
| | cultured in vitro | GSM4160536 | |
| | (controls and β- | GSM4160537 | |
| | catenin gain-of- | GSM4160538 | |
| | function) | GSM4160539 | |
| | | GSM4160540 | |
| | | GSM4160541 | |
| | | GSM4160542 | |
| | | GSM4160543 | |
| (Gastfriend et al., | Meta-analysis of | | |
| 2021) | human brain | | |
| | single cells | | |
| | across multiple | | |
| | developmental | | |
| | stages and brain | | |
| | regions | | |
| | (enumerated | | |
| | below) | | |
| Allen Institute, | Adult neocortex | | portal.brain-map.org/atlases-and- |
| 2019 | | | data/rnaseq/human-multiple- |
| | | | cortical-areas-smart-seq |
| (Polioudakis et | GW17-18 | | solo.bmap.ucla.edu/shiny/webapp/ |
| al., 2019) | neocortex | | |
| (Han et al., 2020) | Adult temporal | GSM3980129 | www.ncbi.nlm.nih.gov/ |
| | lobe and | GSM4008656 | geo/query/acc.cgi?acc=GSE134355 |
| | cerebellum | GSM4008657 | |
| | | GSM4008658 | |
| (La Manno et al., | GW6-11 ventral | | www.ncbi.nlm.nih.gov/ |
| 2016) | midbrain | | geo/query/acc.cgi?acc=GSE76381 |
| (Zhong et al., | GW16-27 | | www.ncbi.nlm.nih.gov/ |
| 2020) | hippocampus | | geo/query/acc.cgi?acc-GSE119212 |

### Results:

Wnt Activation in hPSC-Derived Endothelial Progenitors

We adapted an existing protocol to produce endothelial progenitor cells (EPCs) from hPSCs (Lian et al., 2014; Bao et al., 2016) (FIG. 23A). To achieve mesoderm specification, this method employs an initial activation of Wnt/β-catenin signaling with CHIR 99021 (CHIR), a small molecule inhibitor of glycogen synthase kinase-3 (GSK-3), which results in inhibition of GSK-3β-mediated β-catenin degradation. After 5 days of expansion, the resulting cultures contained a mixed population of CD34⁺CD31⁺ EPCs and CD34-CD31-non-EPCs (FIG. 23B-C). We used magnetic-activated cell sorting (MACS) to isolate CD31⁺ cells from this mixed culture and plated these cells on collagen IV-coated plates in a minimal endothelial cell medium termed hECSR (Nishihara et al., 2020). We first asked whether Wnt3a, a ligand widely used to activate canonical Wnt/β-catenin signaling (Kim et al., 2005, 2008; Liebner et al., 2008; Cecchelli et al., 2014; Praca et al., 2019), could induce GLUT-1 expression in the resulting ECs. After 6 days of treatment, we observed a significant increase in the fraction of GLUT-1⁺ ECs in Wnt3a-treated cultures compared to controls (FIG. 23D-E). Consistent with previous observations (Nishihara et al., 2020), we also detected a population of calponin⁺ smooth muscle protein 22-α⁺ putative smooth muscle-like cells (SMLCs) outside the endothelial colonies (FIG. 23F) and these SMLCs expressed GLUT-1 in both control and Wnt3a-treated conditions (FIG. 23D).

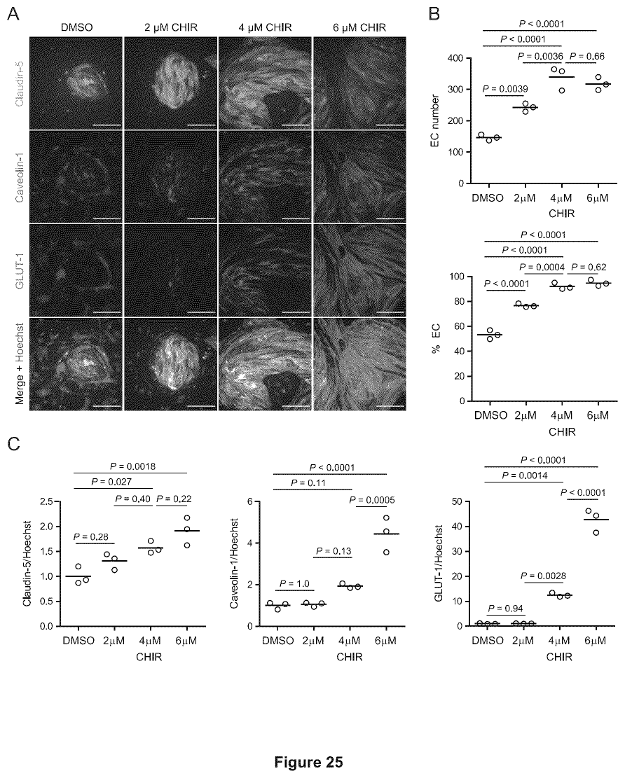

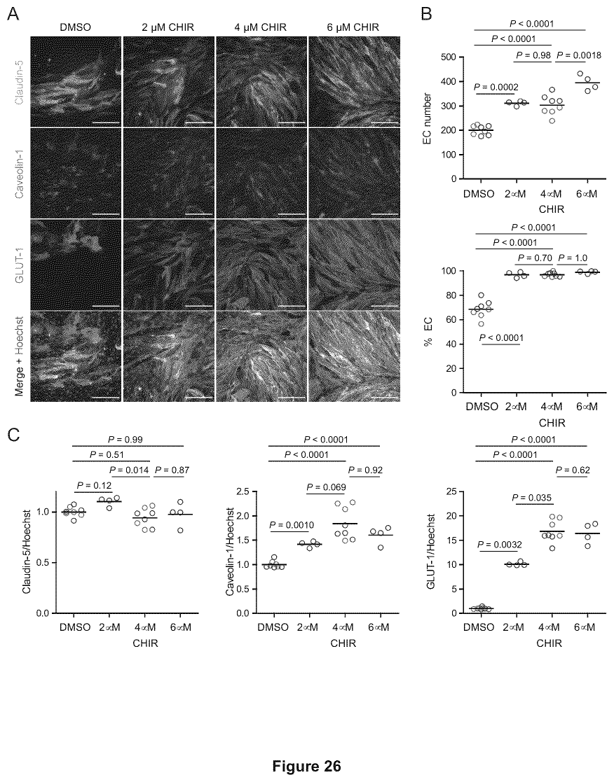

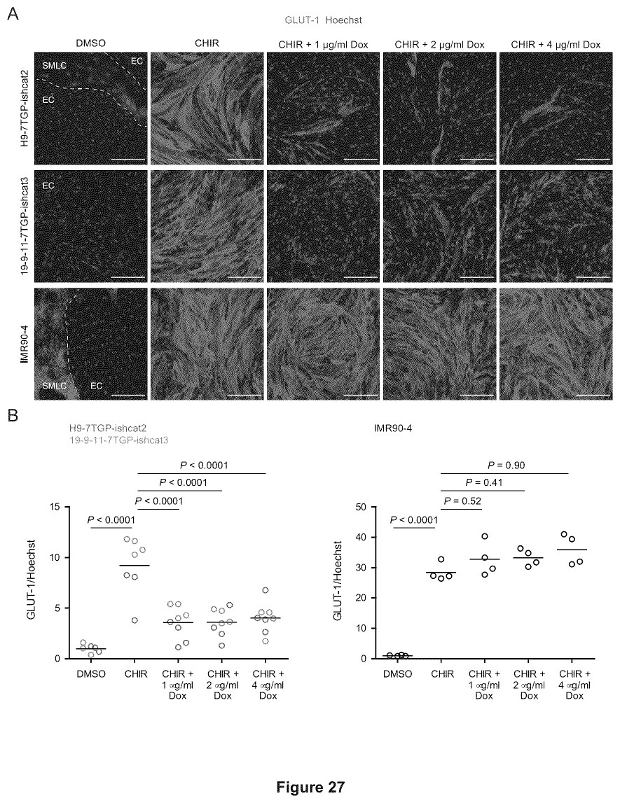

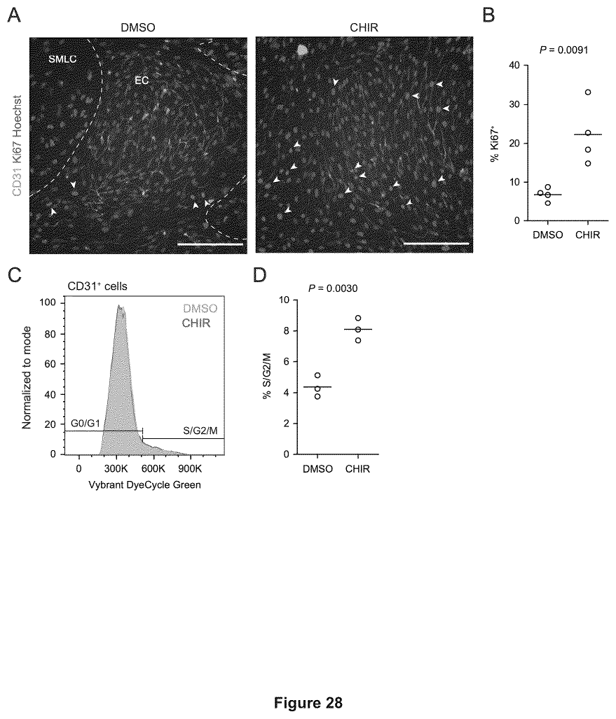

Based on these promising results with Wnt3a, we next tested several additional strategies for Wnt activation and, in addition to GLUT-1, evaluated expression of two other key proteins: claudin-5, which is known to be upregulated in CNS ECs in response to Wnt (Benz et al., 2019), and caveolin-1, given the low rate of caveolin-mediated transcytosis in CNS compared to non-CNS ECs (Reese and Karnovsky, 1967; Andreone et al., 2017). First, we tested Wnt7a and Wnt7b, the ligands primarily responsible for Wnt activation in CNS ECs in vivo (Daneman et al., 2009; Cho et al., 2017). We also tested Wnt ligands in combination with R-spondin 1 (Rspo1), a potentiator of Wnt signaling that inhibits the RNF43/ZNRF3-mediated negative feedback mechanism by which Frizzled receptors are endocytosed (Kim et al., 2005, 2008; Koo et al., 2012; Clevers et al., 2014). Finally, we tested a low concentration (4 μM) of the GSK-3 inhibitor CHIR because of its ability to activate Wnt signaling in a receptor/co-receptor-independent manner. We found that Wnt7a and the combination of Wnt7a and Wnt7b, but not Wnt7b alone, slightly increased the fraction of GLUT-1⁺ ECs, while Rspo1 did not affect EC purity or expression of GLUT-1, claudin-5 or caveolin-1 (FIG. 24A-C). Interestingly, Wnt7a, but not Wnt3a, also increased the proportion of ECs compared to SMLCs (FIG. 24A,C). By contrast, 4 μM CHIR robustly induced GLUT-1 expression in approximately 90% of ECs while increasing EC purity to a level similar to that achieved with Wnt7a. Furthermore, CHIR led to an approximately 1.5-fold increase in average claudin-5 abundance and a nearly 30-fold increase in GLUT-1 abundance, but also a 4-fold increase in caveolin-1 (FIG. 24A,C). We therefore titrated CHIR to determine an optimal concentration for EC expansion, purity, GLUT-1 induction, and claudin-5 upregulation while limiting the undesirable non-CNS-like increase in caveolin-1 abundance. Although 2 μM CHIR did not lead to increased caveolin-1 expression compared to vehicle control (DMSO), it also did not elevate claudin-5 or GLUT-1 expression compared to control and was less effective in increasing EC number and EC purity than 4 μM CHIR (FIG. 25). On the other hand, 6 μM CHIR further increased GLUT-1 abundance but also further increased caveolin-1 abundance and did not improve EC number, EC purity, or claudin-5 expression (FIG. 25). Therefore, we conducted further experiments using 4 μM CHIR. We confirmed that the CHIR-mediated increases in EC purity, EC number, and caveolin-1 and GLUT-1 expression were conserved in an additional hPSC line, although claudin-5 upregulation was not apparent (FIG. 26). We also used two hPSC lines with doxycycline-inducible expression of short hairpin RNAs targeting CTNNB1 (β-catenin) to confirm that CHIR-mediated upregulation of GLUT-1 in ECs was β-catenin-dependent. Indeed, doxycycline treatment in combination with CHIR significantly reduced GLUT-1 abundance in ECs derived from these hPSC lines (FIG. 27). Finally, we confirmed that CHIR-mediated increases in EC number were the result of increased EC proliferation (FIG. 28). Together, these results suggest that Wnt pathway activation, either with ligands or CHIR, is capable of inducing CNS-like phenotypes in hPSC-derived endothelial progenitors.

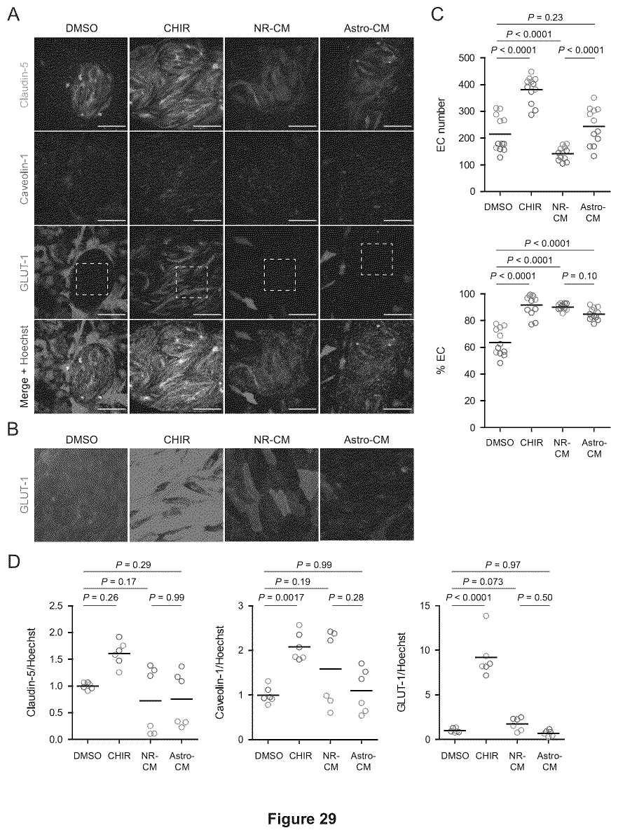

In the CNS, neural progenitors and astrocytes are the primary sources of Wnt ligands resulting in induction and maintenance of EC barrier properties. Because the relatively weak response to Wnt ligands observed in our system is potentially attributable to poor potency associated with the recombinant proteins, we reasoned that relevant cellular sources of Wnt ligands might be more effective in activating Wnt in EPCs. To this end, we differentiated hPSCs to neural rosettes, which are radially organized Pax6⁺ neural progenitors, and astrocytes according to established protocols (Ebert et al., 2013; Lippmann et al., 2014; Sareen et al., 2014; Canfield et al., 2017). Importantly, RNA-seq data from the literature suggest that both hPSC-derived neural rosettes and astrocytes express WNT7A (Vatine et al., 2016; Shang et al., 2018). We collected neural rosette-conditioned medium (NR-CM) and astrocyte-conditioned medium (Astro-CM) and treated EPCs with these media for 6 days. Similar to our observations with Wnt7a, both NR-CM and Astro-CM significantly increased the proportion of ECs compared to SMLCs (FIG. 29A,C). NR-CM, but not Astro-CM, also induced weak GLUT-1 expression in ECs, reminiscent of the Wnt7a-induced phenotype, although this induction was much weaker than in the CHIR-treated cells (FIG. 29B,D). NR-CM and Astro-CM had variable effects with respect to caveolin-1 and claudin-5 expression (FIG. 29D). In summary, NR-CM performed similarly to Wnt7a in weakly inducing GLUT-1 expression and increasing EC purity. The comparatively stronger response to CHIR may suggest either that the potency or concentration of ligands is insufficient, or that the EPCs lack the full machinery of receptors and co-receptors necessary to transduce the Wnt ligand signal (analyzed further below).

### Effects of CHIR-Mediated Wnt Activation in Endothelial Progenitors

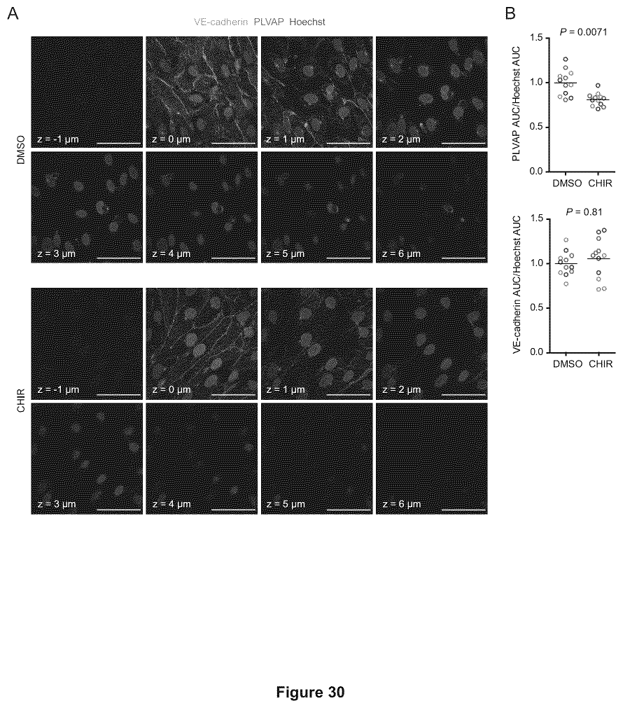

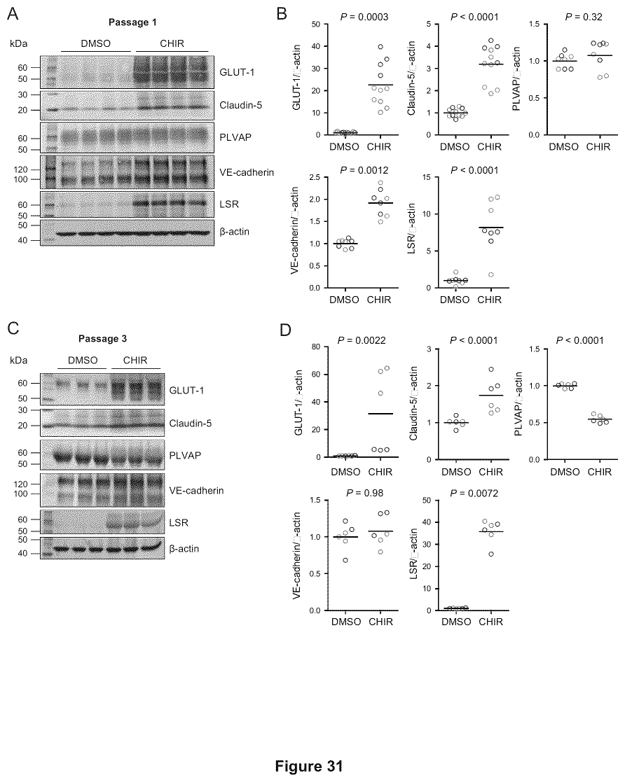

Since CHIR elicited the most robust Wnt-mediated response, we next asked whether other aspects of the CNS EC barrier phenotype were CHIR-regulated. PLVAP, a protein that forms bridges across both caveolae and fenestrae (Herrnberger et al., 2012), is one such canonically Wnt-downregulated protein. We therefore first evaluated PLVAP expression in Passage 1 control (DMSO) or CHIR-treated ECs using confocal microscopy (FIG. 30A). We observed numerous PLVAP⁺ punctate vesicle-like structures in both conditions, with CHIR treatment reducing PLVAP abundance by approximately 20% (FIG. 30A-B). This effect was not apparent in Western blots of Passage 1 ECs, likely due to the relatively modest effect (FIG. 31A-B). However, after two more passages (FIG. 23A), Passage 3 ECs demonstrated a robust downregulation of PLVAP in CHIR-treated cells compared to controls (FIG. 31C-D). We also used Western blotting to confirm CHIR-mediated upregulation of GLUT-1 and claudin-5 both at Passage 1 and Passage 3 (FIG. 31A-D). We next evaluated expression of the tricellular tight junction protein LSR (angulin-1) because of its enrichment in CNS versus non-CNS ECs, and the temporal similarity between LSR induction and the early stage of Wnt-mediated CNS barriergenesis (Sohet et al., 2015). We found that CHIR treatment led to a strong increase in LSR expression in both Passage 1 and Passage 3 ECs (FIG. 31A-D), suggesting that Wnt signaling upregulates multiple necessary components of the CNS EC bicellular and tricellular junctions.

CHIR treatment produced two apparently competing changes in ECs related to vesicular transport: an expected downregulation of PLVAP and an unexpected upregulation of caveolin-1. We therefore asked whether the rate of total fluid-phase endocytosis differed between CHIR-treated and control ECs, using a fluorescently-labeled 10 kDa dextran as a tracer. After incubating Passage 1 cultures with dextran for 2 h at 37° C., we used flow cytometry to gate CD31⁺ ECs and assess total dextran accumulation (FIG. 32A-B). We first confirmed that the process of dextran internalization required the membrane fluidity of an endocytosis-dependent process by carrying out the assay at 4° C.; this condition indeed yielded a substantially decreased dextran signal compared to 37° C. (FIG. 32B). In ECs incubated at 37° C., CHIR treatment did not change the geometric mean dextran signal compared to DMSO (FIG. 32B,C), but did cause a broadening of the distribution of dextran intensities, indicative of sub-populations of cells with decreased and increased dextran uptake (FIG. 32B,D). Importantly, these results were consistent across three independent differentiations (FIG. 32C-D). Thus, despite the generally uniform elevation of caveolin-1 and decrease of PLVAP observed by immunocytochemistry in CHIR-treated ECs, our functional assay suggests neither an overall increase nor decrease in total fluid-phase endocytosis. Instead, it indicates that CHIR increases the heterogeneity of the EC population with respect to the rate of endocytosis. We also compared the paracellular barrier properties of DMSO- and CHIR-treated ECs. Because Passage 1 cultures contain SMLCs that preclude formation of a confluent endothelial monolayer, we evaluated paracellular barrier properties of Passage 3 ECs that had undergone selective dissociation and replating (see Materials and Methods), a strategy that effectively purifies the cultures (Nishihara et al., 2020). CHIR-treated Passage 3 ECs had elevated transendothelial electrical resistance (TEER) (FIG. 32E) and decreased permeability to the small molecule tracer sodium fluorescein (FIG. 32F). Together, these results are consistent with CHIR-mediated increases to tight junction protein expression (e.g., claudin-5 and LSR) and suggest Wnt activation leads to functional improvements to paracellular barrier in this system.

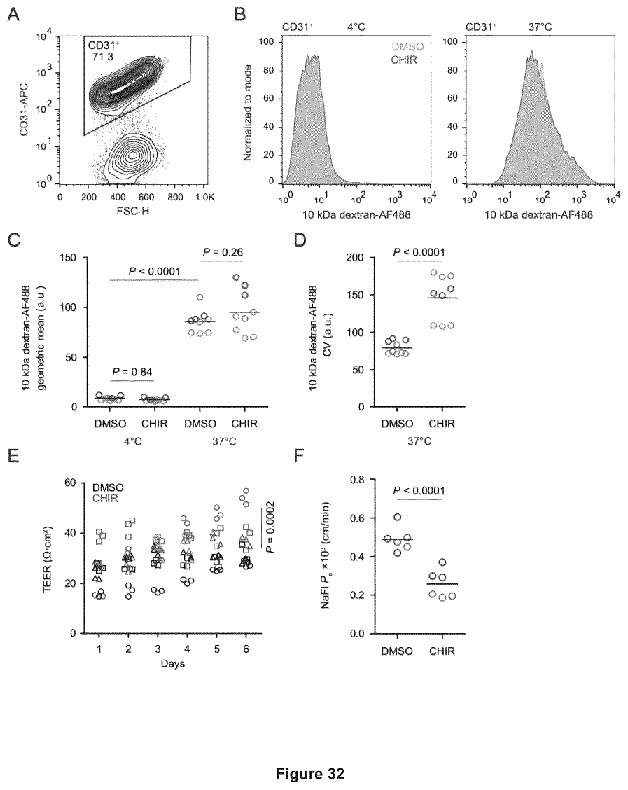

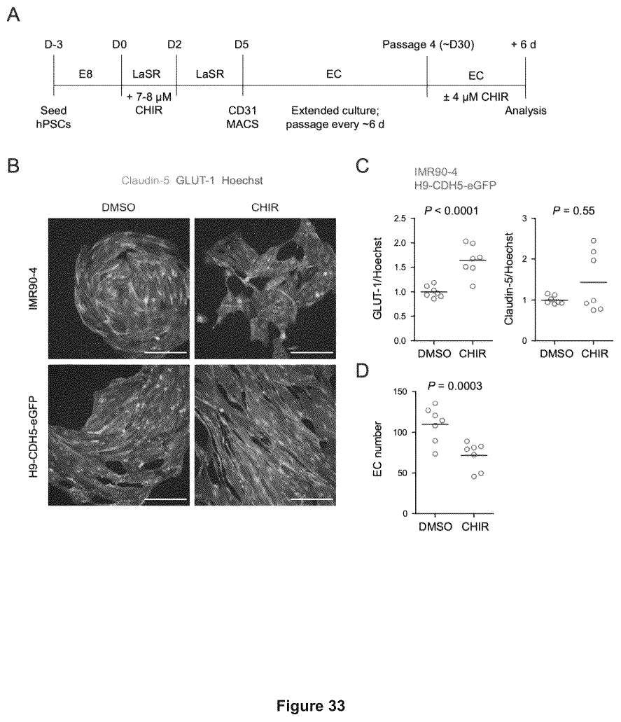

Given the relatively weak responses to Wnt activation in adult mouse liver ECs in vivo (Munji et al., 2019) and adult mouse brain ECs cultured in vitro (Sabbagh and Nathans, 2020), we sought to determine whether the immature, potentially more plastic state of hPSC-derived endothelial progenitors contributed to the relatively robust CHIR-mediated response we observed. To test this hypothesis, we matured hPSC-derived ECs in vitro for 4 passages (until approximately day 30) prior to initiating CHIR treatment for 6 days (FIG. 33A). The resulting Passage 5 DMSO-treated ECs, which are analogous to EECM-BMEC-like cells we previously reported (Nishihara et al., 2020), did not have detectable GLUT-1 expression (FIG. 33B). Compared to DMSO controls, the resulting CHIR-treated Passage 5 ECs exhibited an approximately 1.5-fold increase in GLUT-1 abundance (FIG. 33B-C), a markedly weaker response than the 10- to 40-fold increases routinely observed using the same immunocytochemistry-based assay when CHIR treatment was initiated immediately after MACS (FIGS. 24-26; FIG. 29). Furthermore, CHIR treatment in matured ECs had variable effects on claudin-5 expression (FIG. 33B-C) and led to a slight decrease in EC number (FIG. 33D), rather than the increases observed in both of these properties when treatment was initiated immediately after MACS (FIG. 25; FIG. 29). Together, these data suggest that early, naïve endothelial progenitors are more responsive to Wnt activation than more mature ECs derived by the same differentiation protocol.

### Comprehensive Profiling of the Wnt-Regulated Endothelial Transcriptome

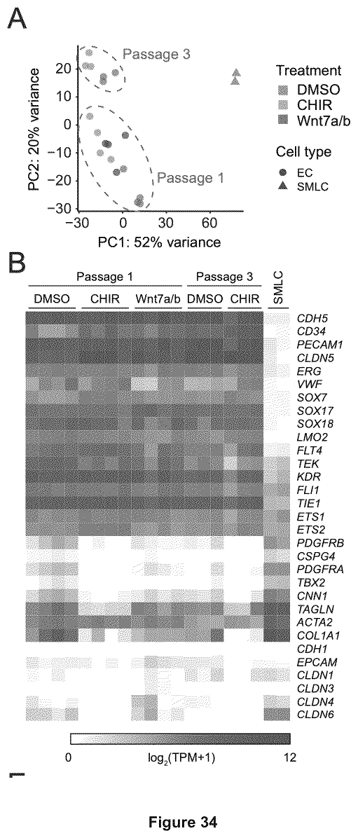

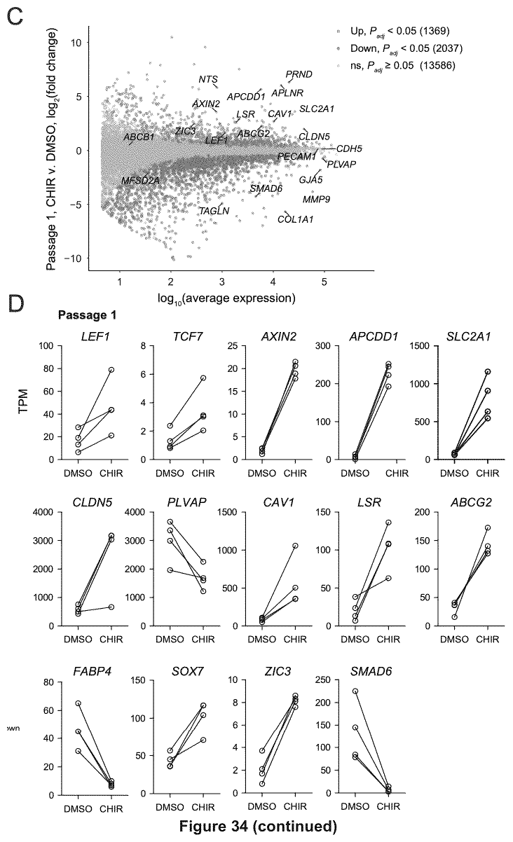

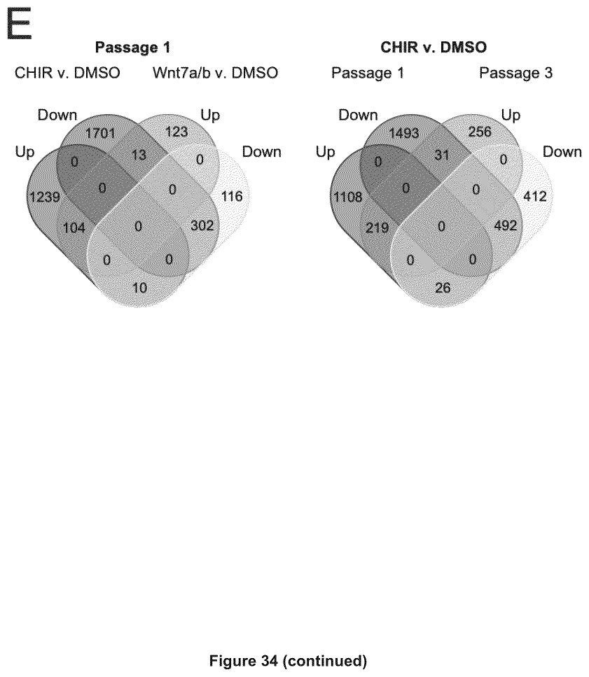

We turned next to RNA-sequencing as an unbiased method to assess the impacts of Wnt activation on the EC transcriptome. We performed four independent differentiations and analyzed Passage 1 ECs treated with DMSO, CHIR, or Wnt7a and Wnt7b (Wnt7a/b), using fluorescence-activated cell sorting (FACS) to isolate CD31⁺ ECs from the mixed EC/SMLC cultures. We also sequenced the SMLCs from DMSO-treated cultures at Passage 1 from two of these differentiations. DMSO- and CHIR-treated ECs at Passage 3 from three of these differentiations were also sequenced. Principal component analysis of the resulting whole-transcriptome profiles revealed that the two cell types (ECs and SMLCs) segregated along principal component (PC) 1, which explained 52% of the variance. In ECs, the effects of passage number and treatment were reflected in PC 2, which explained 20% of the variance (FIG. 34A). We next validated the endothelial identity of our cells; we observed that canonical endothelial marker genes (including CDH5, CD34, PECAM1, CLDN5, ERG, and FLI1) were enriched in ECs compared to SMLCs and had high absolute abundance, on the order of 100-1,000 transcripts per million (TPM) (FIG. 34B). SMLCs expressed mesenchymal (mural/fibroblast)-related transcripts (including PDGFRB, CSPG4, PDGFRA, TBX2, CNN1, and COLIA1), which ECs generally lacked, although we did observe slight enrichment of some of these genes in Passage 1 DMSO-treated ECs, likely reflective of a small amount of SMLC contamination despite CD31 FACS (FIG. 34B). SMLCs also expressed SLC2A1 consistent with protein-level observations (FIG. 23D). We also observed little to no expression of the epithelial genes CDH1, EPCAM, CLDN1, CLDN3 (Castro Dias et al., 2019), CLDN4, and CLDN6, reflecting the definitive endothelial nature of the cells (FIG. 34B).

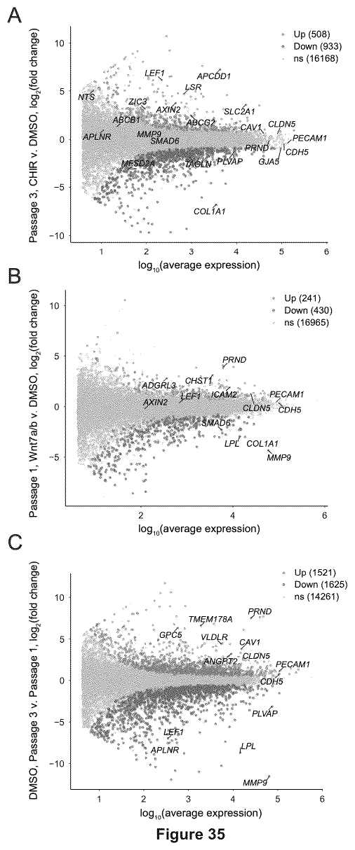

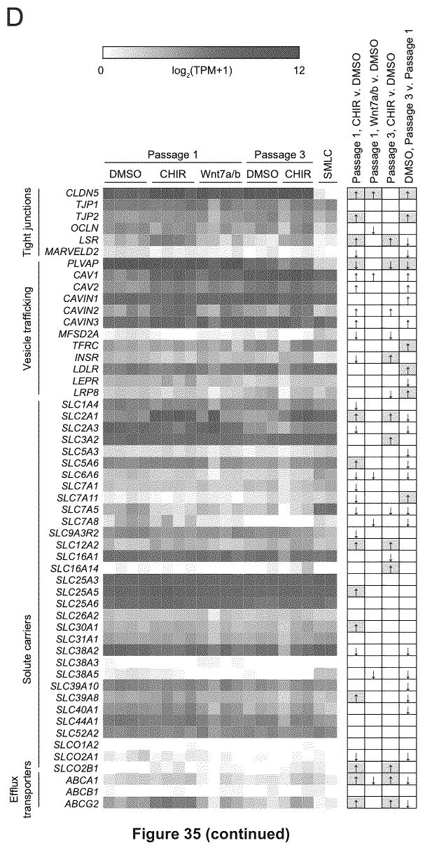

First comparing CHIR- and DMSO-treated ECs at Passage 1, we identified 1,369 significantly upregulated genes and 2,037 significantly downregulated genes (FIG. 34C). CHIR-upregulated genes included SLC2A1, CLDN5, LSR, and CAV1, consistent with protein-level assays. PLVAP was downregulated, as were a number of mesenchymal genes (TAGLN, COLIA1), again reflective of slight contamination of SMLC transcripts in the DMSO-treated EC samples (FIG. 34C-D). Additionally, important downstream effectors of Wnt signaling were upregulated, including the transcription factors LEF1 and TCF7, the negative regulator AXIN2, and the negative regulator APCDD1, which is known to modulate Wnt-regulated barriergenesis in retinal endothelium (Mazzoni et al., 2017) (FIG. 34C-D). We also found that the transcription factors ZIC3, which is highly enriched in brain and retinal ECs in vivo and downstream of Frizzled4 signaling (Wang et al., 2012; Sabbagh et al., 2018), and SOX7, which acts cooperatively with SOX17 and SOX18 in retinal angiogenesis (Zhou et al., 2015), were upregulated by CHIR in our system (FIG. 34D). Additional CHIR-upregulated genes included ABCG2 (encoding the efflux transporter Breast Cancer Resistance Protein, BCRP), and APLN, a tip cell marker enriched in postnatal day 7 murine brain ECs compared to those of other organs, and subsequently downregulated in adulthood (Sabbagh et al., 2018; Sabbagh and Nathans, 2020) (FIG. 34C). Finally, we detected CHIR-mediated downregulation of the fatty acid-binding protein-encoding FABP4, which is depleted in brain ECs compared to those of peripheral organs (Sabbagh et al., 2018). We also observed similar downregulation of SMAD6, which is depleted in brain ECs compared to lung ECs and is a putative negative regulator of BMP-mediated angiogenesis (Mouillesseaux et al., 2016; Vanlandewijck et al., 2018) (FIG. 34D). Many of these CHIR-mediated gene expression changes persisted at Passage 3, including SLC2A1, LSR, LEF1, AXIN2, APCDD1, ZIC3, and ABCG2 upregulation and PLVAP downregulation (FIG. 34E; FIG. 35A).

We made similar comparisons (i) between Wnt7a/b-treated and control (DMSO-treated) ECs at Passage 1, and (ii) between control ECs at Passage 3 versus Passage 1 (FIG. 34E; FIG. 35B-C). Consistent with the weak response observed by immunocytochemistry, there were fewer Wnt7a/b-mediated gene expression changes compared to those elicited by CHIR, with 241 upregulated and 420 downregulated genes (FIG. 35B). In general, however, these changes were consistent with CHIR-mediated changes, with 104 concordantly upregulated genes, 302 concordantly downregulated genes, and only 23 discordantly regulated genes (FIG. 34E). Of note, treatment with Wnt7a/b, but not CHIR, upregulated SOX17, a Wnt target gene required for BBB function (Corada et al., 2018). Extended culture to Passage 3 in the absence of exogeneous Wnt activation led to 1,521 upregulated genes, including CLDN5 and CAV1, consistent with previously-reported protein-level observations in EECM-BMEC-like cells (Nishihara et al., 2020), which are analogous to Passage 3 DMSO-treated cells. We also observed 1,625 downregulated genes, including PLVAP (FIG. 35C). SLC2A1, however, was not upregulated at Passage 3 (FIG. 35C), concordant with absence of GLUT-1 protein expression in the control ECs (FIG. 33B). To further understand the strengths and limitations of this model system both as a readout of early developmental changes in CNS ECs (Passage 1 cells) or as a source of CNS-like ECs for use in downstream modeling applications, we evaluated absolute transcript abundance and effects of treatment or passage number on 53 characteristic CNS EC genes encompassing tight junction components, vesicle trafficking machinery, solute carriers, and ATP-binding cassette (ABC) efflux transporters selected based on high expression in human brain endothelial cells from a meta-analysis of single cell RNA-seq data (Gastfriend et al., 2021) (FIG. 35D). While ECs expressed CLDN5, TJP1, TJP2, OLCN, and LSR, they lacked MARVELD2 (encoding tricellulin) under all conditions. ECs under all conditions also lacked MFSD2A and, despite CHIR-mediated downregulation of PLVAP, retained high absolute expression of this and other caveolae-associated genes. Finally, while many solute carriers and ABC transporters were expressed (SLC2A1, SLC3A2, SLC16A1, SLC38A2, ABCG2), others expressed at the in vivo human BBB were not (SLC5A3, SLC7A11, SLC38A3, SLCO1A2, ABCB1) (FIG. 35D). Thus, while CHIR treatment yields ECs with certain elements of CNS-like character, additional molecular signals are likely necessary to improve other aspects of the in vivo CNS EC phenotype.

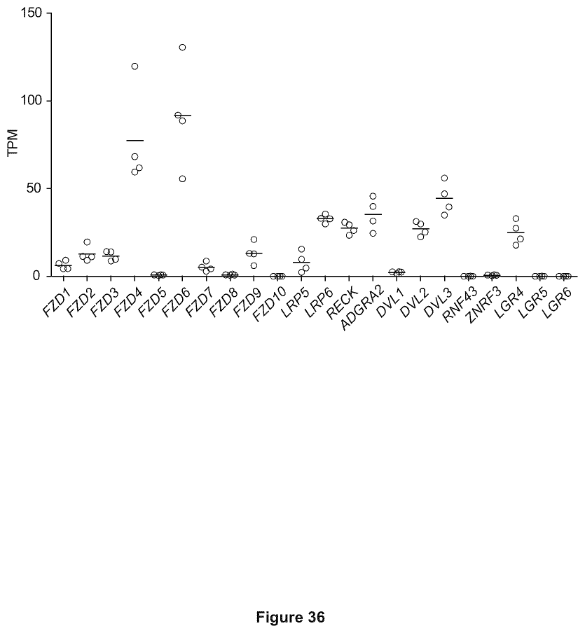

To partially address the hypothesis that the weak response of ECs to Wnt7a/b, NR-CM, and Astro-CM is due to a lack of necessary Wnt receptors and/or co-receptors, we used RNA-seq data from Passage 1 DMSO-treated ECs to evaluate expression of transcripts encoding these and other components of the canonical Wnt signaling pathway. FZD4 and FZD6 were highly expressed and enriched compared to all other Frizzleds (FIG. 36), consistent with data from murine brain ECs in vivo (Daneman et al., 2009). RECK and ADGRA2 (GPR124) were moderately expressed at a level similar to LRP6 (on the order of 40 TPM), while little to no LRP5 was expressed (FIG. 36). Taken together, however, these data suggest that the hPSC-derived ECs express much of the machinery necessary to transduce the signal from Wnt7a/b ligands, but possibilities remain that the proteins encoded by the evaluated transcripts are absent, or at too low an abundance, for a robust response, motivating the use of CHIR to bypass the cell surface Wnt pathway components for robust induction of barriergenesis via β-catenin stabilization.

### The Wnt-Regulated Endothelial Transcriptome in Multiple Contexts

To globally assess whether CHIR-mediated gene expression changes in our system are characteristic of the responses observed in ECs in vivo and similar to those observed in other in vitro contexts, we compared our RNA-seq dataset to those of studies that employed a genetic strategy for β-catenin stabilization (the Ctnnblflex3 allele) in adult mouse ECs in several contexts: (i) pituitary ECs, which acquire some BBB-like properties upon β-catenin stabilization (Wang et al., 2019); (ii) liver ECs, which exhibit little to no barriergenic response to β-catenin stabilization (Munji et al., 2019); (iii) brain ECs briefly cultured in vitro, which rapidly lose their BBB-specific gene expression profile even with β-catenin stabilization (Sabbagh and Nathans, 2020), and offer the most direct comparison to our in vitro model system. Upon recombination, the Ctnnblflex3 allele produces a dominant mutant β-catenin lacking residues that are phosphorylated by GSK-3β to target β-catenin for degradation (Harada et al., 1999); as such, this strategy for ligand- and receptor-independent Wnt activation by β-catenin stabilization is directly analogous to CHIR treatment.

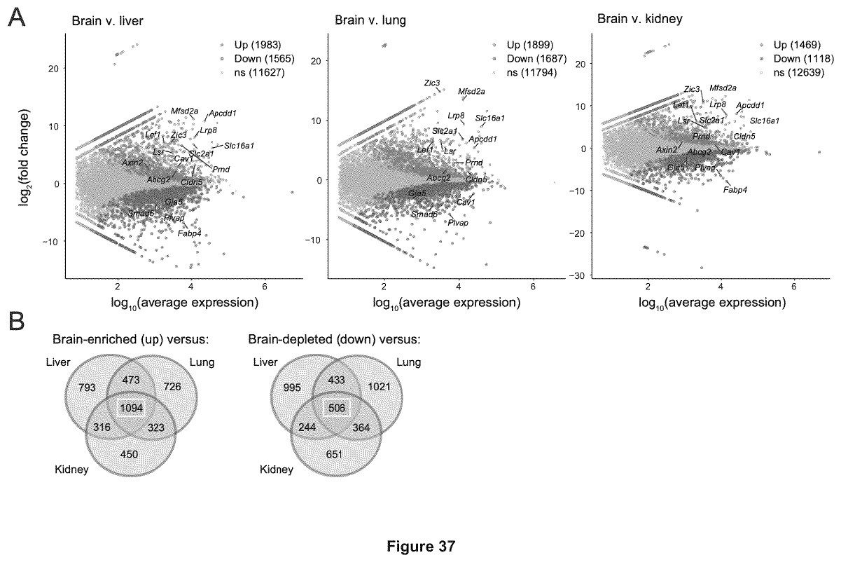

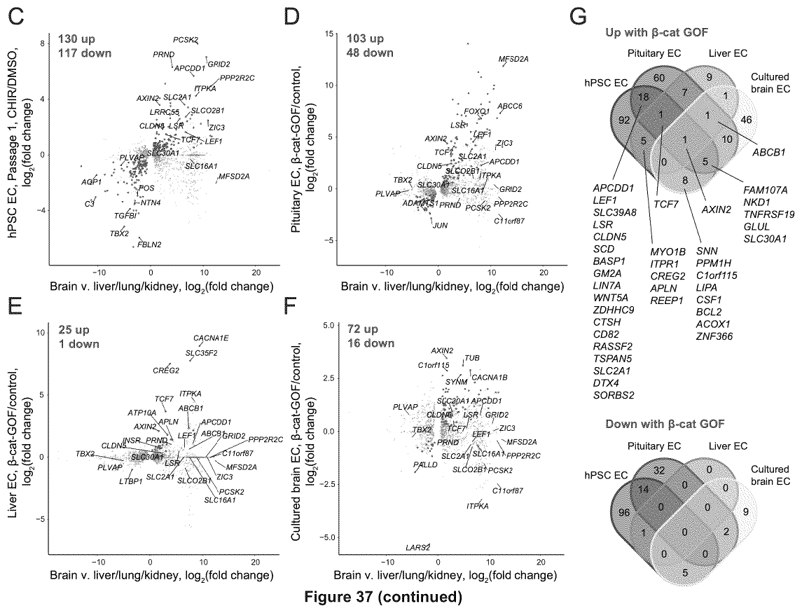

We first used literature RNA-seq data from postnatal day 7 murine brain, liver, lung, and kidney ECs (Sabbagh et al., 2018) to define core sets of genes in brain ECs that are differentially expressed compared to all three of the other organs (FIG. 37A-B). Using the resulting sets of 1094 brain-enriched and 506 brain-depleted genes, we asked how many genes in our Passage 1 ECs were concordantly-regulated by CHIR: 130 of the brain-enriched genes were CHIR-upregulated and 116 of the brain-depleted genes were CHIR-downregulated (FIG. 37C). In pituitary ECs with β-catenin stabilization, 102 of the brain enriched genes were upregulated with and 48 of the brain depleted genes were downregulated (FIG. 37D). Compared with the pituitary ECs, there were far fewer concordantly-regulated genes in liver ECs with β-catenin stabilization, with 25 upregulated and 1 downregulated (FIG. 37E). Finally, cultured primary mouse brain ECs with β-catenin stabilization exhibited 72 upregulated and 16 downregulated genes (FIG. 37F). The only gene concordantly-regulated in all four comparisons was the canonical Wnt target AXIN2. Several additional genes were concordantly upregulated in three of four, including TCF7, FAM107A, NKD1, TNFRSF19, GLUL, SLC30A1, and ABCB1, which was the only gene concordantly regulated in all comparisons except the hPSC-derived ECs (FIG. 37G). Several canonical target genes were shared by the hPSC-derived EC and pituitary EC systems, including APCDD1, LEF1, CLDN5, and SLC2A1; also in this category were LSR, the zinc/manganese transporter SLC39A8, and 12 additional genes (FIG. 37G). Notably, the caveolae inhibitor MFSD2A was robustly upregulated by β-catenin in pituitary ECs, but not in any other context (FIG. 37C-F), suggesting other brain-derived factors may cooperate with Wnt to regulate expression of this important inhibitor of caveolin-mediated transcytosis. In sum, the data suggest that the hPSC-derived ECs responded to Wnt activation in a fashion that led to modest induction of CNS transcriptional programs and that the response was most similar to the pituitary β-catenin stabilization model. Importantly, this analysis also supports the hypothesis that immature endothelium is highly responsive to Wnt activation where mature (adult) endothelium is largely refractory except in regions proximal to barrier-forming regions.

### Discussion

The Wnt/β-catenin signaling pathway plays a central role in CNS angiogenesis and in establishing the unique properties of CNS ECs (Liebner et al., 2008; Stenman et al., 2008; Daneman et al., 2009; Kuhnert et al., 2010; Cullen et al., 2011; Vanhollebeke et al., 2015; Cho et al., 2017). In this work, we investigated the role of Wnt/β-catenin signaling on induction of BBB properties in a human EC model, using naïve endothelial progenitors derived from hPSCs. We reasoned that these immature EPCs (Lian et al., 2014) would be similar to the immature endothelium in the perineural vascular plexus and thus competent to acquire CNS EC phenotypes in response to Wnt activation. We evaluated several strategies to activate Wnt, including the widely used ligand Wnt3a (Liebner et al., 2008), the neural progenitor- and astrocyte-derived ligands Wnt7a and Wnt7b, which are the two Wnt ligands primarily responsible for the Wnt-dependent effects of CNS angiogenesis and barriergenesis observed in vivo (Daneman et al., 2009; Cho et al., 2017), neural rosette- and astrocyte-CM as putative cellular sources of Wnt ligands, and the GSK-3 inhibitor CHIR.

We found that CHIR treatment robustly induced several canonical CNS EC molecular phenotypes, including a marked induction of GLUT-1, upregulation of claudin-5, and downregulation of PLVAP, which correlated with differential gene expression in RNA-seq data. We also observed a functional decrease in paracellular permeability. Further, using RNA-seq and Western blotting, we also identified LSR (angulin-1) as CHIR-induced in this system, supporting the notion that this highly CNS EC-enriched tricellular tight junction protein (Daneman et al., 2010a; Sohet et al., 2015) is Wnt-regulated. In RNA-seq data, we observed differential expression of known CNS EC-enriched/depleted and Wnt-regulated genes including upregulated LEF1, AXIN2, APCDD1, ABCG2, SOX7, and ZIC3 and downregulated PLVAP, FABP4, and SMAD6. These RNA-seq data should therefore be useful in generating hypotheses of BBB-associated genes regulated by Wnt activation in ECs, for future functional studies. Our work also defines an important set of phenotypes for which Wnt activation in ECs is not sufficient in our system: in the context of vesicle trafficking, we observed caveolin-1 (CAV1) upregulation, no change in mean functional endocytosis, virtually no expression of MFSD2A, and high absolute PLVAP abundance despite CHIR-mediated downregulation. Given roles of brain pericytes in regulating PLVAP, MFSD2A, and functional transcytosis (Armulik et al., 2010; Daneman et al., 2010b; Ben-Zvi et al., 2014; Stebbins et al., 2019), and the observation that MFSD2A is Wnt-regulated in pituitary ECs in vivo (Wang et al., 2019), where pericytes are present, it is plausible that pericyte-derived cues are necessary in addition to Wnts to achieve the characteristically low rate of CNS EC pinocytosis. Next, while ABCG2 (BCRP) was Wnt-induced in our system, other hallmark efflux transporters were not Wnt-regulated and either expressed at low levels (e.g., ABCC4, encoding MRP-4) or not expressed (e.g. ABCB1, encoding P-glycoprotein). Notably however, Abcb1a was Wnt-regulated in the three other β-catenin stabilization experiments from the literature that we evaluated (Munji et al., 2019; Wang et al., 2019; Sabbagh and Nathans, 2020). Thus, pericyte-derived cues, astrocyte-derived cues, and/or activation of the pregnane X or other nuclear receptors may be important for complete acquisition of the complement of CNS EC efflux transporters (Bauer et al., 2004; Berezowski et al., 2004; Praca et al., 2019).

While several recombinant Wnt ligands and neural rosette-CM elevated GLUT-1 expression in ECs, the magnitude of this effect was small compared to the robust induction of GLUT-1 observed with CHIR treatment. While we observed moderate transcript-level expression RECK and ADGRA2 (GPR124) in Passage 1 ECs, it is possible that protein-level expression of these necessary Wnt7 coreceptors, or additional components necessary for Wnt signal transduction, are not of sufficient abundance. For example, absence of LRP5 is a potential factor in the muted response to Wnt ligands and CM because LRP5 and LRP6 likely have non-redundant functions, as evidenced by defects in retinal barrier formation in Lrp5-knockout mice (Zhou et al., 2014). Presence of GPR124 in naïve endothelial progenitors is consistent with ubiquitous expression in ECs in the mouse embryo that is subsequently downregulated in non-CNS endothelium; however, GPR124 enrichment in CNS ECs can be observed as early as E12.5 (Kuhnert et al., 2010), leaving open the possibility that during development other neural tissue-derived signals upregulate or maintain RECK and GPR124 expression. Furthermore, while ligand potency or concentration may also play a role in the weak response, we observed a consistent and highly potent EC-purifying effect (i.e., reduction or elimination of the contaminating SMLCs observed in control Passage 1 cultures) with Wnt7a and both neural rosette- and astrocyte-CM. CHIR also achieved this purifying effect and increased EC number, suggesting that Wnt signaling plays a role in suppressing proliferation of mesoderm-derived mural cells in this system.

We also directly addressed the hypothesis that immature ECs are more plastic, that is, more competent to acquire BBB properties upon Wnt activation, than mature ECs. This hypothesis is supported by existing observations that ectopic expression of Wnt7a is sufficient to induce GLUT-1 expression in non-CNS regions of the mouse embryo (Stenman et al., 2008), but β-catenin stabilization in adult mouse liver and lung ECs produces only a slight effect (Munji et al., 2019). We repeated our CHIR treatment paradigm in hPSC-derived ECs after an extended period of in vitro culture, and observed much weaker induction of GLUT-1 and no pro-proliferative effect. Thus, our results support this hypothesis and suggest that the loss of BBB developmental plasticity in ECs is an intrinsic, temporally-controlled process rather than a result of the peripheral organ environment. Interestingly, ECs in non-BBB-forming regions of the CNS (i.e., CVOs), and in the anterior pituitary, which is directly proximal to the CNS, retain some of their plasticity in adulthood (Wang et al., 2019), possibly as the result of a delicate balance between Wnt ligands and Wnt-inhibitory factors in these regions. Our model should facilitate additional systematic examination of factors that may enhance or attenuate EC Wnt responsiveness.

Finally, our work establishes an improved hPSC-based model for investigating mechanisms of BBB development in naïve ECs. hPSCs are an attractive model system to complement in vivo animal studies because they (i) are human, (ii) permit investigation of developmental processes in contrast to primary or immortalized cells, (iii) are highly scalable, (iv) can be derived from patients to facilitate disease modeling and autologous coculture systems, and (v) are genetically tractable. While widely used hPSC-based BBB models are useful for measuring molecular permeabilities and have been employed to understand genetic contributions to barrier dysfunction (Vatine et al., 2016, 2019; Lim et al., 2017), they have not been shown to proceed through a definitive endothelial progenitor intermediate (Lippmann et al., 2012; Lu et al., 2021) and express epithelial-associated genes (Qian et al., 2017; Delsing et al., 2018; Vatine et al., 2019; Lu et al., 2021). Thus, new models with developmentally relevant differentiation trajectories and definitive endothelial phenotype are needed for improved understanding of developmental mechanisms. Motivated in part by prior use of endothelial cells derived from hematopoietic progenitors in human cord blood to generate BBB models (Boyer-Di Ponio et al., 2014; Cecchelli et al., 2014), we and others recently showed that hPSC-derived naïve endothelial progenitors or ECs are good candidates for such a system (Praca et al., 2019; Nishihara et al., 2020; Roudnicky et al., 2020a, 2020b). For example, Praca et al. showed that a combination of VEGF, Wnt3a, and retinoic acid directed EPCs to brain capillary-like ECs with moderate transendothelial electrical resistance (TEER) of ˜60×cm². We previously showed that BBB-like paracellular barrier characteristics are induced in hPSC-EPC-derived ECs after extended culture in a minimal medium. These so-called EECM-BMEC-like cells had TEER and small molecule permeability similar to primary human brain ECs, well-developed tight junctions, and an immune cell adhesion molecule profile similar to brain ECs in vivo (Nishihara et al., 2020). In this study, we showed it was possible to use the small molecule Wnt agonist CHIR to induce additional hallmarks of CNS EC phenotype in hPSC-EPC-derived ECs, including canonical GLUT-1, claudin-5, and PLVAP effects (both Passage 1 and 3 CHIR-treated ECs). However, it is important to note that despite the improvements in CNS EC character with CHIR treatment, further improvements to functional endocytosis, and efflux transporter and solute carrier phenotype should be targets of future study and may be facilitated by cocultures and/or additional molecular factors. Along these lines, the Passage 1 CHIR-treated CNS-like ECs would be at a differentiation stage well suited to investigate cues subsequent to Wnt signaling that may be key for the induction of additional CNS EC properties. Alternatively, the Passage 3 CHIR-treated CNS-like ECs may be suitable for other BBB modeling applications. In summary, our work has defined the EC response to Wnt activation in a simplified, human system and established a new hPSC-derived in vitro model that will facilitate improved understanding of endothelial barriergenesis.

### EXAMPLE 2 REFERENCES

Andreone B J, Chow B W, Tata A, Lacoste B, Ben-Zvi A, Bullock K, Deik A A, Ginty D D, Clish C B, Gu C. 2017. Blood-Brain Barrier Permeability Is Regulated by Lipid Transport-Dependent Suppression of Caveolae-Mediated Transcytosis. Neuron 94:581-594.e5. doi:10.1016/j.neuron.2017.03.043
Armulik A, Genové G, Mae M, Nisancioglu M H, Wallgard E, Niaudet C, He L, Norlin J, Lindblom P, Strittmatter K, Johansson B R, Betsholtz C. 2010. Pericytes regulate the blood-brain barrier. Nature 468:557-561. doi:10.1038/nature09522
Bao X, Bhute V J, Han T, Qian T, Lian X, Palecek S P. 2017. Human pluripotent stem cell-derived epicardial progenitors can differentiate to endocardial-like endothelial cells 191-201. doi:10.1002/btm2.10062
Bao X, Lian X, Palecek S P. 2016. Directed Endothelial Progenitor Differentiation from Human Pluripotent Stem Cells Via Wnt Activation Under Defined Conditions. Methods Mol Biol 1481:183-96. doi:10.1007/978-1-4939-6393-5_17
Bauer B, Hartz A M S, Fricker G, Miller D S. 2004. Pregnane X receptor up-regulation of P-glycoprotein expression and transport function at the blood-brain barrier. Mol Pharmacol 66:413-419. doi:10.1124/mol.66.3.
Ben-Zvi A, Lacoste B, Kur E, Andreone B J, Mayshar Y, Yan H, Gu C. 2014. Mfsd2a is critical for the formation and function of the blood-brain barrier. Nature 509:507-11. doi:10.1038/nature13324
Benz F, Wichitnaowarat V, Lehmann M, Germano R F, Mihova D, Macas J, Adams R H, Mark Taketo M, Plate K H, Guërit S, Vanhollebeke B, Liebner S. 2019. Low wnt/β-catenin signaling determines leaky vessels in the subfornical organ and affects water homeostasis in mice. Elife 8:1-29. doi:10.7554/eLife.43818
Berezowski V, Landry C, Dehouck M P, Cecchelli R, Fenart L. 2004. Contribution of glial cells and pericytes to the mRNA profiles of P-glycoprotein and multidrug resistance-associated proteins in an in vitro model of the blood-brain barrier. Brain Res 1018:1-9. doi:10.1016/j.brainres.2004.05.092
Boyer-Di Ponio J, El-Ayoubi F, Glacial F, Ganeshamoorthy K, Guillevic O, Couraud P O, Driancourt C, Godet M, Perrie N, Uzan G, Boyer-Di Ponio J, El-Ayoubi F, Glacial F, Ganeshamoorthy K, Driancourt C, Godet M, Perrière N, Guillevic O, Olivier Couraud P, Uzan G, Ponio J B, El-Ayoubi F, Glacial F, Ganeshamoorthy K, Guillevic O, Couraud P O, Driancourt C, Godet M, Perrie N, Uzan G. 2014. Instruction of circulating endothelial progenitors in vitro towards specialized blood-brain barrier and arterial phenotypes. PLoS One 9. doi:10.1371/journal.pone.0084179
Canfield S G, Stebbins M J, Morales B S, Asai S W, Vatine G D, Svendsen C N, Palecek S P, Shusta E V. 2017. An isogenic blood-brain barrier model comprising brain endothelial cells, astrocytes, and neurons derived from human induced pluripotent stem cells. J Neurochem 140:874-888. doi:10.1111/jnc.13923
Castro Dias M, Coisne C, Lazarevic I, Baden P, Hata M, Iwamoto N, Miguel D, Francisco F, Vanlandewijck M, He L, Baier F A, Stroka D, Bruggmann R, Lyck R, En G, Deutsch U, Betsholtz C, Furuse M, Shoichiro T, Engelhardt B, Castro Dias M, Coisne C, Lazarevic I, Baden P, Hata M, Iwamoto N, Francisco D M F, Vanlandewijck M, He L, Baier F A, Stroka D, Bruggmann R, Lyck R, Enzmann G, Deutsch U, Betsholtz C, Furuse M, Tsukita S, Engelhardt B. 2019. Claudin-3-deficient C57BL/6J mice display intact brain barriers. Sci Rep 9:1-16. doi:10.1038/s41598-018-36731-3
Cecchelli R, Aday S, Sevin E, Almeida C, Culot M, Dehouck L, Coisne C, Engelhardt B, Dehouck M P, Ferreira L. 2014. A stable and reproducible human blood-brain barrier model derived from hematopoietic stem cells. PLoS One 9. doi:10.1371/journal.pone.0099733
Chen G, Gulbranson D R, Hou Z, Bolin J M, Ruotti V, Probasco M D, Smuga-Otto K, Howden S E, Diol N R, Propson N E, Wagner R, Lee G O, Antosiewicz-Bourget J, Teng J M C, Thomson J A. 2011. Chemically defined conditions for human iPSC derivation and culture. Nat Methods 8:424-429. doi:10.1038/nmeth.1593
Cho C, Smallwood P M, Nathans J. 2017. Reck and Gpr124 Are Essential Receptor Cofactors for Wnt7a/Wnt7b-Specific Signaling in Mammalian CNS Angiogenesis and Blood-Brain Barrier Regulation. Neuron 95:1056-1073.e5. doi:10.1016/j.neuron.2017.07.031
Clevers H, Loh K M, Nusse R. 2014. Stem cell signaling. An integral program for tissue renewal and regeneration: Wnt signaling and stem cell control. Science 346:1248012. doi:10.1126/science.1248012
Corada M, Orsenigo F, Bhat G P, Conze L L, Breviario F, Cunha S I, Claesson-welsh L, Beznoussenko G V, Mironov A A, Bacigaluppi M, Martino G, Pitulescu M E, Adams R H, Magnusson P, Dejana E. 2018. Fine-Tuning of Sox17 and Canonical Wnt Coordinates the Permeability Properties of the Blood-Brain Barrier 511-525. doi:10.1161/CIRCRESAHA.118.313316
Cullen M, Elzarrad M K, Seaman S, Zudaire E, Stevens J, Yang M Y, Li X, Chaudhary A, Xu L, Hilton M B, Logsdon D, Hsiao E, Stein E V., Cuttitta F, Haines D C, Nagashima K, Tessarollo L, St. Croix B. 2011. GPR124, an orphan G protein-coupled receptor, is required for CNS-specific vascularization and establishment of the blood-brain barrier. Proc Natl Acad Sci 108:5759-5764. doi:10.1073/pnas.1017192108
Daneman R, Agalliu D, Zhou L, Kuhnert F, Kuo C J, Barres B A. 2009. Wnt/beta-catenin signaling is required for CNS, but not non-CNS, angiogenesis. Proc Natl Acad Sci USA 106:641-6. doi:10.1073/pnas.0805165106
Daneman R, Engelhardt B. 2017. Brain barriers in health and disease. Neurobiol Dis 107:1-3. doi:10.1016/j.nbd.2017.05.008
Daneman R, Zhou L, Agalliu D, Cahoy J D, Kaushal A, Barres B A. 2010a. The Mouse Blood-Brain Barrier Transcriptome: A New Resource for Understanding the Development and Function of Brain Endothelial Cells. PLoS One 5:e13741. doi:10.1371/journal.pone.0013741
Daneman R, Zhou L, Kebede A A, Barres B A. 2010b. Pericytes are required for blood-brain barrier integrity during embryogenesis. Nature 468:562-566. doi:10.1038/nature09513
Delsing L, Dönnes P, Sanchez J, Clausen M, Voulgaris D, Falk A, Herland A, Brolén G, Zetterberg H, Hicks R, Synnergren J. 2018. Barrier Properties and Transcriptome Expression in Human iPSC-Derived Models of the Blood-Brain Barrier. Stem Cells 36:1816-1827. doi:10.1002/stem.2908
Durinck S, Spellman P T, Birney E, Huber W. 2009. Mapping identifiers for the integration of genomic datasets with the R/Bioconductor package biomaRt. Nat Protoc 4:1184-1191. doi:10.1038/nprot.2009.97
Ebert A D, Shelley B C, Hurley A M, Onorati M, Castiglioni V, Patitucci T N, Svendsen S P, Mattis V B, McGivern J V., Schwab A J, Sareen D, Kim H W, Cattaneo E, Svendsen C N. 2013. EZ spheres: A stable and expandable culture system for the generation of pre-rosette multipotent stem cells from human ESCs and iPSCs. Stem Cell Res 10:417-427. doi:10.1016/j.scr.2013.01.009
Engelhardt B, Liebner S. 2014. Novel insights into the development and maintenance of the blood-brain barrier. Cell Tissue Res. doi:10.1007/s00441-014-1811-2
Eubelen M, Bostaille N, Cabochette P, Gauquier A, Tebabi P, Dumitru A C, Koehler M, Gut P, Alsteens D, Stainier D Y R, Garcia-Pino A, Vanhollebeke B. 2018. A molecular mechanism for Wnt ligand-specific signaling. Science (80-) 361. doi:10.1126/science.aat1178
Gastfriend B D, Foreman K L, Katt M E, Palecek S P, Shusta E V. 2021. Integrative analysis of the human brain mural cell transcriptome. J Cereb Blood Flow Metab In press.:0271678X2110137. doi:10.1177/0271678X211013700
Guérit S, Fidan E, Macas J, Czupalla C J, Figueiredo R, Vijikumar A, Yalcin B H, Thom S, Winter P, Gerhardt H, Devraj K, Liebner S. 2021. Astrocyte-derived Wnt growth factors are required for endothelial blood-brain barrier maintenance. Prog Neurobiol 199. doi:10.1016/j.pneurobio.2020.101937
Han X, Zhou Z, Fei L, Sun H, Wang R, Chen Y, Chen H, Wang J, Tang H, Ge W, Zhou Y, Ye F, Jiang M, Wu J, Xiao Y, Jia X, Zhang T, Ma X, Zhang Q, Bai X, Lai S, Yu C, Zhu L, Lin R, Gao Y, Wang M, Wu Y, Zhang J, Zhan R, Zhu S, Hu H, Wang C, Chen M, Huang H, Liang T, Chen J, Wang W, Zhang D, Guo G. 2020. Construction of a human cell landscape at single-cell level, Nature. Springer US. doi:10.1038/s41586-020-2157-4
Harada N, Tamai Y, Ishikawa T O, Sauer B, Takaku K, Oshima M, Taketo M M. 1999. Intestinal polyposis in mice with a dominant stable mutation of the β-catenin gene. EMBO J 18:5931-5942. doi:10.1093/emboj/18.21.5931
He L, Vanlandewijck M, Mae M A, Andrae J, Ando K, Del Gaudio F, Nahar K, Lebouvier T, Laviña B, Gouveia L, Sun Y, Raschperger E, Segerstolpe Å, Liu J, Gustafsson S, Räsänen M, Zarb Y, Mochizuki N, Keller A, Lendahl U, Betsholtz C. 2018. Single-cell RNA sequencing of mouse brain and lung vascular and vessel-associated cell types. Sci data 5:180160. doi:10.1038/sdata.2018.160
Herrnberger L, Seitz R, Kuespert S, Bosl M R, Fuchshofer R, Tamm E R. 2012. Lack of endothelial diaphragms in fenestrae and caveolae of mutant Plvap-deficient mice. Histochem Cell Biol 138:709-724. doi:10.1007/s00418-012-0987-3
Jaffe R B, Yu J, Vodyanik M A, Smuga-Otto K, Antosiewicz-Bourget J, Frane J L, Tian S, Nie J, Jonsdottir G A, Ruotti V, Stewart R, Slukvin I I, Thomson J A. 2008. Induced Pluripotent Stem Cell Lines Derived From Human Somatic Cells. Obstet Gynecol Sury 63:154-155. doi:10.1097/01.ogx.0000305193.72586.39
Kim K-A, Kakitani M, Zhao J, Oshima T, Tang T, Binnerts M, Liu Y, Boyle B, Park E, Emtage P, Funk W D, Tomizuka K. 2005. Mitogenic influence of human R-spondinl on the intestinal epithelium. Science 309:1256-9. doi:10.1126/science.1112521
Kim K-A, Wagle M, Tran K, Zhan X, Dixon M A, Liu S, Gros D, Korver W, Yonkovich S, Tomasevic N, Binnerts M, Abo A. 2008. R-Spondin Family Members Regulate the Wnt Pathway by a Common Mechanism. Mol Biol Cell 19:2588-2596. doi:10.1091/mbc.e08-02-0187
Koo B K, Spit M, Jordens I, Low T Y, Stange D E, Van De Wetering M, Van Es J H, Mohammed S, Heck A J R, Maurice M M, Clevers H. 2012. Tumour suppressor RNF43 is a stem-cell E3 ligase that induces endocytosis of Wnt receptors. Nature 488:665-669. doi:10.1038/nature11308
Kreitzer F R, Salomonis N, Sheehan A, Huang M, Park J S, Spindler M J, Lizarraga P, Weiss W A, So P, Conklin B R. 2013. A robust method to derive functional neural crest cells from human pluripotent stem cells. Am J Stem Cells 2:119-31.
Kuhnert F, Mancuso M R, Shamloo A, Wang H-THT, Choksi V, Florek M, Su H, Fruttiger M, Young W L, Heilshorn S C, Kuo C J. 2010. Essential regulation of CNS angiogenesis by the orphan G protein-coupled receptor GPR124. Science (80-) 330:985-989. doi:10.1126/science.1196554
La Manno G, Gyllborg D, Codeluppi S, Nishimura K, Salto C, Zeisel A, Borm L E, Stott S R W, Toledo E M, Villaescusa J C, Lonnerberg P, Ryge J, Barker R A, Arenas E, Linnarsson S. 2016. Molecular Diversity of Midbrain Development in Mouse, Human, and Stem Cells. Cell 167:566-580.e19. doi:10.1016/j.cell.2016.09.027
Laksitorini M D, Yathindranath V, Xiong W, Hombach-Klonisch S, Miller D W. 2019. Modulation of Wnt/β-catenin signaling promotes blood-brain barrier phenotype in cultured brain endothelial cells. Sci Rep 9:1-13. doi:10.1038/s41598-019-56075-w Langmead B, Salzberg S L. 2012. Fast gapped-read alignment with Bowtie 2. Nat Methods 9:357-359. doi:10.1038/nmeth.1923
Li B, Dewey C N. 2011. RSEM: accurate transcript quantification from RNA-Seq data with or without a reference genome. BMC Bioinformatics 12:323.
Lian X, Bao X, Al-Ahmad A, Liu J, Wu Y, Dong W, Dunn K K, Shusta E V., Palecek S P. 2014. Efficient differentiation of human pluripotent stem cells to endothelial progenitors via small-molecule activation of WNT signaling. Stem Cell Reports 3:804-816. doi:10.1016/j.stemcr.2014.09.005
Lian X, Zhang J, Zhu K, Kamp T J, Palecek S P. 2013. Insulin inhibits cardiac mesoderm, not mesendoderm, formation during cardiac differentiation of human pluripotent stem cells and modulation of canonical wnt signaling can rescue this inhibition. Stem Cells 31:447-457. doi:10.1002/stem.1289
Liebner S, Corada M, Bangsow T, Babbage J, Taddei A, Czupalla C J, Reis M, Felici A, Wolburg H, Fruttiger M, Taketo M M, Von Melchner H, Plate K H, Gerhardt H, Dejana E. 2008. Wnt/β-catenin signaling controls development of the blood-brain barrier. J Cell Biol 183:409-417. doi:10.1083/jcb.200806024
Lim R G, Quan C, Reyes-Ortiz A M, Lutz S E, Kedaigle A J, Gipson T A, Wu J, Vatine G D, Stocksdale J, Casale M S, Svendsen C N, Fraenkel E, Housman D E, Agalliu D, Thompson LM. 2017. Huntington's Disease iPSC-Derived Brain Microvascular Endothelial Cells Reveal WNT-Mediated Angiogenic and Blood-Brain Barrier Deficits. Cell Rep 19:1365-1377. doi:10.1016/j.celrep.2017.04.021
Lippmann E S, Azarin S M, Kay J E, Nessler R A, Wilson H K, Al-Ahmad A, Palecek S P, Shusta E V. 2012. Derivation of blood-brain barrier endothelial cells from human pluripotent stem cells. Nat Biotechnol 30:783-791. doi:10.1038/nbt.2247
Lippmann E S, Azarin S M, Palecek S P, Shusta E V. 2020. Commentary on human pluripotent stem cell-based blood-brain barrier models. Fluids Barriers CNS 17:4-9. doi:10.1186/s12987-020-00222-3
Lippmann E S, Estevez-Silva M C, Ashton R S. 2014. Defined human pluripotent stem cell culture enables highly efficient neuroepithelium derivation without small molecule inhibitors. Stem Cells 32:1032-1042. doi:10.1002/stem.1622
Love M I, Huber W, Anders S. 2014. Moderated estimation of fold change and dispersion for RNA-seq data with DESeq2. Genome Biol 15:550. doi:10.1186/s13059-014-0550-8
Lu T M, Houghton S, Magdeldin T, Barcia Duran J G, Minotti A P, Snead A, Sproul A, Nguyen D H T, Xiang J, Fine H A, Rosenwaks Z, Studer L, Rafii S, Agalliu D, Redmond D, Lis R. 2021. Pluripotent stem cell-derived epithelium misidentified as brain microvascular endothelium requires ETS factors to acquire vascular fate. Proc Natl Acad Sci USA 118. doi:10.1073/pnas.2016950118
Mazzoni J, Smith J R, Shahriar S, Cutforth T, Ceja B, Agalliu D. 2017. The Wnt Inhibitor Apcdd1 Coordinates Vascular Remodeling and Barrier Maturation of Retinal Blood Vessels. Neuron 96:1055-1069.e6. doi:10.1016/j.neuron.2017.10.025
Mouillesseaux K P, Wiley D S, Saunders L M, Wylie L A, Kushner E J, Chong D C, Citrin K M, Barber A T, Park Y, Kim J D J J D, Samsa L A, Kim J D J J D, Liu J, Jin S W, Bautch V L. 2016. Notch regulates BMP responsiveness and lateral branching in vessel networks via SMAD6. Nat Commun 7:1-12. doi:10.1038/ncomms13247
Munji R N, Soung A L, Weiner G A, Sohet F, Semple B D, Trivedi A, Gimlin K, Kotoda M, Korai M, Aydin S, Batugal A, Cabangcala A C, Schupp P G, Oldham M C, Hashimoto T, Noble-Haeusslein L J, Daneman R. 2019. Profiling the mouse brain endothelial transcriptome in health and disease models reveals a core blood-brain barrier dysfunction module. Nat Neurosci 22:1892-1902. doi:10.1038/s41593-019-0497-x
Nishihara H, Gastfriend B D, Soldati S, Perriot S, Mathias A, Sano Y, Shimizu F, Gosselet F, Kanda T, Palecek S P, Du Pasquier R, Shusta E V., Engelhardt B. 2020. Advancing human induced pluripotent stem cell-derived blood-brain barrier models for studying immune cell interactions. FASEB J 34:16693-16715. doi:10.1096/fj 0.202001507RR
Obermeier B, Daneman R, Ransohoff R M. 2013. Development, maintenance and disruption of the blood-brain barrier. Nat Med. doi:10.1038/nm.3407
Paolinelli R, Corada M, Ferrarini L, Devraj K, Artus C, Czupalla C J, Rudini N, Maddaluno L, Papa E, Engelhardt B, Couraud P O, Liebner S, Dejana E. 2013. Wnt Activation of Immortalized Brain Endothelial Cells as a Tool for Generating a Standardized Model of the Blood Brain Barrier In Vitro. PLoS One 8. doi:10.1371/journal.pone.0070233
Phoenix T N, Patmore D M, Boop S, Boulos N, Jacus M O, Patel Y T, Roussel M F, Finkelstein D, Goumnerova L, Perreault S, Wadhwa E, Cho Y J, Stewart C F, Gilbertson R J. 2016. Medulloblastoma Genotype Dictates Blood Brain Barrier Phenotype. Cancer Cell 29:508-522. doi:10.1016/j.ccell.2016.03.002
Polioudakis D, de la Torre-Ubieta L, Langerman J, Elkins A G, Shi X, Stein J L, Vuong C K, Nichterwitz S, Gevorgian M, Opland C K, Lu D, Connell W, Ruzzo E K, Lowe J K, Hadzic T, Hinz F I, Sabri S, Lowry W E, Gerstein M B, Plath K, Geschwind D H. 2019. A Single-Cell Transcriptomic Atlas of Human Neocortical Development during Mid-gestation. Neuron 103:785-801.e8. doi:10.1016/j.neuron.2019.06.011
Praça C, Rosa S C, Sevin E, Cecchelli R, Dehouck M-PP, Ferreira L S. 2019. Derivation of Brain Capillary-like Endothelial Cells from Human Pluripotent Stem Cell-Derived Endothelial Progenitor Cells. Stem Cell Reports 13: 599-611. doi:10.1016/j.stemcr.2019.08.002
Profaci C P, Munji R N, Pulido R S, Daneman R. 2020. The blood-brain barrier in health and disease: Important unanswered questions. J Exp Med 217:1-16. doi:10.1084/jem.20190062
Qian T, Maguire S E, Canfield S G, Bao X, Olson W R, Shusta E V, Palecek S P. 2017. Directed differentiation of human pluripotent stem cells to blood-brain barrier endothelial cells. Sci Adv 3:e1701679. doi:10.1126/sciadv.1701679
Reese T S, Karnovsky M J. 1967. Fine structural localization of a blood-brain barrier to exogenous peroxidase. J Cell Biol 34:207-217. doi:10.1083/jcb.34.1.207
Roudnicky F, Kim B K, Lan Y, Schmucki R, Küppers V, Christensen K, Graf M, Patsch C, Burcin M, Meyer C A, Westenskow P D, Cowan C A. 2020a. Identification of a combination of transcription factors that synergistically increases endothelial cell barrier resistance. Sci Rep 10:1-9. doi:10.1038/s41598-020-60688-x
Roudnicky F, Zhang J D, Kim B K, Pandya N J, Lan Y, Sach-Peltason L, Ragelle H, Strassburger P, Gruener S, Lazendic M, Uhles S, Revelant F, Eidam O, Sturm G, Kueppers V, Christensen K, Goldstein L D, Tzouros M, Banfai B, Modrusan Z, Graf M, Patsch C, Burcin M, Meyer C A, Westenskow P D, Cowan C A. 2020b. Inducers of the endothelial cell barrier identified through chemogenomic screening in genome-edited hPSC-endothelial cells. Proc Natl Acad Sci USA 117:19854-19865. doi:10.1073/PNAS.1911532117
Sabbagh M F, Heng J S, Luo C, Castanon R G, Nery J R, Rattner A, Goff L A, Ecker J R, Nathans J. 2018. Transcriptional and epigenomic landscapes of CNS and non-CNS vascular endothelial cells. Elife 7:1-44. doi:10.7554/eLife.36187
Sabbagh M F, Nathans J. 2020. A genome-wide view of the de-differentiation of central nervous system endothelial cells in culture. Elife 9:1-19. doi:10.7554/eLife.51276
Sareen D, Gowing G, Sahabian A, Staggenborg K, Paradis R, Avalos P, Latter J, Ornelas L, Garcia L, Svendsen C N. 2014. Human induced pluripotent stem cells are a novel source of neural progenitor cells (iNPCs) that migrate and integrate in the rodent spinal cord. J Comp Neurol 522:2707-2728. doi:10.1002/cne.23578
Schindelin J, Arganda-carreras I, Frise E, Kaynig V, Longair M, Pietzsch T, Preibisch S, Rueden C, Saalfeld S, Schmid B, Tinevez J, White D J, Hartenstein V, Eliceiri K, Tomancak P, Cardona A. 2019. Fiji: an open-source platform for biological-image analysis 9. doi:10.1038/nmeth.2019
Shang Z, Chen D, Wang Q, Wang SSSSS, Deng Q, Wu L, Liu C, Ding X, Wang SSSSS, Zhong J, Zhang D, Cai X, Zhu S, Yang H, Liu L, Fink J L, Chen F, Liu X, Gao Z, Xu X. 2018. Single-cell RNA-seq reveals dynamic transcriptome profiling in human early neural differentiation. Gigascience 7:1-19. doi:10.1093/gigascience/giy117
Sohet F, Lin C, Munji R N, Lee S Y, Ruderisch N, Soung A, Arnold T D, Derugin N, Vexler Z S, Yen F T, Daneman R. 2015. LSR/angulin-1 is a tricellular tight junction protein involved in blood-brain barrier formation. J Cell Biol 208:703-711. doi:10.1083/jcb.201410131
Song H W, Foreman K L, Gastfriend B D, Kuo J S, Palecek S P, Shusta E V. 2020. Transcriptomic comparison of human and mouse brain microvessels. Sci Rep 10:12358. doi:10.1038/s41598-020-69096-7
Stebbins M J, Gastfriend B D, Canfield S G, Lee M-SS, Richards D, Faubion M G, Li W-JJ, Daneman R, Palecek S P, Shusta E V. 2019. Human pluripotent stem cell-derived brain pericyte-like cells induce blood-brain barrier properties. Sci Adv 5:eaau7375. doi:10.1126/sciadv.aau7375
Stebbins M J, Wilson H K, Canfield S G, Qian T, Palecek S P, Shusta E V. 2016. Differentiation and characterization of human pluripotent stem cell-derived brain microvascular endothelial cells. Methods 101:93-102. doi:10.1016/j.ymeth.2015.10.016
Stenman J M, Rajagopal J, Carroll T J, Ishibashi M, McMahon J, McMahon A P. 2008. Canonical Wnt Signaling Regulates Organ-Specific Assembly and Differentiation of CNS Vasculature. Science (80-) 322:1247-1250. doi:10.1126/science.1164594
Syvänen S, Lindhe Ö, Palner M, Kornum B R, Rahman O, Långström B, Knudsen G M, Hammarlund-Udenaes M, Lindhe O, Palner M, Kornum B R, Rahman O, Långström B, Knudsen G M, Hammarlund-Udenaes M. 2009. Species differences in blood-brain barrier transport of three positron emission tomography radioligands with emphasis on P-glycoprotein transport. Drug Metab Dispos 37:635-643. doi:10.1124/dmd.108.024745
Tran K A, Zhang X, Predescu D, Huang X, MacHado RF, Göthert JR, Malik A B, Valyi-Nagy T, Zhao Y-YY. 2016. Endothelial β-Catenin Signaling Is Required for Maintaining Adult Blood-Brain Barrier Integrity and Central Nervous System Homeostasis. Circulation 133:177-86. doi:10.1161/CIRCULATIONAHA.115.015982
Uchida Y, Ohtsuki S, Katsukura Y, Ikeda C, Suzuki T, Kamiie J, Terasaki T. 2011. Quantitative targeted absolute proteomics of human blood-brain barrier transporters and receptors. J Neurochem 117:333-345. doi:10.1111/j.1471-4159.2011.07208.x
Umans R A, Henson H E, Mu F, Parupalli C, Ju B, Peters J L, Lanham K A, Plavicki J S, Taylor M R. 2017. CNS angiogenesis and barriergenesis occur simultaneously. Dev Biol 425:101-108. doi:10.1016/j.ydbio.2017.03.017
Vallon M, Yuki K, Nguyen T D, Chang J, Yuan J, Siepe D, Miao Y, Essler M, Noda M, Garcia K C, Kuo C J. 2018. A RECK-WNT7 Receptor-Ligand Interaction Enables Isoform-Specific Regulation of Wnt Bioavailability. Cell Rep 25:339-349.e9. doi:10.1016/j.celrep.2018.09.045
Vanhollebeke B, Stone O A, Bostaille N, Cho C, Zhou Y, Maquet E, Gauquier A, Cabochette P, Fukuhara S, Mochizuki N, Nathans J, Stainier D Y R. 2015. Tip cell-specific requirement for an atypical Gpr124- and Reck-dependent Wnt/β-catenin pathway during brain angiogenesis. Elife 4:1-25. doi:10.7554/eLife.06489
Vanlandewijck M, He L, Mae M A, Andrae J, Ando K, Del Gaudio F, Nahar K, Lebouvier T, Laviña B, Gouveia L, Sun Y, Raschperger E, Rasanen M, Zarb Y, Mochizuki N, Keller A, Lendahl U, Betsholtz C. 2018. A molecular atlas of cell types and zonation in the brain vasculature. Nature 554:475-480. doi:10.1038/nature25739
Vatine G D, Al-Ahmad A, Barriga B K, Svendsen S, Salim A, Garcia L, Garcia V J, Ho R, Yucer N, Qian T, Lim R G, Wu J, Thompson L M, Spivia W R, Chen Z, Van Eyk J, Palecek S P, Refetoff S, Shusta E V, Svendsen C N. 2016. Modeling Psychomotor Retardation using iPSCs from MCT8-Deficient Patients Indicates a Prominent Role for the Blood-Brain Barrier. Cell Stem Cell 1-13. doi:10.1016/j.stem.2017.04.002
Vatine G D, Barrile R, Workman M J, Chen Z, Eyk J Van, Svendsen C N, Vatine G D, Barrile R, Workman M J, Sances S, Barriga B K, Spivia W R, Chen Z, Eyk J Van, Svendsen C N. 2019. Human iPSC-Derived Blood-Brain Barrier Chips Resource Chips Enable Disease Modeling and Personalized Medicine Applications. Stem Cell 24:995-1005.e6. doi:10.1016/j.stem.2019.05.011
Wang Y, Rattner A, Zhou Y, Williams J, Smallwood P M, Nathans J. 2012. Norrin/Frizzled4 signaling in retinal vascular development and blood brain barrier plasticity. Cell 151:1332-1344. doi:10.1016/j.cell.2012.10.042
Wang Y, Sabbagh M F, Gu X, Rattner A, Williams J, Nathans J. 2019. Beta-catenin signaling regulates barrier-specific gene expression in circumventricular organ and ocular vasculatures. Elife 8:1-36. doi:10.7554/eLife.43257
Workman M J, Svendsen C N. 2020. Recent advances in human iPSC-derived models of the blood-brain barrier. Fluids Barriers CNS 17:1-10. doi:10.1186/s12987-020-00191-7
Yates A D, Achuthan P, Akanni W, Allen J J J, Allen J J J, Alvarez-Jarreta J, Amode M R, Armean I M, Azov A G, Bennett R, Bhai J, Billis K, Boddu S, Marugán JC, Cummins C, Davidson C, Dodiya K, Fatima R, Gall A, Giron C G, Gil L, Grego T, Haggerty L, Haskell E, Hourlier T, Izuogu O G, Janacek S H, Juettemann T, Kay M, Lavidas I, Le T, Lemos D, Martinez J G, Maurel T, McDowall M, McMahon A, Mohanan S, Moore B, Nuhn M, Oheh D N, Parker A, Parton A, Patricio M, Sakthivel M P, Abdul Salam A I, Schmitt B M, Schuilenburg H, Sheppard D, Sycheva M, Szuba M, Taylor K, Thormann A, Threadgold G, Vullo A, Walts B, Winterbottom A, Zadissa A, Chakiachvili M, Flint B, Frankish A, Hunt S E, Ilsley G, Kostadima M, Langridge N, Loveland J E, Martin F J, Morales J, Mudge J M, Muffato M, Perry E, Ruffier M, Trevanion S J, Cunningham F, Howe K L, Zerbino D R, Flicek P. 2019. Ensembl 2020. Nucleic Acids Res 48:D682-D688. doi:10.1093/nar/gkz966
Ye X, Wang Y, Cahill H, Yu M, Badea T C, Smallwood P M, Peachey N S, Nathans J. 2009. Norrin, Frizzled-4, and Lrp5 Signaling in Endothelial Cells Controls a Genetic Program for Retinal Vascularization. Cell 139:285-298. doi:10.1016/j.cell.2009.07.047
Zhong S, Ding W, Sun L, Lu Y, Dong H, Fan X, Liu Z, Chen R, Zhang S, Ma Q, Tang F, Wu Q, Wang X. 2020. Decoding the development of the human hippocampus. Nature 577:531-536. doi:10.1038/s41586-019-1917-5
Zhou Y, Wang Y, Tischfield M, Williams J, Smallwood P M, Rattner A, Taketo M M, Nathans J. 2014. Canonical WNT signaling components in vascular development and barrier formation. J Clin Invest 124:3825-3846. doi:10.1172/JCI76431
Zhou Y, Williams J, Smallwood P M, Nathans J. 2015. Sox7, Sox17, and Sox18 cooperatively regulate vascular development in the mouse retina. PLoS One 10:1-22. doi: 10.1371/journal.pone.0143650

1. A method of producing a population of extended endothelial culture method (EECM)-derived brain microvascular endothelial cells (BMECs) (EECM-BMECs) from a population of endothelial progenitor cells, the method comprising:
(a) culturing a cell population of CD34+CD31+ endothelial progenitor cells in serum-free endothelial medium on collagen coated surface until confluent;
(b) selectively passaging the endothelial cells of (a) in serum-free endothelial medium on collagen surface for at least two passages;
(c) culturing the selectively passaged cells of (b) in serum-free endothelial medium until confluent;
wherein the confluent monolayer is a population of CD31+ EECM-BMECs that express vascular endothelial (VE)-cadherin, ICAM-2, PECAM-1, and three or more adhesion molecules selected from the group consisting of ICAM-1, VCAM-1, E-selectin, P-selectin, and CD99.

2. The method of claim 1, wherein the CD31+ EECM-BMECs express one or more blood-brain barrier markers selected from the group consisting of occludin, claudin-5, zonula occludens-1 (ZO-1), Von Willebrand factor (vWF), and caveolin-1.

3. (canceled)

4. The method of claim 1, wherein culturing in step (a) comprises:
(i) culturing the cell population in serum-free endothelial medium on collagen coated surface in the presence of a ROCK inhibitor, optionally for 24 hours; and
(ii) removing the ROCK inhibitor and culturing the cells of (i) in serum-free endothelial medium on collagen coated surface until confluent.

5. (canceled)

6. The method of claim 1, wherein the cells are cultured on a surface comprising a permeable support within a tissue culture system.

7. The method of claim 1, wherein in steps (b)-(c):
the serum-free endothelial medium comprises conditioned medium from smooth muscle-like cells (SMLCs); or
the cells are co-cultured with smooth muscle-like cells (SMLCs),
and wherein the resulting EECM-BMECs express VCAM-1 on their surface.

8.-9. (canceled)

10. The method of claim 1, wherein the CD31+ EECM-BMECs express the adhesion molecules ICAM-1, VCAM-1, E-selectin, P-selectin, and CD99 and the blood-brain barrier markers occludin, claudin-5, ZO-1, vWF, and caveolin-1.

11. The method of claim 1, wherein the CD31+ EECM-BMECs further express ALCAM or MCAM.

12. The method of claim 1, wherein selectively passaging comprises:
a) detaching and collecting the endothelial cells from the culture plate, and wherein non-endothelial cells are not-detached from the tissue culture plate; or
b) enriching for PECAM-1+ endothelial cells as opposed to PECAM-1-smooth muscle-like cells.

13.-14. (canceled)

15. The method of claim 1, wherein culturing of the CD31+ EECM-BMECs with at least one pro-inflammatory cytokine increases the expression of ICAM-1, P-selectin, VCAM-1 or a combination thereof on the surface of the CD31+ EECM-BMECs cells.

16. The method of claim 1, wherein the endothelial progenitor cells of step (a) are differentiated from pluripotent stem cells.

17. A homogenous population of CD31⁺ EECM-BMECs obtained by the method of claim 1.

18. The CD31+ EECM-BMECs of claim 17, wherein the CD31+ EECM-BMECs are characterized by:
(a) a lower permeability as compared to naïve endothelial cells;
(b) increased expression of claudin-5 as compared to naïve endothelial cells;
(c) improved localization of occludin and claudin-5 as compared to naïve endothelial cells; or
(d) any combination of (a)-(c).

19. An in vitro blood-brain barrier model for studying immune cell migration and regulation comprising a confluent monolayer of the CD31+ EECM-BMECs of claim 17 cultured on a surface, optionally a collagen coated permeable membrane, within a system.

20. (canceled)

21. The in vitro blood-brain barrier model of claim 19, wherein the model has a permeability of sodium fluorescein of less than 1×10−3 cm/min.

22. Use of the in vitro blood-brain barrier model of claim 19 for identifying compounds that may alter the immunological properties or barrier properties of the blood-brain barrier.

23. Use of the in vitro blood-brain barrier model of claim 19 for studying multi-step immune cell extravasation across the blood-brain barrier.

24. A method of identifying therapeutic targets for the treatment of neuroinflammatory or neurodegenerative diseases or disorders, the method comprising:
(a) contacting the in vitro blood-brain barrier model of claim 19 with a therapeutic target; and
(b) determining the disruption and/or restoration of the blood-brain barrier model.

25. An isogenic blood-brain barrier model for a subject having a neuroinflammatory or neurodegenerative disease, the blood-brain barrier model comprising a confluent monolayer of the CD31+ EECM-BMECs of claim 17 cultured on permeable surface within a tissue culture system, wherein the CD31+ EECM-BMECs are derived from endothelial progenitor cells differentiated from pluripotent stem cells derived from the subject.

26. A method for producing a cell population comprising smooth muscle-like cells from a cell population comprising CD34+CD31+ endothelial progenitor cells, the method comprising the steps of:
(a) culturing a cell population of CD34+CD31+ endothelial progenitor cells in serum-free endothelial medium on collagen coated surface until confluent;
(b) selectively passaging the non-endothelial cells of (a);
(c) culturing the non-endothelial cells of (b) for about 6 days to about 10 days to produce PECAM-1-/α-smooth muscle actin+ smooth muscle-like cells.

27.-29. (canceled)

30. The method of claim 1, wherein step (a) comprises culturing the CD34+CD31+ endothelial progenitor cells in serum-free endothelial medium comprising a activator of Wnt/β-catenin signaling.

31. The method of claim 30, wherein the activator of Wnt/β-catenin signaling is a Gsk3 inhibitor, CHIR99021, or one or more Wnt ligands.

32.-35. (canceled)

36. A method of producing a population of derived extended endothelial cultured method brain microvascular endothelial cells (EECM-BMECs) from human pluripotent stem cells, the method comprising:
(a) contacting cultured human pluripotent stem cells with activator of Wnt/β-catenin signaling for a period of about 2 days;
b) culturing the cells of (a) in the absence of the activator for two to three days;
(c) separating the CD34+CD31+ endothelial progenitor cells from step (b) from the CD34−CD31− non-EPCs; and
(d) culturing the separated CD34+CD31+ endothelial progenitor cells on coated plates in medium comprising the activator of Wnt/β-catenin signaling for about 3 days to about 10 days to provide a confluent monolayer;
(e) selectively passaging the cells of step (d) in serum-free endothelial medium on coated plates in medium comprising the activator on coated plates for at least one additional passage to obtain a confluent population of CD31+GLUT1+EECM-BMECs having a canonical barrier phenotype.

37.-48. (canceled)
